(19) United States
(12) Patent Application Publication
(10) Pub. No.: US 2024/0116933 A1
(43) Pub. Date: Apr. 11, 2024

(54) INDAZOLE AND PYRAZOLOPYRIDINE COMPOUNDS AS INHIBITORS OF NLRP3 INFLAMMASOME

(71) Applicant: BioAge Labs, Inc., Richmond, CA (US)

(72) Inventor: George HARTMAN, Lansdale, PA (US)

(21) Appl. No.: 18/470,105

(22) Filed: Sep. 19, 2023

Related U.S. Application Data
(60) Provisional application No. 63/376,359, filed on Sep. 20, 2022.

(51) Int. Cl. C07D 471/04; C07D 231/56

(52) U.S. Cl. CPC C07D 471/04; C07D 231/56

(57) ABSTRACT

The present disclosure relates to compounds that act as inhibitors of NLRP3 inflammasomes; pharmaceutical compositions comprising the compounds; and methods of treating disorders associated with inflammation and inflammaging, including neurosensory diseases and other diseases associated with aging.

### RELATED APPLICATIONS

This application claims priority to U.S. Provisional Application No. 63/376,359 filed Sep. 20, 2022, the content of which is hereby incorporated by reference in its entirety.

### BACKGROUND

Aging frailty poses a very concerning problem for the overall health and well-being of individuals and is characterized as a syndrome of multisystem physiological dysregulation. Aging frailty is a geriatric syndrome characterized by weakness, low physical activity, slowed motor performance, exhaustion, and unintentional weight loss (Yao, X. et al., Clinics in Geriatric Medicine 27(1): 79-87 (2011)). Furthermore, there are many studies showing a direct correlation between aging frailty and inflammation (Hubbard, R. E., et al., Biogerontology 11(5):635-641 (2010)). Immunosenescence is characterized by a low grade, chronic systemic inflammatory state known as inflammaging (Franceshi, C. et al., Annals of the New York Academy of Sciences 908:244-254 (2000)). This heightened inflammatory state or chronic inflammation found in aging and aging frailty leads to immune dysregulation and a complex remodeling of both innate and adaptive immunity.

Inhibiting the NLRP3 inflammasome, an oligomeric protein complex that includes ASC and caspase-1, mediates inflammation in an extensive number of preclinical models (Schwaid, A. G., J. Med. Chem. 2021, 64(1), 101-122). At the same time, the NLRP3 inflammasome is part of a larger pro-inflammatory pathway, whose modulation is also being explored. NLRP3 is an inflammasome sensor protein that has been well studied in a number of disease contexts. Many different indications are associated with the NLRP3 inflammasome including diseases related to aging, cryopyrin-associated periodic syndrome (CAPS), nonalcoholic steatohepatitis (NASH), gout, coronary artery disease, Crohn's disease, osteoarthritis, rheumatoid arthritis, Alzheimer's disease, Parkinson's disease, intestinal disorders, acute respiratory distress syndrome (ARDS), amyotrophic lateral sclerosis (ALS), cancer, and dermatological diseases.

Inflammation, as well as activation of the NLRP3 inflammasome, have also been shown to result in hearing loss (Nakanishi, H., et al., Frontiers in Neurology, 2020, 11, 1-7; Nakanishi, H., et al., PNAS, 2017, E7766-E7775). The inflammation-related hearing loss can be age-dependent (Fischer, N., et al., Gerontology, 2019, 1-7), noise-induced (Le Prell, C. G., et al. Current Opinion in Physiology, 2020, 18, 32-36), and the result of a viral infection such as Zika virus and coronavirus (Yee, K. T., et al., Hearing Research, 2020, 395, 1-15).

The NLRP3 inflammasome is therefore a promising drug target. The breadth of the indications it is implicated in speaks to the need for therapeutics that target the NLRP3 inflammasome.

### SUMMARY

Provided here are compounds that inhibit the NLRP3 inflammasome. As such, these compounds are useful in the treatment of a variety of indications, including inflammaging and neurosensory diseases. Also provided herein is a pharmaceutical composition comprising a compound described herein, or a pharmaceutically acceptable salt thereof, and a pharmaceutically acceptable carrier. In another aspect, provided herein is a method of inhibiting NLRP3 inflammasome in a subject in need thereof comprising administering to the subject a therapeutically effective amount of a compound described herein.

### DETAILED DESCRIPTION

Provided here are compounds that inhibit the NLRP3 inflammasome. As such, these compounds, as well as pharmaceutical compositions that comprise these compounds, are useful in the treatment of a variety of indications, including inflammaging and other age-related diseases, such as Alzheimer's disease.

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises alleviating or preventing the symptoms of inflammaging and age-related disorders.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, “inflammaging” is defined as chronic sterile inflammation that is associated with numerous age-related diseases.

As used herein, “age-related disorder” refers to disorders that are associated with the aging process. Stated alternatively, age-related disorders are diseases associated with the elderly. Non-limiting examples of age-related diseases include atherosclerosis and cardiovascular disease, cancer, arthritis, cataracts, osteoporosis, type 2 diabetes, hypertension, and Alzheimer's disease. The incidence of all of these diseases increases exponentially with age.

As used herein, “neurosensory diseases” refers to disorders that affect the brain as well as the nerves found throughout the human body and the spinal cord. In some embodiments, the neurosensory diseases are age-related. In certain embodiments, the neurosensory diseases are a result of a traumatic brain injury.

### Compounds

Provided herein are compounds that are inhibitors of the NLRP3 inflammasome and are thus useful in the treatment of inflammatory disorders, including cancer and other proliferation diseases.

In an aspect, provided herein is a compound selected from the group consisting of a compound in Table 1.

TABLE 1

| | Compound |
|---|---|
| Structure | No. |
| 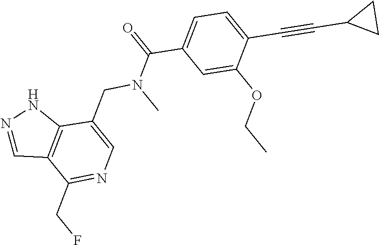 [SMILES: CCOc1cc(C(=O)N(C)Cc2cnc(CF)c3cn[nH]c23)ccc1C#CC1CC1] | 001 |
| 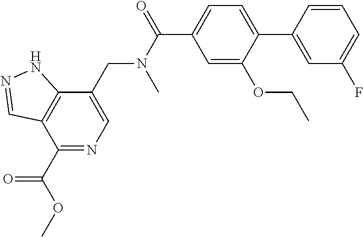 [SMILES: CCOc1cc(C(=O)N(C)Cc2cnc(C(=O)OC)c3cn[nH]c23)ccc1-c1cccc(F)c1] | 002 |
| 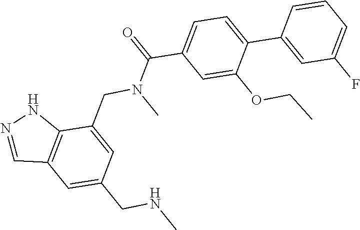 [SMILES: CCOc1cc(C(=O)N(C)Cc2cc(CNC)cc3cn[nH]c23)ccc1-c1cccc(F)c1] | 003 |
| 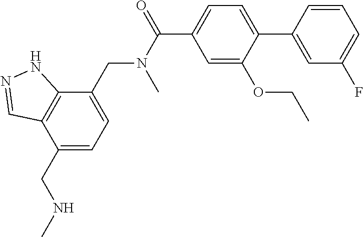 [SMILES: CCOc1cc(C(=O)N(C)Cc2ccc(CNC)c3cn[nH]c23)ccc1-c1cccc(F)c1] | 004 |
| 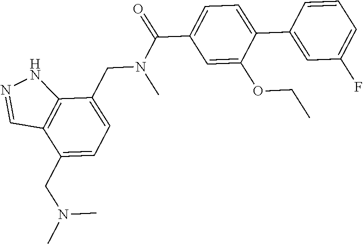 [SMILES: CCOc1cc(C(=O)N(C)Cc2ccc(CN(C)C)c3cn[nH]c23)ccc1-c1cccc(F)c1] | 005 |
| 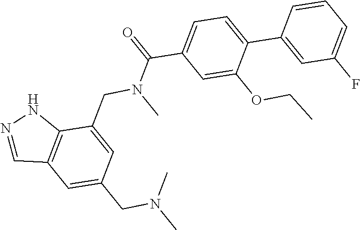 [SMILES: CCOc1cc(C(=O)N(C)Cc2cc(CN(C)C)cc3cn[nH]c23)ccc1-c1cccc(F)c1] | 006 |
| 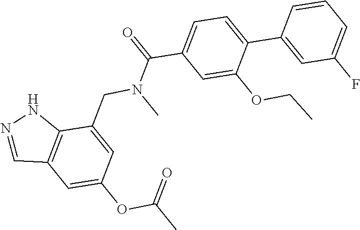 [SMILES: CCOc1cc(C(=O)N(C)Cc2cc(OC(C)=O)cc3cn[nH]c23)ccc1-c1cccc(F)c1] | 007 |
| 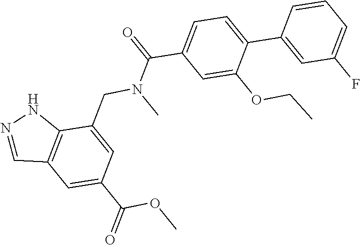 [SMILES: CCOc1cc(C(=O)N(C)Cc2cc(C(=O)OC)cc3cn[nH]c23)ccc1-c1cccc(F)c1] | 008 |
| 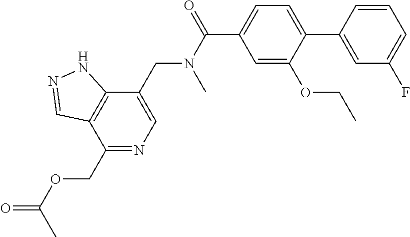 [SMILES: CCOc1cc(C(=O)N(C)Cc2cnc(COC(C)=O)c3cn[nH]c23)ccc1-c1cccc(F)c1] | 009 |
| 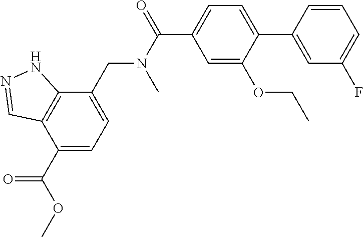 [SMILES: CCOc1cc(C(=O)N(C)Cc2ccc(C(=O)OC)c3cn[nH]c23)ccc1-c1cccc(F)c1] | 010 |
| 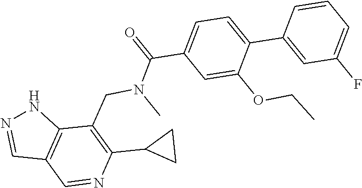 [SMILES: CCOc1cc(C(=O)N(C)Cc2c(C3CC3)ncc3cn[nH]c23)ccc1-c1cccc(F)c1] | 011 |

or a pharmaceutically acceptable salt thereof.

In another aspect, provided herein is a compound selected from the group consisting of a compound in Table 2.

TABLE 2

| | Compound |
|---|---|
| Structure | No. |
| 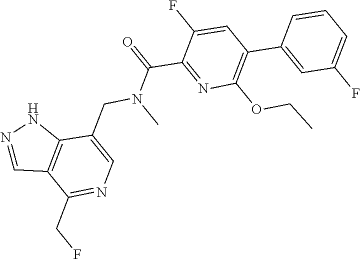 [SMILES: CCOc1nc(C(=O)N(C)Cc2cnc(CF)c3cn[nH]c23)c(F)cc1-c1cccc(F)c1] | 012 |
| 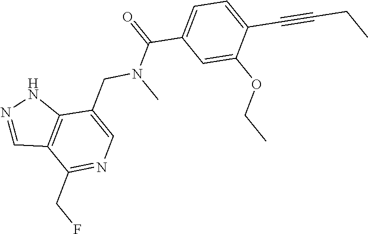 [SMILES: CCC#Cc1ccc(C(=O)N(C)Cc2cnc(CF)c3cn[nH]c23)cc1OCC] | 013 |
| 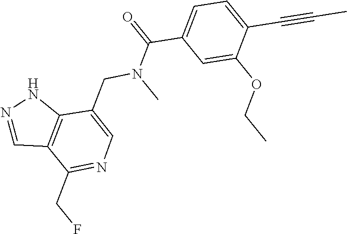 [SMILES: CC#Cc1ccc(C(=O)N(C)Cc2cnc(CF)c3cn[nH]c23)cc1OCC] | 014 |
| 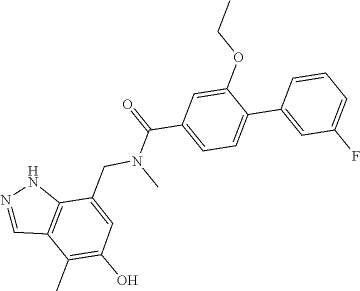 [SMILES: CCOc1cc(C(=O)N(C)Cc2cc(O)c(C)c3cn[nH]c23)ccc1-c1cccc(F)c1] | 015 |
| 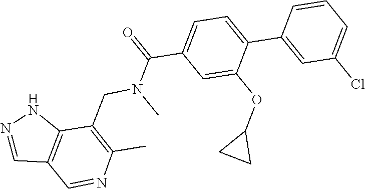 [SMILES: Cc1ncc2cn[nH]c2c1CN(C)C(=O)c1ccc(-c2cccc(Cl)c2)c(OC2CC2)c1] | 016 |
| 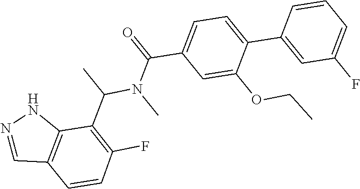 [SMILES: CCOc1cc(C(=O)N(C)C(C)c2c(F)ccc3cn[nH]c23)ccc1-c1cccc(F)c1] | 017 |
| 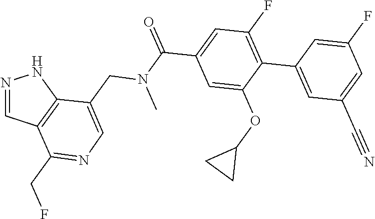 [SMILES: CN(Cc1cnc(CF)c2cn[nH]c12)C(=O)c1cc(F)c(-c2cc(F)cc(C#N)c2)c(OC2CC2)c1] | 018 |
| 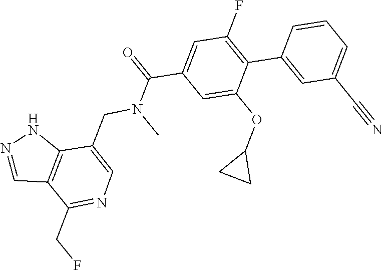 [SMILES: CN(Cc1cnc(CF)c2cn[nH]c12)C(=O)c1cc(F)c(-c2cccc(C#N)c2)c(OC2CC2)c1] | 019 |
| 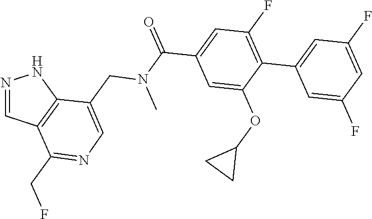 [SMILES: CN(Cc1cnc(CF)c2cn[nH]c12)C(=O)c1cc(F)c(-c2cc(F)cc(F)c2)c(OC2CC2)c1] | 020 |
| 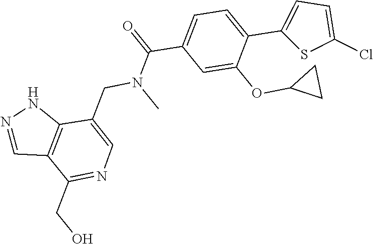 [SMILES: CN(Cc1cnc(CO)c2cn[nH]c12)C(=O)c1ccc(-c2ccc(Cl)s2)c(OC2CC2)c1] | 021 |
| 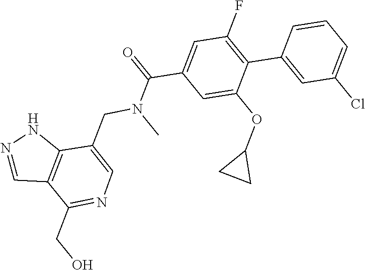 [SMILES: CN(Cc1cnc(CO)c2cn[nH]c12)C(=O)c1cc(F)c(-c2cccc(Cl)c2)c(OC2CC2)c1] | 022 |
| 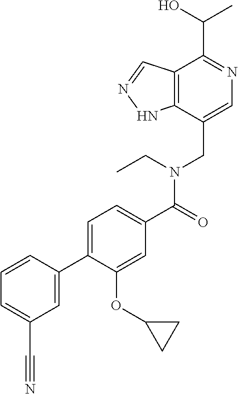 [SMILES: CCN(Cc1cnc(C(C)O)c2cn[nH]c12)C(=O)c1ccc(-c2cccc(C#N)c2)c(OC2CC2)c1] | 023 |
| 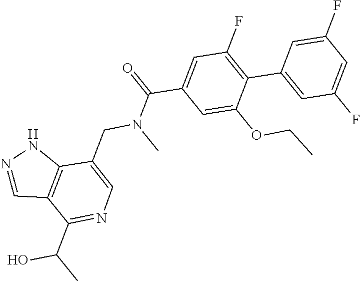 [SMILES: CCOc1cc(C(=O)N(C)Cc2cnc(C(C)O)c3cn[nH]c23)cc(F)c1-c1cc(F)cc(F)c1] | 024 |
| 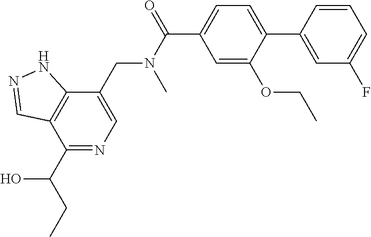 [SMILES: CCOc1cc(C(=O)N(C)Cc2cnc(C(O)CC)c3cn[nH]c23)ccc1-c1cccc(F)c1] | 025 |
| 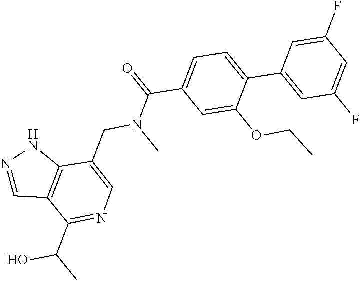 [SMILES: CCOc1cc(C(=O)N(C)Cc2cnc(C(C)O)c3cn[nH]c23)ccc1-c1cc(F)cc(F)c1] | 026 |
| 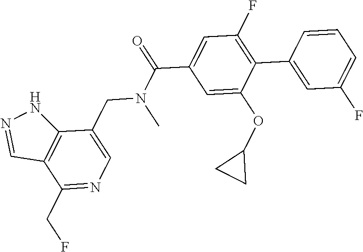 [SMILES: CN(Cc1cnc(CF)c2cn[nH]c12)C(=O)c1cc(F)c(-c2cccc(F)c2)c(OC2CC2)c1] | 027 |
| 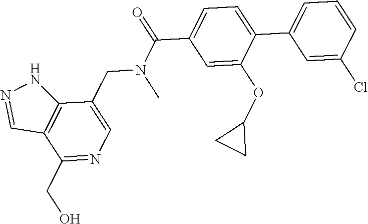 [SMILES: CN(Cc1cnc(CO)c2cn[nH]c12)C(=O)c1ccc(-c2cccc(Cl)c2)c(OC2CC2)c1] | 028 |
| 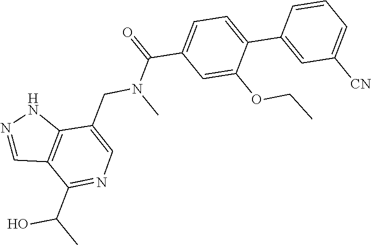 [SMILES: CCOc1cc(C(=O)N(C)Cc2cnc(C(C)O)c3cn[nH]c23)ccc1-c1cccc(C#N)c1] | 029 |
| 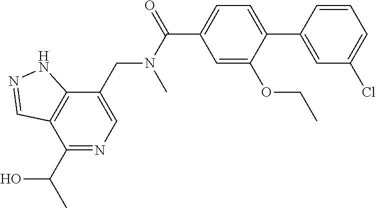 [SMILES: CCOc1cc(C(=O)N(C)Cc2cnc(C(C)O)c3cn[nH]c23)ccc1-c1cccc(Cl)c1] | 030 |
| 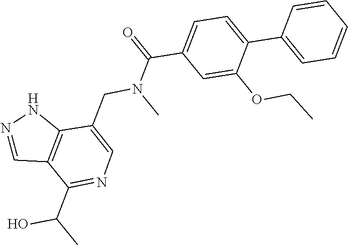 [SMILES: CCOc1cc(C(=O)N(C)Cc2cnc(C(C)O)c3cn[nH]c23)ccc1-c1ccccc1] | 031 |
| 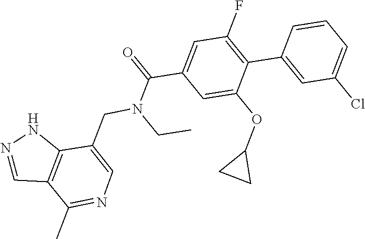 [SMILES: CCN(Cc1cnc(C)c2cn[nH]c12)C(=O)c1cc(F)c(-c2cccc(Cl)c2)c(OC2CC2)c1] | 032 |
| 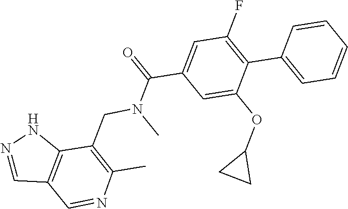 [SMILES: Cc1ncc2cn[nH]c2c1CN(C)C(=O)c1cc(F)c(-c2ccccc2)c(OC2CC2)c1] | 033 |
| 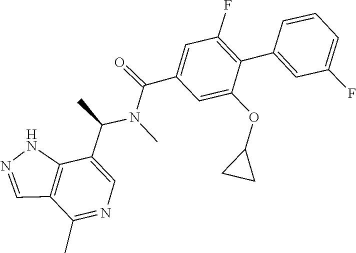 [SMILES: Cc1ncc([C@@H](C)N(C)C(=O)c2cc(F)c(-c3cccc(F)c3)c(OC3CC3)c2)c2[nH]ncc12] | 034 |
| 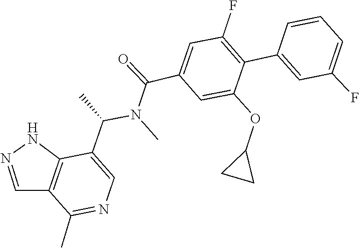 [SMILES: Cc1ncc([C@H](C)N(C)C(=O)c2cc(F)c(-c3cccc(F)c3)c(OC3CC3)c2)c2[nH]ncc12] | 035 |
| 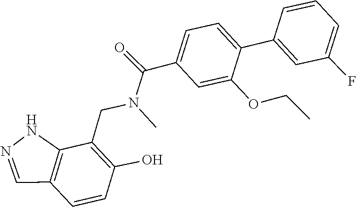 [SMILES: CCOc1cc(C(=O)N(C)Cc2c(O)ccc3cn[nH]c23)ccc1-c1cccc(F)c1] | 036 |
| 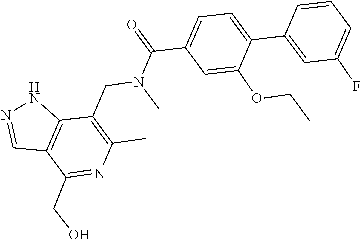 [SMILES: CCOc1cc(C(=O)N(C)Cc2c(C)nc(CO)c3cn[nH]c23)ccc1-c1cccc(F)c1] | 037 |
| 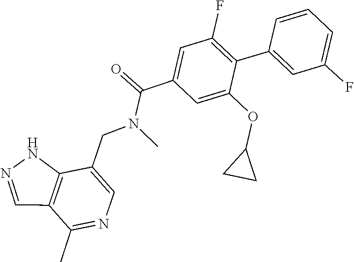 [SMILES: Cc1ncc(CN(C)C(=O)c2cc(F)c(-c3cccc(F)c3)c(OC3CC3)c2)c2[nH]ncc12] | 038 |
| 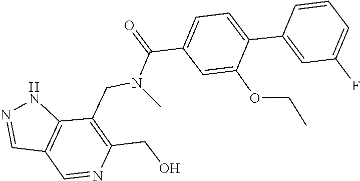 [SMILES: CCOc1cc(C(=O)N(C)Cc2c(CO)ncc3cn[nH]c23)ccc1-c1cccc(F)c1] | 039 |
| 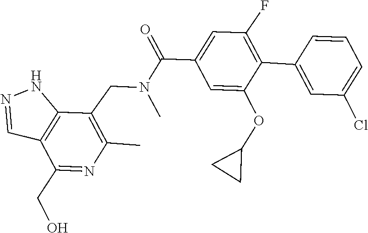 [SMILES: Cc1nc(CO)c2cn[nH]c2c1CN(C)C(=O)c1cc(F)c(-c2cccc(Cl)c2)c(OC2CC2)c1] | 040 |
| 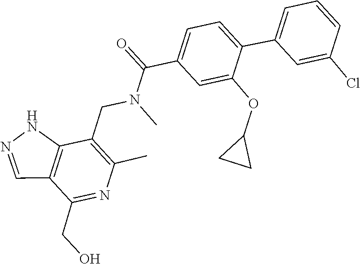 [SMILES: Cc1nc(CO)c2cn[nH]c2c1CN(C)C(=O)c1ccc(-c2cccc(Cl)c2)c(OC2CC2)c1] | 041 |
| 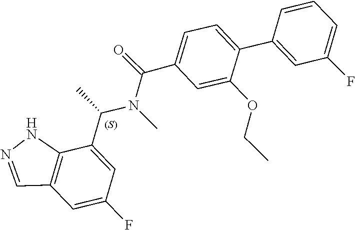 | 042 |
| 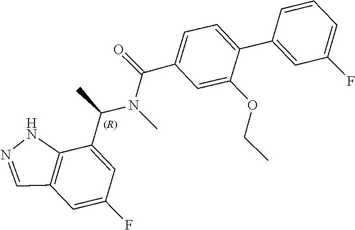 | 043 |
| 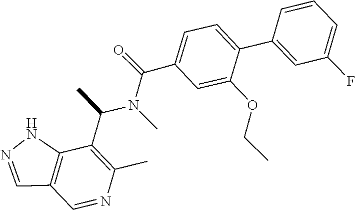 [SMILES: CCOc1cc(C(=O)N(C)[C@H](C)c2c(C)ncc3cn[nH]c23)ccc1-c1cccc(F)c1] | 044 |
| 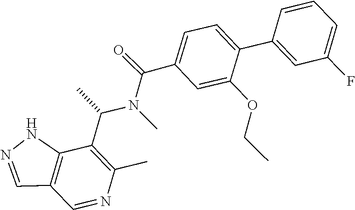 [SMILES: CCOc1cc(C(=O)N(C)[C@@H](C)c2c(C)ncc3cn[nH]c23)ccc1-c1cccc(F)c1] | 045 |
| 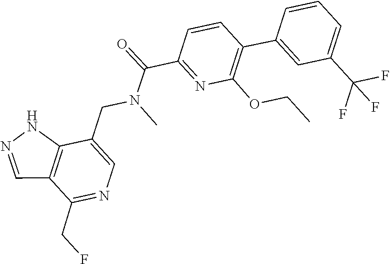 [SMILES: CCOc1nc(C(=O)N(C)Cc2cnc(CF)c3cn[nH]c23)ccc1-c1cccc(C(F)(F)F)c1] | 046 |
| 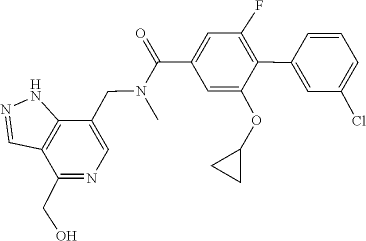 [SMILES: CN(Cc1cnc(CO)c2cn[nH]c12)C(=O)c1cc(F)c(-c2cccc(Cl)c2)c(OC2CC2)c1] | 047 |
| 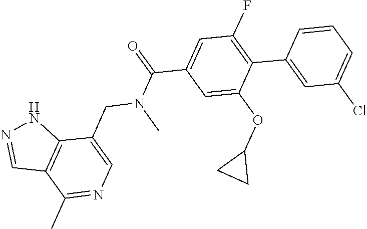 [SMILES: Cc1ncc(CN(C)C(=O)c2cc(F)c(-c3cccc(Cl)c3)c(OC3CC3)c2)c2[nH]ncc12] | 048 |
| 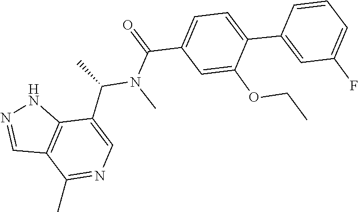 [SMILES: CCOc1cc(C(=O)N(C)[C@@H](C)c2cnc(C)c3cn[nH]c23)ccc1-c1cccc(F)c1] | 049 |
| 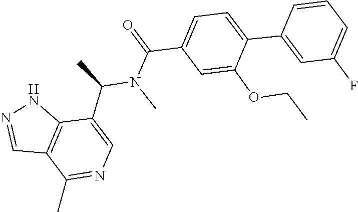 [SMILES: CCOc1cc(C(=O)N(C)[C@H](C)c2cnc(C)c3cn[nH]c23)ccc1-c1cccc(F)c1] | 050 |
| 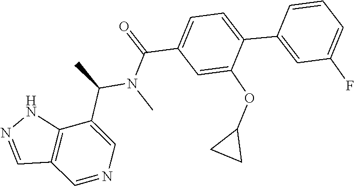 [SMILES: C[C@H](c1cncc2cn[nH]c12)N(C)C(=O)c1ccc(-c2cccc(F)c2)c(OC2CC2)c1] | 051 |
| 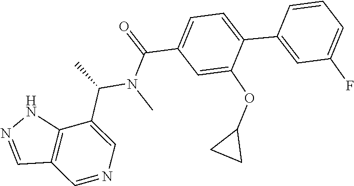 [SMILES: C[C@@H](c1cncc2cn[nH]c12)N(C)C(=O)c1ccc(-c2cccc(F)c2)c(OC2CC2)c1] | 52 |
| 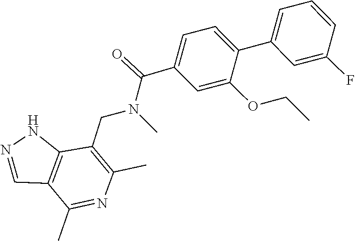 [SMILES: CCOc1cc(C(=O)N(C)Cc2c(C)nc(C)c3cn[nH]c23)ccc1-c1cccc(F)c1] | 053 |
| 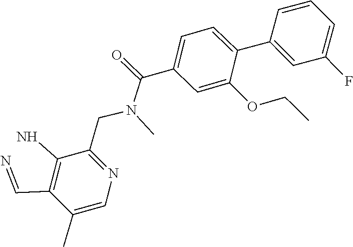 | 054 |
| 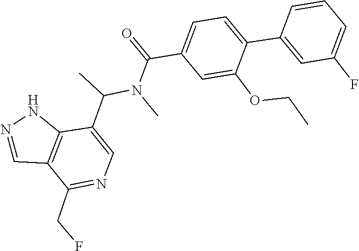 [SMILES: CCOc1cc(C(=O)N(C)C(C)c2cnc(CF)c3cn[nH]c23)ccc1-c1cccc(F)c1] | 055 |
| 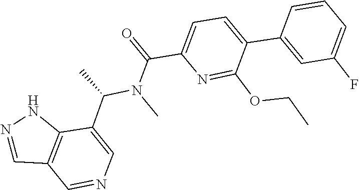 [SMILES: CCOc1nc(C(=O)N(C)[C@@H](C)c2cncc3cn[nH]c23)ccc1-c1cccc(F)c1] | 056 |
| 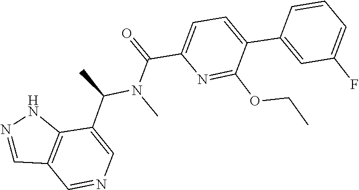 [SMILES: CCOc1nc(C(=O)N(C)[C@H](C)c2cncc3cn[nH]c23)ccc1-c1cccc(F)c1] | 057 |
| 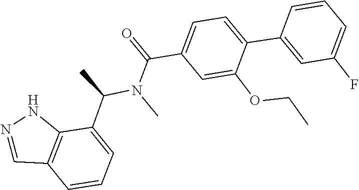 [SMILES: CCOc1cc(C(=O)N(C)[C@H](C)c2cccc3cn[nH]c23)ccc1-c1cccc(F)c1] | 058 |
| 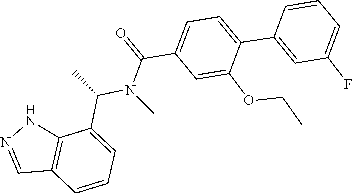 [SMILES: CCOc1cc(C(=O)N(C)[C@@H](C)c2cccc3cn[nH]c23)ccc1-c1cccc(F)c1] | 059 |
| 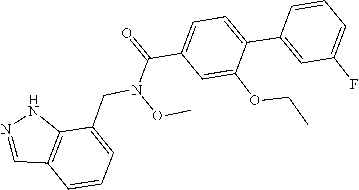 [SMILES: CCOc1cc(C(=O)N(Cc2cccc3cn[nH]c23)OC)ccc1-c1cccc(F)c1] | 060 |
| 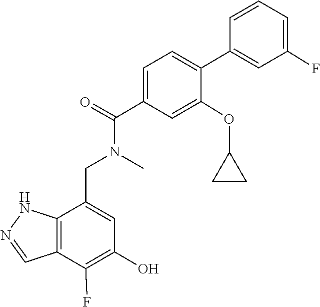 [SMILES: CN(Cc1cc(O)c(F)c2cn[nH]c12)C(=O)c1ccc(-c2cccc(F)c2)c(OC2CC2)c1] | 061 |
| 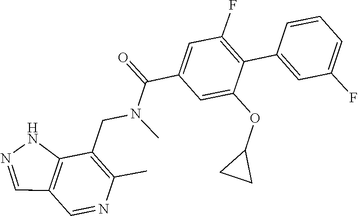 [SMILES: Cc1ncc2cn[nH]c2c1CN(C)C(=O)c1cc(F)c(-c2cccc(F)c2)c(OC2CC2)c1] | 062 |
| 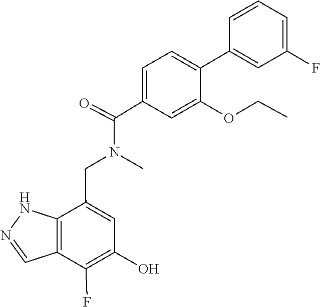 [SMILES: CCOc1cc(C(=O)N(C)Cc2cc(O)c(F)c3cn[nH]c23)ccc1-c1cccc(F)c1] | 063 |
| 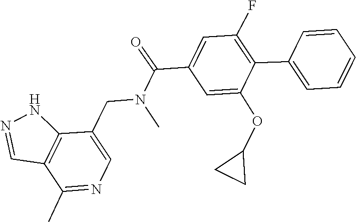 [SMILES: Cc1ncc(CN(C)C(=O)c2cc(F)c(-c3ccccc3)c(OC3CC3)c2)c2[nH]ncc12] | 064 |
| 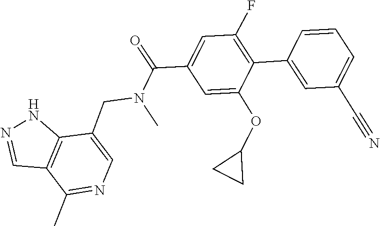 [SMILES: Cc1ncc(CN(C)C(=O)c2cc(F)c(-c3cccc(C#N)c3)c(OC3CC3)c2)c2[nH]ncc12] | 065 |
| 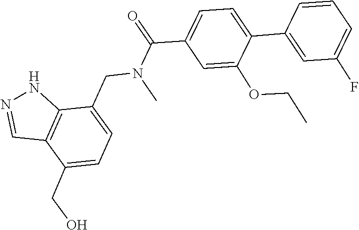 [SMILES: CCOc1cc(C(=O)N(C)Cc2ccc(CO)c3cn[nH]c23)ccc1-c1cccc(F)c1] | 066 |
| 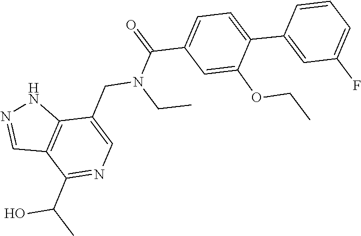 [SMILES: CCOc1cc(C(=O)N(CC)Cc2cnc(C(C)O)c3cn[nH]c23)ccc1-c1cccc(F)c1] | 067 |
| 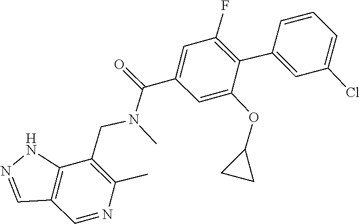 [SMILES: Cc1ncc2cn[nH]c2c1CN(C)C(=O)c1cc(F)c(-c2cccc(Cl)c2)c(OC2CC2)c1] | 068 |
| 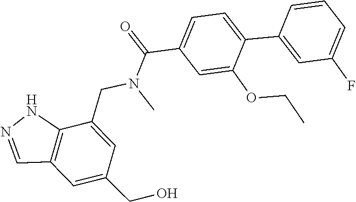 [SMILES: CCOc1cc(C(=O)N(C)Cc2cc(CO)cc3cn[nH]c23)ccc1-c1cccc(F)c1] | 069 |
| 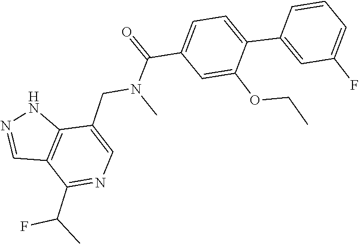 [SMILES: CCOc1cc(C(=O)N(C)Cc2cnc(C(C)F)c3cn[nH]c23)ccc1-c1cccc(F)c1] | 070 |
| 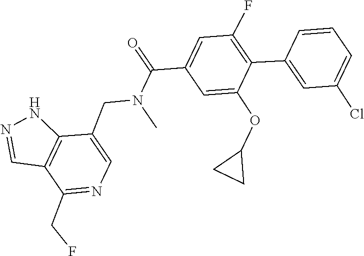 [SMILES: CN(Cc1cnc(CF)c2cn[nH]c12)C(=O)c1cc(F)c(-c2cccc(Cl)c2)c(OC2CC2)c1] | 071 |
| 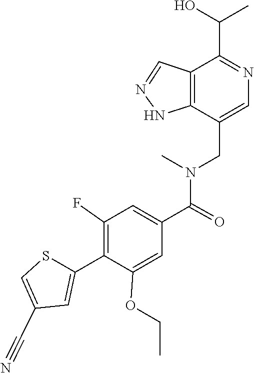 | 072 |
| 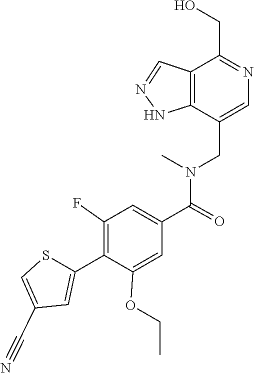 | 073 |
| 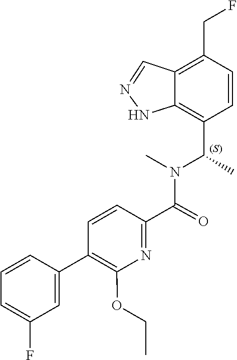 | 074 |
| 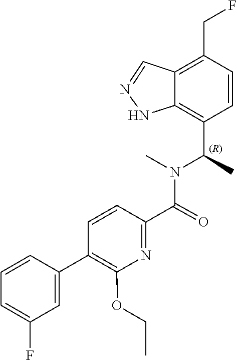 | 075 |
| 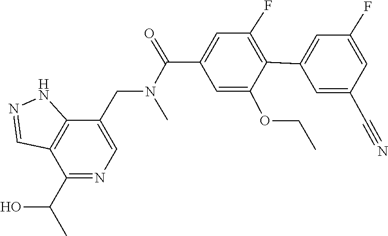 [SMILES: CCOc1cc(C(=O)N(C)Cc2cnc(C(C)O)c3cn[nH]c23)cc(F)c1-c1cc(F)cc(C#N)c1] | 076 |
| 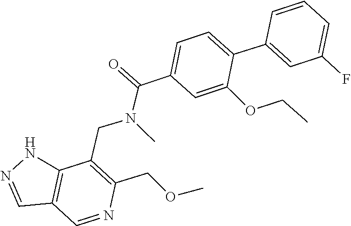 [SMILES: CCOc1cc(C(=O)N(C)Cc2c(COC)ncc3cn[nH]c23)ccc1-c1cccc(F)c1] | 077 |
| 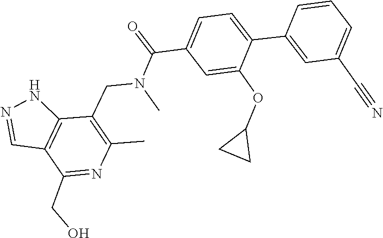 [SMILES: Cc1nc(CO)c2cn[nH]c2c1CN(C)C(=O)c1ccc(-c2cccc(C#N)c2)c(OC2CC2)c1] | 078 |
| 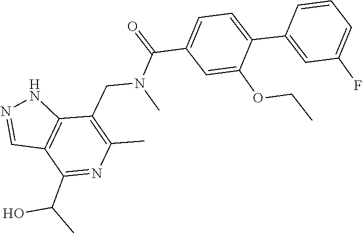 [SMILES: CCOc1cc(C(=O)N(C)Cc2c(C)nc(C(C)O)c3cn[nH]c23)ccc1-c1cccc(F)c1] | 079 |
| 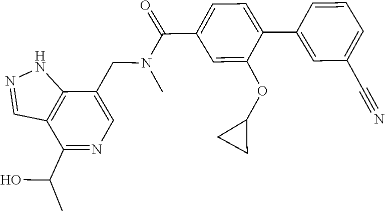 [SMILES: CC(O)c1ncc(CN(C)C(=O)c2ccc(-c3cccc(C#N)c3)c(OC3CC3)c2)c2[nH]ncc12] | 080 |
| 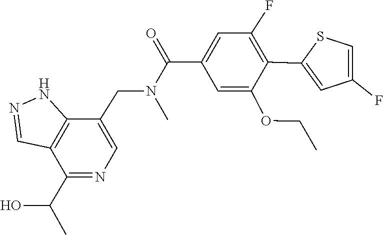 [SMILES: CCOc1cc(C(=O)N(C)Cc2cnc(C(C)O)c3cn[nH]c23)cc(F)c1-c1cc(F)cs1] | 081 |
| 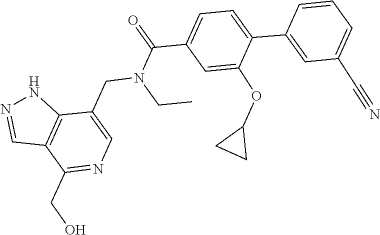 [SMILES: CCN(Cc1cnc(CO)c2cn[nH]c12)C(=O)c1ccc(-c2cccc(C#N)c2)c(OC2CC2)c1] | 082 |
| 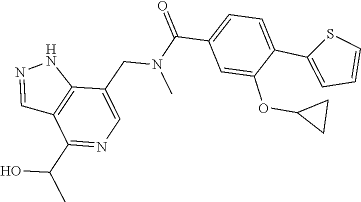 [SMILES: CC(O)c1ncc(CN(C)C(=O)c2ccc(-c3cccs3)c(OC3CC3)c2)c2[nH]ncc12] | 083 |
| 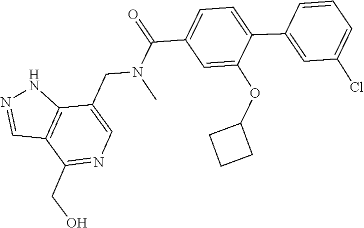 [SMILES: CN(Cc1cnc(CO)c2cn[nH]c12)C(=O)c1ccc(-c2cccc(Cl)c2)c(OC2CCC2)c1] | 084 |
| 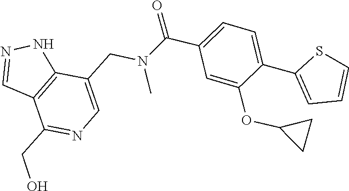 [SMILES: CN(Cc1cnc(CO)c2cn[nH]c12)C(=O)c1ccc(-c2cccs2)c(OC2CC2)c1] | 085 |
| 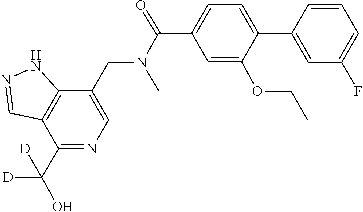 [SMILES: [2H]C([2H])(O)c1ncc(CN(C)C(=O)c2ccc(-c3cccc(F)c3)c(OCC)c2)c2[nH]ncc12] | 086 |
| 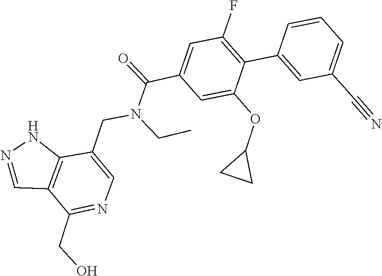 [SMILES: CCN(Cc1cnc(CO)c2cn[nH]c12)C(=O)c1cc(F)c(-c2cccc(C#N)c2)c(OC2CC2)c1] | 087 |
| 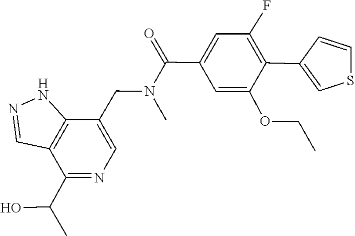 [SMILES: CCOc1cc(C(=O)N(C)Cc2cnc(C(C)O)c3cn[nH]c23)cc(F)c1-c1ccsc1] | 088 |
| 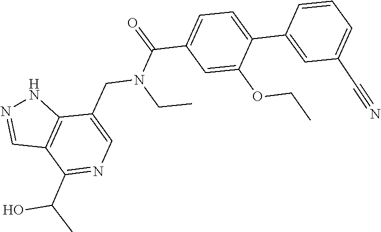 [SMILES: CCOc1cc(C(=O)N(CC)Cc2cnc(C(C)O)c3cn[nH]c23)ccc1-c1cccc(C#N)c1] | 089 |
| 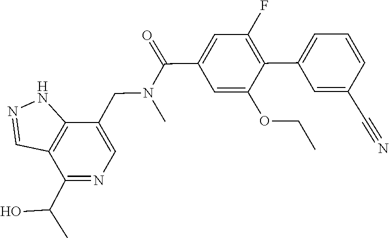 [SMILES: CCOc1cc(C(=O)N(C)Cc2cnc(C(C)O)c3cn[nH]c23)cc(F)c1-c1cccc(C#N)c1] | 090 |
| 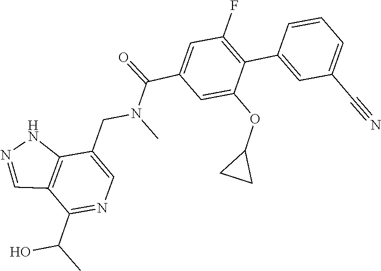 [SMILES: CC(O)c1ncc(CN(C)C(=O)c2cc(F)c(-c3cccc(C#N)c3)c(OC3CC3)c2)c2[nH]ncc12] | 091 |
| 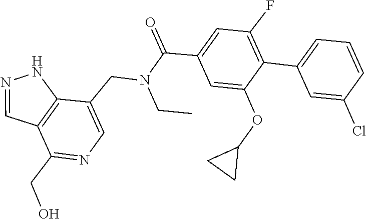 [SMILES: CCN(Cc1cnc(CO)c2cn[nH]c12)C(=O)c1cc(F)c(-c2cccc(Cl)c2)c(OC2CC2)c1] | 092 |
| 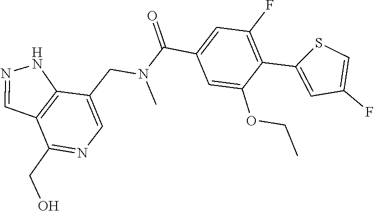 [SMILES: CCOc1cc(C(=O)N(C)Cc2cnc(CO)c3cn[nH]c23)cc(F)c1-c1cc(F)cs1] | 093 |
| 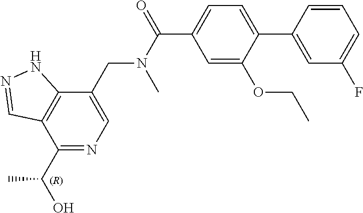 | 094 |
| 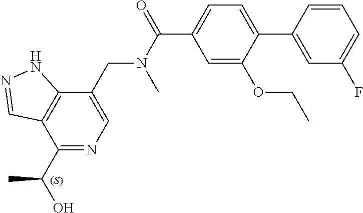 | 095 |
| 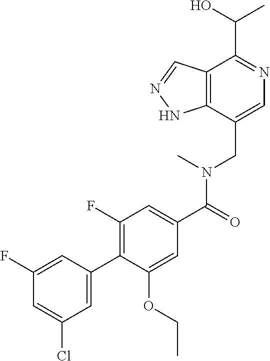 [SMILES: CCOc1cc(C(=O)N(C)Cc2cnc(C(C)O)c3cn[nH]c23)cc(F)c1-c1cc(F)cc(Cl)c1] | 096 |
| 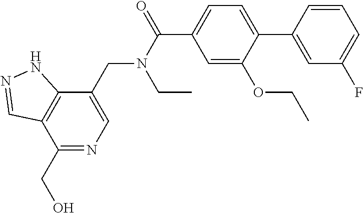 [SMILES: CCOc1cc(C(=O)N(CC)Cc2cnc(CO)c3cn[nH]c23)ccc1-c1cccc(F)c1] | 097 |
| 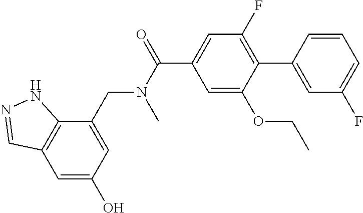 [SMILES: CCOc1cc(C(=O)N(C)Cc2cc(O)cc3cn[nH]c23)cc(F)c1-c1cccc(F)c1] | 098 |
| 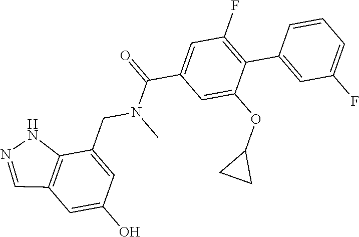 [SMILES: CN(Cc1cc(O)cc2cn[nH]c12)C(=O)c1cc(F)c(-c2cccc(F)c2)c(OC2CC2)c1] | 099 |
| 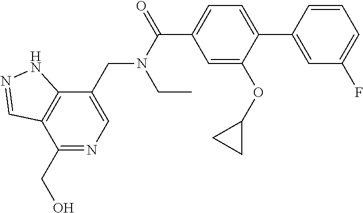 [SMILES: CCN(Cc1cnc(CO)c2cn[nH]c12)C(=O)c1ccc(-c2cccc(F)c2)c(OC2CC2)c1] | 100 |
| 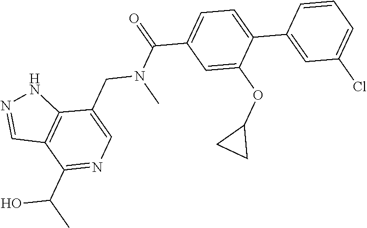 [SMILES: CC(O)c1ncc(CN(C)C(=O)c2ccc(-c3cccc(Cl)c3)c(OC3CC3)c2)c2[nH]ncc12] | 101 |
| 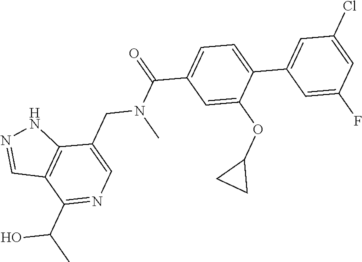 [SMILES: CC(O)c1ncc(CN(C)C(=O)c2ccc(-c3cc(F)cc(Cl)c3)c(OC3CC3)c2)c2[nH]ncc12] | 102 |
| 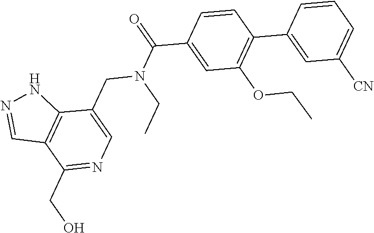 [SMILES: CCOc1cc(C(=O)N(CC)Cc2cnc(CO)c3cn[nH]c23)ccc1-c1cccc(C#N)c1] | 103 |
| 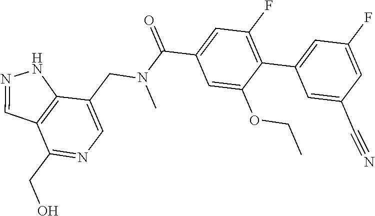 [SMILES: CCOc1cc(C(=O)N(C)Cc2cnc(CO)c3cn[nH]c23)cc(F)c1-c1cc(F)cc(C#N)c1] | 104 |
| 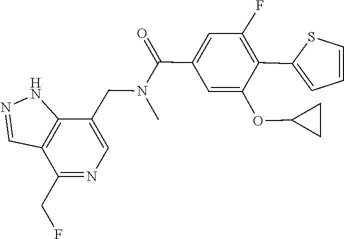 [SMILES: CN(Cc1cnc(CF)c2cn[nH]c12)C(=O)c1cc(F)c(-c2cccs2)c(OC2CC2)c1] | 105 |
| 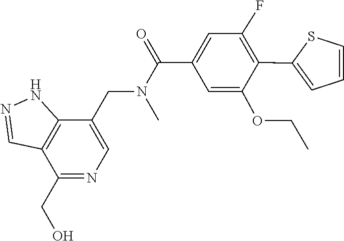 [SMILES: CCOc1cc(C(=O)N(C)Cc2cnc(CO)c3cn[nH]c23)cc(F)c1-c1cccs1] | 106 |
| 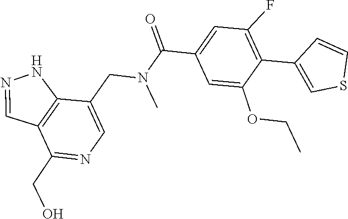 [SMILES: CCOc1cc(C(=O)N(C)Cc2cnc(CO)c3cn[nH]c23)cc(F)c1-c1ccsc1] | 107 |
| 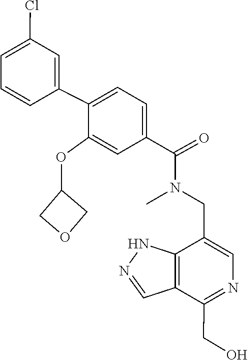 [SMILES: CN(Cc1cnc(CO)c2cn[nH]c12)C(=O)c1ccc(-c2cccc(Cl)c2)c(OC2COC2)c1] | 108 |
| 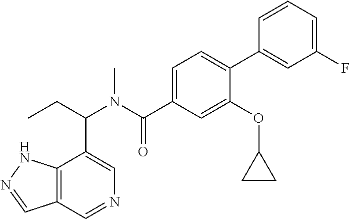 [SMILES: CCC(c1cncc2cn[nH]c12)N(C)C(=O)c1ccc(-c2cccc(F)c2)c(OC2CC2)c1] | 109 |
| 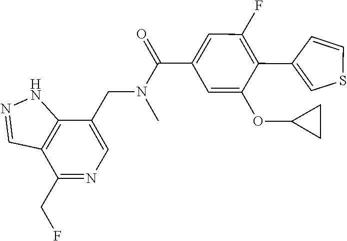 [SMILES: CN(Cc1cnc(CF)c2cn[nH]c12)C(=O)c1cc(F)c(-c2ccsc2)c(OC2CC2)c1] | 110 |
| 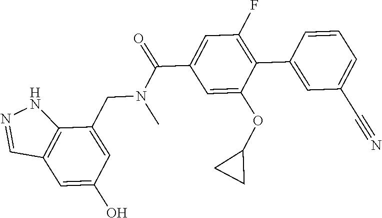 [SMILES: CN(Cc1cc(O)cc2cn[nH]c12)C(=O)c1cc(F)c(-c2cccc(C#N)c2)c(OC2CC2)c1] | 111 |
| 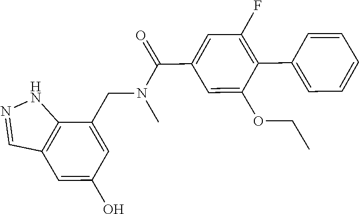 [SMILES: CCOc1cc(C(=O)N(C)Cc2cc(O)cc3cn[nH]c23)cc(F)c1-c1ccccc1] | 112 |
| 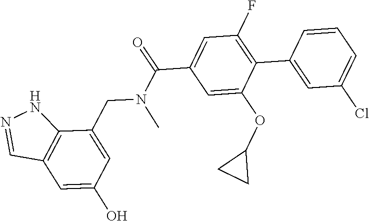 [SMILES: CN(Cc1cc(O)cc2cn[nH]c12)C(=O)c1cc(F)c(-c2cccc(Cl)c2)c(OC2CC2)c1] | 113 |
| 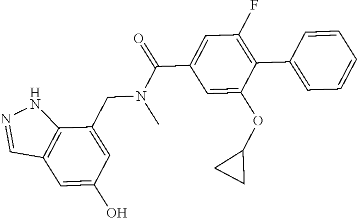 [SMILES: CN(Cc1cc(O)cc2cn[nH]c12)C(=O)c1cc(F)c(-c2ccccc2)c(OC2CC2)c1] | 114 |
| 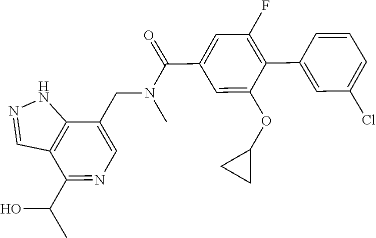 [SMILES: CC(O)c1ncc(CN(C)C(=O)c2cc(F)c(-c3cccc(Cl)c3)c(OC3CC3)c2)c2[nH]ncc12] | 115 |
| 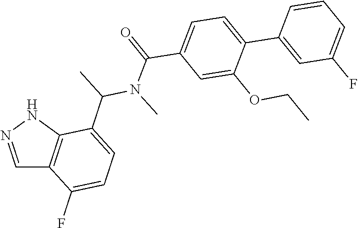 [SMILES: CCOc1cc(C(=O)N(C)C(C)c2ccc(F)c3cn[nH]c23)ccc1-c1cccc(F)c1] | 116 |
| 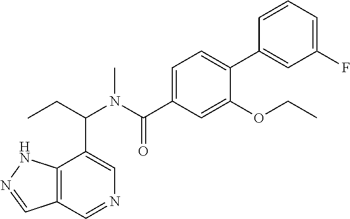 [SMILES: CCOc1cc(C(=O)N(C)C(CC)c2cncc3cn[nH]c23)ccc1-c1cccc(F)c1] | 117 |
| 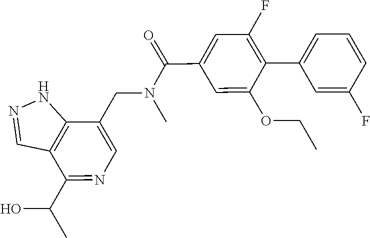 [SMILES: CCOc1cc(C(=O)N(C)Cc2cnc(C(C)O)c3cn[nH]c23)cc(F)c1-c1cccc(F)c1] | 118 |
| 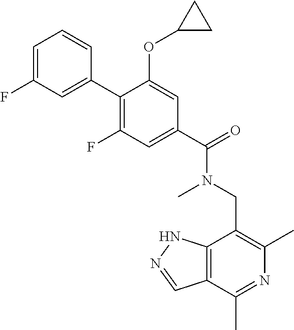 [SMILES: Cc1nc(C)c2cn[nH]c2c1CN(C)C(=O)c1cc(F)c(-c2cccc(F)c2)c(OC2CC2)c1] | 119 |
| 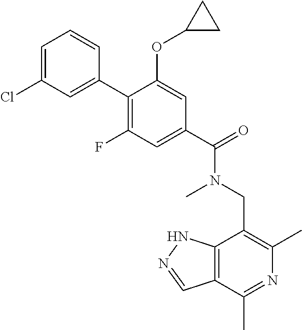 [SMILES: Cc1nc(C)c2cn[nH]c2c1CN(C)C(=O)c1cc(F)c(-c2cccc(Cl)c2)c(OC2CC2)c1] | 120 |
| 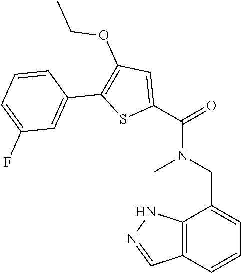 [SMILES: CCOc1cc(C(=O)N(C)Cc2cccc3cn[nH]c23)sc1-c1cccc(F)c1] | 121 |
| 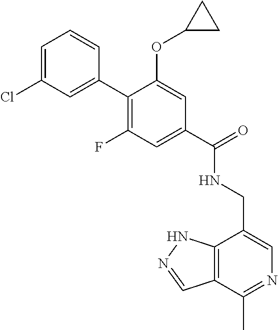 [SMILES: Cc1ncc(CNC(=O)c2cc(F)c(-c3cccc(Cl)c3)c(OC3CC3)c2)c2[nH]ncc12] | 122 |
| 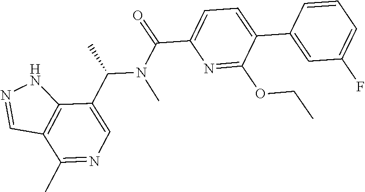 [SMILES: CCOc1nc(C(=O)N(C)[C@@H](C)c2cnc(C)c3cn[nH]c23)ccc1-c1cccc(F)c1] | 123 |
| 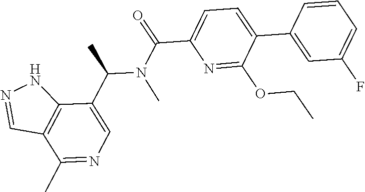 [SMILES: CCOc1nc(C(=O)N(C)[C@H](C)c2cnc(C)c3cn[nH]c23)ccc1-c1cccc(F)c1] | 124 |
| 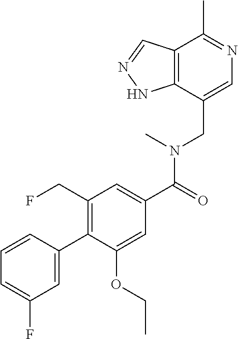 [SMILES: CCOc1cc(C(=O)N(C)Cc2cnc(C)c3cn[nH]c23)cc(CF)c1-c1cccc(F)c1] | 125 |
| 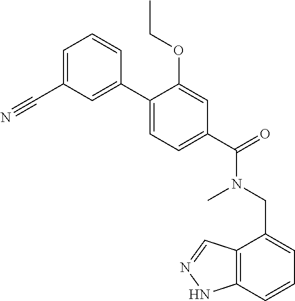 [SMILES: CCOc1cc(C(=O)N(C)Cc2cccc3[nH]ncc23)ccc1-c1cccc(C#N)c1] | 126 |
| 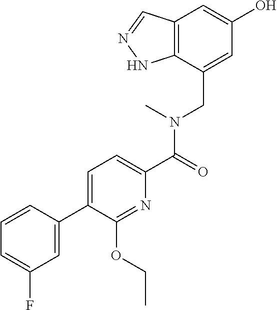 [SMILES: CCOc1nc(C(=O)N(C)Cc2cc(O)cc3cn[nH]c23)ccc1-c1cccc(F)c1] | 128 |
| 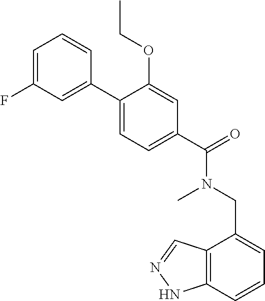 [SMILES: CCOc1cc(C(=O)N(C)Cc2cccc3[nH]ncc23)ccc1-c1cccc(F)c1] | 129 |
| 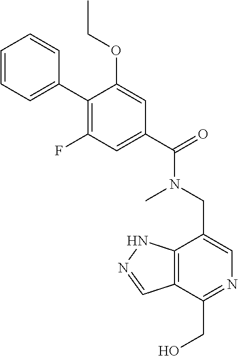 [SMILES: CCOc1cc(C(=O)N(C)Cc2cnc(CO)c3cn[nH]c23)cc(F)c1-c1ccccc1] | 130 |
| 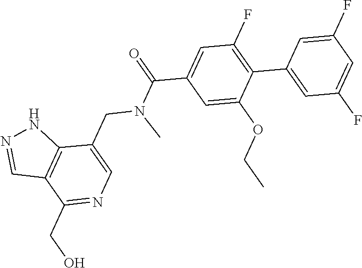 [SMILES: CCOc1cc(C(=O)N(C)Cc2cnc(CO)c3cn[nH]c23)cc(F)c1-c1cc(F)cc(F)c1] | 131 |
| 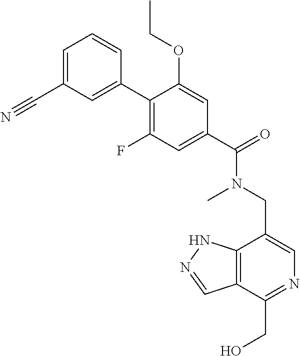 [SMILES: CCOc1cc(C(=O)N(C)Cc2cnc(CO)c3cn[nH]c23)cc(F)c1-c1cccc(C#N)c1] | 132 |
| 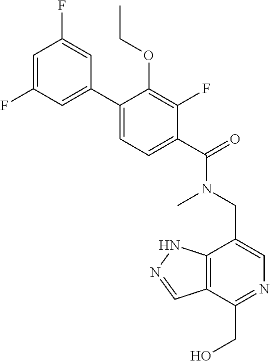 [SMILES: CCOc1c(-c2cc(F)cc(F)c2)ccc(C(=O)N(C)Cc2cnc(CO)c3cn[nH]c23)c1F] | 133 |
| 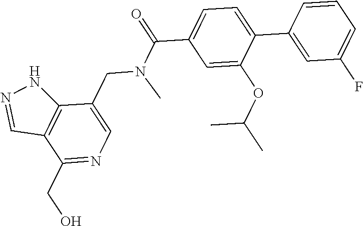 [SMILES: CC(C)Oc1cc(C(=O)N(C)Cc2cnc(CO)c3cn[nH]c23)ccc1-c1cccc(F)c1] | 134 |
| 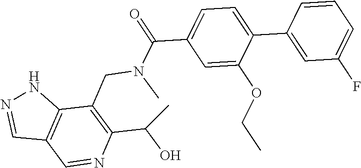 [SMILES: CCOc1cc(C(=O)N(C)Cc2c(C(C)O)ncc3cn[nH]c23)ccc1-c1cccc(F)c1] | 135 |
| 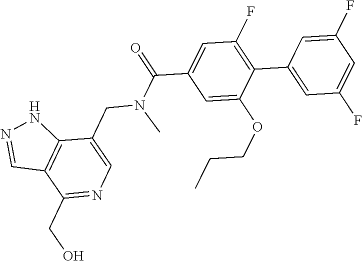 [SMILES: CCCOc1cc(C(=O)N(C)Cc2cnc(CO)c3cn[nH]c23)cc(F)c1-c1cc(F)cc(F)c1] | 136 |
| 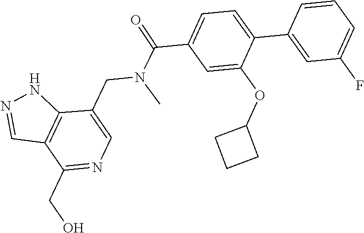 [SMILES: CN(Cc1cnc(CO)c2cn[nH]c12)C(=O)c1ccc(-c2cccc(F)c2)c(OC2CCC2)c1] | 137 |
| 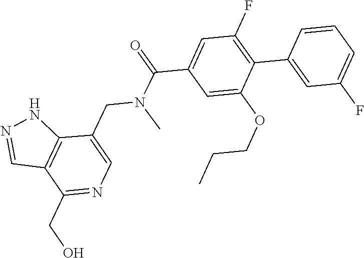 [SMILES: CCCOc1cc(C(=O)N(C)Cc2cnc(CO)c3cn[nH]c23)cc(F)c1-c1cccc(F)c1] | 138 |
| 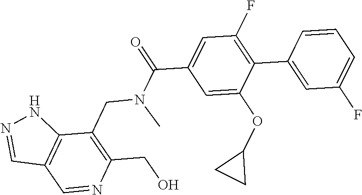 [SMILES: CN(Cc1c(CO)ncc2cn[nH]c12)C(=O)c1cc(F)c(-c2cccc(F)c2)c(OC2CC2)c1] | 139 |
| 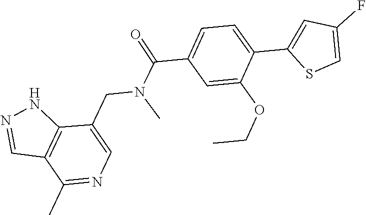 [SMILES: CCOc1cc(C(=O)N(C)Cc2cnc(C)c3cn[nH]c23)ccc1-c1cc(F)cs1] | 140 |
| 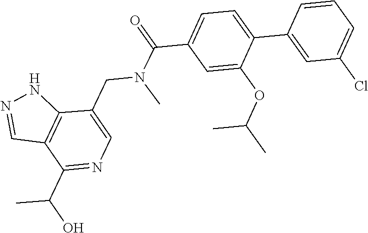 [SMILES: CC(C)Oc1cc(C(=O)N(C)Cc2cnc(C(C)O)c3cn[nH]c23)ccc1-c1cccc(Cl)c1] | 141 |
| 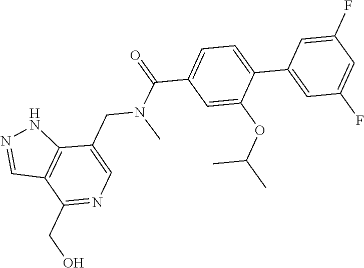 [SMILES: CC(C)Oc1cc(C(=O)N(C)Cc2cnc(CO)c3cn[nH]c23)ccc1-c1cc(F)cc(F)c1] | 142 |
| 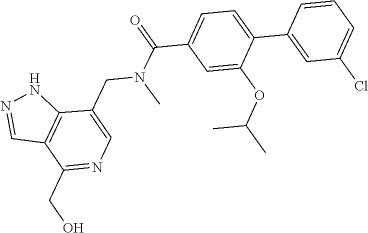 [SMILES: CC(C)Oc1cc(C(=O)N(C)Cc2cnc(CO)c3cn[nH]c23)ccc1-c1cccc(Cl)c1] | 143 |
| 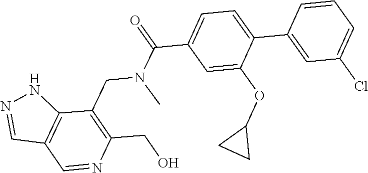 [SMILES: CN(Cc1c(CO)ncc2cn[nH]c12)C(=O)c1ccc(-c2cccc(Cl)c2)c(OC2CC2)c1] | 144 |
| 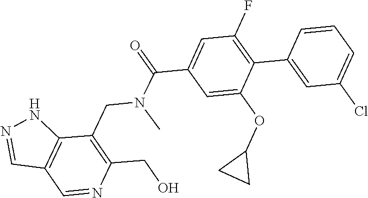 [SMILES: CN(Cc1c(CO)ncc2cn[nH]c12)C(=O)c1cc(F)c(-c2cccc(Cl)c2)c(OC2CC2)c1] | 145 |
| 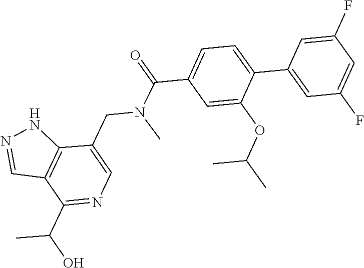 [SMILES: CC(C)Oc1cc(C(=O)N(C)Cc2cnc(C(C)O)c3cn[nH]c23)ccc1-c1cc(F)cc(F)c1] | 146 |
| 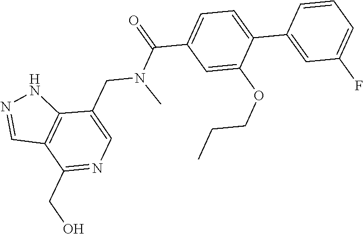 [SMILES: CCCOc1cc(C(=O)N(C)Cc2cnc(CO)c3cn[nH]c23)ccc1-c1cccc(F)c1] | 147 |
| 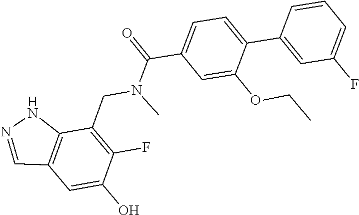 [SMILES: CCOc1cc(C(=O)N(C)Cc2c(F)c(O)cc3cn[nH]c23)ccc1-c1cccc(F)c1] | 148 |
| 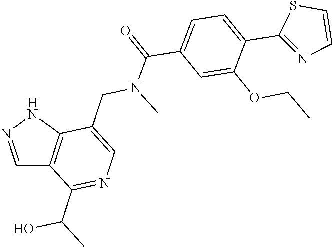 [SMILES: CCOc1cc(C(=O)N(C)Cc2cnc(C(C)O)c3cn[nH]c23)ccc1-c1nccs1] | 149 |
| 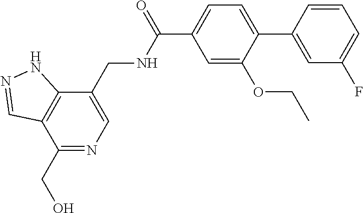 [SMILES: CCOc1cc(C(=O)NCc2cnc(CO)c3cn[nH]c23)ccc1-c1cccc(F)c1] | 150 |
| 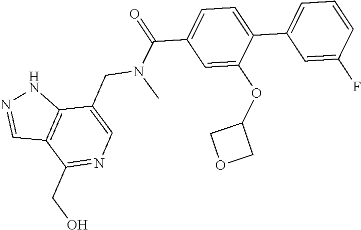 [SMILES: CN(Cc1cnc(CO)c2cn[nH]c12)C(=O)c1ccc(-c2cccc(F)c2)c(OC2COC2)c1] | 151 |
| 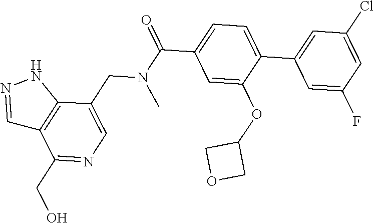 [SMILES: CN(Cc1cnc(CO)c2cn[nH]c12)C(=O)c1ccc(-c2cc(F)cc(Cl)c2)c(OC2COC2)c1] | 152 |
| 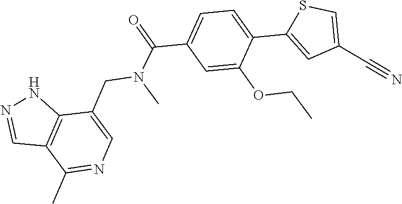 [SMILES: CCOc1cc(C(=O)N(C)Cc2cnc(C)c3cn[nH]c23)ccc1-c1cc(C#N)cs1] | 153 |
| 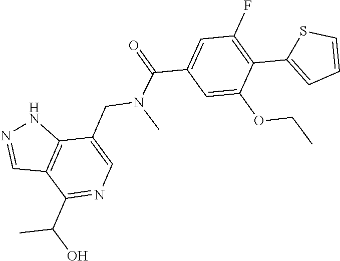 [SMILES: CCOc1cc(C(=O)N(C)Cc2cnc(C(C)O)c3cn[nH]c23)cc(F)c1-c1cccs1] | 154 |
| 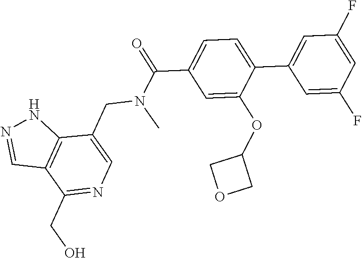 [SMILES: CN(Cc1cnc(CO)c2cn[nH]c12)C(=O)c1ccc(-c2cc(F)cc(F)c2)c(OC2COC2)c1] | 155 |
| 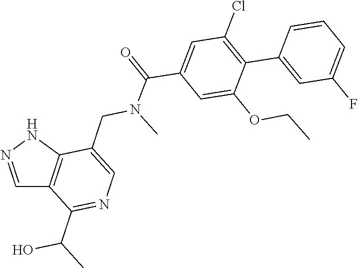 [SMILES: CCOc1cc(C(=O)N(C)Cc2cnc(C(C)O)c3cn[nH]c23)cc(Cl)c1-c1cccc(F)c1] | 156 |
| 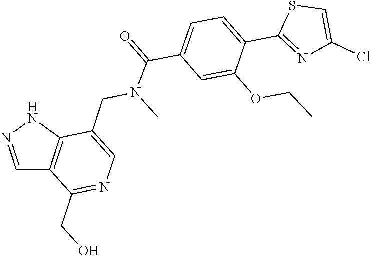 [SMILES: CCOc1cc(C(=O)N(C)Cc2cnc(CO)c3cn[nH]c23)ccc1-c1nc(Cl)cs1] | 157 |
| 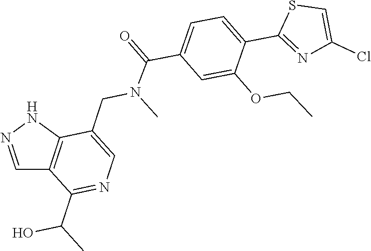 [SMILES: CCOc1cc(C(=O)N(C)Cc2cnc(C(C)O)c3cn[nH]c23)ccc1-c1nc(Cl)cs1] | 158 |
| 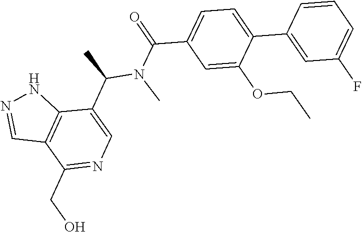 [SMILES: CCOc1cc(C(=O)N(C)[C@H](C)c2cnc(CO)c3cn[nH]c23)ccc1-c1cccc(F)c1] | 159 |
| 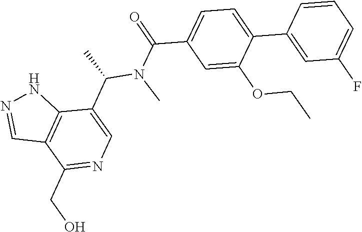 [SMILES: CCOc1cc(C(=O)N(C)[C@@H](C)c2cnc(CO)c3cn[nH]c23)ccc1-c1cccc(F)c1] | 160 |
| 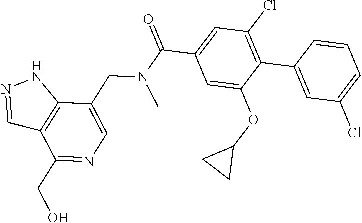 [SMILES: CN(Cc1cnc(CO)c2cn[nH]c12)C(=O)c1cc(Cl)c(-c2cccc(Cl)c2)c(OC2CC2)c1] | 161 |
| 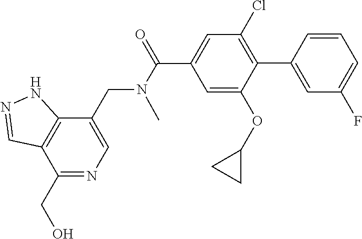 [SMILES: CN(Cc1cnc(CO)c2cn[nH]c12)C(=O)c1cc(Cl)c(-c2cccc(F)c2)c(OC2CC2)c1] | 162 |
| 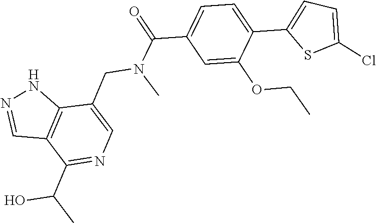 [SMILES: CCOc1cc(C(=O)N(C)Cc2cnc(C(C)O)c3cn[nH]c23)ccc1-c1ccc(Cl)s1] | 163 |
| 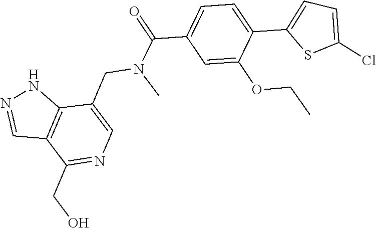 [SMILES: CCOc1cc(C(=O)N(C)Cc2cnc(CO)c3cn[nH]c23)ccc1-c1ccc(Cl)s1] | 164 |
| 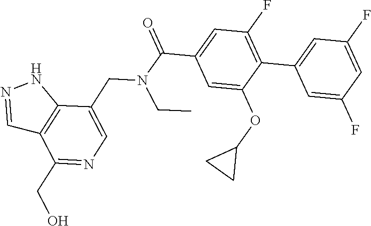 [SMILES: CCN(Cc1cnc(CO)c2cn[nH]c12)C(=O)c1cc(F)c(-c2cc(F)cc(F)c2)c(OC2CC2)c1] | 165 |
| 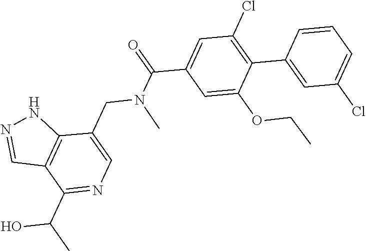 [SMILES: CCOc1cc(C(=O)N(C)Cc2cnc(C(C)O)c3cn[nH]c23)cc(Cl)c1-c1cccc(Cl)c1] | 166 |
| 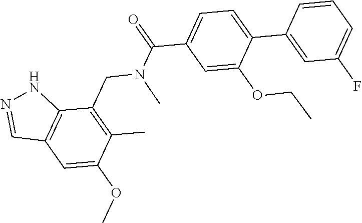 [SMILES: CCOc1cc(C(=O)N(C)Cc2c(C)c(OC)cc3cn[nH]c23)ccc1-c1cccc(F)c1] | 167 |
| 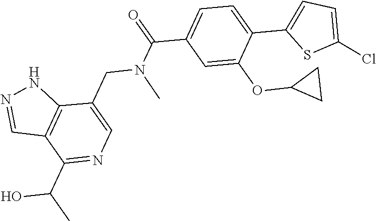 [SMILES: CC(O)c1ncc(CN(C)C(=O)c2ccc(-c3ccc(Cl)s3)c(OC3CC3)c2)c2[nH]ncc12] | 168 |
| 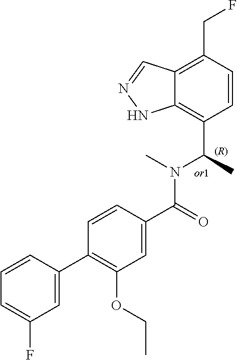 | 169 |
| 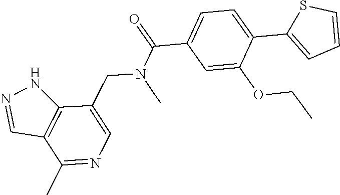 [SMILES: CCOc1cc(C(=O)N(C)Cc2cnc(C)c3cn[nH]c23)ccc1-c1cccs1] | 170 |
| 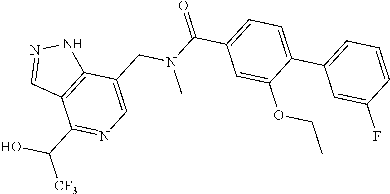 [SMILES: CCOc1cc(C(=O)N(C)Cc2cnc(C(O)C(F)(F)F)c3cn[nH]c23)ccc1-c1cccc(F)c1] | 171 |
| 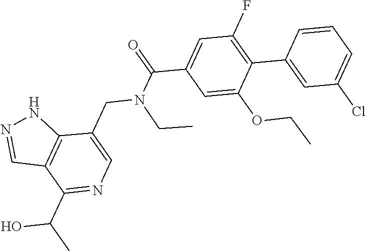 [SMILES: CCOc1cc(C(=O)N(CC)Cc2cnc(C(C)O)c3cn[nH]c23)cc(F)c1-c1cccc(Cl)c1] | 172 |
| 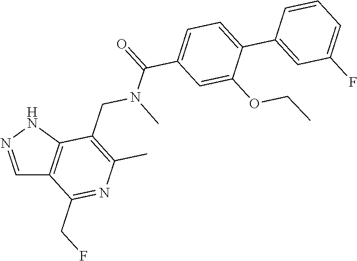 [SMILES: CCOc1cc(C(=O)N(C)Cc2c(C)nc(CF)c3cn[nH]c23)ccc1-c1cccc(F)c1] | 173 |
| 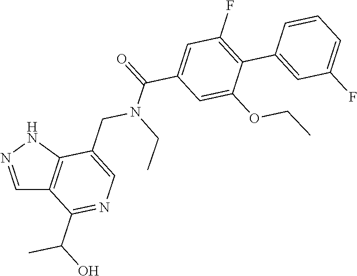 [SMILES: CCOc1cc(C(=O)N(CC)Cc2cnc(C(C)O)c3cn[nH]c23)cc(F)c1-c1cccc(F)c1] | 174 |
| 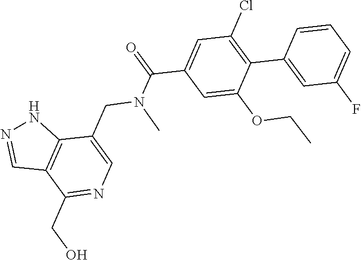 [SMILES: CCOc1cc(C(=O)N(C)Cc2cnc(CO)c3cn[nH]c23)cc(Cl)c1-c1cccc(F)c1] | 175 |
| 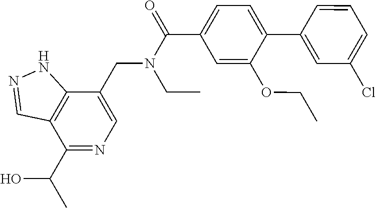 [SMILES: CCOc1cc(C(=O)N(CC)Cc2cnc(C(C)O)c3cn[nH]c23)ccc1-c1cccc(Cl)c1] | 176 |
| 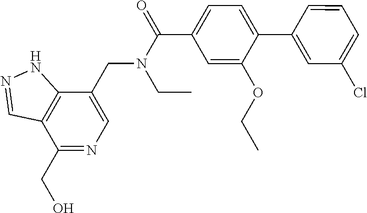 [SMILES: CCOc1cc(C(=O)N(CC)Cc2cnc(CO)c3cn[nH]c23)ccc1-c1cccc(Cl)c1] | 177 |
| 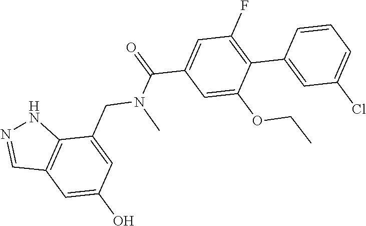 [SMILES: CCOc1cc(C(=O)N(C)Cc2cc(O)cc3cn[nH]c23)cc(F)c1-c1cccc(Cl)c1] | 178 |
| 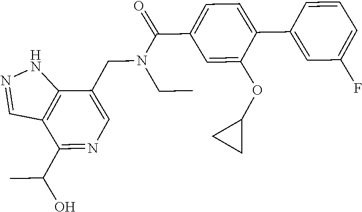 [SMILES: CCN(Cc1cnc(C(C)O)c2cn[nH]c12)C(=O)c1ccc(-c2cccc(F)c2)c(OC2CC2)c1] | 179 |
| 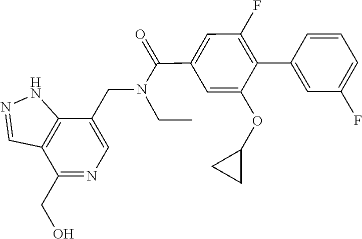 [SMILES: CCN(Cc1cnc(CO)c2cn[nH]c12)C(=O)c1cc(F)c(-c2cccc(F)c2)c(OC2CC2)c1] | 180 |
| 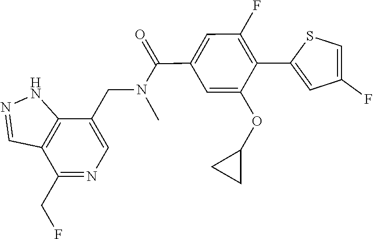 [SMILES: CN(Cc1cnc(CF)c2cn[nH]c12)C(=O)c1cc(F)c(-c2cc(F)cs2)c(OC2CC2)c1] | 181 |
| 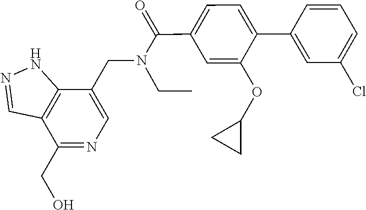 [SMILES: CCN(Cc1cnc(CO)c2cn[nH]c12)C(=O)c1ccc(-c2cccc(Cl)c2)c(OC2CC2)c1] | 182 |
| 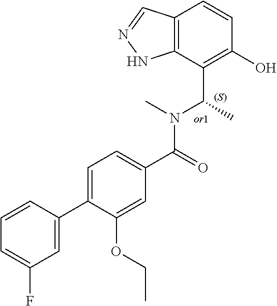 | 183 |
| 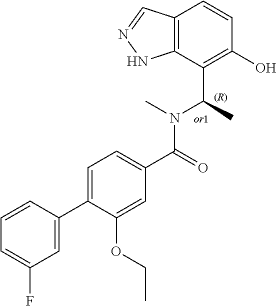 | 184 |

or a pharmaceutically acceptable salt thereof.

In an embodiment, the compound of Table 2 is selected from

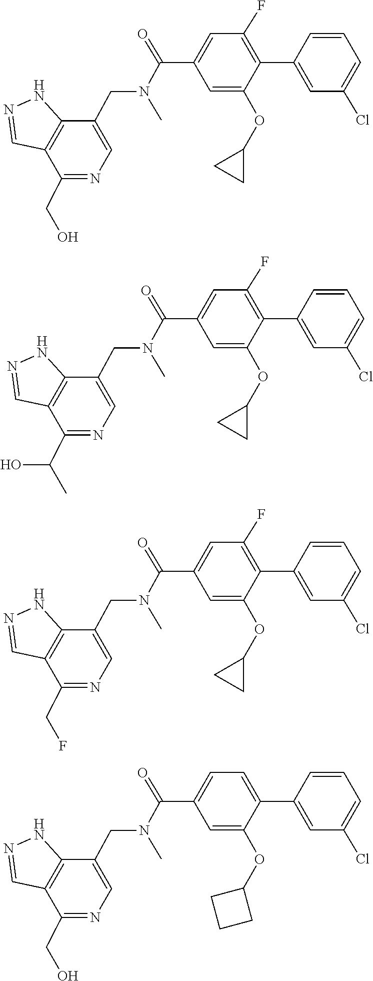 [SMILES: CN(Cc1cnc(CO)c2cn[nH]c12)C(=O)c1cc(F)c(-c2cccc(Cl)c2)c(OC2CC2)c1 ; CC(O)c1ncc(CN(C)C(=O)c2cc(F)c(-c3cccc(Cl)c3)c(OC3CC3)c2)c2[nH]ncc12 ; CN(Cc1cnc(CF)c2cn[nH]c12)C(=O)c1cc(F)c(-c2cccc(Cl)c2)c(OC2CC2)c1 ; CN(Cc1cnc(CO)c2cn[nH]c12)C(=O)c1ccc(-c2cccc(Cl)c2)c(OC2CCC2)c1]

or a pharmaceutically acceptable salt thereof.

In yet another aspect, provided herein is a compound selected from the group consisting of a compound in Table 3.

TABLE 3

| | Com- |
|---|---|
| | pound |
| Structure | No. |
| 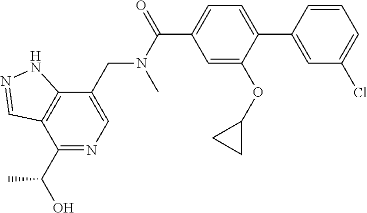 [SMILES: C[C@@H](O)c1ncc(CN(C)C(=O)c2ccc(-c3cccc(Cl)c3)c(OC3CC3)c2)c2[nH]ncc12] | 185 |
| 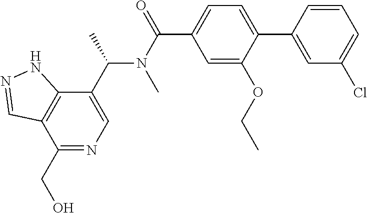 [SMILES: CCOc1cc(C(=O)N(C)[C@@H](C)c2cnc(CO)c3cn[nH]c23)ccc1-c1cccc(Cl)c1] | 186 |
| 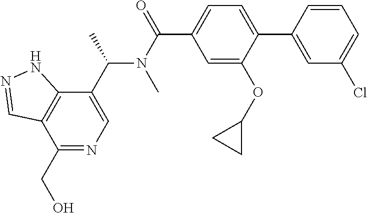 [SMILES: C[C@@H](c1cnc(CO)c2cn[nH]c12)N(C)C(=O)c1ccc(-c2cccc(Cl)c2)c(OC2CC2)c1] | 187 |
| 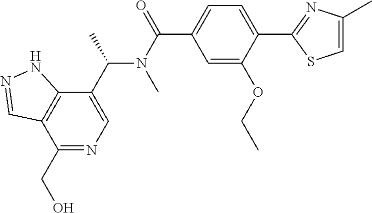 [SMILES: CCOc1cc(C(=O)N(C)[C@@H](C)c2cnc(CO)c3cn[nH]c23)ccc1-c1nc(C)cs1] | 188 |

or a pharmaceutically acceptable salt thereof.

In an embodiment, the compound is selected from the group consisting of a compound in Table 4.

TABLE 4

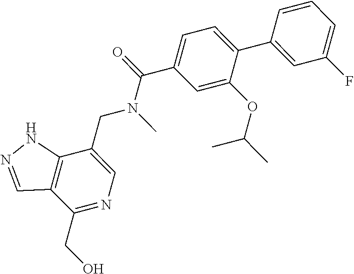 [SMILES: CC(C)Oc1cc(C(=O)N(C)Cc2cnc(CO)c3cn[nH]c23)ccc1-c1cccc(F)c1]

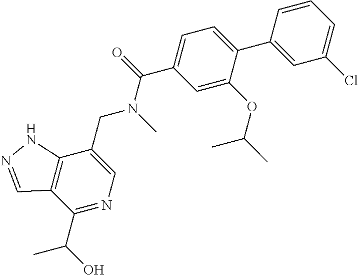 [SMILES: CC(C)Oc1cc(C(=O)N(C)Cc2cnc(C(C)O)c3cn[nH]c23)ccc1-c1cccc(Cl)c1]

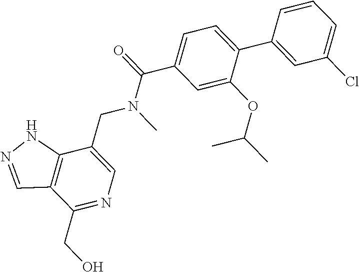 [SMILES: CC(C)Oc1cc(C(=O)N(C)Cc2cnc(CO)c3cn[nH]c23)ccc1-c1cccc(Cl)c1]

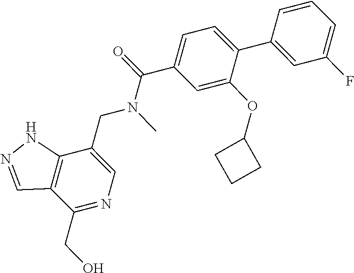 [SMILES: CN(Cc1cnc(CO)c2cn[nH]c12)C(=O)c1ccc(-c2cccc(F)c2)c(OC2CCC2)c1]

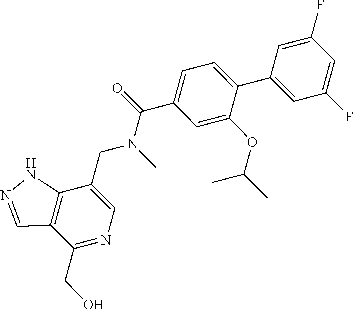 [SMILES: CC(C)Oc1cc(C(=O)N(C)Cc2cnc(CO)c3cn[nH]c23)ccc1-c1cc(F)cc(F)c1]

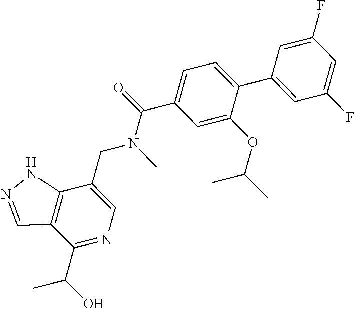 [SMILES: CC(C)Oc1cc(C(=O)N(C)Cc2cnc(C(C)O)c3cn[nH]c23)ccc1-c1cc(F)cc(F)c1]

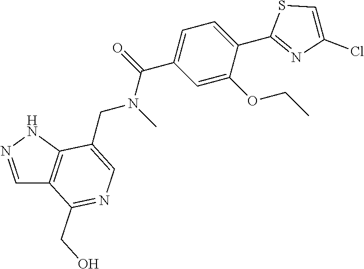 [SMILES: CCOc1cc(C(=O)N(C)Cc2cnc(CO)c3cn[nH]c23)ccc1-c1nc(Cl)cs1]

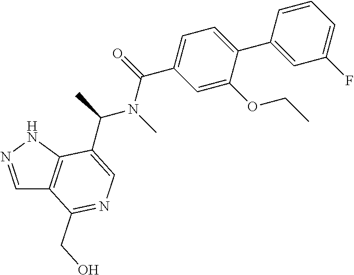 [SMILES: CCOc1cc(C(=O)N(C)[C@H](C)c2cnc(CO)c3cn[nH]c23)ccc1-c1cccc(F)c1]

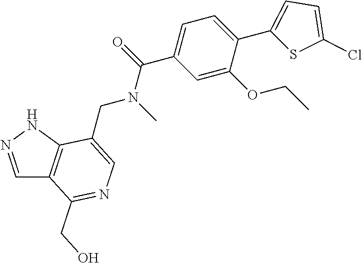 [SMILES: CCOc1cc(C(=O)N(C)Cc2cnc(CO)c3cn[nH]c23)ccc1-c1ccc(Cl)s1]

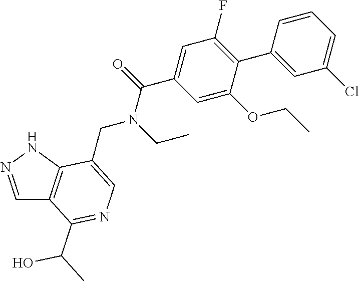 [SMILES: CCOc1cc(C(=O)N(CC)Cc2cnc(C(C)O)c3cn[nH]c23)cc(F)c1-c1cccc(Cl)c1]

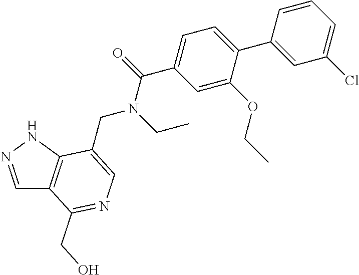 [SMILES: CCOc1cc(C(=O)N(CC)Cc2cnc(CO)c3cn[nH]c23)ccc1-c1cccc(Cl)c1]

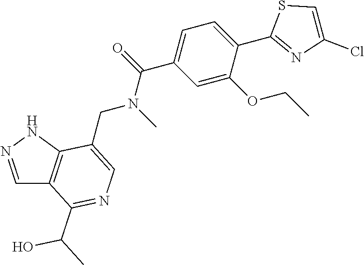 [SMILES: CCOc1cc(C(=O)N(C)Cc2cnc(C(C)O)c3cn[nH]c23)ccc1-c1nc(Cl)cs1]

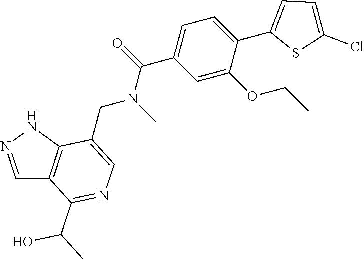 [SMILES: CCOc1cc(C(=O)N(C)Cc2cnc(C(C)O)c3cn[nH]c23)ccc1-c1ccc(Cl)s1]

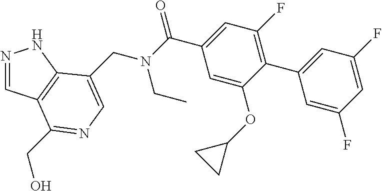 [SMILES: CCN(Cc1cnc(CO)c2cn[nH]c12)C(=O)c1cc(F)c(-c2cc(F)cc(F)c2)c(OC2CC2)c1]

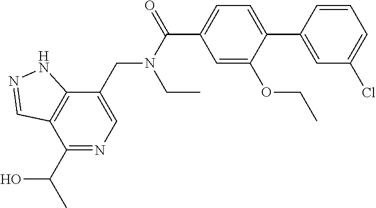 [SMILES: CCOc1cc(C(=O)N(CC)Cc2cnc(C(C)O)c3cn[nH]c23)ccc1-c1cccc(Cl)c1]

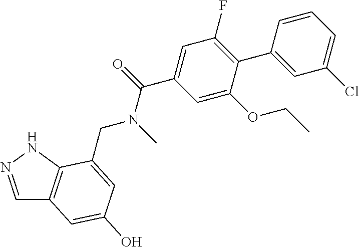 [SMILES: CCOc1cc(C(=O)N(C)Cc2cc(O)cc3cn[nH]c23)cc(F)c1-c1cccc(Cl)c1]

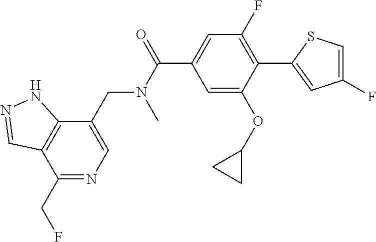 [SMILES: CN(Cc1cnc(CF)c2cn[nH]c12)C(=O)c1cc(F)c(-c2cc(F)cs2)c(OC2CC2)c1]

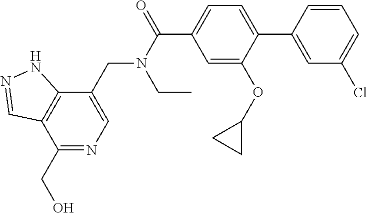 [SMILES: CCN(Cc1cnc(CO)c2cn[nH]c12)C(=O)c1ccc(-c2cccc(Cl)c2)c(OC2CC2)c1]

or a pharmaceutically acceptable salt thereof.

In another embodiment, the compound is selected from the group consisting of a compound in Table 5.

TABLE 5

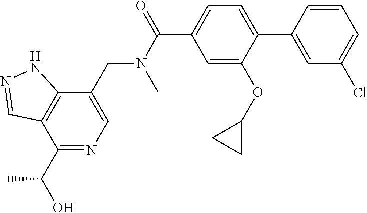 [SMILES: C[C@@H](O)c1ncc(CN(C)C(=O)c2ccc(-c3cccc(Cl)c3)c(OC3CC3)c2)c2[nH]ncc12]

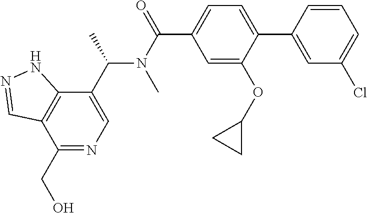 [SMILES: C[C@@H](c1cnc(CO)c2cn[nH]c12)N(C)C(=O)c1ccc(-c2cccc(Cl)c2)c(OC2CC2)c1]

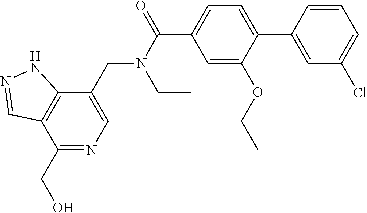 [SMILES: CCOc1cc(C(=O)N(CC)Cc2cnc(CO)c3cn[nH]c23)ccc1-c1cccc(Cl)c1]

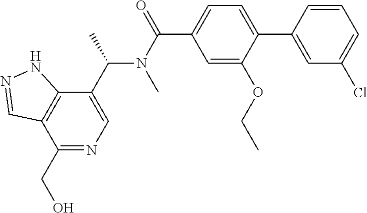 [SMILES: CCOc1cc(C(=O)N(C)[C@@H](C)c2cnc(CO)c3cn[nH]c23)ccc1-c1cccc(Cl)c1]

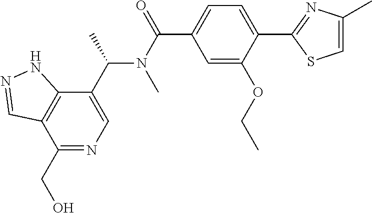 [SMILES: CCOc1cc(C(=O)N(C)[C@@H](C)c2cnc(CO)c3cn[nH]c23)ccc1-c1nc(C)cs1]

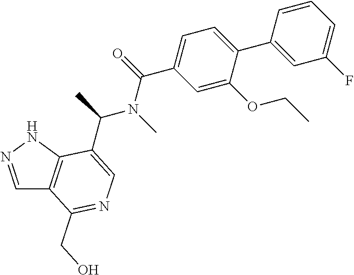 [SMILES: CCOc1cc(C(=O)N(C)[C@H](C)c2cnc(CO)c3cn[nH]c23)ccc1-c1cccc(F)c1]

or a pharmaceutically acceptable salt thereof.

In another aspect, provided herein is a pharmaceutical composition comprising a compound described herein, or a pharmaceutically acceptable salt thereof, and a pharmaceutically acceptable carrier.

The compounds disclosed herein may exist as tautomers and optical isomers (e.g., enantiomers, diastereomers, diastereomeric mixtures, racemic mixtures, and the like). The absolute stereochemistry is specified according to the Cahn-Ingold-Prelog R-S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30). Certain examples contain chemical structures that are depicted or labelled as an (R*) or (S*). When (R*) or (S*) is used in the name of a compound or in the chemical representation of the compound, it is intended to convey that the compound is a pure single isomer at that stereocenter; however, absolute configuration of that stereocenter has not been established. Thus, a compound designated as (R*) refers to a compound that is a pure single isomer at that stereocenter with an absolute configuration of either (R) or (S), and a compound designated as (S*) refers to a compound that is a pure single isomer at that stereocenter with an absolute configuration of either (R) or (S).

It is generally well known in the art that any compound that will be converted in vivo to provide a compound disclosed herein is a prodrug within the scope of the present disclosure.

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Methods of Treatment

In an aspect, provided herein is a method of inhibiting NLRP3 inflammasome in a subject in need thereof, comprising administering to the individual a therapeutically effective amount of a compound disclosed herein.

In another aspect, provided herein is a method of treating inflammation in a subject in need thereof, comprising administering to the individual a therapeutically effective amount of a compound disclosed herein.

In another aspect, provided herein is a method of treating inflammaging in a subject in need thereof, comprising administering to the individual a therapeutically effective amount of a compound disclosed herein.

In another aspect, provided herein is a method of treating cryopyrin-associated periodic syndrome (CAPS) in a subject in need thereof, comprising administering to the individual a therapeutically effective amount of a compound disclosed herein.

In an embodiment, the CAPS is selected from the group consisting of familial cold autoinflammatory syndrome, Muckle-Wells syndrome, and neonatal-onset multisystem inflammatory disease.

In another aspect, provided herein is a method of treating a dermatologic disease in a subject in need thereof, comprising administering to the individual a therapeutically effective amount of a compound disclosed herein.

In an embodiment, the dermatologic disease is selected from the group consisting of psoriasis, urticaria, skin photoaging, and eczema.

In yet another aspect, provided herein is a method of treating a neurosensory disease in a subject in need thereof, comprising administering to the individual a therapeutically effective amount of a compound disclosed herein.

In an embodiment, the neurosensory disease is selected from the group consisting of amyotrophic lateral sclerosis (ALS), traumatic brain injury, Parkinson's disease, and Alzheimer's disease.

In another aspect, provided herein is a method of treating inflammation in a subject in need thereof comprising administering to the subject a therapeutically effective amount of a compound of Formula I:

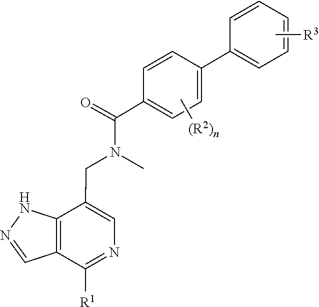

(I)
or a pharmaceutically acceptable salt thereof;
   wherein
R¹ is C₁₋₆ alkyl optionally substituted with a substituent selected from halo, OH, OC₁₋₃ alkyl, NH₂, N(H)C₁₋₃ alkyl, N(C₁₋₆ alkyl)₂, C(O)C₁₋₃ alkyl, and C₃₋₆ cycloalkyl;
each R² is independently selected from halo, C₁₋₃ haloalkyl, OH, OC₁₋₃ alkyl, OC₃₋₆ cycloalkyl, and C₃₋₆ cycloalkyl;
R³ is selected from H, halo, C₁₋₃ haloalkyl, OH, OC₁₋₃ alkyl, OC₃₋₆ cycloalkyl, CN, and C₃₋₆ cycloalkyl; and
n is 0, 1, or 2.

In an embodiment,
R¹ is C₁₋₆ alkyl optionally substituted with OH;
each R² is independently selected from halo and C₃₋₆ cycloalkyl;
R³ is halo; and
n is 1 or 2.

In another aspect, provided herein is a method of treating inflammaging and neurosensory diseases in a subject in need thereof comprising administering to the subject a therapeutically effective amount of a compound of Formula I, or a pharmaceutically acceptable salt thereof, or a pharmaceutical composition comprising a compound of Formula I and at least one pharmaceutically acceptable carrier.

In an embodiment, the neurosensory disease is selected from the group consisting of amyotrophic lateral sclerosis (ALS), traumatic brain injury, Parkinson's disease, and Alzheimer's disease.

In yet another aspect, provided herein is a method of inhibiting NLRP3 inflammasome in a subject in need thereof comprising administering to the subject a therapeutically effective amount of a compound of Formula I, or a pharmaceutically acceptable salt thereof, or a pharmaceutical composition comprising a compound of Formula I and at least one pharmaceutically acceptable carrier.

In another embodiment, the compound of Formula I is selected from

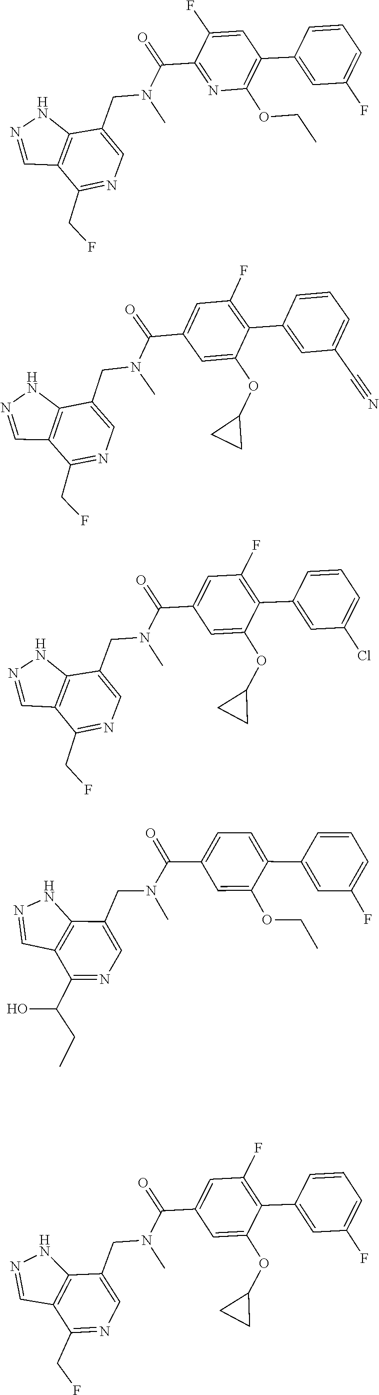 [SMILES: CCOc1nc(C(=O)N(C)Cc2cnc(CF)c3cn[nH]c23)c(F)cc1-c1cccc(F)c1 ; CN(Cc1cnc(CF)c2cn[nH]c12)C(=O)c1cc(F)c(-c2cccc(C#N)c2)c(OC2CC2)c1 ; CN(Cc1cnc(CF)c2cn[nH]c12)C(=O)c1cc(F)c(-c2cccc(Cl)c2)c(OC2CC2)c1 ; CCOc1cc(C(=O)N(C)Cc2cnc(C(O)CC)c3cn[nH]c23)ccc1-c1cccc(F)c1 ; CN(Cc1cnc(CF)c2cn[nH]c12)C(=O)c1cc(F)c(-c2cccc(F)c2)c(OC2CC2)c1]

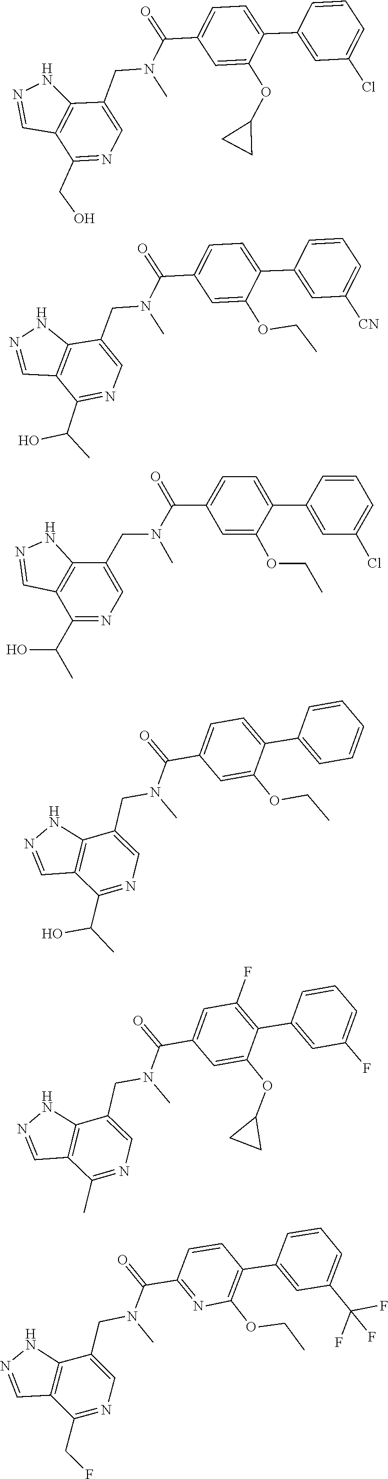 [SMILES: CN(Cc1cnc(CO)c2cn[nH]c12)C(=O)c1ccc(-c2cccc(Cl)c2)c(OC2CC2)c1 ; CCOc1cc(C(=O)N(C)Cc2cnc(C(C)O)c3cn[nH]c23)ccc1-c1cccc(C#N)c1 ; CCOc1cc(C(=O)N(C)Cc2cnc(C(C)O)c3cn[nH]c23)ccc1-c1cccc(Cl)c1 ; CCOc1cc(C(=O)N(C)Cc2cnc(C(C)O)c3cn[nH]c23)ccc1-c1ccccc1 ; Cc1ncc(CN(C)C(=O)c2cc(F)c(-c3cccc(F)c3)c(OC3CC3)c2)c2[nH]ncc12 ; CCOc1nc(C(=O)N(C)Cc2cnc(CF)c3cn[nH]c23)ccc1-c1cccc(C(F)(F)F)c1]

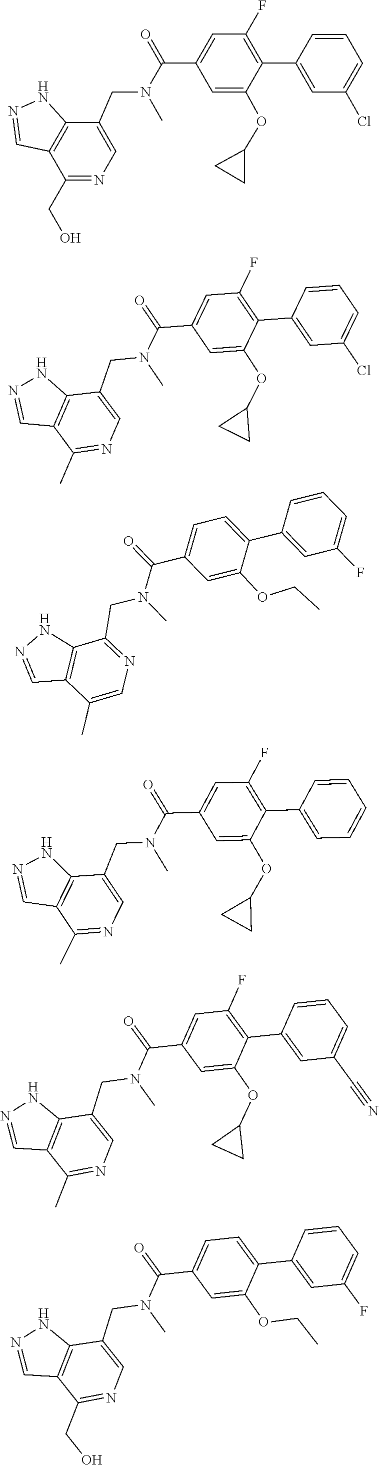 [SMILES: CN(Cc1cnc(CO)c2cn[nH]c12)C(=O)c1cc(F)c(-c2cccc(Cl)c2)c(OC2CC2)c1 ; Cc1ncc(CN(C)C(=O)c2cc(F)c(-c3cccc(Cl)c3)c(OC3CC3)c2)c2[nH]ncc12 ; CCOc1cc(C(=O)N(C)Cc2ncc(C)c3cn[nH]c23)ccc1-c1cccc(F)c1 ; Cc1ncc(CN(C)C(=O)c2cc(F)c(-c3ccccc3)c(OC3CC3)c2)c2[nH]ncc12 ; Cc1ncc(CN(C)C(=O)c2cc(F)c(-c3cccc(C#N)c3)c(OC3CC3)c2)c2[nH]ncc12 ; CCOc1cc(C(=O)N(C)Cc2cnc(CO)c3cn[nH]c23)ccc1-c1cccc(F)c1]

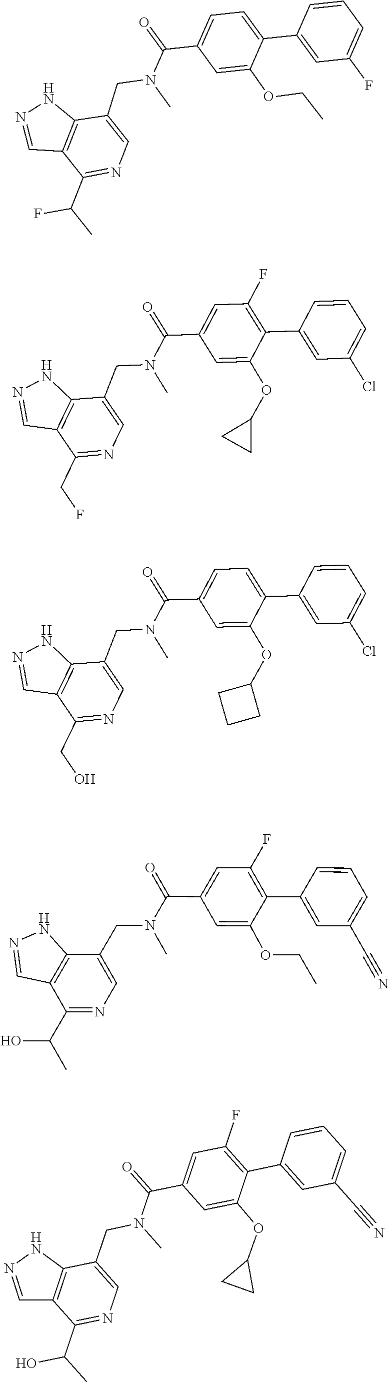 [SMILES: CCOc1cc(C(=O)N(C)Cc2cnc(C(C)F)c3cn[nH]c23)ccc1-c1cccc(F)c1 ; CN(Cc1cnc(CF)c2cn[nH]c12)C(=O)c1cc(F)c(-c2cccc(Cl)c2)c(OC2CC2)c1 ; CN(Cc1cnc(CO)c2cn[nH]c12)C(=O)c1ccc(-c2cccc(Cl)c2)c(OC2CCC2)c1 ; CCOc1cc(C(=O)N(C)Cc2cnc(C(C)O)c3cn[nH]c23)cc(F)c1-c1cccc(C#N)c1 ; CC(O)c1ncc(CN(C)C(=O)c2cc(F)c(-c3cccc(C#N)c3)c(OC3CC3)c2)c2[nH]ncc12]

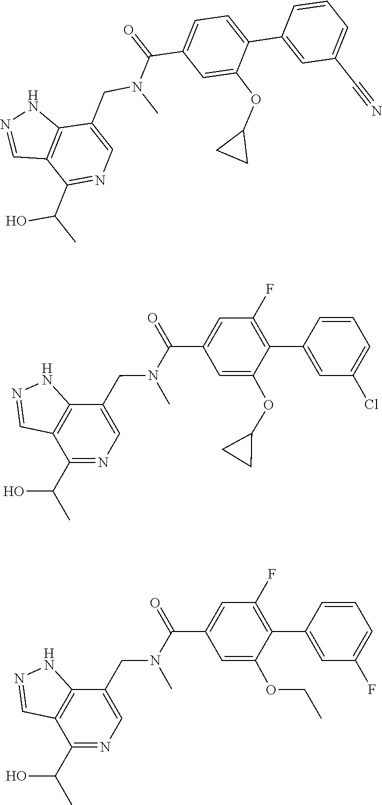 [SMILES: CC(O)c1ncc(CN(C)C(=O)c2ccc(-c3cccc(C#N)c3)c(OC3CC3)c2)c2[nH]ncc12 ; CC(O)c1ncc(CN(C)C(=O)c2cc(F)c(-c3cccc(Cl)c3)c(OC3CC3)c2)c2[nH]ncc12 ; CCOc1cc(C(=O)N(C)Cc2cnc(C(C)O)c3cn[nH]c23)cc(F)c1-c1cccc(F)c1]

or a pharmaceutically acceptable salt thereat.

In yet another embodiment, the compound of Formula I is selected from

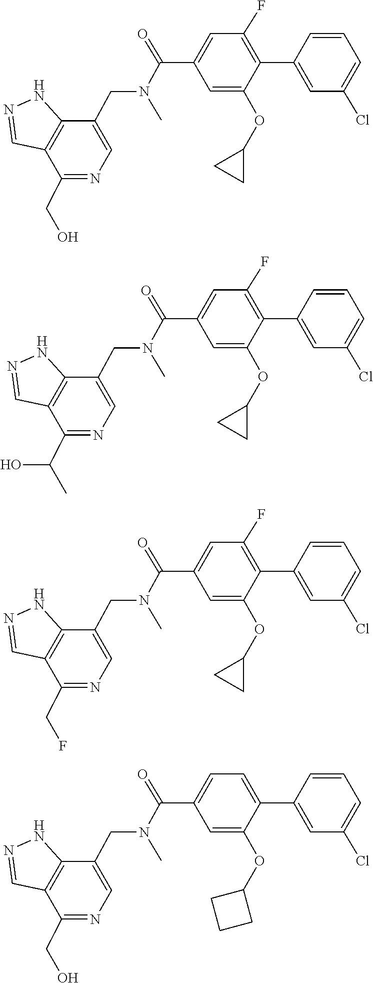 [SMILES: CN(Cc1cnc(CO)c2cn[nH]c12)C(=O)c1cc(F)c(-c2cccc(Cl)c2)c(OC2CC2)c1 ; CC(O)c1ncc(CN(C)C(=O)c2cc(F)c(-c3cccc(Cl)c3)c(OC3CC3)c2)c2[nH]ncc12 ; CN(Cc1cnc(CF)c2cn[nH]c12)C(=O)c1cc(F)c(-c2cccc(Cl)c2)c(OC2CC2)c1 ; CN(Cc1cnc(CO)c2cn[nH]c12)C(=O)c1ccc(-c2cccc(Cl)c2)c(OC2CCC2)c1]

or a pharmaceutically acceptable salt thereof.

Also provided herein is a method of using the compounds provided herein for treatment or amelioration of aging or an aging-related condition negatively impacting longevity or quality of life, wherein the aging-related condition negatively impacting longevity or quality of life is selected from the group consisting of inflammation, anemia, hyperglycemia, dyslipidemia, hyperinsulinemia, insulin resistance, immunosuppression, liver disease, iron overload, hypertrigliceridemia, impaired skin integrity, wound healing, scarring, pain, allergies, sleep disorders and problems, gastrointestinal disorders and problems, Th1-type inflammation, Th2-type inflammation, an inflammatory disease involving T-cell dependent B cell proliferation, T-cell dependent B cell proliferation, allergy, asthma, atherosclerosis, autoimmunity, hypercholesterolemia, chronic inflammation, chronic obstructive pulmonary disease (COPD), Crohn's disease, cutaneous responses to tissue damage, fibrosis, hematological oncology, metabolic diseases, cardiovascular disease, organ transplantation, psoriasis, liver fibrosis, dermatitis, pulmonary fibrosis, pulmonary responses to respiratory infections, restenosis, rheumatoid arthritis, sarcoidosis, stromal biology in tumors, systemic lupus erythematosus (SLE), ulcerative colitis, vascular inflammation, and diseases that are driven or exacerbated by one or more factors selected from the group consisting of alpha smooth muscle actin (αSMA), CD40, CD69, collagen I, collagen III, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCL10), interferon-inducible T cell alpha chemoattractant (I-TAC/CXCL11), interleukin (IL)-1, IL-1.alpha., IL-2, IL-6, IL-8 (CXCL8), IL-10, IL-17A, IL-17F, keratin 8/81, macrophage colony-stimulating factor (M-CSF), matrix metalloproteinase (MMP)-1, MMP-9, monocyte chemoattractant protein 1 (MCP-1), monokine induced by gamma interferon (MIG/CXCL9), plasminogen activation inhibitor 1 (PAI-1), prostaglandin E2 (PGE2), serum amyloid A, T or B cell proliferation, tissue plasminogen activator (tPA), tumor necrosis factor alpha (TNF.alpha.), vascular cell adhesion molecule (VCAM-1), and vascular endothelial growth factor 2 (VEGFR2), comprising: administering to a subject in need thereof a compound provided herein.

In an aspect, provided herein is a method of reversing a normal aging process in subject comprising administering to the subject a therapeutically effective amount of a compound provided herein or a pharmaceutically acceptable salt thereof.

In another aspect, provided herein is a method of reversing a normal aging process in subject comprising administering to the subject a pharmaceutical composition comprising a therapeutically effective amount of a compound provided herein or a pharmaceutically acceptable salt thereof.

In yet another aspect, provided herein is a method of extending lifespan of a subject comprising administering to the subject a therapeutically effective amount of a compound provided herein or a pharmaceutically acceptable salt thereof.

In still another aspect, provided herein is a method of extending lifespan of a subject comprising administering to the subject a pharmaceutical composition comprising a therapeutically effective amount of a compound provided herein or a pharmaceutically acceptable salt thereof.

In another aspect, provided herein is a method to slow down and mitigate the aging process in a subject comprising administering to the subject a therapeutically effective amount of a compound provided herein or a pharmaceutically acceptable salt thereof.

In another aspect, provided herein is a method of inhibiting or modulating the pro-inflammatory pathway in a cell comprising contacting the cell with a compound provided herein, or a pharmaceutically acceptable salt thereof. In yet another aspect, provided herein is a method of inhibiting or modulating NLRP3 in a cell comprising contacting the cell with a compound provided herein, or a pharmaceutically acceptable salt thereof.

Treatment of a cell (in vitro or in vivo) that expresses a NLRP3 inflammasome with a compound provided herein can result in inhibiting the pro-inflammatory pathway and inhibiting downstream events related to the signaling pathway such as inflammation or inflammaging.

In another aspect, provided herein is a method of treating a neurosensory disease in a subject in need thereof, comprising administering to the individual a therapeutically effective amount of a compound disclosed herein.

In an embodiment, the neurosensory disease is selected from the group consisting of hearing loss, hearing injury, and ocular disease. In an embodiment, the ocular disease is retinal and optic nerve injury.

In yet another aspect, provided herein is a method of treating an inflammatory disorder in a subject in need thereof, comprising administering to the individual a therapeutically effective amount of a compound disclosed herein.

In an embodiment, the inflammatory disorder is selected from the group consisting of allergy, asthma, atopic dermatitis, atherosclerosis, autoimmune diseases, coeliac disease, chronic inflammation, glomerulonephritis, hepatitis, inflammatory bowel disease, preperfusion injury, SARS-CoV-2 infection, transplant rejection, heart disease, diabetes, arthritis, Crohn's disease, ulcerative colitis, non-alcoholic steatohepatitis (NASH), gout, coronary artery disease, rheumatoid arthritis, intestinal disorders, and acute respiratory distress syndrome (ARDS).

In another embodiment, the inflammatory disorder is a neuroinflammatory disease. In yet another embodiment, the inflammatory disorder is inner ear inflammation.

In an embodiment, a chronic inflammation comprises a tissue inflammation. Tissue inflammation is a chronic inflammation that is confined to a particular tissue or organ. In an embodiment, a tissue inflammation comprises, e.g., a skin inflammation, ocular inflammation, a muscle inflammation, a tendon inflammation, a ligament inflammation, a bone inflammation, a cartilage inflammation, a lung inflammation, a heart inflammation, a liver inflammation, a pancreatic inflammation, a kidney inflammation, a bladder inflammation, a stomach inflammation, an intestinal inflammation, a neuron inflammation, and a brain inflammation.

In another embodiment, a chronic inflammation comprises a systemic inflammation. Although the processes involved are identical to tissue inflammation, systemic inflammation is not confined to a particular tissue but in fact overwhelms the body, involving the endothelium and other organ systems. When it is due to infection, the term sepsis is applied, with the terms bacteremia being applied specifically for bacterial sepsis and viremia specifically to viral sepsis. Vasodilation and organ dysfunction are serious problems associated with widespread infection that may lead to septic shock and death.

In yet another embodiment, a chronic inflammation comprises an arthritis. Arthritis includes a group of conditions involving damage to the joints of the body due to the inflammation of the synovium including, without limitation osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease), and Still's disease. Arthritis can affect a single joint (monoarthritis), two to four joints (oligoarthritis) or five or more joints (polyarthritis) and can be either an auto-immune disease or a non-autoimmune disease.

In still another aspect, provided herein is a method of treating an age-related disorder in a subject in need thereof, comprising administering to the individual a therapeutically effective amount of a compound disclosed herein.

In an embodiment, the age-related disorder is selected from the group consisting of neurodegeneration, cardiovascular disease, insulin resistance, diabetes, osteoporosis, osteoarthritis, cognitive decline, dementia, frailty, cataracts, arthritis, obesity, hypertension, angina, congestive heart failure, dyslipidemia, myocardial infarction, vascular disease, respiratory disease, kidney disease, cerebrovascular disease, peripheral vascular disease, Alzheimer's disease, cardiac diastolic dysfunction, benign prostatic hypertrophy, aortic aneurysm, and emphysema.

In another aspect, provided herein is a method of treating a metabolic condition in a subject in need thereof, comprising administering to the individual a therapeutically effective amount of a compound disclosed herein.

In an embodiment, the metabolic condition is selected from the group consisting of diabetes, obesity, cystic fibrosis, and hyperthyroidism.

In yet another aspect, provided herein is a method of treating a neurodegenerative disease in a subject in need thereof, comprising administering to the individual a therapeutically effective amount of a compound disclosed herein.

In an embodiment, the neurodegenerative disease is selected from the group consisting of Alzheimer's disease, Parkinson's disease, Huntington's disease, multiple sclerosis (MS), amyotrophic lateral sclerosis (ALS), and Batten disease.

In an aspect, provided herein is a method of treating a disease or disorder of the inner ear in a subject in need thereof, comprising administering to the individual a therapeutically effective amount of a compound disclosed herein.

In an embodiment, the disease or disorder of the inner ear is selected from the group consisting of hearing loss, hearing impairment, vertigo, Meniere's disease, and tinnitus. In another embodiment, the disease of the inner ear is hearing loss. In yet another embodiment, the disease of the inner ear is hearing impairment.

In another embodiment, the hearing loss is age-related, noise-induced, or the result of a viral infection. In yet another embodiment, the viral infection is Zika virus or coronavirus.

Potency of the inhibitor can also be determined by IC₅₀ value. A compound with a lower IC₅₀ value, as determined under substantially similar conditions, is a more potent inhibitor relative to a compound with a higher IC₅₀ value.

In an embodiment of the methods, the subject is a human.

In another aspect, the disclosure provides a compound disclosed herein, or a pharmaceutically acceptable salt thereof, for use in the manufacture of a medicament for treating or preventing a disease in which NLRP3 inflammasome plays a role.

In an aspect, provided herein is a method of treating a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer.

Therefore, in an aspect, provided herein is a method of treating cancer in a subject in need thereof comprising administering to the subject a therapeutically effective amount of a compound disclosed herein, or a pharmaceutically acceptable salt thereof.

In an embodiment, the cancer is selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia.

In another embodiment, the cancer is selected from the group consisting of myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

In an embodiment, the cancer is selected from the group consisting of prostate cancer, colon cancer, lung cancer, squamous cell cancer of the head and neck, esophageal cancer, hepatocellular carcinoma, melanoma, sarcoma, gastric cancer, pancreatic cancer, ovarian cancer, breast cancer.

In an embodiment, the cancer is selected from the group consisting of tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in treating are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In another aspect, provided herein is the use of one or more compounds of the disclosure in the manufacture of a medicament for the treatment of cancer, including without limitation the various types of cancer disclosed herein.

In some embodiments, the compounds of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

### Administration/Dosages/Formulations

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility.

The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compounds of the present disclosure can be administered intratympanically, wherein a long, narrow, bore needle is passed through the ear canal and through the eardrum to administer medications into the middle ear space where they are absorbed by the inner ear.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means a sufficient amount of the compound so as to decrease the symptoms of a disorder in a subject. As is well understood in the medical arts a therapeutically effective amount of a compound of this disclosure will be at a reasonable benefit/risk ratio applicable to any medical treatment.

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present disclosure may range from about 0.1 mg/Kg to about 500 mg/Kg, alternatively from about 1 to about 50 mg/Kg. In general, treatment regimens according to the present disclosure comprise administration to a patient in need of such treatment from about 10 mg to about 1000 mg of the compound(s) of this disclosure per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed;

the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present disclosure.

### Kits

In an aspect, provided herein is a kit comprising a compound capable of inhibiting NLRP3 inflammasome activity selected from one or more compounds of disclosed herein, or pharmaceutically acceptable salts thereof, and instructions for use in treating a disorder associated with NLRP3 inflammasomes.

In another aspect, the disclosure provides a kit comprising a compound capable of inhibiting NLRP3 inflammasome activity selected from a compound disclosed herein, or a pharmaceutically acceptable salt thereof.

In yet another aspect, provided herein is a kit comprising a compound disclosed herein, or a pharmaceutically acceptable salt thereof for the treatment of any of the indications disclosed herein.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Example 1: Synthetic Procedures

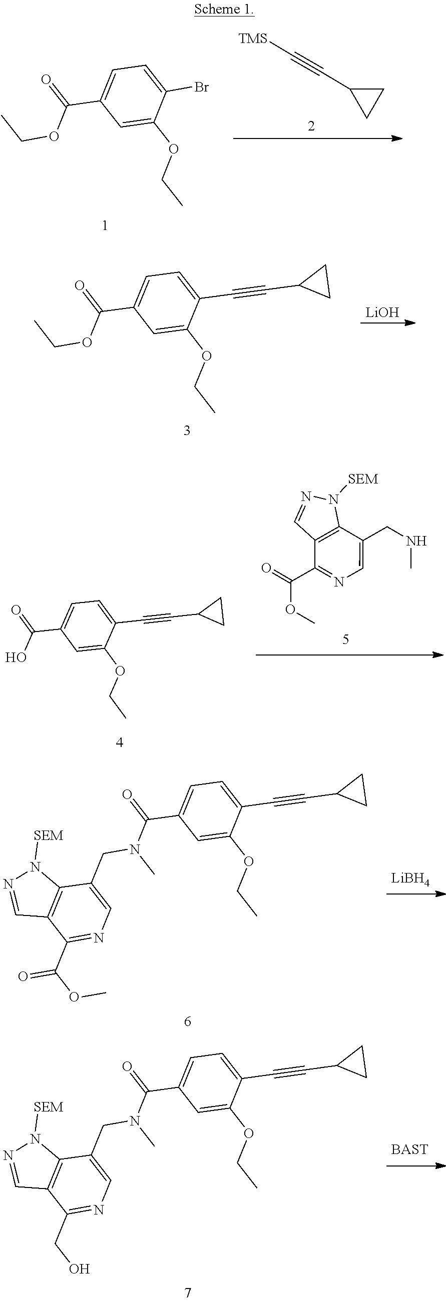

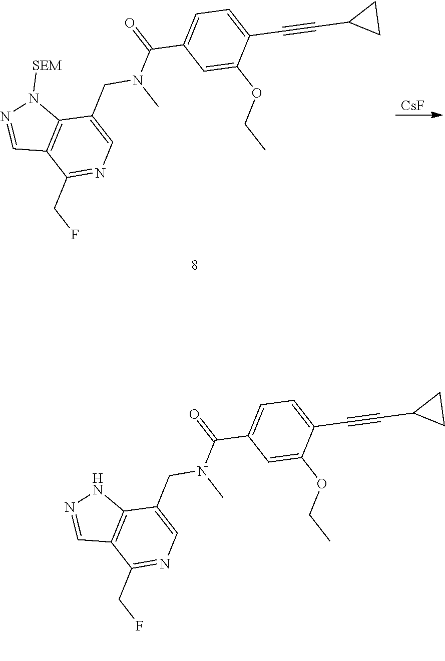 [SMILES: CCOc1cc(C(=O)N(C)Cc2cnc(CF)c3cnn(COCC[Si](C)(C)C)c23)ccc1C#CC1CC1 ; "CsF" ; CCOc1cc(C(=O)N(C)Cc2cnc(CF)c3cn[nH]c23)ccc1C#CC1CC1]

To a solution of Compound 1 (1 g, 3.66 mmol, 1 eq) and 2 Compound 2 (506.30 mg, 3.66 mmol, 1 eq) in DMF (10 mL) in glass-bottle (40 mL) was added TBAF (1 M, 4.03 mL, 1.1 eq), TEA (740.98 mg, 7.32 mmol, 1.02 mL, 2 eq), CuI (139.46 mg, 732.27 umol, 0.2 eq) and Pd(PPh₃)₂Cl₂ (256.99 mg, 366.14 umol, 0.1 eq). The mixture was stirred at 100° C. for 12 hr under N₂ atmosphere. The reaction mixture was poured into water (60 mL), and extracted with EtOAc (50 mL×5). The combined organic layers were over [Na₂SO₄], filtered and concentrated under reduced pressure to give a residue. The residue was purified by column chromatography (SiO₂, PE/EtOAc=1/0 to 5/1), the purified solution was concentrated on vacuum to give Compound 3 (280 mg, 1.08 mmol, 29.61% yield) as a brown oil. LCMS: Rt=0.530 min, m/z=259.1 (M+H)⁺

To a solution of Compound 3 (280 mg, 1.08 mmol, 1 eq) in EtOH (3 mL) and H₂O (1 mL) in round-bottom flask (50 mL) was added LiOH·H₂O (136.46 mg, 3.25 mmol, 3 eq). The mixture was stirred at 20° C. for 1 hr. The organic solvents was evaporated under vacuum, the resulting mixture was acidified by 1N HCl to pH4, filtered, the filter cake was washed by water (20 mL), then dissolved in EtOH (20 mL), and concentrated on vacuum to give Compound 4 (240 mg, 1.04 mmol, 96.16% yield) as a yellow solid which was used in the next step. LCMS: Rt=0.307 min, m/z=229.1 (M−H)⁺

To a solution of Compound 4 (131.39 mg, 570.63 umol, 1 eq) and Compound 5 (200 mg, 570.63 umol, 1 eq) in DCM (10 mL) in round-bottom flask (50 mL) was added EDCI (164.09 mg, 855.95 umol, 1.5 eq) and DMAP (6.97 mg, 57.06 umol, 0.1 eq). The mixture was stirred at 20° C. for 1 hr. The reaction mixture was concentrated on vacuum to give residue. The residue was purified by column chromatography (SiO₂, PE/EtOAc=5/1 to 0/1), the purified solution was concentrated on vacuum to give Compound 6 (285 mg, 506.46 umol, 88.75% yield) as a yellow oil. LCMS: Rt=0.549 min, m/z=563.3 (M+H)⁺

To a solution of Compound 6 (285 mg, 506.46 umol, 1 eq) in MeOH (5 mL) was added LiBH₄ (33.10 mg, 1.52 mmol, 3 eq) at 0° C. The mixture was stirred at 20° C. for 2 hr.

The reaction mixture was poured into saturated NH₄Cl aqueous (20 mL) and extracted by extracted by EtOAc (30 mL×3), the combined organic phase was dried and concentrated under vacuum to give a residue. The residue was purified by column chromatography (SiO₂, EtOAc:Methanol=I/O to 10/1), the purified solution was concentrated on vacuum to give Compound 7 (150 mg, 280.52 umol, 55.39% yield) as a yellow oil. LCMS: Rt=0.452 min, m/z=535.3 (M+H)⁺

To a solution of Compound 7 (150 mg, 280.52 umol, 1 eq) in DCM (10 mL) in glass bottle (40 mL) was added BAST (124.13 mg, 561.04 umol, 122.90 uL, 2 eq) at 0° C. The mixture was stirred at 20° C. for 10 min. The reaction mixture was cooled in ice bath, then poured into saturated NaHCO₃ aqueous (20 mL) and extracted by DCM (30 mL×3), the combined organic phase was dried and concentrated under vacuum to give a residue. The residue was purified by prep-TLC (SiO₂, PE:EtOAc=0:1), the purified solution was concentrated on vacuum to give Compound 8 (40 mg, 74.53 umol, 26.57% yield) as a colorless oil. LCMS: Rt=0.530 min, m/z=537.3 (M+H)⁺

To a solution of Compound 8 (35 mg, 65.21 umol, 1 eq) in DMF (1 mL) in glass-bottle (8 mL) was added CsF (99.06 mg, 652.12 umol, 24.04 uL, 10 eq). The mixture was stirred at 100° C. for 12 hr. The mixture was stirred at 100° C. for 60 hr. The mixture was stirred at 120° C. for 24 hr. The reaction mixture was filtered. The filtrate was purified by prep-HPLC (column: Water xbridge 150*25 mm 10 um; mobile phase: [water (NH₄HCO₃)-ACN]; B %: 34%-64%, 8 min). The purified solution was lyophilized to give 001 (5.9 mg, 14.52 umol, 22.26% yield, 100% purity) as a white solid. LCMS: Rt=0.379 min, m/z=407.0 (M+H)⁺ 1H NMR (400 MHz, chloroform-d) δ=12.28-12.15 (m, 1H), 8.45-8.39 (m, 1H), 8.26-8.21 (m, 1H), 7.39-7.34 (m, 1H), 6.95-6.90 (m, 2H), 5.93-5.69 (m, 2H), 4.95-4.86 (m, 2H), 4.15-4.04 (m, 2H), 3.01-2.92 (m, 3H), 1.49 (br s, 1H), 1.49-1.44 (m, 3H), 0.93-0.87 (m, 2H), 0.87 (br s, 2H).

Compound 012 was prepared in a similar manner to Scheme 1 using TFA instead of CsF and was obtained as an off-white solid (57 mg, 123.90 umol, 72.57% yield, and 99% purity). LCMS: Rt=0.423 min, m/z=456.4 (M+H⁺) ¹H NMR: (400 MHz, chloroform-d) δ=12.09-11.81 (m, 1H), 8.44 (d, J=2.6 Hz, 1H), 8.29 (s, 1H), 7.50 (d, J=8.9 Hz, 1H), 7.45-7.40 (m, 1H), 7.38-7.33 (m, 2H), 7.16-7.09 (m, 1H), 5.93-5.77 (m, 2H), 5.01 (s, 2H), 4.44-4.35 (m, 2H), 3.05 (s, 3H), 1.37 (t, J=7.1 Hz, 3H).

Compound 013 was prepared in a similar manner to Scheme 1 using DAST instead of BAST and was obtained as a white solid (2.3 mg, 5.83 umol, 11.77% yield, 100% purity). LCMS: Rt=0.351 min, m/z=395.2 (M+H+). 1H NMR: (chloroform-d): δ 12.20 (1H, br s), 8.41 (1H, d, J=2.5 Hz), 8.24 (1H, s), 7.39 (1H, d, J=8.3 Hz), 6.88-6.98 (2H, m), 5.69-5.91 (2H, m), 4.91 (2H, s), 4.10 (2H, q, J=7.0 Hz), 2.97 (3H, s), 2.49 (2H, q, J=7.5 Hz), 1.47 (3H, t, J=7.0 Hz), 1.23-1.28 (3H, m).

Compound 014 was prepared in a similar manner to Scheme 1 and was obtained as a white solid (6.8 mg, 17.83 umol, 22.67% yield, 99.2% purity). LCMS: Rt=0.472 min, m/z=379.0 (M+H)^{+ 1}H NMR (400 MHz, chloroform-d) δ=12.29-12.14 (m, 1H), 8.28-8.18 (m, 2H), 7.39 (d, J=7.9 Hz, 1H), 6.99-6.87 (m, 2H), 5.15-5.07 (m, 2H), 4.95-4.86 (m, 2H), 4.11 (q, J=6.9 Hz, 2H), 3.01-2.94 (m, 3H), 2.12 (s, 3H), 1.50-1.44 (m, 3H).

Compound 018 was prepared in a similar manner to Scheme 1 and was obtained as a white solid (10.01 mg, 20.37 umol, 35.18% yield, and 100% purity). LCMS: Rt=0.385 min, m/z=492.1 (M+H+). HNMR: (400 MHz, chloroform-d) δ=8.51 (br s, 1H), 8.33 (s, 1H), 7.47 (s, 1H), 7.41-7.28 (m, 3H), 6.92 (d, J=9.3 Hz, 1H), 6.03-5.86 (m, 2H), 4.98 (s, 2H), 3.86-3.72 (m, 1H), 3.09 (s, 3H), 0.84 (br d, J=5.9 Hz, 2H), 0.72 (br s, 2H).

Compound 019 was prepared in a similar manner to Scheme 1 and was obtained as a white solid (6.5 mg, 13.66 umol, 14.99% yield, 99.5% purity). LCMS: Rt=0.390 min, m/z=474.0 (M+H)⁺ 1H NMR (400 MHz, chloroform-d) δ ppm 12.13-12.39 (m, 1H) 8.46-8.52 (m, 1H) 8.27-8.35 (m, 1H) 7.64-7.68 (m, 2H) 7.57-7.61 (m, 1H) 7.50-7.55 (m, 1H) 7.25-7.26 (m, 1H) 6.90-6.93 (m, 1H) 5.83-6.02 (m, 2H) 4.95-5.01 (m, 2H) 3.72-3.81 (m, 1H) 3.06-3.12 (m, 3H) 0.79-0.84 (m, 2H) 0.68-0.74 (m, 2H).

Compound 020 was prepared in a similar manner to Scheme 1 and was obtained as a white solid (7 mg, 14.45 umol, 12.69% yield). ¹H-NMR: (400 MHz, chloroform-d) δ=12.64-11.79 (m, 1H), 8.44 (d, J=2.3 Hz, 1H), 8.28 (s, 1H), 7.23 (s, 1H), 6.91-6.81 (m, 4H), 5.91-5.77 (m, 2H), 4.96 (s, 2H), 3.76 (tt, J=2.9, 5.8 Hz, 1H), 3.07 (s, 3H), 0.83-0.79 (m, 2H), 0.75-0.70 (m, 2H). LCMS: Rt=0.419 min, m/z=484.9 (M+H⁺).

Compound 022 was prepared in a similar manner to Scheme 1 and was obtained as a white solid (6.5 mg, 13.41 umol, 32.88% yield, 99.6% purity). LCMS: Rt=0.435 min, m/z=483.2 (M+H)⁺ 1H NMR (400 MHz, chloroform-d) δ=12.62-11.60 (m, 1H), 8.44 (d, J=2.3 Hz, 1H), 8.31-8.25 (m, 1H), 7.36-7.32 (m, 3H), 7.25-7.20 (m, 2H), 6.91-6.86 (m, 1H), 5.94-5.76 (m, 2H), 4.99-4.92 (m, 2H), 3.80-3.71 (m, 1H), 3.10-3.04 (m, 3H), 0.83-0.76 (m, 2H), 0.75-0.68 (m, 2H).

Compound 027 was prepared in a similar manner to Scheme 1 and was obtained as a white solid (15.5 mg, 32.76 umol, 48.88% yield, 98.6% purity). LCMS: Rt=0.424 min, m/z=467.1 (M+H)⁺ 1H NMR (400 MHz, chloroform-d) δ=8.45 (d, J=2.5 Hz, 1H), 8.28 (s, 1H), 7.43-7.32 (m, 1H), 7.24 (s, 1H), 7.12 (br d, J=7.4 Hz, 1H), 7.09-7.04 (m, 2H), 6.89 (d, J=8.9 Hz, 1H), 5.95-5.79 (m, 2H), 4.96 (s, 2H), 3.76 (br d, J=3.0 Hz, 1H), 3.08 (s, 3H), 0.91-0.86 (m, 2H), 0.80 (br d, J=5.5 Hz, 2H).

Compound 046 was prepared in a similar manner to Scheme 1 and was obtained as a white solid (45 mg, 92.32 umol, 38.02% yield, 100% purity). LCMS: Rt=0.445 min, m/z=488.1 (M+H)^{+ 1}H NMR (400 MHz, chloroform-d) δ ppm 12.11-12.36 (m, 1H) 8.40-8.45 (m, 1H) 8.26-8.31 (m, 1H) 7.87-7.92 (m, 1H) 7.74-7.80 (m, 2H) 7.62-7.67 (m, 1H) 7.54-7.59 (m, 1H) 7.51 (d, J=7.63 Hz, 1H) 5.75-5.92 (m, 2H) 4.93-5.01 (m, 2H) 4.42 (q, J=7.13 Hz, 2H) 3.22 (s, 3H) 1.33-1.41 (m, 3H).

Compound 071 was prepared in a similar manner to Scheme 1 using TFA instead of CsF and was obtained as a white solid (5.4 mg, 11.08 umol, 22.65% yield, 99.1% purity). LCMS: Rt=0.432 min, m/z=483.2 (M+H)^{+ 1}H NMR (400 MHz, chloroform-d) δ=1 8.44 (d, J=2.8 Hz, 1H), 8.28 (s, 1H), 7.38-7.31 (m, 3H), 7.23 (s, 2H), 6.89 (d, J=9.0 Hz, 1H), 5.93-5.76 (m, 2H), 4.96 (s, 2H), 3.75 (td, J=3.0, 5.8 Hz, 1H), 3.07 (s, 3H), 0.84-0.77 (m, 2H), 0.75-0.69 (m, 2H).

Compound 082 was prepared in a similar manner to Scheme 1 using TFA instead of CsF and was obtained as a white solid (10.38 mg, 21.98 umol, 43.80% yield, 99% purity). LCMS: Rt=0.370 min, m/z=478.1 (M+Na⁺) ¹H NMR: (400 MHz, chloroform-d) δ=12.35 (br d, J=5.4 Hz, 1H), 8.48-8.19 (m, 2H), 7.77 (s, 1H), 7.69 (d, J=7.9 Hz, 1H), 7.63 (d, J=7.8 Hz, 1H), 7.55-7.48 (m, 1H), 7.41 (d, J=1.0 Hz, 1H), 7.32 (d, J=7.8 Hz, 1H), 7.09 (dd, J=1.3, 7.7 Hz, 1H), 5.18 (s, 2H), 5.01 (s, 2H), 3.77 (td, J=2.9, 5.9 Hz, 1H), 3.34 (q, J=7.0 Hz, 2H), 1.32 (t, J=7.1 Hz, 3H), 0.85-0.79 (m, 2H), 0.78-0.72 (m, 2H).

Compound 092 was prepared in a similar manner to Scheme 1 using TFA instead of CsF and was obtained as a white solid (20.4 mg, 41.22 umol, 36.81% yield, 100% purity). LCMS: Rt=0.416 min, m/z=495.1 (M+H⁺) 1H NMR (400 MHz, chloroform-d) δ=12.97-11.80 (m, 1H), 8.37-8.28 (m, 2H), 7.38-7.32 (m, 3H), 7.23 (br s, 1H), 7.17 (s, 1H), 6.84 (br d, J=8.9 Hz, 1H), 5.25-5.15 (m, 2H), 4.99 (s, 2H), 3.73 (br d, J=2.6 Hz, 1H), 3.35 (q, J=6.6 Hz, 2H), 1.33 (br t, J=6.8 Hz, 3H), 0.82-0.76 (m, 2H), 0.72 (br s, 2H).

Compound 093 was prepared in a similar manner to Scheme 1 and was obtained as a white solid (4.2 mg, 8.98 umol, 26.43% yield, 98% purity). LCMS: Rt=0.383 min, m/z=459.0 (M+H)⁺ 1H NMR (400 MHz, chloroform-d) δ=12.48-11.84 (m, 1H), 8.26 (br d, J=4.6 Hz, 2H), 7.49-7.38 (m, 1H), 6.91-6.77 (m, 3H), 5.13 (s, 2H), 4.98-4.82 (m, 2H), 4.19 (q, J=7.0 Hz, 2H), 3.03 (s, 3H), 1.54 (t, J=7.0 Hz, 3H).

Compound 105 was prepared in a similar manner to Scheme 1 and was obtained as a white solid (5.8 mg, 12.16 umol, 23.71% yield, and 95.3% purity). LCMS: Rt=0.384 min, m/z=454.13 (M+H⁺) ¹H NMR: (400 MHz, chloroform-d) δ=12.37-12.07 (m, 1H), 8.45 (d, J=2.5 Hz, 1H), 8.27 (s, 1H), 7.59 (br s, 1H), 7.45 (d, J=4.9 Hz, 1H), 7.27-7.25 (m, 1H), 7.19-7.08 (m, 1H), 6.98-6.86 (m, 1H), 5.94-5.78 (m, 2H), 4.95 (s, 2H), 3.91-3.83 (m, 1H), 3.07 (s, 3H), 0.91-0.85 (m, 4H).

Compound 110 was prepared in a similar manner to Scheme 1 and was obtained as a white solid (3.7 mg, 8.06 umol, 15.71% yield, 99% purity). LCMS: Rt=0.372 min, m/z=455.1 (M+H⁺). HNMR: (400 MHz, chloroform-d) δ=12.53-12.11 (m, 1H), 8.48 (s, 1H), 8.29 (s, 1H), 7.56 (d, J=1.3 Hz, 1H), 7.37-7.31 (m, 2H), 7.25 (s, 1H), 6.90 (d, J=10.0 Hz, 1H), 6.07-5.80 (m, 2H), 4.96 (s, 2H), 3.80 (d, J=3.4 Hz, 1H), 3.08 (s, 3H), 0.83 (t, J=5.0 Hz, 2H), 0.81-0.77 (m, 2H).

Compound 173 was prepared in a similar manner to Scheme 1 using TFA instead of CsF and was obtained as a white solid (14.2 mg, 31.05 umol, 36.06% yield, 98.5% purity). LCMS: Rt=0.373 min, m/z=451.3 (M+H⁺) ¹H NMR (400 MHz, chloroform-d) δ=12.24 (br s, 1H), 8.35 (d, J=2.1 Hz, 1H), 7.40-7.28 (m, 4H), 7.09-7.00 (m, 3H), 5.86 (s, 1H), 5.89-5.70 (m, 1H), 5.74 (s, 1H), 5.11 (s, 2H), 4.07 (q, J=7.0 Hz, 2H), 3.04 (s, 3H), 2.81 (s, 3H), 1.38 (t, J=6.9 Hz, 3H).

Compound 183 was prepared in a similar manner to Scheme 1 using TFA instead of CsF and was obtained as a white solid (29.0 mg, 60.80 umol, 25.33% yield, 99.5% purity). LCMS: Rt=0.393 min, m/z=477.0 (M+H⁺) ¹H NMR: ¹H NMR (400 MHz, chloroform-d) δ=12.64-12.08 (m, 1H), 8.30 (br d, J=13.6 Hz, 2H), 7.45 (s, 1H), 7.38 (s, 1H), 7.36-7.30 (m, 4H), 7.08-7.04 (m, 1H), 5.17 (s, 2H), 5.03-4.98 (m, 2H), 3.75 (tt, J=3.0, 5.8 Hz, 1H), 3.38-3.31 (m, 2H), 1.32 (t, J=7.1 Hz, 3H), 0.84-0.73 (m, 4H).

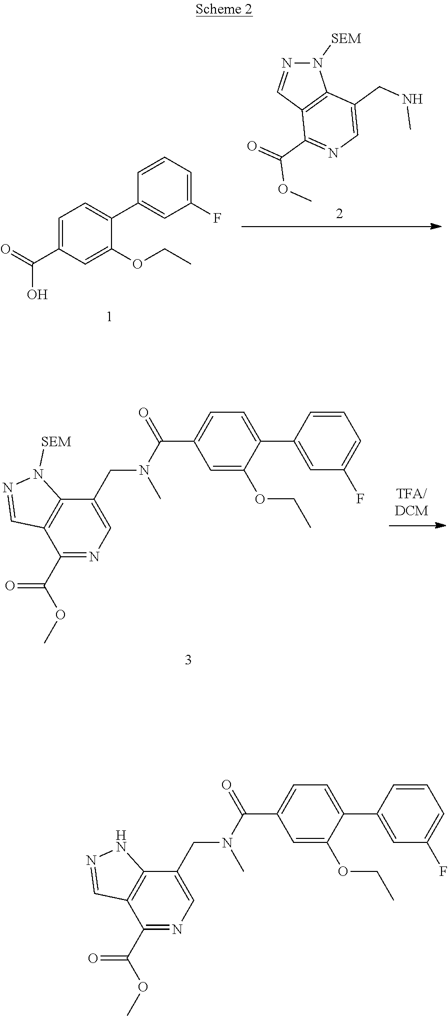 [SMILES: CCOc1cc(C(=O)O)ccc1-c1cccc(F)c1 ; CNCc1cnc(C(=O)OC)c2cnn(COCC[Si](C)(C)C)c12 ; CCOc1cc(C(=O)N(C)Cc2cnc(C(=O)OC)c3cnn(COCC[Si](C)(C)C)c23)ccc1-c1cccc(F)c1 ; CCOc1cc(C(=O)N(C)Cc2cnc(C(=O)OC)c3cn[nH]c23)ccc1-c1cccc(F)c1]

To a solution of compound 1 (200 mg, 768.46 umol, 1 eq) and compound 2 (323.20 mg, 922.16 umol, 1.2 eq) in DCM (4 mL) was added EDCI (220.97 mg, 1.15 mmol, 1.5 eq) and DMAP (9.39 mg, 76.85 umol, 0.1 eq). The mixture was stirred at 20° C. for 12 hr. The reaction mixture was filtered and concentrated under reduced pressure to give a residue. The residue was purified by prep-TLC (SiO₂, PE:EtOAc=0:1). Compound 3 (450 mg, 607.36 umol, 79.04% yield, 80% purity) was obtained as a yellow oil. LCMS: Rt=0.525 min, m/z=593.9 (M+H⁺)

To a solution of compound 3 (150 mg, 253.07 umol, 1 eq) in DCM (2 mL) was added TFA (3.08 g, 27.01 mmol, 2 mL, 106.74 eq). The mixture was stirred at 20° C. for 2 hr. The reaction mixture was concentrated under reduced pressure to give a residue. The residue was purified by prep-HPLC (column: Phenomenex luna C18 150*25 mm*10 um; mobile phase: [water (FA)-ACN]; B %: 40%-70c)/0.10 min). Then the purified solution was lyophilized to give the product. 002 (24.6 mg, 52.98 umol, 20.94% yield, 99.6% purity) was obtained as a white solid. LCMS: Rt=0.455 min, m/z=462.17 (M+H⁺) ¹H NMR: (400 MHz, DMSO-d₆) δ=14.10-13.56 (m, 1H), 8.60 (br s, 1H), 8.44 (br s, 1H), 7.43 (br d, J=6.3 Hz, 2H), 7.40-7.30 (m, 2H), 7.25-7.08 (m, 3H), 5.07 (br s, 2H), 4.13 (br s, 2H), 3.99 (s, 3H), 3.07 (br s, 3H), 1.37-1.19 (m, 3H).

Compound 021 was prepared in a similar manner to Scheme 2 using CsF instead of TFA and was obtained as a green solid (10.2 mg, 21.75 umol, 26.07% yield, 100% purity). LCMS: Rt=0.361 min, m/z=469.0 (M+H)⁺ 1H NMR (400 MHz, chloroform-d) δ ppm 12.20-12.70 (m, 1H) 8.26-8.38 (m, 2H) 7.60-7.64 (m, 1H) 7.43-7.46 (m, 1H) 7.29 (d, J=4.00 Hz, 1H) 7.09 (dd, J=8.07, 1.06 Hz, 1H) 6.89-6.93 (m, 1H) 5.17-5.23 (m, 2H) 4.97 (s, 2H) 3.86-3.95 (m, 1H) 3.05-3.09 (m, 3H) 0.88-0.93 (m, 4H).

Compound 028 was prepared in a similar manner to Scheme 2 and was obtained as a white solid (17.3 mg, 37.15 umol, 36.72% yield, 99.4% purity). LCMS: Rt=0.401 min, m/z=463.4 (M+H)⁺ 1H NMR (400 MHz, chloroform-d) δ=8.42-8.32 (m, 2H), 7.46-7.43 (m, 2H), 7.37-7.29 (m, 4H), 7.11 (d, J=7.8 Hz, 1H), 5.23 (s, 2H), 4.98 (s, 2H), 3.80-3.75 (m, 1H), 3.09 (s, 3H), 0.85-0.79 (m, 2H), 0.78-0.72 (m, 2H).

Compound 032 was prepared in a similar manner to Scheme 2 and was obtained as a white solid (3.5 mg, 7.31 umol, 7.42% yield, 100% purity). LCMS: Rt=0.408 min, m/z=478.16 (M+H⁺) ¹H NMR: ¹H NMR (400 MHz, chloroform-d) δ=12.46-12.28 (m, 1H), 8.34-8.34 (m, 1H), 8.40-8.33 (m, 1H), 8.27 (s, 1H), 7.36-7.33 (m, 3H), 7.23 (br d, J=4.0 Hz, 1H), 7.18 (s, 1H), 6.85 (d, J=8.9 Hz, 1H), 4.97 (s, 2H), 3.77-3.71 (m, 1H), 3.34 (q, J=7.0 Hz, 2H), 3.02 (s, 3H), 1.33 (t, J=7.1 Hz, 3H), 0.81-0.71 (m, 4H).

Compound 033 was prepared in a similar manner to Scheme 2 and was obtained as a white solid (10.2 mg, 23.46 umol, 43.85% yield, and 99% purity). LCMS: Rt=0.452 min, m/z=450.9 (M+H⁺). 1H NMR: 1H NMR (chloroform-d): δ 12.05-12.27 (1H, m), 9.05 (1H, s), 8.22 (1H, s), 7.33-7.44 (5H, m), 7.23 (1H, s), 6.86-6.91 (1H, m), 5.12 (2H, s), 3.67-3.81 (1H, m), 3.08 (3H, s), 2.87 (3H, s), 0.75-0.81 (2H, m), 0.68-0.74 (2H, m).

Compound 038 was prepared in a similar manner to Scheme 2 and was obtained as a white solid (53.1 mg, 118.40 umol, 42.83% yield, 100% purity). LCMS: Rt=0.388 min, m/z=449.4 (M+H)⁺ 1H NMR (400 MHz, chloroform-d) δ=8.57 (s, 1H), 8.50 (s, 1H), 7.42-7.34 (m, 1H), 7.24 (s, 1H), 7.15-7.04 (m, 3H), 6.93-6.88 (m, 1H), 4.98 (s, 2H), 3.80-3.72 (m, 1H), 3.13 (s, 6H), 0.85-0.78 (m, 2H), 0.76-0.70 (m, 2H).

Compound 048 was prepared in a similar manner to Scheme 2 and was obtained as a white solid (15.4 mg, 32.86 umol, 27.94% yield, 99.2% purity). LCMS: Rt=0.391 min, m/z=465.3 (M+H)⁺ 1H NMR (400 MHz, chloroform-d) δ=12.27-11.83 (m, 1H), 8.31-8.13 (m, 2H), 7.38-7.32 (m, 3H), 7.23 (s, 2H), 6.88 (dd, J=1.1, 9.0 Hz, 1H), 4.92 (s, 2H), 3.75 (tt, J=2.9, 5.9 Hz, 1H), 3.10-3.03 (m, 3H), 2.92-2.85 (m, 3H), 0.83-0.76 (m, 2H), 0.76-0.68 (m, 2H).

Compound 055 was prepared in a similar manner to Scheme 2 and was obtained as a white solid (3.8 mg, 8.18 umol, 23.76% yield, and 97% purity). LCMS: Rt=0.452 min, m/z=450.9 (M+H+). 1H NMR: (chloroform-d): δ 12.22 (1H, br s), 8.33-8.43 (2H, m), 7.28-7.41 (4H, m), 7.00-7.11 (3H, m), 6.49 (1H, q, J=7.0 Hz), 5.71-5.93 (2H, m), 3.99-4.15 (2H, m), 2.81 (3H, s), 1.88 (3H, d, J=7.3 Hz), 1.37 (3H, t, J=7.0 Hz).

Compound 062 was prepared in a similar manner to Scheme 2 and was obtained as a colorless oil (11.5 mg, 25.54 umol, 38.90% yield, 99.6% purity). LCMS: Rt=0.382 min, m/z=449.1 (M+H⁺).

Compound 064 was prepared in a similar manner to Scheme 2 and was obtained as a white solid (53.6 mg, 123.14 umol, 57.54% yield, 98.9% purity). LCMS: Rt=0.372 min, m/z=431.2 (M+H+) ¹H NMR (400 MHz, chloroform-d) δ ppm 0.69-0.75 (m, 2H) 0.76-0.82 (m, 2H) 3.09-3.18 (m, 6H) 3.70-3.80 (m, 1H) 4.96-5.02 (m, 2H) 6.91 (dd, J=9.01, 1.38 Hz, 1H) 7.24 (s, 1H) 7.32-7.36 (m, 2H) 7.36-7.39 (m, 1H) 7.39-7.45 (m, 2H) 8.51 (s, 1H) 8.62 (s, 1H).

Compound 065 was prepared in a similar manner to Scheme 2 and was obtained as a white solid (16.7 mg, 35.23 umol, 22.93% yield, 96.1% purity). LCMS: Rt=0.356 min, m/z=456.4 (M+H+) ¹H NMR (400 MHz, chloroform-d) δ=12.66-12.38 (m, 1H), 8.50-8.45 (m, 1H), 8.42 (s, 1H), 7.68-7.64 (m, 2H), 7.61-7.57 (m, 1H), 7.56-7.51 (m, 1H), 7.25 (s, 1H), 6.91 (s, 1H), 5.01-4.89 (m, 2H), 3.80-3.73 (m, 1H), 3.13-3.08 (m, 3H), 3.07-3.02 (m, 3H), 0.85-0.78 (m, 2H), 0.75-0.68 (m, 2H).

Compound 068 was prepared in a similar manner to Scheme 2 and was obtained as a yellow oil (13.6 mg, 28.14 umol, 23.93% yield, 96.2% purity). LCMS: Rt=0.407 min, m/z=465.3 (M+H+). ¹H NMR: (400 MHz, chloroform-d) δ=13.01-12.56 (m, 1H), 9.41 (s, 1H), 8.46 (s, 1H), 7.35 (t, J=4.3 Hz, 3H), 7.23 (s, 2H), 6.90 (d, J=9.0 Hz, 1H), 5.14 (s, 2H), 3.75 (tt, J=2.9, 5.8 Hz, 1H), 3.12 (s, 3H), 3.01 (s, 3H), 1.25 (t, J=7.0 Hz, 1H), 0.85-0.76 (m, 2H), 0.75-0.70 (m, 2H).

Compound 078 was prepared in a similar manner to Scheme 2 and was obtained as a white solid (10.5 mg, 22.46 umol, 29.08% yield, 100% purity). LCMS: Rt=0.338 min, m/z=468.3 (M+H⁺) ¹H NMR (400 MHz, chloroform-d) δ=12.33 (br s, J=0.8 Hz, 1H), 8.22 (s, 1H), 7.77 (s, 1H), 7.69 (td, J=1.4, 7.9 Hz, 1H), 7.63 (td, J=1.3, 7.8 Hz, 1H), 7.51 (t, J=7.8 Hz, 1H), 7.45 (d, J=1.5 Hz, 1H), 7.32 (d, J=7.4 Hz, 1H), 7.12 (dd, J=1.5, 7.8 Hz, 1H), 5.12-5.10 (m, 4H), 3.78 (td, J=3.0, 5.8 Hz, 1H), 3.07 (s, 3H), 2.87 (s, 3H), 1.92-1.67 (m, 4H), 0.87-0.69 (m, 4H).

Compound 097 was prepared in a similar manner to Scheme 2 and was obtained as a brown solid (19 mg, 42.36 umol, 49.04% yield, and 100% purity). LCMS: Rt=0.783 min, m/z=449.3 (M+H⁺) ¹H-NMR: (400 MHz, chloroform-d) δ=12.42-12.13 (m, 1H), 8.28 (d, J=7.4 Hz, 2H), 7.42-7.35 (m, 2H), 7.34-7.29 (m, 2H), 7.09-6.99 (m, 3H), 5.16 (s, 2H), 5.00 (s, 2H), 4.07 (q, J=6.9 Hz, 2H), 3.34 (br d, J=7.1 Hz, 2H), 1.39 (t, J=7.0 Hz, 3H), 1.30 (t, J=7.1 Hz, 3H).

Compound 100 was prepared in a similar manner to Scheme 2 and was obtained as a white solid (21.1 mg, 43.90 umol, 34.58% yield, and 95.8% purity). LCMS: Rt=0.394 min, m/z=461.1 (M+H⁺) ¹H NMR: (400 MHz, chloroform-d) δ=12.25 (br s, 1H), 8.26 (s, 2H), 7.39 (d, J=1.3 Hz, 1H), 7.37-7.30 (m, 2H), 7.25-7.17 (m, 2H), 7.08-7.00 (m, 2H), 5.14 (br s, 2H), 4.99 (s, 2H), 3.83-3.67 (m, 1H), 3.42-3.25 (m, 2H), 1.31 (br t, J=6.9 Hz, 3H), 0.84-0.78 (m, 2H), 0.78-0.72 (m, 2H).

Compound 103 was prepared in a similar manner to Scheme 2 and was obtained as a white solid (17.3 mg, 37.71 umol, 44.18% yield, 99.3% purity). LCMS: Rt=0.376 min, m/z=456.5 (M+H⁺) 1H NMR (400 MHz, chloroform-d) δ=8.44-8.33 (m, 2H), 7.85 (s, 1H), 7.76 (d, J=8.0 Hz, 1H), 7.63 (d, J=7.6 Hz, 1H), 7.55-7.49 (m, 1H), 7.34 (d, J=7.8 Hz, 1H), 7.05 (d, J=7.8 Hz, 1H), 7.00 (s, 1H), 5.23 (s, 2H), 5.01 (s, 2H), 4.06 (q, J=6.9 Hz, 2H), 3.35 (q, J=6.7 Hz, 2H), 1.36 (t, J=6.9 Hz, 3H), 1.30 (t, J=7.0 Hz, 3H).

Compound 170 was prepared in a similar manner to Scheme 2 and was obtained as a white solid (34.3 mg, 82.69 umol, 27.45% yield, 98% purity). LCMS: Rt=0.357 min, m/z=407.0 (M+H⁺) ¹H NMR: (400 MHz, chloroform-d) δ=8.27 (s, 1H), 8.23 (s, 1H), 7.70 (d, J=7.9 Hz, 1H), 7.60 (dd, J=1.1, 3.7 Hz, 1H), 7.38 (dd, J=1.1, 5.1 Hz, 1H), 7.11 (dd, J=3.8, 5.1 Hz, 1H), 7.09-7.04 (m, 2H), 4.91 (s, 2H), 4.19 (q, J=6.9 Hz, 2H), 3.03 (s, 3H), 2.89 (s, 3H), 1.55 (t, J=6.9 Hz, 3H).

Compound 177 was prepared in a similar manner to Scheme 2 and was obtained as a white solid (20.7 mg, 44.52 umol, 40.77% yield). H-NMR: (400 MHz, chloroform-d) δ=8.34 (s, 1H), 8.26 (s, 1H), 7.49 (s, 1H), 7.39-7.34 (m, 1H), 7.31-7.28 (m, 2H), 7.21 (s, 1H), 7.00-6.95 (m, 1H), 6.93 (s, 1H), 5.16 (s, 2H), 4.94 (s, 2H), 4.04-3.94 (m, 3H), 3.28 (q, J=7.0 Hz, 2H), 1.31 (t, J=6.9 Hz, 3H), 1.24 (t, J=7.0 Hz, 3H).

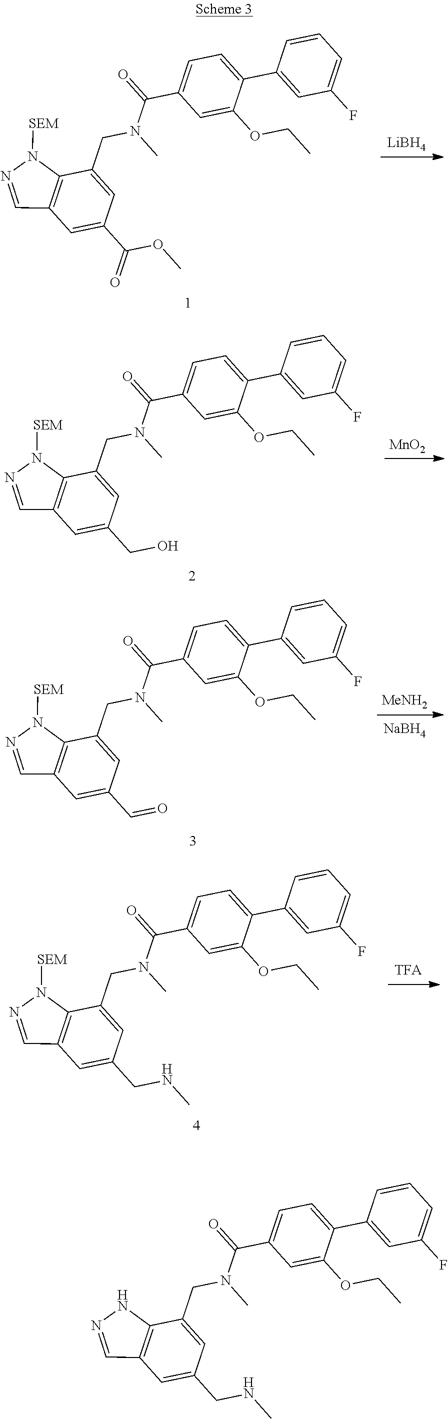

To a solution of Compound 1 (500 mg, 844.96 umol, 1 eq) in MeOH (10 mL) was added LiBH4 (184.03 mg, 8.45 mmol, 10 eq) in 100 ml round-bottom flask (100 mL). The mixture was stirred at 20° C. for 12 hr. The reaction mixture was poured into saturated NH4Cl aqueous (100 mL) and extracted by extracted by EtOAc (100 mL×3), the combined organic phase was dried and concentrated under vacuum to give a residue. The residue was purified by silica gel chromatography (PE:EtOAc=0:0 to 1:1). Compound 2 (87 mg, 154.33 umol, 18.26% yield) was obtained as a white solid. LCMS: Rt=0.633 min, m/z=564.1 (M+H⁺).

To a solution of Compound 2 (87 mg, 154.33 umol, 1 eq) in DCM (2 mL) was added MnO₂ (268.34 mg, 3.09 mmol, 20 eq). The mixture was stirred at 30° C. for 2 hr.

After the reaction was completed, the mixture was filtered and the filtrated was concentrated to give a crude product. The residue was purified by prep-TLC (PE:EtOAc=1:1). Compound 3 (57 mg, 101.47 umol, 65.75% yield) was obtained as a white solid. 1H NMR: (chloroform-d): δ 10.10 (1H, br s), 8.21 (2H, br d, J=13.5 Hz), 7.78-8.03 (1H, m), 7.29-7.46 (3H, m), 6.89-7.22 (4H, m), 5.63-5.99 (2H, m), 5.16-5.44 (2H, m), 3.93-4.34 (2H, m), 3.38-3.70 (2H, m), 3.04-3.28 (3H, m), 1.22-1.41 (3H, m), 0.61-1.02 (2H, m), −0.07 (9H, br s)

Dissolved Compound 3 (57 mg, 101.47 umol, 1 eq) in MeOH (2 mL) in a 100 single-neck bottle was added HOAc (304.69 ug, 5.07 umol, 0.29 uL, 0.05 eq) and MeNH₂ in THF (2 M, 152.21 uL, 3 eq), the reaction mixture was stirred at 20° C. for 2 hr by magnetic stirring apparatus. Then NaBH4 (7.68 mg, 202.95 umol, 2 eq) was added at 0° C., the mixture was stirred at 20° C. for 1 hr by magnetic stirring apparatus. After the reaction was completed, the reaction mixture was poured into NH₄Cl (10 mL), filtered, the filtrate was concentrated under reduced pressure to give a residue. Then the solution was triturated in EtOAc and THF (10 mL) and collected by filtration, the filtrate was concentrated under reduced pressure to give a residue. Compound 4 (57 mg, 98.83 umol, 97.39% yield) was obtained as a yellow oil, which was used into the next step without further purification. LCMS: Rt=0.678 min, m/z=577.2 (M+H⁺).

To a solution of Compound 4 (57 mg, 98.83 umol, 1 eq) in DCM (2 mL) was added TFA (1.26 g, 11.04 mmol, 817.15 uL, 111.68 eq) at 8 mL glass bottle. The mixture was stirred at 20° C. for 2 hr. The reaction mixture was concentrated and under reduced pressure to give a residue. The residue was purified by prep-HPLC (column: Waters xbridge 150*25 mm 10 um; mobile phase: [water (NH₄HCO₃)-ACN]; B %: 34%-64%, 8 min), the purified product was lyophilized. Compound 003 (8.6 mg, 19.26 umol, 19.49% yield, and 100% purity) was obtained as a white solid. LCMS: Rt=0.379 min, m/z=447.4 (M+H⁺). 1H NMR: (chloroform-d): δ 11.57-12.23 (1H, m), 8.08 (1H, s), 7.68 (1H, s), 7.28-7.40 (5H, m), 7.00-7.09 (3H, m), 4.96 (2H, s), 4.06 (2H, q, J=7.0 Hz), 3.89 (2H, s), 3.01 (3H, s), 2.51 (3H, s), 1.36 (3H, t, J=6.9 Hz).

Compound 005 was prepared in a similar manner to Scheme 3 and was obtained as a white solid (16.3 mg, 35.15 umol, 50.55% yield, 99.3% purity). LCMS: Rt=0.387 min, m/z=461.4 (M+H)⁺ 1H NMR (400 MHz, chloroform-d) δ ppm 8.43-8.52 (m, 1H) 8.21-8.25 (m, 1H) 7.26-7.38 (m, 4H) 7.25 (s, 1H) 7.16-7.20 (m, 1H) 6.98-7.07 (m, 3H) 4.94 (s, 2H) 4.00-4.08 (m, 4H) 2.96-3.02 (m, 3H) 2.45-2.52 (m, 6H) 1.31-1.38 (m, 3H).

Compound 006 was prepared in a similar manner to Scheme 3 using dimethyl amine rather than methylamine and was obtained as a white solid (8.3 mg, 17.66 umol, 14.91% yield, and 98% purity). LCMS: Rt=0.346 min, m/z=461.4 (M+H+). 1H NMR: (chloroform-d): δ 11.94 (1H, br s), 8.07 (1H, s), 7.63 (1H, s), 7.28-7.40 (5H, m), 7.00-7.09 (3H, m), 4.96 (2H, s), 4.07 (2H, q, J=6.9 Hz), 3.57 (2H, s), 3.01 (3H, s), 2.30 (6H, s), 1.37 (3H, t, J=7.0 Hz).

Compound 024 was prepared in a similar manner to Scheme 3 using methyl grignard instead of methylamine and was obtained as a white solid (13.8 mg, 28.48 umol, 36.48% yield). LCMS: Rt=0.399 min, m/z=485.1 (M+H⁺) ¹H-NMR: (400 MHz, chloroform-d) δ=12.17 (br s, 1H), 8.33 (s, 1H), 8.27 (s, 1H), 7.00-6.94 (m, 2H), 6.90-6.81 (m, 3H), 5.34 (d, J=6.6 Hz, 1H), 4.95 (s, 2H), 4.07 (q, J=7.0 Hz, 2H), 3.07 (s, 3H), 1.70 (d, J=6.6 Hz, 3H), 1.35 (t, J=6.9 Hz, 3H).

Compound 025 was prepared in a similar manner to Scheme 3 using ethyl grignard instead of methylamine and was obtained as a white solid (4.1 mg, 8.76 umol, 17.31% yield, and 98.8% purity). LCMS: Rt=0.389 min, m/z=463.5 (M+H⁺) ¹H NMR: (400 MHz, chloroform-d) δ=12.36 (br d, J=1.8 Hz, 1H), 8.31 (s, 1H), 8.26 (s, 1H), 7.41-7.28 (m, 4H), 7.11-7.07 (m, 2H), 7.07-7.02 (m, 1H), 5.20-5.13 (m, 1H), 5.02-4.88 (m, 2H), 4.11-4.05 (m, 2H), 3.06 (s, 3H), 1.97-1.82 (m, 2H), 1.38 (t, J=6.9 Hz, 3H), 1.02 (t, J=7.4 Hz, 3H).

Compound 026 was prepared in a similar manner to Scheme 3 using methyl grignard instead of methylamine and was obtained as a white solid (8.8 mg, 18.86 umol, 29.62% yield). LCMS: Rt=0.392 min, m/z=466.9 (M+H⁺) ¹H-NMR: (400 MHz, chloroform-d) δ=12.41 (br s, 1H), 8.35 (s, 1H), 8.29 (s, 1H), 7.36 (d, J=7.6 Hz, 1H), 7.15-7.06 (m, 4H), 6.81 (s, 1H), 5.37 (br d, J=6.6 Hz, 1H), 4.97 (s, 2H), 4.10 (q, J=7.0 Hz, 2H), 3.07 (s, 3H), 1.71 (d, J=6.6 Hz, 3H), 1.40 (t, J=7.0 Hz, 3H).

Compound 029 was prepared in a similar manner to Scheme 3 using methyl grignard instead of methylamine and was obtained as a white solid (6.7 mg, 14.71 umol, 21.54% yield, 100% purity). LCMS: Rt=0.366 min, m/z=456.4 (M+H)⁺ 1H NMR (400 MHz, chloroform-d) δ ppm 12.31-12.57 (m, 1H) 8.37 (s, 1H) 8.26-8.31 (m, 1H) 7.84-7.87 (m, 1H) 7.74-7.80 (m, 1H) 7.61-7.66 (m, 1H) 7.50-7.55 (m, 1H) 7.32-7.36 (m, 1H) 7.09 (s, 2H) 5.35-5.43 (m, 1H) 4.94-4.99 (m, 2H) 4.05-4.12 (m, 2H) 3.06-3.09 (m, 3H) 1.72 (br d, J=6.63 Hz, 3H) 1.37 (t, J=7.00 Hz, 3H).

Compound 030 was prepared in a similar manner to Scheme 3 using methyl grignard instead of methylamine and was obtained as a white solid (98.5% purity). LCMS: Rt=0.393 min, m/z=464.16 (M+H⁺) ¹H NMR: (400 MHz, chloroform-d) δ=12.47-12.21 (m, 1H), 8.32 (s, 1H), 8.26 (s, 1H), 7.59-7.58 (m, 1H), 7.55 (s, 1H), 7.42 (br d, J=6.1 Hz, 1H), 7.38-7.30 (m, 3H), 7.12-7.05 (m, 2H), 5.37-5.31 (m, 1H), 4.95 (s, 2H), 4.07 (q, J=6.8 Hz, 2H), 3.06 (s, 3H), 1.69 (br d, J=6.6 Hz, 3H), 1.38 (t, J=6.9 Hz, 3H).

Compound 031 was prepared in a similar manner to Scheme 3 using methyl grignard instead of methylamine and was obtained as a white solid (16.7 mg, 38.79 umol, 21.75% yield, 100% purity). LCMS: Rt=0.377 min, m/z=431.4 (M+H)⁺ 1H NMR (400 MHz, chloroform-d) δ=13.07-12.39 (m, 1H), 8.47 (s, 1H), 8.40-8.34 (m, 1H), 7.59-7.51 (m, 2H), 7.46-7.32 (m, 4H), 7.13-7.05 (m, 2H), 5.54-5.45 (m, 1H), 5.03-4.93 (m, 2H), 4.06 (q, J=6.9 Hz, 2H), 3.12-3.08 (m, 3H), 1.76 (br d, J=6.5 Hz, 3H), 1.37 (t, J=6.9 Hz, 3H).

Compound 070 was prepared in a similar manner to Scheme 3 using methyl grignard instead of methylamine and was obtained as a white solid (7 mg, 15.23 umol, 44.22% yield, 98.01% purity). LCMS: Rt=0.438 min, m/z=451.2 (M+H)⁺ 1H NMR (400 MHz, chloroform-d) δ ppm 12.14-12.36 (m, 1H) 8.45 (d, J=3.00 Hz, 1H) 8.25 (s, 1H) 7.35 (d, J=7.50 Hz, 2H) 7.28-7.33 (m, 2H) 7.01-7.11 (m, 3H) 5.90-6.11 (m, 1H) 4.88-5.01 (m, 2H) 4.02-4.12 (m, 2H) 3.05 (s, 3H) 1.80-1.90 (m, 3H) 1.38 (t, J=6.94 Hz, 3H).

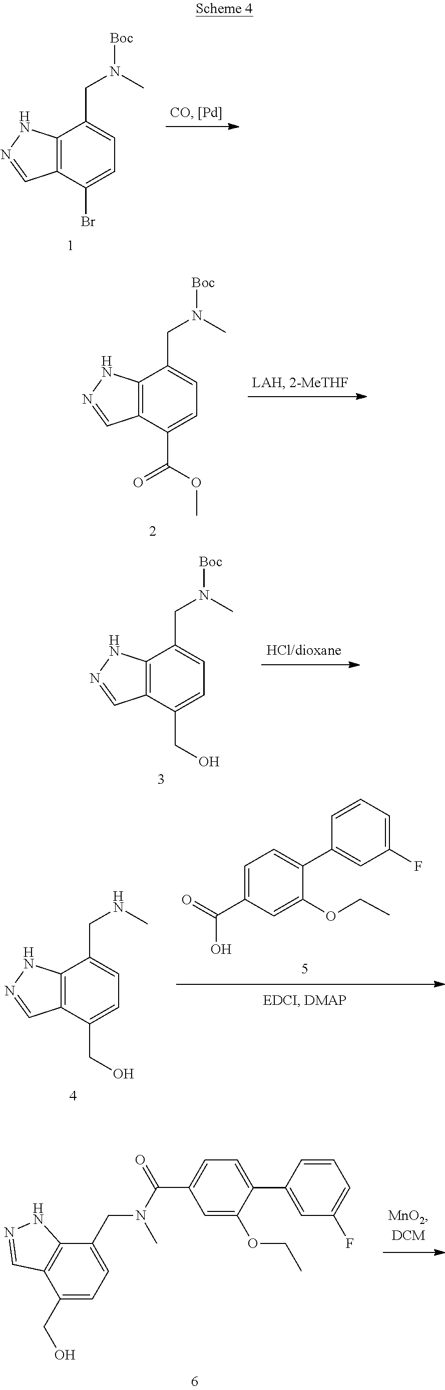

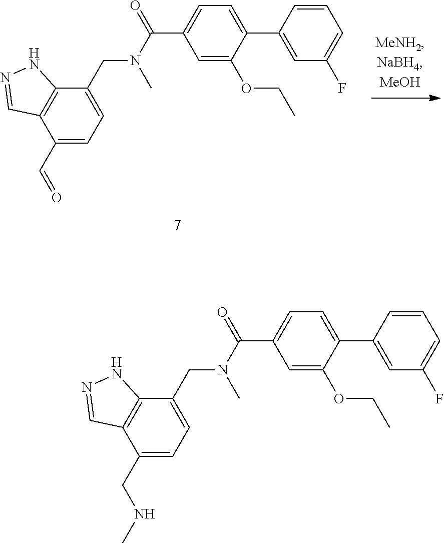 [SMILES: CCOc1cc(C(=O)N(C)Cc2ccc(C=O)c3cn[nH]c23)ccc1-c1cccc(F)c1 ; CCOc1cc(C(=O)N(C)Cc2ccc(CNC)c3cn[nH]c23)ccc1-c1cccc(F)c1]

To a solution of Compound 1 (5 g, 14.70 mmol, 1 eq) in MeOH (60 mL) was added Pd(OAc)₂ (329.95 mg, 1.47 mmol, 0.1 eq) and DPPF (1.63 g, 2.94 mmol, 0.2 eq), TEA (4.46 g, 44.09 mmol, 6.14 mL, 3 eq). The mixture was stirred at 80° C. for 12 hr under CO (50 psi).

The reaction mixture was filtered and the filtrate was concentrated under vacuum to give a residue. The residue was purified by column chromatography (SiO₂, EtOAc/Methanol=100/1 to 5/1). The purified solution was concentrated under vacuum to give a residue. Compound 2 (570 mg, 1.78 mmol, 12.11% yield) was obtained as a yellow solid. LCMS: Rt=0.504 min, m/z=319.9 (M+H)⁺

To a 100 mL three neck flask solution of Compound 2 (570 mg, 1.78 mmol, 1 eq) in 2-MeTHF (10 mL) was added LAH (101.61 mg, 2.68 mmol, 1.5 eq) at 0° C. The mixture was stirred at 0° C. for 12 hr. After the reaction was completed, the resulting mixture was added Na₂SO₄·10H₂O (3 g). Then the mixture was filtered and the filtrate was concentrated to give a crude product. The residue was purified by column chromatography (SiO₂, PE/EtOAc=10/1 to 1/2). The purified solution was concentrated under vacuum to give a residue. Compound 3 (200 mg, 686.47 umol, 38.46% yield) was obtained as a colorless oil. LCMS: Rt=0.367 min, m/z=292.1 (M+H)⁺

To a 100 mL round-bottomed flask solution of Compound 3 (200 mg, 686.47 umol, 1 eq) in EtOAc (2 mL) was added HCl/dioxane (4 M, 2 mL, 11.65 eq). The mixture was stirred at 20° C. for 1 hr. The reaction mixture was concentrated under vacuum to give residue. Compound 4 (130 mg, crude, HCl) was obtained as a white solid which was used in the next step. LCMS: Rt=0.122 min, m/z=161.1 (M−30)⁺

To a solution of Compound 4 (130 mg, 570.95 umol, 1 eq, HCl), Compound 5 (148.60 mg, 570.95 umol, 1 eq) in DCM (5 mL) was added EDCI (164.18 mg, 856.43 umol, 1.5 eq) DMAP (6.98 mg, 57.10 umol, 0.1 eq) and DIPEA (221.37 mg, 1.71 mmol, 298.35 uL, 3 eq). The mixture was stirred at 20° C. for 12 hr. The reaction mixture was poured into water (15 mL) and extracted by EtOAc (10 mL×3), the combined organic phase was dried and concentrated under vacuum to give residue. The residue was purified by column chromatography (SiO₂, PE/EtOAc=10/1 to 1/4). The purified solution was concentrated under vacuum to give a residue. Compound 6 (160 mg, 369.11 umol, 64.65% yield) was obtained as a colorless oil. LCMS: Rt=0.451 min, m/z=434.3 (M+H)⁺

To a 8 mL glass bottle solution of Compound 6 (160 mg, 369.11 umol, 1 eq) in DCM (3 mL) was added MnO₂ (481.34 mg, 5.54 mmol, 15 eq). The mixture was stirred at 20° C. for 2 hr. After the reaction was completed, the mixture was filtered and the filtrated was concentrated to give a crude product. The residue was purified by prep-TLC (PE/EtOAc=1:1, RF=0.3), the purified solution was concentrated under vacuum to give a residue. Compound 7 (120 mg, 278.13 umol, 75.35% yield) was obtained as a colorless oil. LCMS: Rt=0.480 min, m/z=432.3 (M+H)⁺

To a 50 mL round-bottomed flask solution of Compound 7 (30 mg, 69.53 umol, 1 eq) in MeOH (2 mL) was added MeNH₂ (2 M, 104.30 uL, 3 eq) and AcOH (208.78 ug, 3.48 umol, 1.99e-1 uL, 0.05 eq) at 20° C., the mixture was stirred at 20° C. for 2 hr, Then the mixture was added NaBH₄ (5.26 mg, 139.06 umol, 2 eq) at 0° C. The mixture was stirred at 20° C. for 2 hr. After the reaction was completed, the reaction mixture was poured into NH₄Cl (5 mL) and control the temperature between 1020° C., then the mixture was added Na₂CO₃ (1 g), the solution was filtered, the filtrate was concentrated under reduced pressure to give a residue. Then the solution was triturated in DCM and MeOH and collected by filtration, the filtrate was concentrated under reduced pressure to give a residue. The residue was purified by prep-HPLC (column: Welch Ultimate C18 150*25 mm*5 um; mobile phase: [water (FA)-ACN]; B %: 18%-48%, 10 min), the purified solution was lyophilized to give a white solid. 004 (21.6 mg, 46.58 umol, 67.00% yield, 96.3% purity) was obtained as a white solid, LCMS: Rt=0.381 min, m/z=447.4 (M+H)⁺ 1H NMR (400 MHz, chloroform-d) δ ppm 8.39-8.48 (m, 1H) 8.22-8.28 (m, 1H) 7.22-7.36 (m, 5H) 7.16-7.20 (m, 1H) 6.97-7.04 (m, 3H) 4.82-4.90 (m, 2H) 4.21-4.32 (m, 2H) 4.02 (q, J=6.84 Hz, 2H) 2.92-3.01 (m, 3H) 2.48-2.59 (m, 3H) 1.28-1.38 (m, 3H).

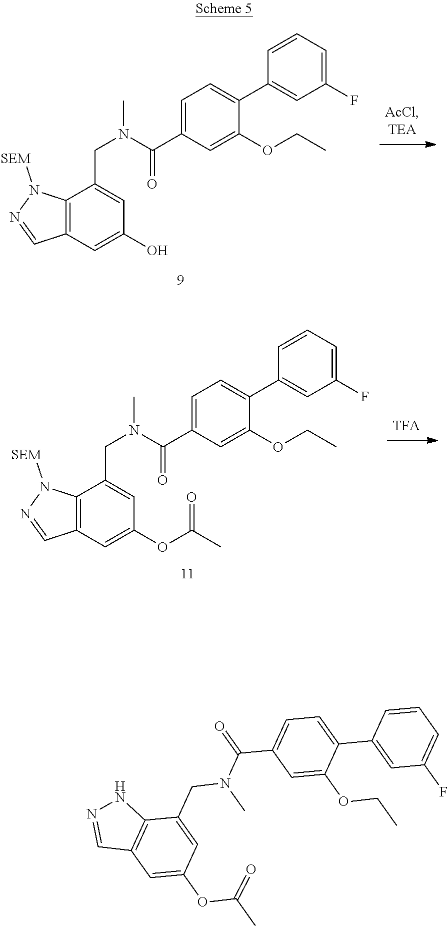 [SMILES: CCOc1cc(C(=O)N(C)Cc2cc(O)cc3cnn(COCC[Si](C)(C)C)c23)ccc1-c1cccc(F)c1 ; CCOc1cc(C(=O)N(C)Cc2cc(OC(C)=O)cc3cnn(COCC[Si](C)(C)C)c23)ccc1-c1cccc(F)c1 ; CCOc1cc(C(=O)N(C)Cc2cc(OC(C)=O)cc3cn[nH]c23)ccc1-c1cccc(F)c1]

To a 8 mL glass bottle was added a solution of compound 9 (50 mg, 90.96 umol, 1 eq) in DCM (2 mL). Then Et₃N (18.41 mg, 181.92 umol, 25.32 uL, 2 eq) and acetyl chloride (8.57 mg, 109.15 umol, 7.79 uL, 1.2 eq) was added into the the above mixture. The mixture was stirred at 20° C. for 12 hr. The mixture was poured into H₂O (20 mL). The aqueous phase was extracted with EtOAc (20 mL×2). The combined organic was dried over Na₂SO₄, concentrated under vacuum. The residue was used directly for the next step without any purification. Compound 11 (53 mg, 89.57 umol, 98.47% yield, N/A purity) was obtained as off-white solid. LCMS: Rt=0.690 min, m/z=474.3 (M+H⁺).

To a solution of compound 11 (73 mg, 123.36 umol, 1 eq) in DCM (2 mL) was added TFA (1.54 g, 13.51 mmol, 1 mL, 109.48 eq) in a 100 mL round-bottom flask. The mixture was stirred at 20° C. for 2 hr. The mixture was concentrated under reduced pressure to give a residue. The residue was purified by prep-HPLC (column: Phenomenex C18 150*25 mm*10 um; mobile phase: [water(NH₄HCO₃)-ACN]; B %: 36%-66%, 8 min). Compound 007 (7.7 mg, 16.02 umol, 12.98% yield, 96% purity) was obtained as a white solid. LCMS: Rt=0.590 min, m/z=462.2 (M+H⁺). 1H NMR: (chloroform-d): δ 12.02 (1H, br s), 8.10 (1H, s), 7.48 (1H, d, J=1.8 Hz), 7.29-7.40 (4H, m), 7.00-7.09 (4H, m), 4.94 (2H, s), 4.06 (2H, q, J=6.9 Hz), 3.03 (3H, s), 2.36 (3H, s), 1.37 (3H, t, J=6.9 Hz).

Compound 009 was prepared in a similar manner to Scheme 5 and was isolated as a white solid (31 mg, 62.33 umol, 37.82% yield, 95.8% purity). LCMS: Rt=0.393 min, m/z=477.4 (M+H)⁺ 1H NMR (400 MHz, chloroform-d) δ=12.51-11.86 (m, 1H), 8.28 (s, 1H), 8.23 (s, 1H), 7.33-7.26 (m, 2H), 7.25-7.22 (m, 1H), 7.21-7.19 (m, 1H), 7.02 (br d, J=1.5 Hz, 2H), 6.97-6.94 (m, 1H), 5.51-5.48 (m, 2H), 4.87 (s, 2H), 4.03-3.96 (m, 2H), 2.98 (s, 3H), 2.17-2.14 (m, 3H), 1.33-1.27 (m, 3H).

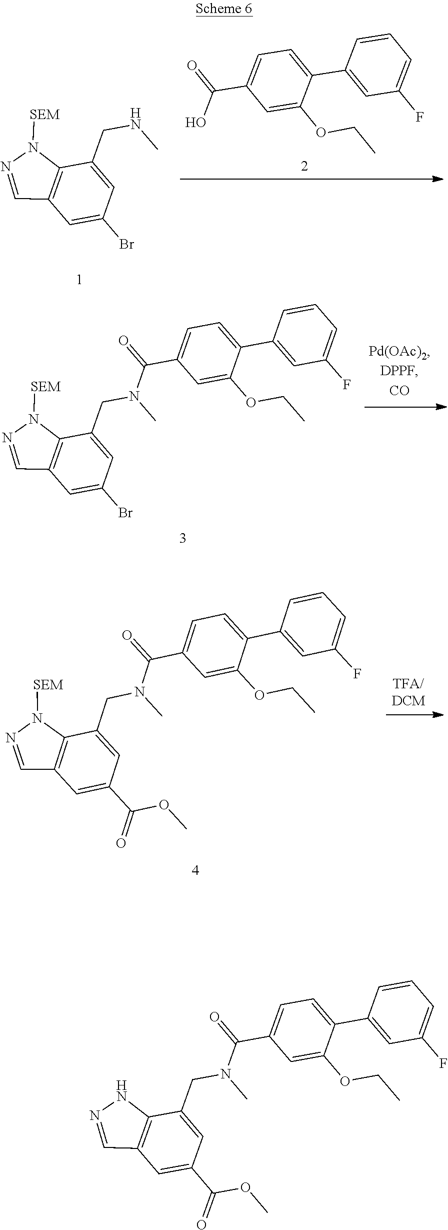 [SMILES: CNCc1cc(Br)cc2cnn(COCC[Si](C)(C)C)c12 ; CCOc1cc(C(=O)O)ccc1-c1cccc(F)c1 ; CCOc1cc(C(=O)N(C)Cc2cc(Br)cc3cnn(COCC[Si](C)(C)C)c23)ccc1-c1cccc(F)c1 ; CCOc1cc(C(=O)N(C)Cc2cc(C(=O)OC)cc3cnn(COCC[Si](C)(C)C)c23)ccc1-c1cccc(F)c1 ; CCOc1cc(C(=O)N(C)Cc2cc(C(=O)OC)cc3cn[nH]c23)ccc1-c1cccc(F)c1]

To a solution of compound 1 (2.85 g, 7.68 mmol, 1 eq) and compound 2 (2 g, 7.68 mmol, 1 eq) in DCM (20 mL) in a 40 mL glass bottle were added EDCI (2.21 g, 11.53 mmol, 1.5 eq) and DMAP (93.88 mg, 768.46 umol, 0.1 eq), the mixture was stirred at 20° C. for 12 hr. The reaction mixture was filtered and concentrated under reduced pressure to give a residue. The residue was purified by column chromatography (SiO₂, PE:EtOAc=2:1). Compound 3 (3.8 g, 6.20 mmol, 80.72% yield) was obtained as white solid. LCMS: Rt=0.610 min, m/z=612.6 (M+H⁺).

To a solution of compound 3 (3.7 g, 6.04 mmol, 1 eq) in MeOH (40 mL) in a 250 mL hydrogenated bottle was added Pd(OAc)2 (271.20 mg, 1.21 mmol, 0.2 eq), TEA (1.83 g, 18.12 mmol, 2.52 mL, 3 eq) and DPPF (669.67 mg, 1.21 mmol, 0.2 eq), the mixture was stirred at 80° C. for 12 hr under CO (50 psi). The reaction mixture was filtered through a pad of celite. The filtered concentrated under vacuum to give a residue. The residue was purified by silica gel chromatography (PE:EtOAc=0:0 to 2:1). Compound 4 was obtained as a yellow solid. LCMS: Rt=0.605 min, m/z=592.3 (M+H⁺).

To a solution of compound 4 (200 mg, 337.98 umol, 1 eq) in DCM (2 mL) was added TFA (1.54 g, 13.51 mmol, 1 mL, 39.96 eq), the mixture was stirred at 20° C. for 1 hr. The reaction mixture was concentrated and under reduced pressure to give a residue. The residue was purified by prep-HPLC (neutral condition, column: Welch Xtimate C18 150*25 mm*5 um; mobile phase: [water(NH₄HCO₃)-ACN]; B %: 44%-74%, 2 min), the purified product was lyophilized and give 008 (140 mg, 302.46 umol, 89.49% yield, 99.7% purity) as white solid. LCMS: Rt=0.531 min, m/z=462.2 (M+H⁺). ¹H NMR: 1H NMR (400 MHz, chloroform-d) δ=12.35-12.10 (m, 1H), 8.56 (d, J=1.1 Hz, 1H), 8.23 (s, 1H), 7.99 (s, 1H), 7.41-7.29 (m, 4H), 7.11-7.02 (m, 3H), 5.00 (s, 2H), 4.07 (q, J=6.8 Hz, 2H), 3.98 (s, 3H), 3.04 (s, 3H), 1.37 (t, J=7.0 Hz, 3H).

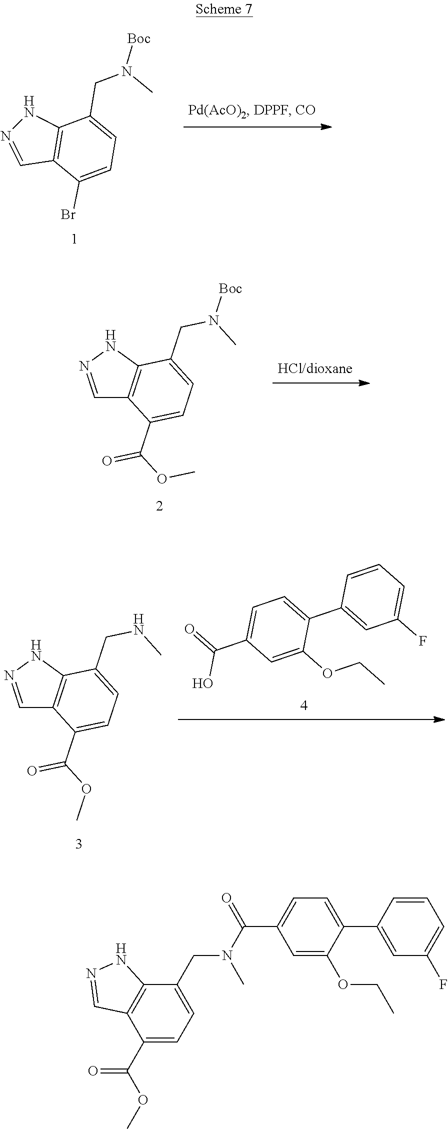

To a solution of Compound 1 (5 g, 14.70 mmol, 1 eq) in MeOH (60 mL) was added Pd(OAc)₂ (329.95 mg, 1.47 mmol, 0.1 eq), DPPF (1.63 g, 2.94 mmol, 0.2 eq) and TEA (4.46 g, 44.09 mmol, 6.14 mL, 3 eq). The mixture was stirred at 80° C. for 12 hr under CO (50 psi). The reaction mixture was filtered and the filtrate was concentrated under vacuum to give a residue. The residue was purified by column chromatography (SiO₂, PE/EtOAc=100/1 to 5/1). The purified solution was concentrated under vacuum to give a residue. Compound 2 (1 g, 3.13 mmol, 21.30% yield) was obtained as a yellow solid. LCMS: Rt=0.485 min, m/z=219.7 (M−100)⁺ 1H NMR (400 MHz, chloroform-d) δ ppm 11.59-12.41 (m, 1H) 8.56 (s, 1H) 7.85-7.90 (m, 1H) 7.23-7.26 (m, 1H) 4.65-4.69 (m, 2H) 4.03 (s, 3H) 2.81 (s, 3H) 1.49-1.54 (m, 9H)

To a 100 mL round-bottomed flask solution of Compound 2 (500 mg, 1.57 mmol, 1 eq) in EtOAc (3 mL) was added HCl/dioxane (4 M, 6 mL, 15.33 eq). The mixture was stirred at 20° C. for 2 hr. The mixture was stirred at 20° C. for 1 hr. The reaction mixture was concentrated under vacuum to give residue. Compound 3 (450 mg, crude, HCl) was obtained as a white solid which was used in the next step. LCMS: Rt=0.225 min, m/z=220.2 (M+H)⁺

To a 100 mL round-bottomed flask solution of Compound 3 (450 mg, 1.76 mmol, 1 eq, HCl), Compound 4 (458.02 mg, 1.76 mmol, 1 eq) in DCM (8 mL) was added EDCI (506.06 mg, 2.64 mmol, 1.5 eq), DMAP (21.50 mg, 175.99 umol, 0.1 eq) and DIPEA (1.14 g, 8.80 mmol, 1.53 mL, 5 eq). The mixture was stirred at 20° C. for 12 hr. The reaction mixture was poured into water (50 mL) and extracted by EtOAc (50 mL×3), the combined organic phase was dried and concentrated under vacuum to give residue. The residue was purified by prep-HPLC (column: Phenomenex luna C18 150*25 mm*10 um; mobile phase: [water (FA)-ACN]; B %: 51%-81%, 10 min), the purified solution was lyophilized to give a white solid. 010 (500 mg, 1.07 mmol, 60.83% yield, 98.8% purity) was obtained as a white solid, LCMS: Rt=0.502 min, m/z=462.3 (M+H)⁺ 1H NMR (400 MHz, chloroform-d) δ ppm 8.59-8.64 (m, 1H) 7.92 (d, J=7.25 Hz, 1H) 7.40 (s, 1H) 7.33-7.36 (m, 2H) 7.28-7.32 (m, 2H) 7.01-7.09 (m, 3H) 5.03 (s, 2H) 4.06-4.10 (m, 2H) 4.05 (s, 3H) 2.99-3.04 (m, 3H) 1.37 (t, J=6.94 Hz, 3H).

Compound 066 was prepared in a similar manner as Scheme 7 with an additional step of reducing the ester by dissolving the esterified compound in 2-MeTHF (5 mL), and adding LAH (16.45 mg, 433.38 umol, 2 eq) at 0° C. After the reaction was completed, the resulting mixture was added Na₂SO₄·10H₂O (5 g). Then the mixture was filtered and the filtrate was concentrated to give a crude product. The residue was purified by prep-HPLC (column: Phenomenex luna C18 150*25 mm*10 um; mobile phase: [water (FA)-ACN]; B %: 49%-79%, 10 min), the purified solution was lyophilized to give a white solid. 066 (6.1 mg, 13.92 umol, 6.42% yield, 98.9% purity) was obtained as a white solid. LCMS: Rt=0.458 min, m/z=434.3 (M+H)⁺ 1H NMR (400 MHz, chloroform-d) δ ppm 8.25-8.29 (m, 1H) 7.29-7.40 (m, 4H) 7.24-7.26 (m, 1H) 7.12-7.15 (m, 1H) 7.00-7.08 (m, 3H) 5.05-5.10 (m, 2H) 4.95-4.98 (m, 2H) 4.03-4.10 (m, 2H) 2.99-3.02 (m, 3H) 1.37 (t, J=7.00 Hz, 3H).

Compound 069 was prepared in a similar manner as Scheme 7 with an additional step of reducing the ester by dissolving the esterified compound in MeOH (2 mL), and adding LiBH4 (18.41 mg, 844.96 umol, 5 eq) at 0° C. The reaction mixture was poured into saturated NH4Cl aqueous (10 mL) and extracted by extracted by EtOAc (10 mL×3), the combined organic phase was dried and concentrated under vacuum to give a residue. The residue was purified by prep-TLC (PE:EtOAc=1:1). Compound 2 (30 mg, 53.22 umol, 31.49% yield) was obtained as a white oil. LCMS: Rt=0.553 min, m/z=446.2 (M+H+).

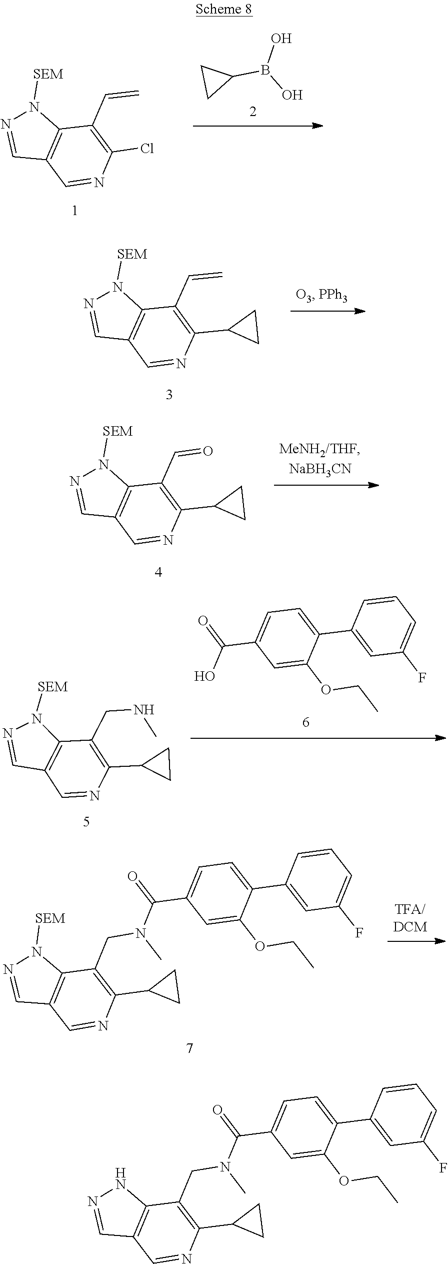

Dissolved Compound 1 (200.00 mg, 645.44 umol, 1 eq) and Compound 2 (166.32 mg, 1.94 mmol, 3 eq) in dioxane (5 mL) and H₂O (0.5 mL) in a 40 mL bottle, and added Cs₂CO₃ (630.89 mg, 1.94 mmol, 3 eq) and Pd(dppf)Cl₂·CH₂Cl₂ (52.71 mg, 64.54 umol, 0.1 eq), the reaction mixture was stirred at 100° C. for 12 hr under N₂. After the reaction was completed and cooled to room temperature, the reaction mixture was filtered by EtOAc (50 mL), the filtrate was concentrated on a rotary evaporator to give a crude product. The crude product was purified by prep-TLC (PE:EtOAc=1:1, Rf=0.7). The purified solution was concentrated on vacuum to give Compound 3 (68 mg, 215.54 umol, 33.39% yield) as a yellow oil. LCMS: Rt=0.405 min, m/z=316.2 (M+H)⁺

To a solution of Compound 3 (74.00 mg, 234.56 umol, 1 eq) in MeOH (8 mL) and DCM (8 mL) in three-necked flask (100 mL) was bubbled with OZONE (11.26 mg, 234.56 umol, 1 eq) under 15 psi at −78° C. for 20 min, then the mixture was purged with nitrogen for 10 min, PPh₃ (123.04 mg, 469.12 umol, 2 eq) was dissolved in DCM (8 mL) was added into the reaction mixture at −78° C. for 10 min. The mixture was stirred at 20° C. for 1 hr. After the reaction was completed, the mixture was concentrated to give a residue. The residue was purified by prep-TLC (SiO₂, PE/EtOAc=1:1). The purified solution was concentrated on vacuum to give Compound 4 (35 mg, 110.25 umol, 47.00% yield) as a white solid. 1H NMR (400 MHz, chloroform-d) δ=11.07 (s, 1H), 9.23 (s, 1H), 8.39 (s, 1H), 5.81 (s, 2H), 3.73-3.67 (m, 2H), 3.30-3.23 (m, 1H), 1.40-1.35 (m, 2H), 1.20-1.14 (m, 2H), 1.01-0.95 (m, 2H), 0.00-0.01 (m, 9H)

To a solution of Compound 4 (35.00 mg, 110.25 umol, 1 eq) in MeOH (2 mL) in round-bottom flask (50 mL) was added AcOH (662.08 ug, 11.03 umol, 6.31e-1 uL, 0.1 eq) and MeNH₂ in THF (2 M, 330.75 uL, 6 eq), The mixture was stirred at 20° C. for 2 hr. Then NaBH₃CN (10.39 mg, 165.38 umol, 1.5 eq) was added into the reaction mixture at 0° C. The mixture was stirred at 20° C. for 3 hr. After the reaction was completed, the reaction mixture was poured into NH₄Cl (0.1 mL), filtered, the filtrate was concentrated under reduced pressure to give a residue. Then the solution was triturated in Dichloromethane (10 mL) and collected by filtration, the filtrate was concentrated under reduced pressure to give Compound 5 (60 mg, crude) as a yellow oil which was used in the next step. LCMS: Rt=0.573 min, m/z=333.0 (M+H)⁺

To a solution of Compound 5 (60 mg, 180.44 umol, 1 eq) and Compound 6 (46.96 mg, 180.44 umol, 1 eq) in DCM (3 mL) in round-bottom flask (50 mL) was added EDCI (51.89 mg, 270.66 umol, 1.5 eq) and DMAP (2.20 mg, 18.04 umol, 0.1 eq). The mixture was stirred at 20° C. for 2 hr. The reaction mixture was concentrated on vacuum to give residue. The residue was purified by prep-TLC (SiO₂, PE/EtOAc=1:1). The purified solution was concentrated on vacuum to give Compound 7 (30 mg, 52.20 umol, 28.93% yield) as a colorless oil. LCMS: Rt=0.482 min, m/z=575.6 (M+H)⁺

To a solution of Compound 7 (30.00 mg, 52.20 umol, 1 eq) in DCM (0.5 mL) in round-bottom flask (100 mL) was added TFA (770.00 mg, 6.75 mmol, 500.00 uL, 129.38 eq). The mixture was stirred at 20° C. for 1 hr. The reaction mixture was concentrated to give a residue. The residue was basified with Et₃N to pH8. The residue was purified by prep-HPLC (column: Phenomenex luna C18 150*25 mm*10 um; mobile phase: [water (FA)-ACN]; B %: 26%-56%, 9 min). The purified solution was lyophilized to give 011 (7 mg, 15.24 umol, 29.21% yield, 96.8% purity) as a white solid. LCMS: Rt=0.402 min, m/z=445.1 (M+H)⁺ 1H NMR (400 MHz, chloroform-d) δ=9.03 (br s, 1H), 8.20 (br s, 1H), 7.41-7.34 (m, 2H), 7.33-7.30 (m, 2H), 7.09-7.00 (m, 3H), 5.29-5.23 (m, 2H), 4.07 (q, J=6.9 Hz, 2H), 3.11 (s, 3H), 2.46-2.37 (m, 1H), 1.40-1.35 (m, 3H), 1.33-1.26 (m, 4H).

Compound 016 was prepared in a similar manner to Scheme 8 and was obtained as a white solid (45.2 mg, 99.11 umol, 95.34% yield, and 98% purity). LCMS: Rt=0.404 min, m/z=447.2 (M+H⁺) ¹H NMR: (400 MHz, chloroform-d) δ=12.15 (br s, 1H), 9.01 (s, 1H), 8.19 (s, 1H), 7.47-7.42 (m, 2H), 7.36-7.30 (m, 4H), 7.11-7.07 (m, 1H), 5.12 (s, 2H), 3.87-3.68 (m, 1H), 3.06 (s, 3H), 2.84 (s, 3H), 0.85-0.79 (m, 2H), 0.78-0.72 (m, 2H).

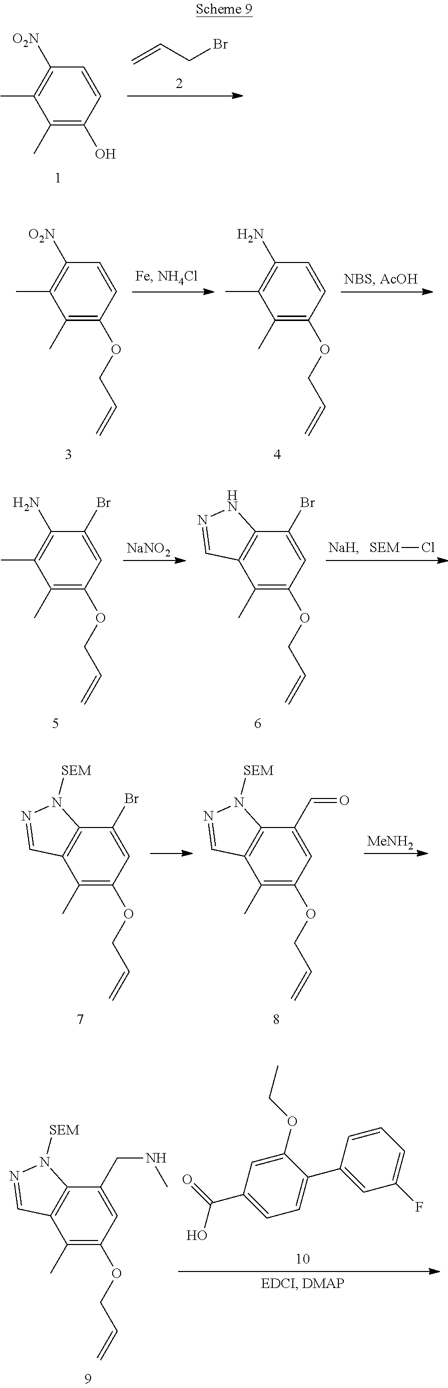

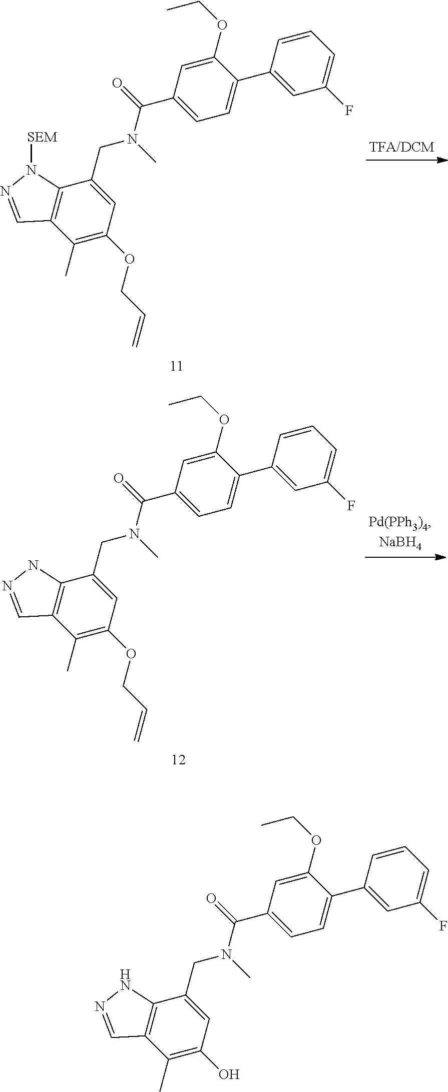 [SMILES: C=CCOc1cc(CN(C)C(=O)c2ccc(-c3cccc(F)c3)c(OCC)c2)c2c(cnn2COCC[Si](C)(C)C)c1C ; C=CCOc1cc(CN(C)C(=O)c2ccc(-c3cccc(F)c3)c(OCC)c2)c2[nH]ncc2c1C ; CCOc1cc(C(=O)N(C)Cc2cc(O)c(C)c3cn[nH]c23)ccc1-c1cccc(F)c1]

To a solution of compound 1 (5 g, 29.91 mmol, 1 eq) and compound 2 (3.62 g, 29.91 mmol, 1 eq) in acetone (50 mL) was added K₂CO₃ (8.27 g, 59.82 mmol, 2 eq) and KI (496.53 mg, 2.99 mmol, 0.1 eq) in a 250 mL three-necked bottle. The mixture was stirred at 60° C. for 12 hr. The reaction mixture was poured into water (200 mL) and extracted by EtOAc (200 mL×3), the combined organic phase was dried and concentrated under vacuum to give residue. Compound 1-allyloxy-2,3-dimethyl-4-nitro-benzene (6.02 g, 29.05 mmol, 97.12% yield) was obtained as a yellow oil, which was used into the next step without further purification. LCMS: Rt=0.475 min, m/z=208.4 (M+H⁺)

To a solution of compound 3 (6.02 g, 29.05 mmol, 1 eq) in EtOH (60 mL) and H₂O (12 mL) was added into 500 mL three-necked bottle, subsequently. The mixture was added Fe (8.11 g, 145.25 mmol, 5 eq), NH₄Cl (15.54 g, 290.51 mmol, 10 eq) in portions at 20° C. The mixture was stirred at 70° C. for 2 hr. The reaction mixture was filtered through a pad of celite, the filtrate was pured into saturated water (500 mL) at 0° C. and extrated with EtOAc (500 mL×3), the combined organic phase was washed with brine (200 mL), dried over anhydrous Na₂SO₄, and concentrated to give a crude product. The residue was purified by silica gel chromatography (PE:EtOAc=10:1). Compound 4 (4.42 g, 24.93 mmol, 85.82% yield, N/A purity) was obtained as brown solid. LCMS: Rt=0.207 min, m/z=178.4 (M+H⁺) ¹H NMR (400 MHz, chloroform-d) δ=6.63 (d, J=8.5 Hz, 1H), 6.55 (d, J=8.5 Hz, 1H), 6.07 (ddt, 1H), 5.41 (qd, J=1.7, 17.3 Hz, 1H), 5.25 (qd, J=1.5, 10.5 Hz, 1H), 4.45 (td, J=1.5, 5.2 Hz, 2H), 3.62 (br. s, 2H), 2.21 (s, 3H), 2.12 (s, 3H).

To a 100 mL three-necked round bottom flask was added a solution of compound 4 (3.3 g, 18.62 mmol, 1 eq) in DCM (30 mL) and HOAc (3 mL). Then Br₂ (2.98 g, 18.62 mmol, 959.82 uL, 1 eq) in DCM (5 mL) as added dropwise into the above mixture at 0° C. The mixture was stirred at 0° C. for 3 hr. The reaction mixture was poured into sat. NaHCO₃ aqueous (300 mL) in portions. The aqueous phase was extracted with EtOAc (200 mL×2). The combined organic phase was washed with H₂O (200 mL×3), brine (200 mL), dried over Na₂SO₄, and concentrated under vacuum. The residue was purified by silica gel column chromatography (PE:EtOAc=90:1). Compound 5 (1.26 g, 3.89 mmol, 20.87% yield, 79% purity) was obtained as brown solid. LCMS: Rt=0.422 min, m/z=256.3, 258.3 (M+H⁺). ¹H NMR (400 MHz, chloroform-d) δ=6.88 (s, 1H), 6.11-6.00 (m, 1H), 5.41 (dd, J=1.0, 17.3 Hz, 1H), 5.27 (dd, J=0.8, 10.5 Hz, 1H), 4.43 (d, J=5.1 Hz, 2H), 3.81 (br s, 2H), 2.17 (d, J=4.6 Hz, 6H).

To a solution of compound 5 (1 g, 3.90 mmol, 1 eq) in HOAc (20 mL) and H₂O (2 mL) in 100 mL single necked round bottom flask was added NaNO₂ (538.77 mg, 7.81 mmol, 2 eq) at 5° C., the mixture was stirred at 20° C. for 12 h. The mixture was poured into water (100 mL) and adjust to pH=8 with saturated NaHCO₃ solution, extracted with EtOAc (100 mL×2), the combined organic phase was washed with brine (100 mL), dried over anhydrous Na₂SO₄, and concentrated to give a crude product. The residue was purified by silica gel column chromatography (PE:EtOAc=10:1). Compound 6 (389 mg, 1.46 mmol, 37.30% yield, N/A purity) was obtained as light brown solid. LCMS: Rt=0.434 min, m/z=267.0, 269.0 (M+H⁺). ¹H NMR (400 MHz, chloroform-d) δ=8.14 (br s, 1H), 7.28 (s, 1H), 6.14-6.04 (m, 1H), 5.47-5.40 (m, 1H), 5.30 (dd, J=1.3, 10.5 Hz, 1H), 4.61-4.52 (m, 2H), 2.47 (s, 3H).

To a solution of compound 6 (276 mg, 1.03 mmol, 1 eq) in 2-MeTHF (5 mL) in 20 mL single-necked round bottom flask was added NaH (82.65 mg, 2.07 mmol, 60% purity, 2 eq) at 0° C., the mixture was stirred at 0° C. for 0.5 h, then SEM-CI (258.39 mg, 1.55 mmol, 274.30 uL, 1.5 eq) was added dropwise, the mixture was warmed to 20° C. and stirred at 20° C. for 2 h. After the reaction was completed, the mixture was poured into satutated NH₄Cl (50 mL) solution at 0° C., and extracted with EtOAc (50 mL×2), the combined organic phase was washed with brine (80 mL), dried over anhydrous Na₂SO₄, and concentrated to give a crude product. The residue was purified by silica gel column chromatography (PE/EtOAc=10/1). Compound 7 (267 mg, 671.90 umol, 65.03% yield, N/A purity) was obtained as brown oil. LCMS: Rt=0.600 min, m/z=396.8, 398.8 (M−H⁺)

To a 25 mL snake tube was added a solution of compound 7 (206 mg, 518.39 umol, 1 eq) in 2-MeTHF (5 mL). Then n-BuLi in Hexane (2.5 M, 414.72 uL, 2 eq) was added into the above mixture at −70° C. under N₂ atmosphere, the mixture was stirred at −70° C. for 1 hr. Then DMF (41.68 mg, 570.23 umol, 43.87 uL, 1.1 eq) was added to the above mixture, the mixture was stirred at −70° C. for 1 h under N₂ atmosphere. The mixture was poured into ice-NH₄Cl (sat. 50 mL) and stirred for 1 min. The aqueous phase was extracted with EtOAc (40 mL×2). The combined organic phase was washed with brine (60 mL), dried with anhydrous Na₂SO₄, filtered and concentrated in vacuum. The residue was purified by prep-TLC (PE:EtOAc=10:1). Compound 8 (30 mg, 86.58 umol, 16.70% yield, N/A purity) was obtained as light brown solid. ¹H NMR (400 MHz, chloroform-d) δ=10.39 (s, 1H), 8.10 (s, 1H), 7.69 (s, 1H), 6.15-6.08 (m, 1H), 6.03 (s, 2H), 5.47 (dd, J=1.5, 17.1 Hz, 1H), 5.32 (dd, J=1.3, 10.5 Hz, 1H), 4.67 (d, J=5.1 Hz, 2H), 3.54-3.48 (m, 3H), 2.59 (s, 3H), 0.90-0.80 (m, 2H), −0.08 (s, 9H).

A compound of compound 8 (45 mg, 129.87 umol, 1 eq) in MeOH (1 mL) was added in MeNH₂ in THF (2 M, 194.81 uL, 3 eq) and HOAc (779.91 ug, 12.99 umol, 7.43e-1 uL, 0.1 eq) in 100 mL single round bottom flask at 20° C. The mixture was stirred at 20° C. for 3 hr. Then the mixture was added in NaBH₄ (9.83 mg, 259.74 umol, 2 eq) at 0° C. The mixture was stirred at 0° C. for 1 hr. After the reaction was completed, the reaction mixture was poured into NH₄Cl (0.1 mL), filtered, the filtrate was concentrated under reduced pressure to give a residue. Then the solution was triturated in EtOAc and THF (50 mL) and collected by filtration, the filtrate was concentrated under reduced pressure to give a residue. No need purification and use to next step. Compound 9 (53 mg, 127.09 umol, 97.86% yield, 86.7% purity) was obtained as light brown solid. LCMS: Rt=0.359 min, m/z=362.4 (M−H⁺) ¹H NMR (400 MHz, chloroform-d) δ=8.01 (s, 1H), 7.12 (s, 1H), 6.21-6.11 (m, 1H), 5.99 (s, 2H), 5.49 (dd, J=1.5, 17.3 Hz, 1H), 5.34 (dd, J=1.4, 10.5 Hz, 1H), 4.68-4.60 (m, 2H), 4.20-4.16 (m, 2H), 3.64-3.54 (m, 2H), 2.58 (s, 1H), 2.54 (s, 3H), 1.03-0.84 (m, 2H), 0.00 (s, 9H).

To a 50 mL single necked round bottom flask was added a solution of compound 9 (53 mg, 127.09 umol, 86.7% purity, 1 eq) in DCM (2 mL) was added compound 10 (33.08 mg, 127.09 umol, 1 eq), EDCI (36.55 mg, 190.64 umol, 1.5 eq) and DMAP (1.55 mg, 12.71 umol, 0.1 eq). The reaction mixture was stirred at 20° C. for 2 hrs. The reaction mixture was concentrated under vacuum. The residue was purified by prep-TLC (PE:EtOAc=2:1). Compound 11 (40 mg, 66.25 umol, 52.12% yield, N/A purity) was obtained as light brown solid. LCMS: Rt=0.645 min, fragment Ms: m/z=486.8 (M+H⁺)

To a solution of compound 11 (40 mg, 66.25 umol, 1 eq) in DCM (2 mL) was added TFA (1.54 g, 13.51 mmol, 1 mL, 203.87 eq) in 100 mL single bottle The mixture was stirred at 20° C. for 2 hr. The reaction mixture was concentrated under reduced pressure to remove residue. The residue was based with Et₃N to pH about 8. The residue was purified by prep-HPLC (column: Phenomenex luna C18 150*25 mm*10 um; mobile phase: [water(FA)-ACN]; B %: 60%-90%, 10 min), lyophilized. Compound 12 (20 mg, 42.24 umol, 63.75% yield, N/A purity) was obtained white solid. LCMS: Rt=0.551 min, m/z=474.5 (M+H⁺)

To a solution of compound 12 (15 mg, 31.68 umol, 1 eq) in DCM (1 mL) was added Pd(PPh₃)₄ (7.32 mg, 6.34 umol, 0.2 eq) and 1,3-dimethylhexahydropyrimidine-2,4,6-trione (14.84 mg, 95.03 umol, 3 eq) in 8 mL bottle under N₂. The mixture was stirred at 20° C. for 5 hr under N₂. The reaction mixture was filtered, the filtrate was concentrated to give a crude product. The residue was purified by prep-HPLC (column: Phenomenex luna C18 150*25 mm*10 um; mobile phase: [water(NH₄HCO₃)-ACN]; B %: 34%-64%, 8 min), lyophilized. 015 (4.6 mg, 10.52 umol, 33.20% yield, 99.1% purity) was obtained as white solid. LCMS: Rt=0.463 min, m/z=434.3 (M+H⁺) ¹H NMR (400 MHz, chloroform-d) δ=8.04 (s, 1H), 7.40-7.28 (m, 4H), 7.10-7.01 (m, 3H), 6.93 (s, 1H), 4.89 (s, 2H), 4.06 (q, J=7.0 Hz, 2H), 3.01 (s, 3H), 2.50 (s, 3H), 1.37 (br t, J=6.8 Hz, 3H).

Compound 036 was prepared in a similar manner to Scheme 9 and was obtained as a white solid (15.1 mg, 36.00 umol, 27.20% yield, 100% purity). LCMS: Rt=0.482 min, m/z=420.3 (M+H)⁺ 1H NMR (400 MHz, chloroform-d) δ=8.02 (s, 1H), 7.58 (d, J=8.6 Hz, 1H), 7.39-7.28 (m, 4H), 7.09-7.00 (m, 3H), 6.87-6.81 (m, 1H), 5.02 (s, 2H), 4.03 (q, J=6.9 Hz, 2H), 3.14 (s, 3H), 1.34 (t, J=6.9 Hz, 3H).

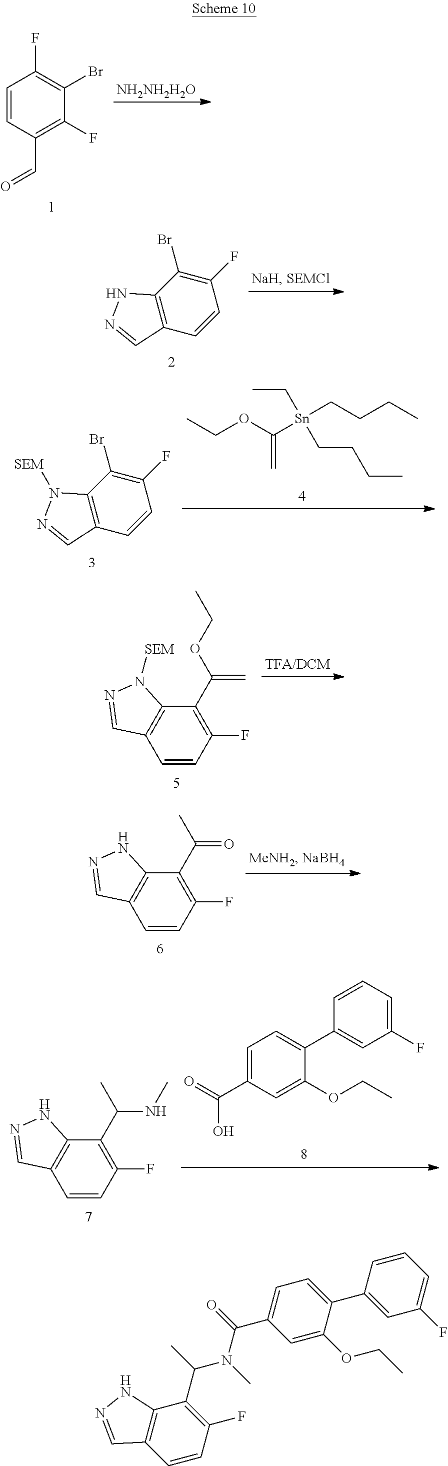

To a solution of Compound 1 (1.8 g, 8.14 mmol, 1 eq) in DMSO (20 mL) was added NH₂NH₂·H₂O (1.43 g, 28.51 mmol, 1.39 mL, 3.5 eq) in 100 mL three-necked flack. The mixture was stirred at 120° C. for 12 hr. The reaction mixture was poured into water (200 mL) and extracted by EtOAc (200 mL×2), the combined organic phases were washed with brine (100 mL), dried over Na₂SO₄ and concentrated under vacuum to give a residue. The combined aqueous phase was acidified by 1N HCl aqueous to pH about 4 and discarded into liquid waste barrels. The residue was purified by silica gel chromatography (PE:EtOAc=0:0 to 1:1). Compound 2 (695 mg, 3.23 mmol, 39.68% yield) was obtained as a white solid. LCMS: Rt=0.361 min, m/z=216.8 (M+H⁺).

To a solution of Compound 1 (695 mg, 3.23 mmol, 1 eq) in THF (10 mL) was added NaH (193.91 mg, 4.85 mmol, 60% purity, 1.5 eq) at 100 mL three-necked flack under 0° C., the mixture was stirred at 0° C. for 1 hr, Then SEM-CI (808.32 mg, 4.85 mmol, 858.09 uL, 1.5 eq) was added into the reaction under 0° C., the reaction mixture was stirred at 20° C. for 2 hr. The reaction mixture was poured into saturated NH₄Cl aqueous solution (100 mL), the mixture was extracted by EtOAc (500 mL×3), and the combined organic phase was dried and concentrated under vacuum to give a residue. The residue was purified by silica gel chromatography (PE:EtOAc=0:0 to 1:1). Compound 3 (787 mg, 2.28 mmol, 70.52% yield) was obtained as a yellow oil. LCMS: Rt=0.556 min, m/z=344.04 (M+H⁺).

To a solution of Compound 3 (787 mg, 2.28 mmol, 1 eq) and Compound 4 (1.23 g, 3.42 mmol, 1.15 mL, 1.5 eq) in dioxane (10 mL) and H₂O (1 mL) at 50 mL single-mouth round bottom flask was added K₂CO 3 (630.02 mg, 4.56 mmol, 2 eq) and Pd(PPh₃)₂Cl₂ (239.97 mg, 341.89 umol, 0.15 eq). The mixture was stirred at 100° C. for 12 hr.

After the reaction was completed and cooled to room temperature, the reaction mixture was diluted with saturated potassium fluoride solution (100 mL) and stirred at 20° C. for 2 h. Then the mixture was filtered, the filtrate was extracted with EtOAc (100 mL×3), the combined organic phase was washed with brine (150 mL×3), dried over anhydrous Na₂SO₄, and concentrated to give a crude product. The residue was purified by silica gel chromatography (PE:EtOAc=0:0 to 10:1). Compound 5 (680 mg, 2.02 mmol, 88.67% yield) was obtained as a yellow oil. ¹H NMR: ¹H NMR (chloroform-d): δ 8.00 (1H, s), 7.65 (1H, dd, J=4.9 and 8.8 Hz), 6.99 (1H, t, J=9.1 Hz), 5.74 (2H, s), 4.42-4.72 (2H, m), 4.13 (2H, q, J=7.1 Hz), 3.49-3.58 (2H, m), 1.40 (3H, t, J=7.0 Hz), 0.84 (3H, s), −0.06 (9H, s)

To a solution of Compound 5 (500 mg, 1.49 mmol, 1 eq) in DCM (5 mL) was added TFA (7.70 g, 67.53 mmol, 5 mL, 45.44 eq) in a 100 ml single-mouth round bottom flask. The mixture was stirred at 20° C. for 2 hr. The reaction mixture was concentrated and under reduced pressure to give a residue. The residue was purified by silica gel chromatography (PE:EtOAc=0:0 to 2:1). Compound 6 (250 mg, 1.40 mmol, 94.43% yield) was obtained as a yellow oil. LCMS: Rt=0.307 min, m/z=179.1 (M+H⁺).

Dissolved Compound 6 in MeOH (2 mL) in a 100 mL single-neck bottle was added HOAc (471.87 ug, 7.86 umol, 4.49e-1 uL, 0.01 eq) and MeNH₂ in THF (2 M, 1.18 mL, 3 eq), the reaction mixture was stirred at 20° C. for 2 hr by magnetic stirring apparatus. Then NaBH₄ (59.45 mg, 1.57 mmol, 2 eq) was added at 0° C., the mixture was stirred at 20° C. for 1 hr by magnetic stirring apparatus. After the reaction was completed, the reaction mixture was poured into NH₄Cl (10 mL), filtered, the filtrate was concentrated under reduced pressure to give a residue. Then the solution was triturated in EtOAc and THF (10 mL) and collected by filtration, the filtrate was concentrated under reduced pressure to give a residue. Compound 7 was obtained as a yellow oil. LCMS: Rt=0.412 min, m/z=194.0 (M+H⁺).

To a solution of Compound 7 (133 mg, 688.33 umol, 1 eq) and Compound 8 (179.15 mg, 688.33 umol, 1 eq) in DCM (5 mL) in a 40 mL glass bottle were added EDCI (197.93 mg, 1.03 mmol, 1.5 eq) and DMAP (8.41 mg, 68.83 umol, 0.1 eq), the mixture was stirred at 20° C. for 12 hr. The mixture was neutralized with Na₂CO₃ at 20° C. and filtered, the filtrate was concentrated to give a crude product. The residue was purified by prep-TLC (SiO₂, PE:EtOAc=3:1) to give 017 (7.4 mg, 16.48 umol, 2.39% yield, 97% purity) as colorless oil, LCMS: Rt=0.543 min, m/z=436.4 (M+H⁺). HNMR: (400 MHz, chloroform-d) δ=12.27-11.82 (m, 1H), 8.07 (s, 1H), 7.67 (dd, J=4.5, 8.8 Hz, 1H), 7.39-7.28 (m, 4H), 7.08-6.99 (m, 3H), 6.98-6.89 (m, 1H), 6.41 (br d, J=7.0 Hz, 1H), 4.26-3.85 (m, 2H), 3.28-2.76 (m, 3H), 1.95 (dd, J=4.1, 7.3 Hz, 3H), 1.36 (t, J=6.9 Hz, 3H).

Compound 116 was prepared in a similar manner to Scheme 10 and was obtained as a white solid (11.0 mg, 25.26 umol, 24.40% yield, 100% purity). LCMS: Rt=0.735 min, m/z=435.9 (M+H⁺) 1H NMR (400 MHz, chloroform-d) δ=8.18 (s, 1H), 7.41-7.33 (m, 2H), 7.33-7.28 (m, 3H), 7.08-7.00 (m, 3H), 6.79 (dd, J=8.1, 9.4 Hz, 1H), 6.48 (d, J=6.6 Hz, 1H), 4.14-3.99 (m, 2H), 2.76 (s, 3H), 1.84-1.78 (m, 3H), 1.37 (t, J=6.9 Hz, 3H).

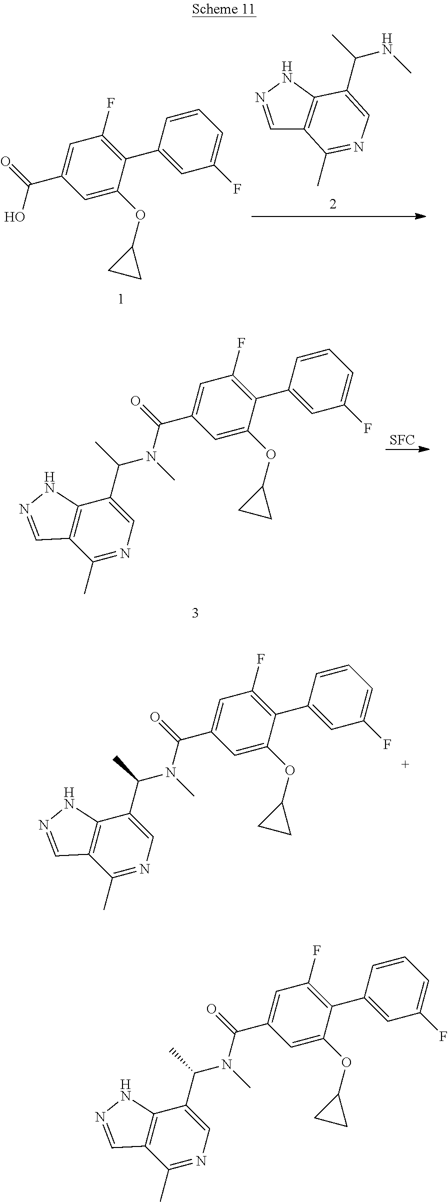

To a solution of Compound 1 (152.57 mg, 525.64 umol, 1 eq) and Compound 2 (100 mg, 525.64 umol, 1 eq) in DCM (5 mL) in round-bottom flask (50 mL) was added EDCI (151.15 mg, 788.46 umol, 1.5 eq) and DMAP (6.42 mg, 52.56 umol, 0.1 eq). The mixture was stirred at 20° C. for 1 hr. The reaction mixture was concentrated on vacuum to give residue. The residue was purified by prep-HPLC (column: Phenomenex luna C18 150*25 mm*10 um; mobile phase: [water (FA)-ACN]; B %: 22%-52%, 9 min). The purified solution was lyophilized to give Compound 3 (85 mg, 173.13 umol, 32.94% yield, 94.2% purity) as a white solid. LCMS: Rt=0.415 min, m/z=462.9 (M+H)⁺

Compound 3 (85 mg, 183.79 umol, 1 eq) was purify by SFC (column: DAICEL CHIRALPAK IC (250 mm*30 mm, 10 um); mobile phase: [ACN/IPA(0.1% NH₃H₂O)]; B %: 45%-45%, A 2.8; 22 min), the solution was concentrated to remove organic solvents. The residual aqueous solution was lyophilized to give 034 (31.6 mg, 68.33 umol, 37.18% yield, 100% purity) as a white solid and 035 (32.5 mg, 69.78 umol, 37.97% yield, 99.3% purity) as a white solid.

Data for 034: LCMS: Rt=0.730 min, m/z=463.2 (M+H)⁺ SFC: Rt=0.591 min 1H NMR (400 MHz, chloroform-d) δ=(400 MHz, chloroform-d) δ=12.18-11.92 (m, 1H), 8.31 (s, 1H), 8.25 (s, 1H), 7.41-7.34 (m, 1H), 7.22-7.19 (m, 1H), 7.14-7.09 (m, 1H), 7.09-7.02 (m, 2H), 6.90-6.84 (m, 1H), 6.48-6.39 (m, 1H), 3.78-3.72 (m, 1H), 2.92-2.87 (m, 3H), 2.86-2.79 (m, 3H), 1.87 (d, J=7.3 Hz, 3H), 0.84-0.76 (m, 2H), 0.75-0.65 (m, 2H).

Data for 035: LCMS: Rt=0.731 min, m/z=463.2 (M+H)⁺ SFC: Rt=0.851 min 1H NMR (400 MHz, chloroform-d) δ=(400 MHz, chloroform-d) δ=8.32 (s, 1H), 8.26 (s, 1H), 7.41-7.34 (m, 1H), 7.21 (s, 1H), 7.12 (d, J=8.1 Hz, 1H), 7.09-7.02 (m, 2H), 6.90-6.84 (m, 1H), 6.47-6.39 (m, 1H), 3.80-3.71 (m, 1H), 2.90 (s, 3H), 2.85-2.80 (m, 3H), 1.89-1.85 (m, 3H), 0.83-0.77 (m, 2H), 0.74-0.64 (m, 2H).

Compounds 042 and 043 were prepared in a similar manner to Scheme 11. Data for 042: (17 mg, 39.04 umol, 34.00% yield), LCMS: Rt=0.522 min, m/z=435.9 (M+H⁺), ¹H NMR: H NMR (400 MHz, chloroform-d) δ=11.84 (br s, 1H), 8.00 (s, 1H), 7.33-7.21 (m, 5H), 7.11 (dd, J=1.4, 9.8 Hz, 1H), 7.01-6.93 (m, 3H), 6.44 (br d, J=6.9 Hz, 1H), 4.06-3.92 (m, 2H), 2.70 (s, 3H), 1.72 (d, J=7.1 Hz, 3H), 1.29 (t, J=6.9 Hz, 3H). Data for 043: (15.5 mg, 35.59 umol, 31.00% yield), LCMS: Rt=0.519 min, m/z=435.9 (M+H⁺) ¹H NMR: (400 MHz, chloroform-d) δ=8.10 (s, 1H), 7.42-7.30 (m, 5H), 7.21 (br d, J=9.4 Hz, 1H), 7.09-7.02 (m, 3H), 6.61-6.46 (m, 1H), 4.15-4.01 (m, 2H), 2.80 (s, 3H), 1.82 (d, J=7.1 Hz, 3H), 1.38 (t, J=6.9 Hz, 3H).

Compounds 044 and 045 were prepared in a similar manner to Scheme 11. Data for 044: (8.2 mg, 18.20 umol, 39.36% yield, and 96% purity), LCMS: Rt=0.689 min, m/z=433.2 (M+H+). 1H NMR: (chloroform-d): δ 12.18-12.47 (1H, m), 8.99 (1H, s), 8.20 (1H, s), 7.29-7.41 (4H, m), 7.02-7.12 (3H, m), 5.68 (1H, q, J=7.2 Hz), 4.00-4.15 (2H, m), 2.95 (3H, s), 2.83 (3H, s), 2.01 (3H, d, J=7.3 Hz), 1.38 (3H, t, J=6.9 Hz). Data for 045: (9.8 mg, 21.53 umol, 46.55% yield, and 95% purity), LCMS: Rt=0.619 min, m/z=433.2 (M+H+). 1H NMR: (chloroform-d): δ 12.07-12.66 (1H, m), 8.99 (1H, s), 8.20 (1H, s), 7.28-7.39 (4H, m), 6.97-7.12 (3H, m), 5.68 (1H, q, J=7.0 Hz), 4.02-4.14 (2H, m), 2.95 (3H, s), 2.83 (3H, s), 2.01 (3H, br d, J=7.3 Hz), 1.38 (3H, br t, J=7.0 Hz).

Compounds 049 and 050 were prepared in a similar manner to Scheme 11. Data for 049: (13.1 mg, 28.84 umol, 34.64% yield, 95% purity), LCMS: Rt=0.409 min, m/z=433.4 (M+H⁺). ¹H NMR: (400 MHz, chloroform-d) δ=12.09 (br s, 1H), 8.31 (s, 1H), 8.24 (s, 1H), 7.43-7.30 (m, 4H), 7.11-7.02 (m, 3H), 6.51-6.41 (m, 1H), 4.18-4.00 (m, 2H), 2.87 (s, 3H), 2.84-2.78 (m, 1H), 1.88 (d, J=7.3 Hz, 3H), 1.45-1.34 (m, 3H). Data for 050: (13.8 mg, 31.17 umol, 37.45% yield, 97% purity), LCMS: Rt=0.406 min, m/z=433.6 (M+H⁺). ¹H NMR: (400 MHz, chloroform-d) δ=12.00 (br s, 1H), 8.22 (s, 1H), 8.14 (s, 1H), 7.33-7.20 (m, 4H), 7.01-6.93 (m, 3H), 6.36 (q, J=7.1 Hz, 1H), 4.07-3.91 (m, 2H), 2.77 (s, 3H), 2.73 (s, 3H), 1.78 (d, J=7.1 Hz, 3H), 1.29 (t, J=6.9 Hz, 3H).

Compounds 051 and 052 were prepared in a similar manner to Scheme 11. Data for 051: (11.1 mg, 25.79 umol, 22.20% yield), LCMS: Rt=0.613 min, m/z=431.3 (M+H+). 1H NMR: (chloroform-d): δ 12.01-12.32 (1H, m), 9.07-9.18 (1H, m), 8.42-8.50 (1H, m), 8.27 (1H, s), 7.32-7.46 (2H, m), 7.01-7.25 (5H, m), 6.51 (1H, br d, J=6.5 Hz), 3.78 (1H, br s), 2.84 (3H, s), 1.90 (3H, br d, J=7.1 Hz), 0.74-0.88 (4H, m). Data for 051: (12.8 mg, 29.73 umol, 25.60% yield), LCMS: Rt=0.613 min, m/z=431.3 (M+H+). 1H NMR: (chloroform-d): δ 12.05-12.31 (1H, m), 8.96-9.32 (1H, m), 8.22-8.57 (2H, m), 7.44 (1H, s), 7.31-7.40 (2H, m), 7.18-7.25 (2H, m), 7.01-7.12 (2H, m), 6.39-6.62 (1H, m), 3.78 (1H, br d, J=3.0 Hz), 2.84 (3H, s), 1.90 (3H, br d, J=7.1 Hz), 0.80-0.88 (4H, m).

Compounds 056 and 057 were prepared in a similar manner to Scheme 11. Data for 056: LCMS: Rt=0.586 min, m/z=420.3 (M+H⁺). ¹H NMR: (400 MHz, chloroform-d) δ=12.11-11.98 (m, 1H), 9.12 (s, 1H), 8.45 (br s, 1H), 8.25 (s, 1H), 7.78-7.72 (m, 1H), 7.49-7.30 (m, 5H), 7.12-7.05 (m, 1H), 4.43 (br d, J=6.5 Hz, 2H), 2.94 (br s, 3H), 1.89 (br d, J=6.5 Hz, 3H), 1.38 (br d, J=6.0 Hz, 3H). Data for 057: LCMS: Rt=0.586 min, m/z=420.3 (M+H⁺) ¹H NMR: (400 MHz, chloroform-d) δ=12.09-11.94 (m, 1H), 9.05 (s, 1H), 8.40-8.34 (m, 1H), 8.18 (s, 1H), 7.67 (br d, J=6.3 Hz, 1H), 7.42-7.22 (m, 5H), 7.04-6.97 (m, 1H), 4.46-4.20 (m, 2H), 2.86 (br s, 3H), 1.82 (br d, J=6.3 Hz, 3H), 1.34-1.27 (m, 3H).

Compounds 058 and 059 were prepared in a similar manner to Scheme 11. Data for 058: LCMS: Rt=0.696 min, m/z=418.2 (M+H⁺). H-NMR: (400 MHz, chloroform-d) δ=12.00-11.76 (m, 1H), 8.13 (s, 1H), 7.78-7.73 (m, 1H), 7.42-7.28 (m, 5H), 7.21-7.15 (m, 1H), 7.07-7.00 (m, 3H), 6.58-6.50 (m, 1H), 4.14-3.99 (m, 2H), 2.76 (s, 3H), 1.84-1.80 (m, 3H), 1.37 (t, J=7.0 Hz, 3H). Data for 059: LCMS: Rt=0.692 min, m/z=418.1 (M+H⁺). H-NMR: (400 MHz, chloroform-d) δ=11.85-11.78 (m, 1H), 8.04 (s, 1H), 7.70-7.65 (m, 1H), 7.33-7.21 (m, 5H), 7.13-7.07 (m, 1H), 6.99-6.92 (m, 3H), 6.46 (q, J=6.9 Hz, 1H), 4.06-3.91 (m, 2H), 2.68 (s, 3H), 1.74 (d, J=7.1 Hz, 3H), 1.29 (t, J=7.0 Hz, 3H).

Compounds 123 and 124 were prepared in a similar manner to Scheme 11. Data for 123: LCMS: Rt=0.615 min, m/z=434.3 (M+H⁺) ¹H NMR: (400 MHz, chloroform-d) δ=13.04-11.60 (m, 1H), 8.39-8.10 (m, 2H), 7.90-7.66 (m, 1H), 7.51-7.28 (m, 4H), 7.14-7.01 (m, 1H), 6.43 (br d, J=6.6 Hz, 1H), 4.72-4.18 (m, 2H), 2.99-2.63 (m, 6H), 1.80-1.61 (m, 3H), 1.35-1.17 (m, 3H) Data for 124: LCMS: Rt=0.614 min, m/z=434.3 (M+H⁺) ¹H NMR: (400 MHz, chloroform-d) δ=12.95-11.89 (m, 1H), 8.33-8.18 (m, 2H), 7.85-7.71 (m, 1H), 7.48-7.31 (m, 4H), 7.12-7.05 (m, 1H), 6.54-6.17 (m, 1H), 4.66-4.23 (m, 2H), 2.93-2.66 (m, 6H), 1.90-1.72 (m, 3H), 1.33 (br s, 3H).

Compounds 094 and 095 were prepared in a similar manner to Scheme 11. Data for 094: LCMS: Rt=0.129 min, m/z=449.2 (M+H⁺). ¹H NMR: (chloroform-d) δ: ppm 1.38 (t, J=6.94 Hz, 3H) 1.68 (d, J=6.63 Hz, 3H) 3.06 (s, 3H) 4.07 (q, J=7.13 Hz, 2H) 4.95 (s, 2H) 5.32 (q, J=6.59 Hz, 1H) 7.01-7.12 (m, 3H) 7.31 (d, J=7.38 Hz, 2H) 7.33-7.41 (m, 2H) 8.20-8.33 (m, 2H) 12.22-12.42 (m, 1H). Data for 095: LCMS: Rt=0.134 min, m/z=449.2 (M+H⁺). ¹H NMR: (chloroform-d) δ: ppm 1.38 (t, J=7.00 Hz, 3H) 1.51-1.60 (m, 5H) 1.66-1.81 (m, 2H) 3.52 (s, 3H) 3.57-3.63 (m, 1H) 3.83-3.92 (m, 1H) 4.11-4.16 (m, 1H) 4.94 (s, 1H) 7.00-7.08 (m, 1H) 7.25-7.29 (m, 2H) 7.30 (d, J=1.13 Hz, 1H) 7.32-7.37 (m, 3H).

Compounds 123 and 124 were prepared in a similar manner to Scheme 11. Data for 123 (34.09 mg, 76.44 umol, 36.82% yield, and 97.2% purity): LCMS: Rt=0.615 min, m/z=434.3 (M+H⁺) ¹H NMR: (400 MHz, chloroform-d) δ=13.04-11.60 (m, 1H), 8.39-8.10 (m, 2H), 7.90-7.66 (m, 1H), 7.51-7.28 (m, 4H), 7.14-7.01 (m, 1H), 6.43 (br d, J=6.6 Hz, 1H), 4.72-4.18 (m, 2H), 2.99-2.63 (m, 6H), 1.80-1.61 (m, 3H), 1.35-1.17 (m, 3H). Data for 124 (30.89 mg, 69.62 umol, 33.53% yield, and 97.7% purity): LCMS: Rt=0.614 min, m/z=434.3 (M+H⁺) ¹H NMR: (400 MHz, chloroform-d) δ=12.95-11.89 (m, 1H), 8.33-8.18 (m, 2H), 7.85-7.71 (m, 1H), 7.48-7.31 (m, 4H), 7.12-7.05 (m, 1H), 6.54-6.17 (m, 1H), 4.66-4.23 (m, 2H), 2.93-2.66 (m, 6H), 1.90-1.72 (m, 3H), 1.33 (br s, 3H).

Compounds 159 and 160 were prepared in a similar manner to Scheme 11. Data for 159 (7.4 mg, 16.50 umol, 37.00% yield): LCMS: Rt=0.648 min, m/z=449.1 (M+H⁺) ¹H NMR: (400 MHz, chloroform-d) δ=12.19 (s, 1H), 8.35-8.25 (m, 1H), 8.18 (s, 1H), 7.45-7.20 (m, 5H), 6.99-6.96 (m, 2H), 6.40 (q, J=6.8 Hz, 1H), 5.06 (s, 2H), 4.07-3.92 (m, 2H), 2.74 (s, 3H), 1.81 (d, J=7.1 Hz, 3H), 1.30 (t, J=7.0 Hz, 3H). Data for 160 (8.4 mg, 18.73 umol, 42.00% yield): LCMS: Rt=0.646 min, m/z=449.1 (M+H⁺) ¹H NMR: (400 MHz, chloroform-d) δ=12.46-11.91 (m, 1H), 8.30 (s, 1H), 8.17 (s, 1H), 7.40 (s, 5H), 6.98 (s, 2H), 6.39 (d, J=7.0 Hz, 1H), 5.05 (s, 2H), 4.07-3.92 (m, 2H), 2.74 (s, 3H), 1.81 (d, J=7.1 Hz, 3H), 1.29 (t, J=6.9 Hz, 3H).

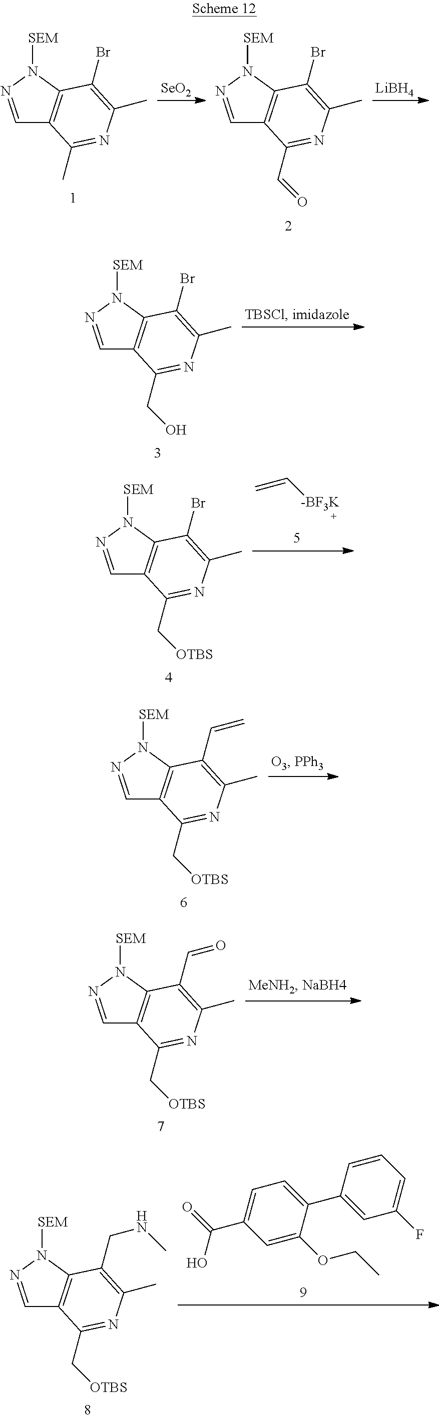

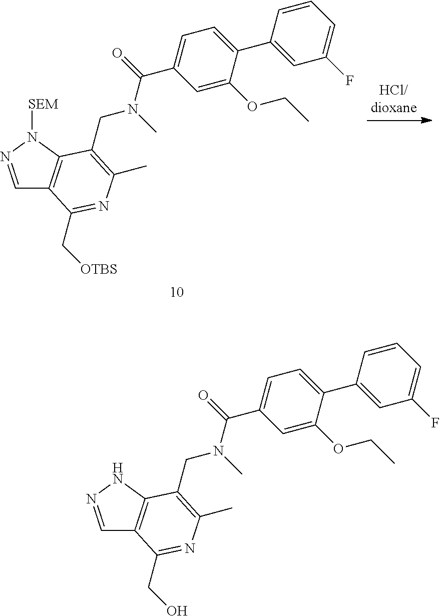 [SMILES: CCOc1cc(C(=O)N(C)Cc2c(C)nc(CO[Si](C)(C)C(C)(C)C)c3cnn(COCC[Si](C)(C)C)c23)ccc1-c1cccc(F)c1 ; CCOc1cc(C(=O)N(C)Cc2c(C)nc(CO)c3cn[nH]c23)ccc1-c1cccc(F)c1]

To a solution of compound 1 (350 mg, 982.23 umol, 1 eq) in dioxane (5 mL) in a 40 mL glass bottle was added SeO₂ (544.94 mg, 4.91 mmol, 534.26 uL, 5 eq), the mixture was stirred at 60° C. for 16 h. The reaction mixture was filtered, the filtrate was concentrated to give a crude product. The residue was purified by silica gel chromatography (PE:EtOAc=10:1). Compound 2 (360 mg, 972.14 umol, 98.97% yield, N/A purity) was obtained as light brown solid. LCMS: Rt=0.559 min, m/z=369.8, 371.8 (M+H⁺). ¹H NMR (400 MHz, chloroform-d) δ=10.20 (s, 1H), 8.73 (s, 1H), 6.11 (s, 2H), 3.63-3.58 (m, 2H), 2.92 (s, 3H), 0.94-0.86 (m, 2H), −0.05 (s, 9H).

To a solution of compound 2 (325 mg, 877.63 umol, 1 eq) in MeOH (7 mL) in a 100 mL single necked round bottom flask was added LiBH₄ (115 mg, 5.28 mmol, 6.02 eq) at 0° C., the mixture was stirred at 0° C. for 0.5 h. After the reaction was completed, the reaction mixture was quenched by saturated NH₄Cl aqueous (50 mL) at 0° C. The mixture was EtOAc extracted with (50 mL×2), the combined organic phase was dried over Na₂SO₄, and concentrated on vacuum to give a crude product. The residue was purified by silica gel chromatography (PE:EtOAc=1:1). Compound 3 (257 mg, 676.44 umol, 77.08% yield, 98% purity) was obtained as white solid. LCMS: Rt=0.131 min, m/z=226.0, 228.0 (M+H⁺).

To a solution of compound 3 (257 mg, 690.24 umol, 1 eq) in DCM (5 mL) in a 50 mL single necked round bottom flask was added imidazole (117.47 mg, 1.73 mmol, 2.5 eq) and TBSCI (208.07 mg, 1.38 mmol, 169.16 uL, 2 eq) at 0° C., the mixture was stirred at 20° C. for 12 h. TLC (PE:EtOAc=10:1, plate 1) showed the desired spot (R_{f}=0.21) was detected. The mixture was filtered, and the filtrate was concentrated to give a crude product. The residue was purified by silica gel column chromatography (PE:EtOAc=10:1). Compound 4 (320 mg, 657.63 umol, 95.28% yield, N/A purity) was obtained as colorless oil. ¹H NMR (400 MHz, chloroform-d) δ=8.44 (s, 1H), 6.07 (s, 2H), 5.07 (s, 2H), 3.66-3.54 (m, 2H), 2.77 (s, 3H), 0.97 (s, 9H), 0.91-0.87 (m, 2H), 0.16 (s, 6H), −0.05 (s, 9H)

To a solution of compound 4 (320 mg, 657.63 umol, 1 eq) and compound 5 (176.18 mg, 1.32 mmol, 2 eq) in dioxane (4 mL) and H₂O (0.4 mL) was added K₂CO₃ (181.78 mg, 1.32 mmol, 2 eq) and Pd(dppf)Cl₂·CH₂Cl₂ (53.70 mg, 65.76 umol, 0.1 eq) under N₂. The mixture was stirred at 100° C. for 16 hrs under N₂. The reaction mixture was filtered through celite pad. The combined filter cake was washed with EtOAc/methanol=10/1, 10 mL×5. The combined filtrate were concentrated to give a crude product. The crude product was purified by silica gel chromatography (PE:EtOAc=10:1). Compound 6 (190 mg, 438.06 umol, 66.61% yield, N/A purity) was obtained as colorless oil.

To a solution of compound 6 (190 mg, 438.06 umol, 1 eq) in DCM (12 mL) and MeOH (12 mL) was treated with ozone (21.03 mg, 438.06 umol, 1 eq) for 30 min (15 psi) at −78° C. in 100 mL three neck round bottom flask, then the mixture was purged with nitrogen for 0.5 h, PPh₃ (229.79 mg, 876.11 umol, 2 eq) was dissolved in DCM (12 mL) was added dropwise to the mixture at −78° C. The mixture was warmed to 20° C. and stirred at 20° C. for 1 h. After the reaction was completed, the mixture was concentrated to give a crude product. The residue was purified by silica gel chromatography (PE:EtOAc=10:1). Compound 7 (140 mg, 321.32 umol, 73.35% yield, N/A purity) was obtained as light brown solid. ¹H NMR (400 MHz, chloroform-d) δ=10.81 (s, 1H), 8.64 (s, 1H), 6.07 (s, 2H), 5.24 (br s, 2H), 3.56-3.49 (m, 2H), 3.02 (s, 3H), 1.06 (s, 9H), 0.96-0.87 (m, 2H), 0.26 (s, 6H), 0.17 (d, J=12.6 Hz, 1H), 0.00 (s, 9H).

A compound of compound 7 (140 mg, 321.32 umol, 1 eq) in MeOH (2 mL) was added in MeNH₂ in THF (2 M, 481.98 uL, 3 eq) and HOAc (1.93 mg, 32.13 umol, 1.84 uL, 0.1 eq) in 50 mL single round bottom flask at 20° C. The mixture was stirred at 20° C. for 3 hr. Then the mixture was added in NaBH₄ (24.31 mg, 642.63 umol, 2 eq) at 0° C. The mixture was stirred at 20° C. for 1 hr. After the reaction was completed, the reaction mixture was poured into NH₄Cl (0.1 mL), filtered, the filtrate was concentrated under reduced pressure to give a residue. Then the solution was triturated in EtOAc and THF (80 mL) and collected by filtration, the filtrate was concentrated under reduced pressure to give compound 8 (150 mg, crude) as light brown oil. LCMS: Rt=0.446 min, m/z=452.1 (M+H⁺)

To an 8 mL bottle was added a solution of compound 8 (50 mg, 94.28 umol, 85% purity, 1 eq) in DCM (1 mL) was added compound 9 (24.54 mg, 94.28 umol, 1 eq), EDCI (27.11 mg, 141.43 umol, 1.5 eq) and DMAP (1.15 mg, 9.43 umol, 0.1 eq). The reaction mixture was stirred at 20° C. for 2 hrs. The reaction mixture was concentrated under vacuum. The residue was purified by prep-TLC (PE:EtOAc=2:1). Compound 10 (47 mg, 67.82 umol, 71.93% yield, N/A purity) was obtained as off-white solid. LCMS: Rt=0.559 min, m/z=694.0 (M+H⁺)

To a solution of compound 10 (42 mg, 60.61 umol, 1 eq) in HCl/dioxane (4 M, 2 mL, 132.00 eq) in a 50 mL single necked round bottom flask. The mixture was stirred at 20° C. for 2 hr. The reaction mixture was concentrated to give the crude product. The crude product was based with Et₃N to pH about 8˜9. The residue was purified by prep-HPLC (column: Waters xbridge 150*25 mm 10 um; mobile phase: [water (NH₄HCO₃)-ACN]; B %: 27%-57%, 11 min), lyophilized. 037 (5.2 mg, 11.43 umol, 18.86% yield, and 98.6% purity) was obtained as white solid. LCMS: Rt=0.389 min, m/z=449.5 (M+H⁺) ¹H NMR (400 MHz, chloroform-d) δ=13.15 (br s, 1H), 8.59 (s, 1H), 7.41-7.37 (m, 2H), 7.33-7.30 (m, 2H), 7.11-7.05 (m, 3H), 7.01 (s, 1H), 5.88 (br s, 1H), 5.38 (br s, 2H), 5.11 (s, 2H), 4.08 (q, J=6.9 Hz, 2H), 3.14 (s, 1H), 3.12 (s, 3H), 1.39 (t, J=6.9 Hz, 3H).

Compound 040 was prepared in a similar manner to Scheme 12 and was isolated as a white solid (6.8 mg, 13.68 umol, 25.30% yield, and 99.6% purity). LCMS: Rt=0.416 min, m/z=495.5 (M+H⁺) ¹H NMR (400 MHz, chloroform-d) δ=12.09 (br s, 1H), 8.11 (s, 1H), 7.30-7.28 (m, 1H), 7.20 (s, 2H), 7.15 (br s, 2H), 6.80 (br d, J=8.4 Hz, 1H), 5.03 (s, 2H), 5.01 (s, 2H), 4.39 (br s, 1H), 3.68 (br d, J=3.1 Hz, 1H), 2.99 (s, 3H), 2.77 (s, 3H), 0.77-0.68 (m, 2H), 0.65 (br s, 2H).

Compound 041 was prepared in a similar manner to Scheme 12 and was isolated as a white solid (13 mg, 27.26 umol, 35.75% yield, and 100% purity). LCMS: Rt=0.375 min, m/z=477.4 (M+H⁺) ¹H NMR (400 MHz, chloroform-d) δ=12.29 (br s, 1H), 8.18 (s, 1H), 7.44 (br d, J=7.4 Hz, 2H), 7.37-7.29 (m, 4H), 7.12-7.06 (m, 1H), 5.10-5.08 (m, 4H), 4.52 (br s, 1H), 3.81-3.74 (m, 1H), 3.06 (s, 3H), 2.84 (s, 3H), 0.85-0.72 (m, 4H).

Compound 053 was prepared in a similar manner to Scheme 12 and was isolated as a white solid (30.9 mg, 70.95 umol, 39.93% yield, and 99.3% purity). LCMS: Rt=0.388 min, m/z=433.4 (M+H⁺) ¹H NMR (400 MHz, chloroform-d) δ=12.09 (br s, 1H), 8.15 (s, 1H), 7.41-7.27 (m, 4H), 7.09-7.01 (m, 3H), 5.07 (s, 2H), 4.07 (q, J=7.0 Hz, 2H), 3.02 (s, 3H), 2.82 (s, 3H), 2.79 (s, 3H), 1.37 (t, J=6.9 Hz, 3H).

Compound 054 was prepared in a similar manner to Scheme 12 and was isolated as a white solid (7.3 mg, 16.70 umol, 20.56% yield, and 99.4% purity). LCMS: Rt=0.443 min, m/z=435.3 (M+H⁺) ¹H NMR (400 MHz, chloroform-d) δ=12.27 (br s, 1H), 8.09 (s, 1H), 7.91 (s, 1H), 7.40-7.34 (m, 2H), 7.33-7.28 (m, 2H), 7.09-7.02 (m, 3H), 5.43 (s, 2H), 4.07 (q, J=6.9 Hz, 2H), 3.31 (s, 3H), 2.56 (s, 3H), 1.39-1.35 (m, 3H).

Compound 079 was prepared in a similar manner to Scheme 12 and was isolated as a white solid (6 mg, 12.97 umol, 18.34% yield, 100% purity). LCMS: Rt=0.390 min, m/z=463.1 (M+H⁺) ¹H NMR (400 MHz, chloroform-d) δ=12.46 (s, 1H), 8.31-8.23 (m, 1H), 7.41-7.34 (m, 2H), 7.33-7.28 (m, 2H), 7.13-7.00 (m, 3H), 5.39-5.24 (m, 1H), 5.10 (s, 2H), 4.08 (q, J=7.1 Hz, 2H), 3.06 (s, 3H), 2.89 (s, 3H), 1.72 (br s, 3H), 1.38 (t, J=6.9 Hz, 3H).

Compound 117 was prepared in a similar manner to Scheme 12 and was isolated as a white solid (15 mg, 34.68 umol, 18.05% yield). LCMS: Rt=0.743 min, m/z=433.2 (M+H⁺) ¹H NMR: (400 MHz, chloroform-d) δ=12.33-11.81 (m, 1H), 9.04 (br s, 1H), 8.34 (br s, 1H), 8.19 (s, 1H), 7.36-7.21 (m, 4H), 7.04-6.90 (m, 3H), 6.19-5.97 (m, 1H), 3.99 (br dd, J=7.2, 17.4 Hz, 2H), 2.76-2.66 (m, 3H), 2.41-2.10 (m, 2H), 1.33-1.26 (m, 3H), 1.17-1.11 (m, 3H).

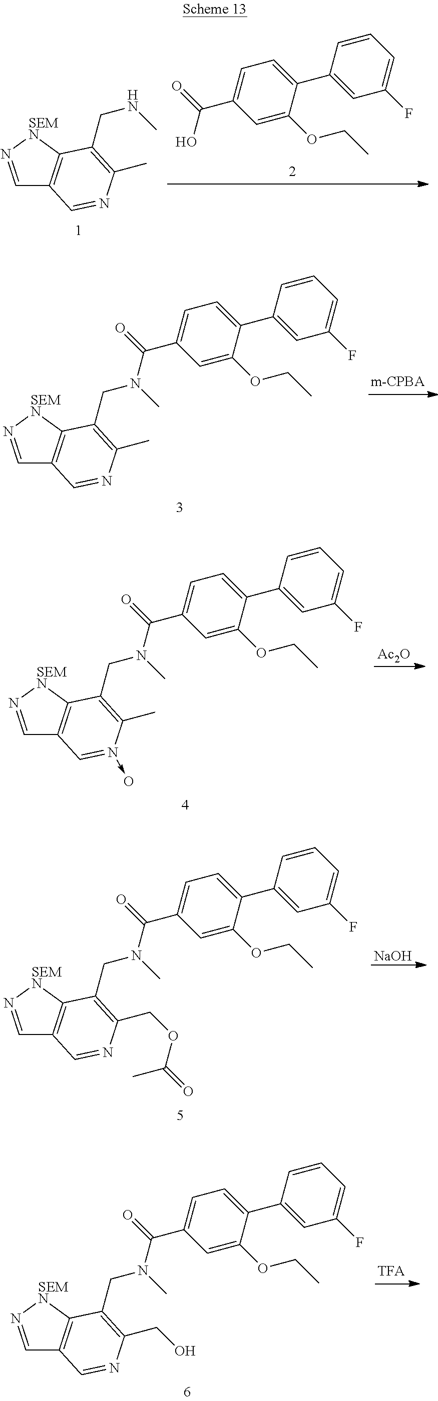

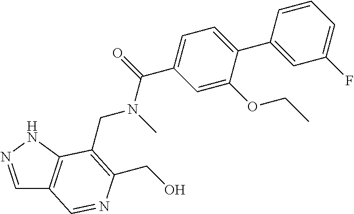 [SMILES: CCOc1cc(C(=O)N(C)Cc2c(CO)ncc3cn[nH]c23)ccc1-c1cccc(F)c1]

Dissolved Compound 1 (169.84 mg, 652.58 umol, 1 eq) in DCM (5 mL) in a 40 mL flask, was added Compound 2 (200 mg, 652.58 umol, 1 eq), EDCI (187.65 mg, 978.86 umol, 1.5 eq), DMAP (7.97 mg, 65.26 umol, 0.1 eq), the reaction mixture was stirred at 20° C. by magnetic stirring apparatus for 16 hr. The reaction mixture was concentrated under vacuum to give a residue. The crude product was purified by column chromatography on silica gel eluted with PE:EtOAc=0:1 (desired compound=0.5), the purified solution was concentrated on vacuum to give colorless oil. Compound 3 (348 mg, 615.17 umol, 94.27% yield, 97% purity) was obtained as colorless oil. LCMS: Rt=0.484 min, m/z=549.4 (M+H⁺)

To a solution of 3 (100 mg, 182.24 umol, 1 eq) in DCM (2 mL) in a 50 mL single-mouth round bottom flask, was added m-CPBA (111.00 mg, 546.72 umol, 85% purity, 3 eq) slowly at 0° C., the mixture was stirred at 20° C. by magnetic stirrer for 1 hr. After the reaction was completed, the reaction mixture was quenched by Na₂SO₃ (20 mL) and extracted by EtOAc (20 mL×3), the combined organic phase was dried and concentrated under vacuum to give a residue. The crude product was purified by prep-HPLC (column: Waters xbridge 150*25 mm 10 um; mobile phase: [water (NH₄HCO₃)-CAN]; B %: 45%-75%, 8 min). After prep-HPLC purification, the eluent was concentrated to remove organic solvents. The residual aqueous solution was lyophilized to give white solid. Compound 4 (42 mg, 73.63 umol, 40.40% yield, 99% purity) was obtained as white solid. LCMS: Rt=0.699 min, m/z=565.1 (M+H⁺)

A solution of Compound 4 (50 mg, 88.54 umol, 1 eq) in Ac₂O (5.45 g, 53.38 mmol, 5 mL, 602.95 eq) in a 40 mL was stirred at 100° C. for 16 hr under N₂. The reaction mixture partitioned between EtOAc (40 ml) and water (40 ml). The separated organic layer was dried over (Na₂SO₄) and evaporated to dryness to give a crude product. The crude product was purified by prep-HPLC (column: Phenomenex luna C18 150*25 mm*10 um; mobile phase: [water (FA)-ACN]; B %: 62%-92%, 8 min). After prep-HPLC purification, the eluent was concentrated to remove organic solvent. The residual aqueous solution was lyophilized to give white solid. Compound 5 (10 mg, 16.48 umol, 18.61% yield) was obtained as white solid. ¹H-NMR: (400 MHz, chloroform-d) δ=9.15 (s, 1H), 8.24 (s, 1H), 7.44 (s, 2H), 7.38-7.34 (m, 2H), 7.20-7.13 (m, 2H), 7.08 (br t, J=8.4 Hz, 1H), 5.99-5.98 (m, 2H), 5.52-5.38 (m, 4H), 3.65-3.60 (m, 2H), 2.91 (s, 3H), 2.19 (s, 3H), 2.06 (s, 2H), 1.45-1.41 (m, 3H), 0.95-0.91 (m, 3H), 0.00 (s, 9H).

Dissolved Compound 5 (7 mg, 11.54 umol, 1 eq) in H₂O (1 mL), THF (1 mL) and EtOH (1 mL) in a 100 mL single-necked round bottom flask was added LiOH·H₂O (2.42 mg, 57.68 umol, 5 eq), the reaction mixture was stirred at 20° C. for 1 hr by magnetic stirring apparatus. After the reaction was completed, the mixture was extracted with EtOAc (25 mL×3), the combined organic phase was washed with brine (50 ml), dried over anhydrous Na₂SO₄, and concentrated to give a crude product. The crude product was used to the next step without purification. Compound 6 (30 mg, crude) was obtained as white solid. LCMS: Rt=0.471 min, m/z=565.5 (M+H⁺)

Dissolved Compound 6 (30 mg, 53.12 umol, 1 eq) in DCM (2 mL) in a 100 mL single-necked round bottom flask, and added TFA (1.54 g, 13.51 mmol, 1 mL, 254.24 eq) at 0° C., the reaction mixture was stirred at 20° C. for 1 hr by magnetic stirring apparatus. After the reaction was completed, remove the solution on a reduce pressure using a rotary evaporator to give a crude product. The crude product was purified by prep-HPLC (column: Phenomenex C18 150*25 mm*10 um; mobile phase: [water (NH₄HCO₃)-ACN]; B %: 28%-58%, 8 min). After prep-HPLC purification, the eluent was concentrated to remove organic solvents. The residual aqueous solution was lyophilized to give brown solid. 039 (2.8 mg, 6.44 umol, 12.13% yield) was obtained as brown solid, LCMS: Rt=0.574 min, m/z=435.3 (M+H⁺) ¹H-NMR: (400 MHz, chloroform-d) δ=12.87-12.67 (m, 1H), 9.21 (br s, 1H), 8.43 (s, 1H), 7.40-7.36 (m, 2H), 7.33-7.31 (m, 2H), 7.11-7.04 (m, 3H), 5.23-5.16 (m, 2H), 5.13 (s, 2H), 4.09 (q, J=7.0 Hz, 2H), 3.11 (s, 3H), 1.38 (s, 3H).

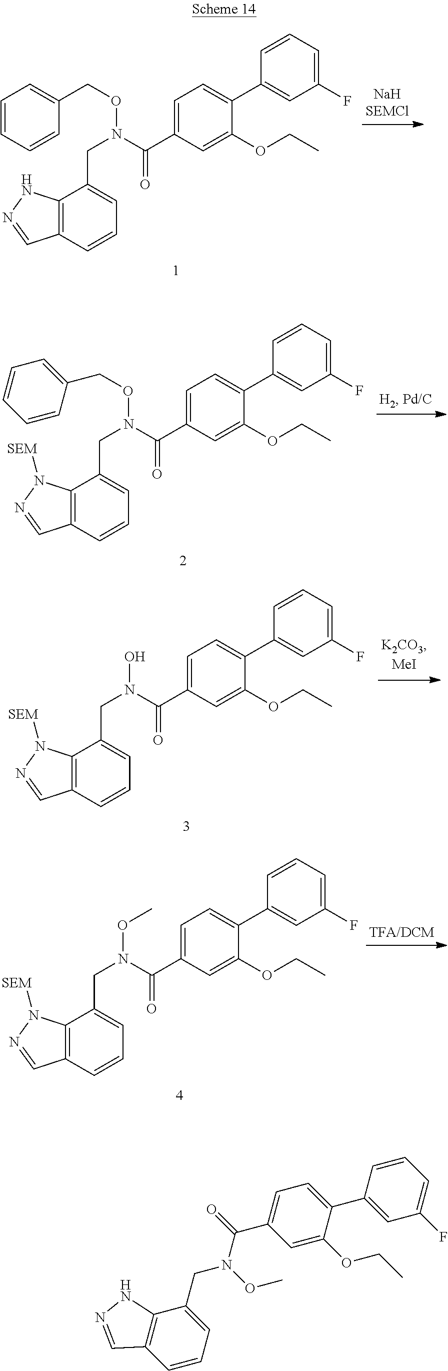 [SMILES: CCOc1cc(C(=O)N(Cc2cccc3cn[nH]c23)OCc2ccccc2)ccc1-c1cccc(F)c1 ; CCOc1cc(C(=O)N(Cc2cccc3cnn(COCC[Si](C)(C)C)c23)OCc2ccccc2)ccc1-c1cccc(F)c1 ; CCOc1cc(C(=O)N(O)Cc2cccc3cnn(COCC[Si](C)(C)C)c23)ccc1-c1cccc(F)c1 ; CCOc1cc(C(=O)N(Cc2cccc3cnn(COCC[Si](C)(C)C)c23)OC)ccc1-c1cccc(F)c1 ; CCOc1cc(C(=O)N(Cc2cccc3cn[nH]c23)OC)ccc1-c1cccc(F)c1]

To a solution of compound 1 (370 mg, 746.66 umol, 1 eq) in 2-MeTHF (30 mL) in a 250 ml three-neck bottle was added NaH (44.80 mg, 1.12 mmol, 60% purity, 1.5 eq) at 0° C. under N₂, the mixture was stirred at 0° C. for 1 hr under N₂. Then SEM-CI (149.38 mg, 895.99 umol, 158.58 uL, 1.2 eq) was added dropwise at 0° C. under N₂, the mixture was stirred at 20° C. by magnetic stirrer for 12 hr under N₂. After the reaction was completed, the reaction mixture was poured into saturated NH₄Cl (100 mL) solution at room temperature, and extracted with EtOAc (100 mL×3), the combined organic phase was washed with brine (100 mL), dried over anhydrous Na₂SO₄, and concentrated to give a crude product. The residue was purified by flash silica gel chromatography (ISCO®; 4 g SepaFlash® Silica Flash Column, Eluent of 0-28% EtOAc/PEgradient @40 mL/min). Compound 2 (430 mg, 687.12 umol, 92.03% yield) was obtained as a colourless oil. LCMS: Rt=0.637 min, m/z=626.3 (M+Na⁺)

To a solution of compound 2 (200 mg, 319.59 umol, 1 eq) in MeOH (20 mL) was added Pd/C (200 mg, 31.96 umol, 10% purity, 0.1 eq) under N₂, the mixture was stirred at 20° C. for 1 hr under H₂ (644.23 ug, 319.59 umol, 1 eq) (15 Psi). After the reaction was completed, the mixture was filtered under N₂ atmosphere, the filtrated was concentrated to give a crude product. The residue was purified by prep-TLC (SiO₂, PE/EtOAc=1/1). Compound 3 (102 mg, 190.41 umol, 59.58% yield) was obtained as a yellow oil. LCMS: Rt=0.590 min, m/z=536.3 (M+H⁺)

To a solution of compound 3 (80 mg, 149.34 umol, 1 eq) in DMF (2 mL) in 8 mL of glass bottle was added K₂CO₃ (41.28 mg, 298.69 umol, 2 eq) and Mel (63.59 mg, 448.03 umol, 27.89 uL, 3 eq). The mixture was stirred at 50° C. for 12 hr. The reaction mixture was poured into water (10 mL) and extracted by EtOAc (20 mL×3), the combined organic phase was dried and concentrated under vacuum to give a residue. The residue was purified by prep-TLC (SiO₂, PE/EtOAc=5/1). Compound 4 (45 mg, 81.86 umol, 54.81% yield) was obtained as a colourless oil. LCMS: Rt=0.625 min, m/z=550.3 (M+H⁺)

To a solution of compound 4 (45 mg, 81.86 umol, 1 eq) in DCM (2 mL) in 100 ml of round-botton flask was added TFA (3.08 g, 27.01 mmol, 2 mL, 329.97 eq) at 20° C. The mixture was stirred at 20° C. for 2 hr. The reaction mixture was concentrated under reduced pressure to give a residue. The residue was purified by prep-HPLC (column: Waters xbridge 150*25 mm 10 um; mobile phase: [water(NH4HCO3)-ACN]; B %: 45%-75%, 8 min). Then the purified solution was lyophilized to give the product. 060 (15.4 mg, 36.71 umol, 44.85% yield, 100% purity) was obtained as a white solid. LCMS: Rt=0.491 min, m/z=420.3 (M+H⁺) ¹H NMR: (400 MHz, chloroform-d) δ=11.60 (br s, 1H), 8.06 (s, 1H), 7.77-7.66 (m, 1H), 7.44-7.39 (m, 1H), 7.35 (d, J=1.4 Hz, 1H), 7.33-7.27 (m, 3H), 7.27-7.18 (m, 2H), 7.11-7.05 (m, 1H), 7.01-6.93 (m, 1H), 5.19 (s, 2H), 4.06-3.98 (m, 2H), 3.32 (s, 3H), 1.31 (t, J=7.0 Hz, 3H).

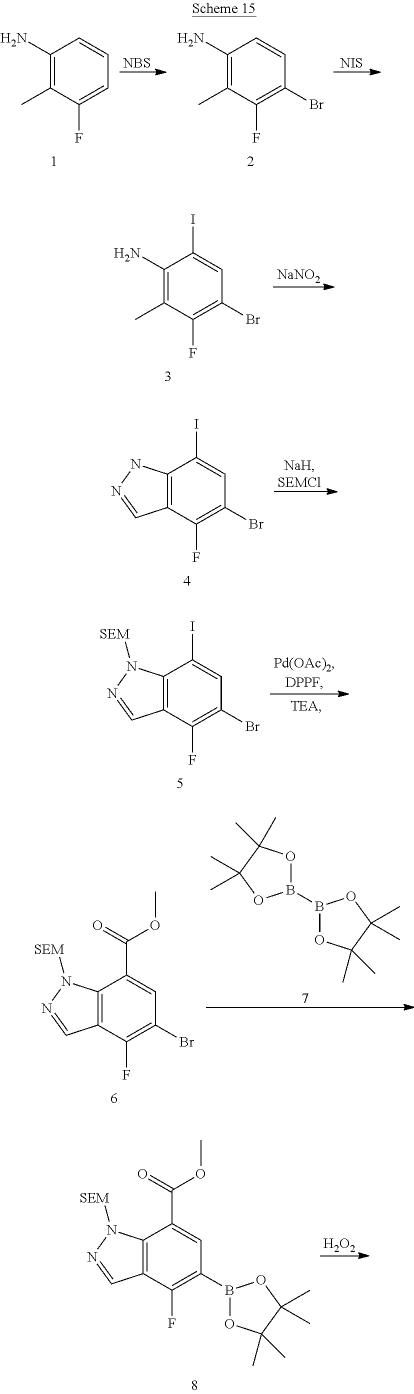

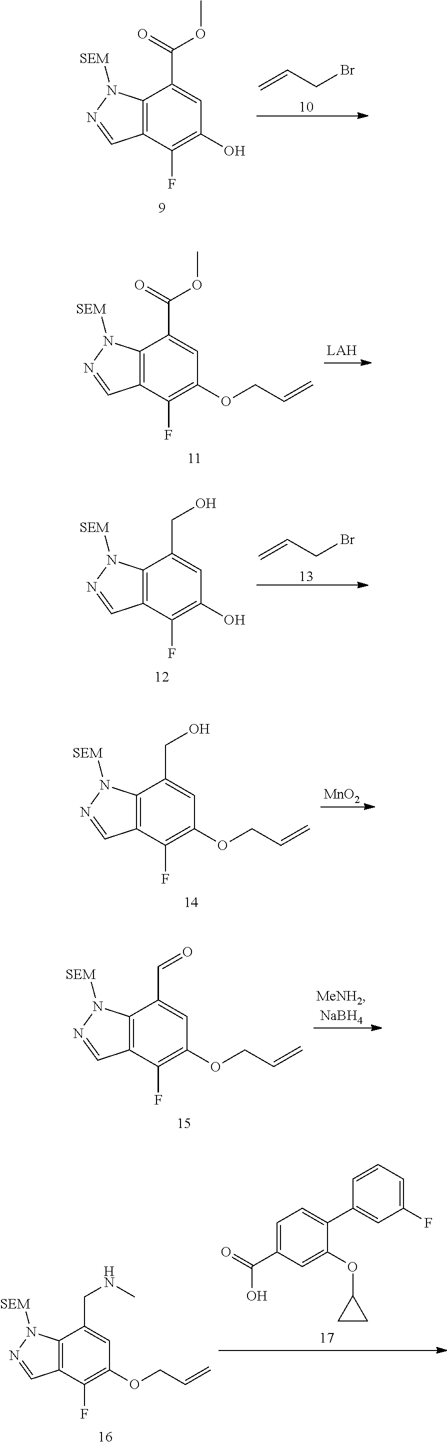

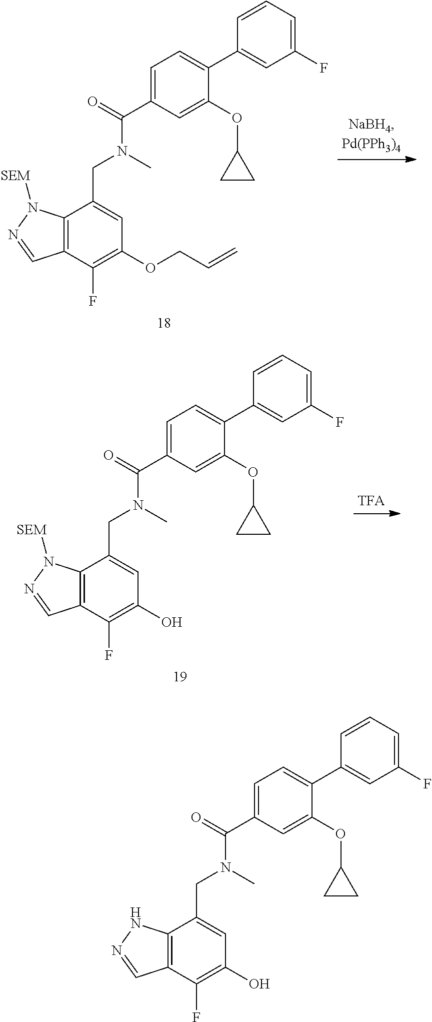 [SMILES: C=CCOc1cc(CN(C)C(=O)c2ccc(-c3cccc(F)c3)c(OC3CC3)c2)c2c(cnn2COCC[Si](C)(C)C)c1F ; CN(Cc1cc(O)c(F)c2cnn(COCC[Si](C)(C)C)c12)C(=O)c1ccc(-c2cccc(F)c2)c(OC2CC2)c1 ; CN(Cc1cc(O)c(F)c2cn[nH]c12)C(=O)c1ccc(-c2cccc(F)c2)c(OC2CC2)c1]

To a solution of Compound 1 (5 g, 39.95 mmol, 4.55 mL, 1 eq) in DCM (50 mL) was added NBS (7.11 g, 39.95 mmol, 1 eq), then the mixture was stirred at 20° C. for 2 hr. The reaction mixture was poured into water (100 mL) and extracted by EtOAc (100 mL×3), the combined organic phase was dried and concentrated under vacuum to give a residue. The residue was purified by column chromatography (SiO₂, PE/EtOAc=10/1), the purified solution was concentrated under vacuum to give Compound 2 (6 g, 29.41 mmol, 73.60% yield) as an orange oil. LCMS: Rt=0.386 min, m/z=204.1 (M+H)^{+ 1}H NMR: (400 MHz, chloroform-d) δ=7.17-7.11 (m, 1H), 6.40-6.36 (m, 1H), 3.83-3.59 (m, 2H), 2.12-2.07 (m, 3H)

To a solution of Compound 2 (6 g, 29.41 mmol, 1 eq) in MeCN (60 mL) was added NIS (6.62 g, 29.41 mmol, 1 eq) under 0° C., then the mixture was stirred at 20° C. for 1 hr. The reaction mixture was poured into water (100 mL) and extracted by EtOAc (100 mL×3), the combined organic phase was dried and concentrated under vacuum to give a residue. The residue was purified by column chromatography (SiO₂, PE/EtOAc=10/1), the purified solution was concentrated under vacuum to give Compound 3 (6 g, 18.19 mmol, 61.84% yield) as a brown oil, LCMS: Rt=0.515 min, m/z=329.8 (M+H)⁺ 1H NMR: (400 MHz, chloroform-d) δ=7.69-7.65 (m, 1H), 4.21 (br s, 2H), 2.19-2.17 (m, 3H)

To a solution of Compound 3 (6 g, 18.19 mmol, 1 eq) in AcOH (60 mL) and H₂O (6 mL) was added NaNO₂ (1.25 g, 18.19 mmol, 1 eq) under 5° C., then the mixture was stirred at 20° C. for 2 hr. The mixture was poured into water (100 mL) and adjust to pH=8 with saturated Na₂CO₃ brine (100 mL), dried over anhydrous Na₂SO₄, and concentrated to give a crude product. The residue was purified by column chromatography (SiO₂, PE/EtOAc=10/1-5/1), the purified solution was concentrated under vacuum to give Compound 4 (4.65 g, 13.64 mmol, 75.00% yield) as a brown solid, 1H NMR: (400 MHz, chloroform-d) δ=8.35-8.28 (m, 1H), 7.84 (br d, J=5.8 Hz, 1H)

To a solution of Compound 4 (4.65 g, 13.64 mmol, 1 eq) in 2-MeTHF (100 mL) was added NaH (818.30 mg, 20.46 mmol, 60% purity, 1.5 eq) under 0° C., the mixture was stirred at 0° C. for 1 hr, Then SEM-CI (3.41 g, 20.46 mmol, 3.62 mL, 1.5 eq) was added into the reaction under 0° C., the reaction mixture was stirred at 20° C. for 1 hr. The reaction mixture was poured into saturated NH₄Cl aqueous solution (500 mL), the mixture was extracted by EtOAc (500 mL×3), the combined organic phase was dried and concentrated under vacuum to give a residue. The residue was purified by column chromatography (SiO₂, PE/EtOAc=5/1), the purified solution was concentrated under vacuum to give Compound 5 (6.2 g, crude) as a brown oil. LCMS: Rt=0.662 min, m/z=354.8 (M+H)⁺

To a solution of Compound 5 (960 mg, 2.04 mmol, 1 eq) in MeOH (20 mL) in glass bottle (75 mL) was added Pd(OAc)₂ (45.74 mg, 203.74 umol, 0.1 eq), DPPF (225.90 mg, 407.49 umol, 0.2 eq) and TEA (412.33 mg, 4.07 mmol, 567.17 uL, 2 eq). The mixture was stirred at 40° C. for 12 hr under CO (30 psi). LCMS (5-95 AB/0.8 min) showed 70% of Compound 5 remained. The reaction mixture was filtered and concentrated under vacuum to give a residue. The residue was purified by column chromatography (SiO₂, PE/EtOAc=10/1 to 5/1), the purified solution was concentrated under vacuum to give Compound 6 (190 mg, 471.09 umol, 23.12% yield) as a yellow oil. LCMS: Rt=0.545 min, m/z=285.0 (M+H)⁺

To a solution of Compound 6 (850 mg, 2.11 mmol, 1 eq) in dioxane (10 mL) in glass bottle (40 mL) was added Compound 7 (535.18 mg, 2.11 mmol, 1 eq), KOAc (413.67 mg, 4.22 mmol, 2 eq) and Pd(dppf)Cl₂·CH₂Cl₂ (172.11 mg, 210.75 umol, 0.1 eq), then the mixture was stirred at 100° C. for 12 hr under N₂. The reaction mixture was filtered under vacuum to give filter liquor, then the filter liquor was concentrated under vacuum to give a residue. The residue was purified by column chromatography (SiO₂, PE/EtOAc=10/1 to 5/1), the purified solution was concentrated under vacuum to give Compound 8 (600 mg, 1.33 mmol, 63.21% yield) as a yellow oil. LCMS: Rt=0.623 min, m/z=333.1 (M+H)⁺

To a solution of Compound 8 (600 mg, 1.33 mmol, 1 eq) in AcOH (6 mL) in round-necked flask (100 mL) was added H₂O₂ (7.08 g, 62.44 mmol, 6 mL, 30% purity, 46.87 eq) at 20° C. slowly. Then the mixture was stirred at 20° C. for 1 h. The reaction mixture was quenched by Na₂SO₃ (60 ml) and extracted by DCM (60 mL×3), the combined organic phase was dried and concentrated under 20° C. to give Compound 9 (430 mg, 1.26 mmol, 94.82% yield) as a yellow oil which was used directly in the next step. LCMS: Rt=0.492 min, m/z=223.2 (M+H)⁺

To a solution of methyl Compound 9 (330 mg, 969.39 umol, 1 eq) in acetone (4 mL) in glass bottle (40 mL) was added K₂CO₃ (267.95 mg, 1.94 mmol, 2 eq), KI (16.09 mg, 96.94 umol, 0.1 eq) and Compound 10 (140.73 mg, 1.16 mmol, 1.2 eq), then the mixture was stirred at 60° C. for 12 hr. The reaction mixture was poured into water (20 mL) and extracted by EtOAc (20 mL×3), the combined organic phase was dried and concentrated under vacuum to give Compound 11 (360 mg, 946.16 umol, 97.60% yield) as a yellow oil which was used directly in the next step. LCMS: Rt=0.553 min, m/z=263.1 (M+H)⁺

To a solution of methyl Compound 11 (360 mg, 946.16 umol, 1 eq) in 2-MeTHF (5 mL) in slenktube (20 mL) was added LAH (53.87 mg, 1.42 mmol, 1.5 eq) at 0° C. slowly, the mixture was stirred at 0° C. for 1 hr. After the reaction was completed, the resulting mixture was added Na₂SO₄·10H₂O (200 mg), then the mixture was filtered and the filtrate was concentrated to give Compound 12 (230 mg, 736.21 umol, 77.81% yield) as a white solid which was used directly in the next step. LCMS: Rt=0.421 min, m/z=195.2 (M+H)⁺

To a solution of Compound 12 (230 mg, 736.21 umol, 1 eq) in acetone (3 mL) in glass bottle (40 mL) was added K₂CO₃ (203.50 mg, 1.47 mmol, 2 eq), KI (12.22 mg, 73.62 umol, 0.1 eq) and Compound 13 (89.06 mg, 736.21 umol, 1 eq), then the mixture was stirred at 60° C. for 12 hr. The reaction mixture was poured into water (20 mL) and extracted by EtOAc (20 mL×3), the combined organic phase was dried and concentrated under vacuum to give residue. The residue was purified by column chromatography (SiO₂, PE/EtOAc=5/1), the purified solution was concentrated under vacuum to give Compound 14 (220 mg, 624.16 umol, 84.78% yield) as a colorless oil. LCMS: Rt=0.495 min, m/z=235.2 (M+H)⁺

To a solution of Compound 14 (220 mg, 624.16 umol, 1 eq) in DCM (3 mL) was added MnO₂ (1.09 g, 12.48 mmol, 20 eq) in glass bottle (40 mL), the mixture was stirred at 20° C. for 12 hr. After the reaction was completed, the mixture was filtered and the filtrated was concentrated to give Compound 15 (190 mg, 542.15 umol, 86.86% yield) as a colorless oil which was used directly in the next step. LCMS: Rt=0.540 min, m/z=233.2 (M+H)⁺

To a solution of Compound 15 (190 mg, 542.15 umol, 1 eq) in MeOH (2 mL) in a 100 mL three-necked flask, was added AcOH (3.26 mg, 54.21 umol, 3.10 uL, 0.1 eq) and MeNH₂ in THF (2 M, 813.22 uL, 3 eq) at 20° C., the mixture was stirred at 20° C. for 1 hr. Then the mixture was added NaBH₄ (41.02 mg, 1.08 mmol, 2 eq) at 0° C., the mixture was stirred at 20° C. for 1 hr. The reaction mixture was quenched by saturated NaHCO₃ aqueous (20 mL) and extracted by dichloromethane (20 mL×3), the combined organic phase was dried and concentrated under vacuum to give Compound 16 (120 mg, 328.30 umol, 60.56% yield) as a colorless oil which was used directly in the next step. LCMS: Rt=0.394 min, m/z=366.3 (M+H)⁺

To a solution of Compound 17 (37.24 mg, 136.79 umol, 1 eq) in DCM (1 mL) in glass bottle (8 mL) was added EDCI (39.34 mg, 205.19 umol, 1.5 eq), DMAP (1.67 mg, 13.68 umol, 0.1 eq) and Compound 16 (50 mg, 136.79 umol, 1 eq). The mixture was stirred at 20° C. for 1 hr The reaction mixture was concentrated on vacuum to give residue. The residue was purified by prep-TLC(SiO₂, PE/EtOAc=1/1), the purified solution was concentrated under vacuum to give Compound 18 (60 mg, 96.81 umol, 70.77% yield) as a colorless oil. LCMS: Rt=0.598 min, m/z=502.3 (M+H)⁺

To a solution of Compound 18 (60 mg, 96.81 umol, 1 eq) in THF (1 mL) in slenktube (20 mL) was added Pd(PPh₃) 4 (11.19 mg, 9.68 umol, 0.1 eq), then the mixture was added NaBH₄ (36.63 mg, 968.10 umol, 10 eq) under 0° C., the mixture was stirred at 20° C. for 1 hr under N₂. The reaction mixture was poured into saturated NH₄Cl (20 mL) and extracted by EtOAc (20 mL×3), the combined organic phase was dried and concentrated under vacuum to give Compound 19 (40 mg, 69.00 umol, 71.27% yield) as a brown oil which was used directly in the next step. LCMS: Rt=0.550 min, m/z=462.3 (M+H)⁺

To a solution of Compound 19 (40 mg, 69.00 umol, 1 eq) in round-bottom flask (100 mL) was added TFA (1.54 g, 13.51 mmol, 1 mL, 195.74 eq) and DCM (0.5 mL), then the mixture was stirred at 20° C. for 1 hr. The reaction mixture was basified by saturated NaHCO₃ aqueous (10 mL), then the reaction mixture was diluted with H₂O (10 mL) and extracted with EA (10 mL×3). The combined organic layers were washed with brine, dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; B %: 44%-74%, 9 min), the purified solution was lyophilized to give Compound 061 (13.7 mg, 29.51 umol, 42.76% yield, 96.8% purity) as a brown solid, LCMS: Rt=0.456 min, m/z=450.2 (M+H)⁺ 1H NMR (400 MHz, chloroform-d) δ=8.12-8.09 (m, 1H), 7.44 (d, J=1.1 Hz, 1H), 7.38-7.34 (m, 1H), 7.32 (d, J=7.6 Hz, 1H), 7.23 (br d, J=7.8 Hz, 1H), 7.19 (dd, J=2.3, 10.2 Hz, 1H), 7.09 (d, J=7.3 Hz, 2H), 7.03 (dt, J=1.7, 8.5 Hz, 1H), 4.89 (s, 2H), 3.80-3.73 (m, 1H), 3.05-3.01 (m, 3H), 0.83-0.78 (m, 2H), 0.77-0.72 (m, 2H).

Compound 063 was prepared in a similar manner to Scheme 15 and was isolated as a yellow solid (26 mg, 58.60 umol, 55.45% yield, 98.6% purity). LCMS: Rt=0.447 min, m/z=438.2 (M+H)⁺ 1H NMR (400 MHz, chloroform-d) δ=8.04-8.01 (m, 1H), 7.31-7.26 (m, 2H), 7.25 (s, 1H), 7.24-7.21 (m, 2H), 7.01-6.99 (m, 1H), 6.99-6.97 (m, 2H), 6.97-6.93 (m, 1H), 4.81-4.79 (m, 2H), 4.02-3.96 (m, 2H), 2.94 (s, 3H), 1.31-1.27 (m, 3H).

Compound 148 was prepared in a similar manner to Scheme 15 and was isolated as a white solid (9.7 mg, 21.51 umol, 12.21% yield, 97% purity). LCMS: Rt=0.488 min, m/z=438.1 (M+H⁺) 1H NMR (400 MHz, chloroform-d) δ=8.03 (s, 1H), 7.41-7.30 (m, 5H), 7.09 (s, 3H), 5.09 (s, 2H), 4.08 (q, J=7.0 Hz, 2H), 3.10-3.08 (m, 3H), 1.40-1.36 (m, 3H).

Compound 178 was prepared in a similar manner to Scheme 15 and was isolated as a white solid (21.8 mg, 48.03 umol, 31.17% yield). H-NMR: (400 MHz, chloroform-d) δ=8.00 (s, 1H), 7.41 (s, 1H), 7.36-7.33 (m, 2H), 7.32-7.28 (m, 1H), 7.11 (d, J=1.3 Hz, 1H), 6.98 (s, 1H), 6.86-6.83 (m, 2H), 4.91 (s, 2H), 4.03 (q, J=7.0 Hz, 2H), 3.02 (s, 3H), 1.30 (t, J=6.9 Hz, 3H). LCMS: Rt=0.486 min, m/z=454.0 (M+H⁺).

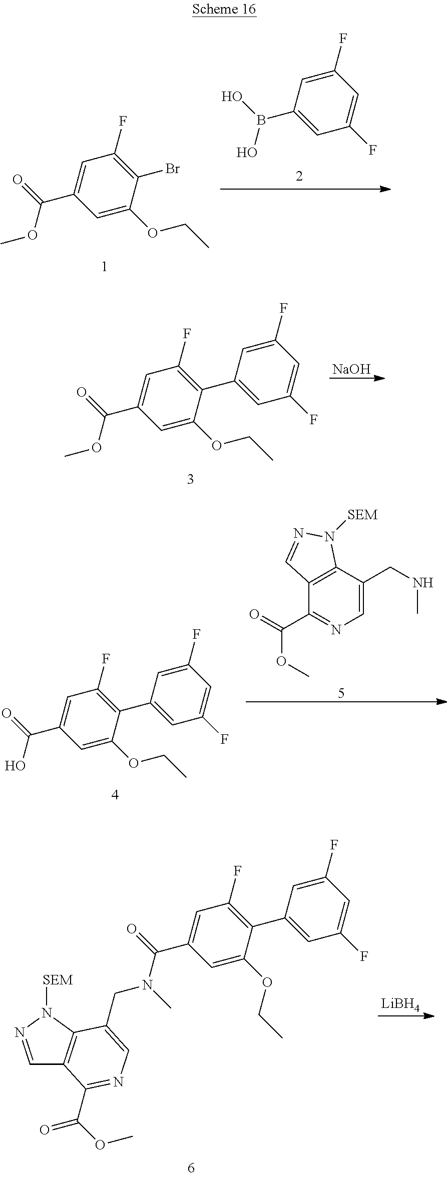

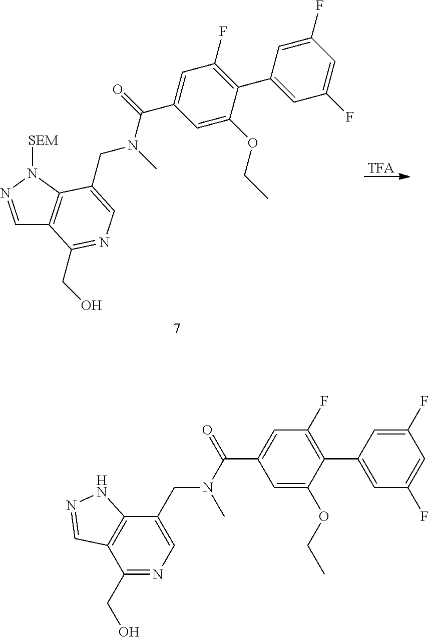 [SMILES: CCOc1cc(C(=O)N(C)Cc2cnc(CO)c3cnn(COCC[Si](C)(C)C)c23)cc(F)c1-c1cc(F)cc(F)c1 ; CCOc1cc(C(=O)N(C)Cc2cnc(CO)c3cn[nH]c23)cc(F)c1-c1cc(F)cc(F)c1 ; "TFA"]

To a solution of Compound 1 (400 mg, 1.44 mmol, 1 eq) and Compound 2 (455.92 mg, 2.89 mmol, 2 eq) in dioxane (10 mL) in glass bottle (40 mL) was added Pd(dppf)Cl₂·CH₂Cl₂ (117.89 mg, 144.36 umol, 0.1 eq) and K₂CO₃ (399.03 mg, 2.89 mmol, 2 eq). The mixture was stirred at 100° C. for 12 hr under N₂ atmosphere. The reaction mixture was poured into water (10 mL), and extracted with EtOAc (20 mL×3). The combined organic layers were over [Na₂SO₄], filtered and concentrated under reduced pressure to give a residue. The residue was purified by column chromatography (SiO₂, PE/EtOAc=0/1 to 5/1), the purified solution was concentrated on vacuum to give Compound 3 (340 mg, 1.10 mmol, 75.91% yield) as a white solid. LCMS: Rt=0.495 min, m/z=311.2 (M+H)⁺

To a solution of Compound 3 (340 mg, 1.10 mmol, 1 eq) in MeOH (5 mL) and H₂O (1.7 mL) in round-bottom flask (100 mL) was added LiOH·H₂O (137.95 mg, 3.29 mmol, 3 eq). The mixture was stirred at 20° C. for 1 hr. THF (5 mL) and NaOH (131.49 mg, 3.29 mmol, 3 eq) was added into the reaction mixture, the reaction mixture was stirred at 20° C. for another 1 hr. The organic solvents was evaporated under vacuum, the resulting mixture was acidified by 1N HCl to pH4, filtered, the filter cake was washed by water (20 mL), then dissolved in MeOH (20 mL), and concentrated on vacuum to give Compound 4 (300 mg, 1.01 mmol, 92.41% yield) as a white solid without further purification and used in the next step. LCMS: Rt=0.398 min, m/z=295.0 (M−H)⁺

To a solution of Compound 4 (300 mg, 1.01 mmol, 1 eq) and Compound 5 (354.93 mg, 1.01 mmol, 1 eq) in DCM (10 mL) in round-bottom flask (50 mL) was added EDCI (291.20 mg, 1.52 mmol, 1.5 eq) and DMAP (12.37 mg, 101.27 umol, 0.1 eq). The mixture was stirred at 20° C. for 12 hr. The reaction mixture was concentrated under vacuum to give residue. The residue was purified by column chromatography (SiO₂, PE/EtOAc=1/0 to 0/1), the purified solution was concentrated on vacuum to give Compound 6 (380 mg, 604.41 umol, 59.68% yield) as a green oil. LCMS: Rt=0.572 min, m/z=629.3 (M+H)⁺

To a solution of Compound 6 (380 mg, 604.41 umol, 1 eq) in MeOH (5 mL) in a round-bottom flask (50 mL) was added LiBH₄ in THF (2 M, 906.61 uL, 3 eq) at 0° C. The mixture was stirred at 20° C. for 2 hr. The reaction mixture was quenched by saturated NH₄Cl aqueous (20 mL) and extracted by EtOAc (30 mL×3), the combined organic phase was dried and concentrated under vacuum to give a residue. The residue was purified by prep-TLC (SiO₂, dichloromethane:Methanol=10:1), the purified solution was concentrated on vacuum to give Compound 7 (210 mg, 349.59 umol, 57.84% yield) as a colorless oil. LCMS: Rt=0.458 min, m/z=601.8 (M+H)⁺

To a solution of Compound 7 (30 mg, 49.94 umol, 1 eq) in DCM (1 mL) in round-bottom flask (50 mL) was added TFA (3.08 g, 27.01 mmol, 2 mL, 540.87 eq). The mixture was stirred at 20° C. for 3 hr. The reaction mixture was concentrated at 20° C. and under reduced pressure to give a residue. The residue was dissolved with dichloromethane (2 mL) and basified with NaHCO₃ (5 mg) to pH about 7 at 20° C., then filtered, the filtrate was concentrated on vacuum to give a residue. The residue was purified by prep-HPLC (column: Phenomenex luna C18 150*25 mm*10 um; mobile phase: [water(FA)-ACN]; B %: 20%-50%, 9 min), the purified solution was lyophilized to give 131 (8.5 mg, 18.07 umol, 36.18% yield, 100% purity) as a white solid. LCMS: Rt=0.363 min, m/z=471.3 (M+H)^{+ 1}H NMR (400 MHz, chloroform-d) δ=8.35-8.28 (m, 2H), 6.95 (br d, J=7.3 Hz, 2H), 6.90-6.80 (m, 3H), 5.21-5.16 (m, 2H), 4.98-4.91 (m, 2H), 4.11-4.01 (m, 2H), 3.10-3.03 (m, 3H), 1.37-1.30 (m, 3H).

Compound 084 was prepared in a similar manner to Scheme 16 using LiOH rather than NaOH. Compound 084 was obtained as a white solid (41.0 mg, 85.96 umol, 52.20% yield, 100% purity). LCMS: Rt=0.422 min, m/z=476.16 (M+H⁺) ¹H NMR: (400 MHz, chloroform-d) δ=12.43-12.15 (m, 1H), 8.28 (d, J=5.6 Hz, 2H), 7.55 (s, 1H), 7.48-7.40 (m, 1H), 7.39-7.29 (m, 3H), 7.07 (dd, J=1.4, 7.8 Hz, 1H), 6.90 (d, J=1.1 Hz, 1H), 5.15 (s, 2H), 4.95 (s, 2H), 4.67 (quin, J=7.0 Hz, 1H), 3.05 (s, 3H), 2.49-2.36 (m, 2H), 2.20-2.06 (m, 2H), 1.91-1.77 (m, 2H).

Compound 085 was prepared in a similar manner to Scheme 16 using LiOH rather than NaOH. Compound 085 was obtained as a white solid (11.3 mg, 24.84 umol, 20.04% yield, and 95.5% purity). LCMS: Rt=0.384 min, m/z=454.13 (M+H⁺) ¹H NMR: (400 MHz, chloroform-d) δ=12.33 (br s, 1H), 8.27 (s, 2H), 7.68 (d, J=7.9 Hz, 1H), 7.53 (d, J=3.6 Hz, 1H), 7.46 (s, 1H), 7.37 (d, J=5.0 Hz, 1H), 7.15-7.05 (m, 2H), 5.14 (s, 2H), 4.95 (s, 2H), 3.96-3.80 (m, 1H), 3.06 (s, 3H), 0.92-0.86 (m, 4H).

Compound 087 was prepared in a similar manner to Scheme 16 using LiOH rather than NaOH. Compound 087 was obtained as a white solid (10.7 mg, 22.04 umol, 38.77% yield, 100% purity). LCMS: Rt=0.389 min, m/z=486.3 (M+H⁺) 1H NMR (400 MHz, chloroform-d) δ=8.49 (s, 1H), 8.41-8.33 (m, 1H), 7.69-7.64 (m, 2H), 7.62-7.57 (m, 1H), 7.56-7.50 (m, 1H), 7.22-7.18 (m, 1H), 6.91-6.84 (m, 1H), 5.30 (s, 2H), 5.05-4.98 (m, 2H), 3.78-3.71 (m, 1H), 3.42-3.33 (m, 2H), 1.38-1.34 (m, 3H), 0.84-0.78 (m, 2H), 0.75-0.68 (m, 2H).

Compound 098 was prepared in a similar manner to Scheme 16 using LiOH rather than NaOH. Compound 087 was obtained as a white solid (15 mg, 34.29 umol, 29.95% yield). ¹H NMR: (400 MHz, DMSO-d₆) δ=12.87-12.73 (m, 1H), 9.15 (br s, 1H), 8.02-7.76 (m, 1H), 7.54-7.38 (m, 1H), 7.28-6.84 (m, 7H), 4.93-4.72 (m, 2H), 3.82-3.56 (m, 2H), 3.09-2.97 (m, 3H), 1.25-1.00 (m, 3H). LCMS: Rt=0.449 min, m/z=438.1 (M+H⁺).

Compound 104 was prepared in a similar manner to Scheme 16 using LiOH rather than NaOH. Compound 104 was obtained as a white solid (14.28 mg, 29.91 umol, 30.29% yield, 100% purity). LCMS: Rt=0.368 min, m/z=478.1 (M+H⁺). HNMR: (400 MHz, chloroform-d) δ=12.46-11.85 (m, 1H), 8.29 (d, J=3.6 Hz, 2H), 7.54 (s, 1H), 7.46-7.34 (m, 2H), 6.91-6.84 (m, 2H), 5.15 (s, 2H), 4.95 (s, 2H), 4.07 (q, J=6.8 Hz, 2H), 3.05 (s, 3H), 1.34 (t, J=6.8 Hz, 3H).

Compound 106 was prepared in a similar manner to Scheme 16 using LiOH rather than NaOH. Compound 106 was obtained as a white solid (11.2 mg, 25.17 umol, 27.11% yield, 99% purity). LCMS: Rt=0.390 min, m/z=441.5 (M+H⁺) 1H NMR (400 MHz, chloroform-d) δ=12.57-11.76 (m, 1H), 8.26 (d, J=6.5 Hz, 2H), 7.66 (br s, 1H), 7.46 (d, J=5.1 Hz, 1H), 7.14 (t, J=4.4 Hz, 1H), 6.92-6.85 (m, 2H), 5.13 (s, 2H), 4.92 (s, 2H), 4.17 (q, J=6.9 Hz, 2H), 3.04 (s, 3H), 1.51 (t, J=6.9 Hz, 3H).

Compound 107 was prepared in a similar manner to Scheme 16 using LiOH rather than NaOH. Compound 107 was obtained as a white solid (10.3 mg, 21.75 umol, 24.82% yield, 93% purity). LCMS: Rt=0.376 min, m/z=441.2 (M+H⁺) 1H NMR (400 MHz, chloroform-d) δ=12.49-11.88 (m, 1H), 8.31-8.21 (m, 2H), 7.66-7.62 (m, 1H), 7.44-7.33 (m, 2H), 6.91-6.81 (m, 2H), 5.13 (s, 2H), 4.93 (s, 2H), 4.08 (q, J=6.9 Hz, 2H), 3.05 (s, 3H), 1.41 (t, J=6.9 Hz, 3H).

Compound 109 was prepared in a similar manner to Scheme 16 using LiOH rather than NaOH. Compound 109 was obtained as a white solid (18 mg, 40.09 umol, 21.83% yield, and 99% purity). LCMS: Rt=0.748 min, m/z=445.3 (M+H⁺) ¹H NMR: (400 MHz, chloroform-d) δ=12.18 (s, 1H), 9.13 (s, 1H), 8.51-8.21 (m, 2H), 7.54-7.33 (m, 3H), 7.27-7.17 (m, 2H), 7.15-6.98 (m, 2H), 6.18 (s, 1H), 3.78 (s, 1H), 2.82 (s, 3H), 2.46-2.22 (m, 2H), 1.29-1.16 (m, 3H), 0.88-0.72 (m, 4H).

Compound 111 was prepared in a similar manner to Scheme 16 using LiOH rather than NaOH. Compound 111 was obtained as a white solid (6.5 mg, 14.24 umol, 20.89% yield, 100% purity). LCMS: Rt=0.420 min, m/z=457.2 (M+H⁺). HNMR: (400 MHz, chloroform-d) δ=11.94-11.43 (m, 1H), 7.98 (s, 1H), 7.64 (br d, J=5.9 Hz, 2H), 7.60-7.48 (m, 1H), 7.48-7.48 (m, 1H), 7.24 (s, 1H), 7.13 (s, 1H), 7.01 (s, 1H), 6.88 (br d, J=8.9 Hz, 1H), 4.92 (s, 2H), 3.72 (br d, J=2.4 Hz, 1H), 3.04 (s, 3H), 0.82-0.72 (m, 2H), 0.67 (br s, 2H).

Compound 112 was prepared in a similar manner to Scheme 16 using LiOH rather than NaOH. Compound 112 was obtained as a white solid (10.2 mg, 23.83 umol, 29.11% yield, 98% purity). LCMS: Rt=0.451 min, m/z=420.0 (M+H⁺) 1H NMR (400 MHz, chloroform-d) δ=8.01 (s, 1H), 7.44-7.34 (m, 5H), 7.14-7.09 (m, 1H), 7.01-6.97 (m, 1H), 6.89-6.82 (m, 2H), 4.91 (s, 2H), 4.08-3.97 (m, 2H), 3.04 (s, 3H), 1.32-1.29 (m, 3H).

Compound 113 was prepared in a similar manner to Scheme 16 using LiOH rather than NaOH. Compound 113 was obtained as a white solid (5.3 mg, 11.38 umol, 13.56% yield, 100% purity). LCMS: Rt=0.499 min, m/z=466.2 (M+H⁺) 1H NMR (400 MHz, chloroform-d) δ=7.99 (s, 1H), 7.33 (s, 3H), 7.23 (s, 2H), 7.12 (s, 1H), 6.98 (s, 1H), 6.87 (d, J=8.8 Hz, 1H), 4.92 (s, 2H), 3.73 (s, 1H), 3.04 (s, 3H), 0.78 (d, J=4.9 Hz, 2H), 0.70 (s, 2H).

Compound 114 was prepared in a similar manner to Scheme 16 using LiOH rather than NaOH and was isolated as a yellow solid (16.5 mg, 38.24 umol, 10.74% yield, 100% purity). LCMS: Rt=0.451 min, m/z=431.16 (M+H⁺) ¹H NMR: (400 MHz, DMSO-d₆) δ=12.78 (br d, J=17.1 Hz, 1H), 9.16 (br s, 1H), 7.89 (br s, 1H), 7.40 (br dd, J=1.1, 5.6 Hz, 1H), 7.33 (br s, 3H), 7.26-7.19 (m, 1H), 7.12-6.98 (m, 1H), 6.97-6.85 (m, 2H), 4.99-4.74 (m, 2H), 3.12-2.99 (m, 3H), 2.05 (s, 1H), 0.85-0.55 (m, 2H), 0.50-0.26 (m, 2H).

Compound 131 was prepared in a similar manner to Scheme 16 and was isolated as a white solid (8.5 mg, 18.07 umol, 36.18% yield, 100% purity). LCMS: Rt=0.363 min, m/z=471.3 (M+H)⁺ 1H NMR (400 MHz, chloroform-d) δ=8.35-8.28 (m, 2H), 6.95 (br d, J=7.3 Hz, 2H), 6.90-6.80 (m, 3H), 5.21-5.16 (m, 2H), 4.98-4.91 (m, 2H), 4.11-4.01 (m, 2H), 3.10-3.03 (m, 3H), 1.37-1.30 (m, 3H).

Compound 151 was prepared in a similar manner to Scheme 16 using LiOH rather than NaOH. Compound 151 was obtained as a white solid (12.82 mg, 27.72 umol, 18.26% yield, 100% purity). LCMS: Rt=0.322 min, m/z=463.1 (M+H⁺) ¹H NMR: (400 MHz, DMSO-d₆) δ ppm 13.38-13.67 (m, 1H) 8.43-8.64 (m, 1H) 8.11-8.25 (m, 1H) 7.38-7.52 (m, 4H) 7.13-7.24 (m, 2H) 6.71-6.85 (m, 1H) 5.61-5.82 (m, 1H) 5.49 (s, 1H) 4.90-5.00 (m, 4H) 4.64-4.88 (m, 2H) 4.29-4.55 (m, 2H) 2.98 (br d, J=2.00 Hz, 3H).

Compound 155 was prepared in a similar manner to Scheme 16 using LiOH rather than NaOH. Compound 155 was obtained as a white solid (12.7 mg, 19.83 umol, 22.65% yield, 98.2% purity). LCMS: Rt=0.326 min, m/z=481.1 (M+H)⁺ 1H NMR (400 MHz, chloroform-d) δ=8.37 (s, 1H), 8.33 (s, 1H), 7.39 (d, J=7.8 Hz, 1H), 7.13 (dd, J=1.3, 7.8 Hz, 1H), 7.09 (dd, J=2.1, 8.3 Hz, 2H), 6.87-6.80 (m, 1H), 6.61 (d, J=1.0 Hz, 1H), 5.28-5.23 (m, 1H), 5.22 (br s, 2H), 5.00-4.97 (m, 2H), 4.96-4.94 (m, 2H), 4.72 (dd, J=5.2, 7.4 Hz, 2H), 3.05 (s, 3H).

Compound 161 was prepared in a similar manner to Scheme 16 using LiOH rather than NaOH. Compound 161 was obtained as a white solid (23.8 mg, 46.13 umol, 52.64% yield, and 96.4% purity). LCMS: Rt=0.419 min, m/z=497.1 (M+H⁺) ¹H NMR: (400 MHz, chloroform-d) δ=12.34-11.99 (m, 1H), 8.28 (s, 2H), 7.35 (d, J=5.3 Hz, 2H), 7.31 (d, J=1.3 Hz, 1H), 7.20 (d, J=0.9 Hz, 1H), 7.17 (d, J=1.3 Hz, 1H), 7.12-7.07 (m, 1H), 5.15 (s, 2H), 4.96 (s, 2H), 3.76-3.65 (m, 1H), 3.08 (s, 3H), 0.79-0.74 (m, 2H), 0.68-0.62 (m, 2H).

Compound 162 was prepared in a similar manner to Scheme 16 using LiOH rather than NaOH. Compound 162 was obtained as a white solid (37 mg, 76.94 umol, 39.18% yield, and 100% purity). LCMS: Rt=0.404 min, m/z=481.1 (M+H⁺) ¹H NMR: ¹H NMR (400 MHz, chloroform-d) δ=12.17 (s, 1H), 8.28 (s, 2H), 7.42-7.34 (m, 1H), 7.31 (d, J=1.1 Hz, 1H), 7.17 (d, J=1.0 Hz, 1H), 7.11-7.03 (m, 1H), 6.99 (d, J=7.8 Hz, 1H), 6.93 (d, J=9.4 Hz, 1H), 5.14 (s, 2H), 4.95 (s, 2H), 3.75-3.67 (m, 1H), 3.08 (s, 3H), 0.82-0.72 (m, 2H), 0.69-0.60 (m, 2H).

Compound 165 was prepared in a similar manner to Scheme 16 using LiOH rather than NaOH. Compound 165 was obtained as a white solid (7 mg, 14.07 umol, 12.60% yield, 99.8% purity). LCMS: Rt=0.481 min, m/z=486.2 (M+H⁺) ¹H NMR: (400 MHz, chloroform-d) δ=8.31 (s, 1H), 8.28 (s, 1H), 7.18 (s, 1H), 6.88 (br d, J=7.2 Hz, 2H), 6.87-6.79 (m, 2H), 5.16 (s, 2H), 4.99 (s, 2H), 3.74 (tt, J=2.8, 6.0 Hz, 1H), 3.34 (q, J=7.2 Hz, 2H), 1.33 (t, J=7.2 Hz, 3H), 0.84-0.76 (m, 2H), 0.76-0.70 (m, 2H).

Compound 175 was prepared in a similar manner to Scheme 16 using LiOH rather than NaOH. Compound 175 was obtained as a white solid (9.2 mg, 19.52 umol, 23.39% yield, 99.5% purity). LCMS: Rt=0.366 min, m/z=468.14 (M+H)⁺ 1H NMR (400 MHz, chloroform-d) δ=12.59-12.17 (m, 1H), 8.38-8.31 (m, 2H), 7.43-7.36 (m, 1H), 7.16-7.13 (m, 1H), 7.11-7.07 (m, 1H), 7.07-7.03 (m, 1H), 7.02-6.97 (m, 1H), 6.93 (s, 1H), 5.21 (s, 2H), 4.96 (s, 2H), 3.99 (q, J=7.0 Hz, 2H), 3.09 (s, 3H), 1.25 (t, J=7.0 Hz, 3H).

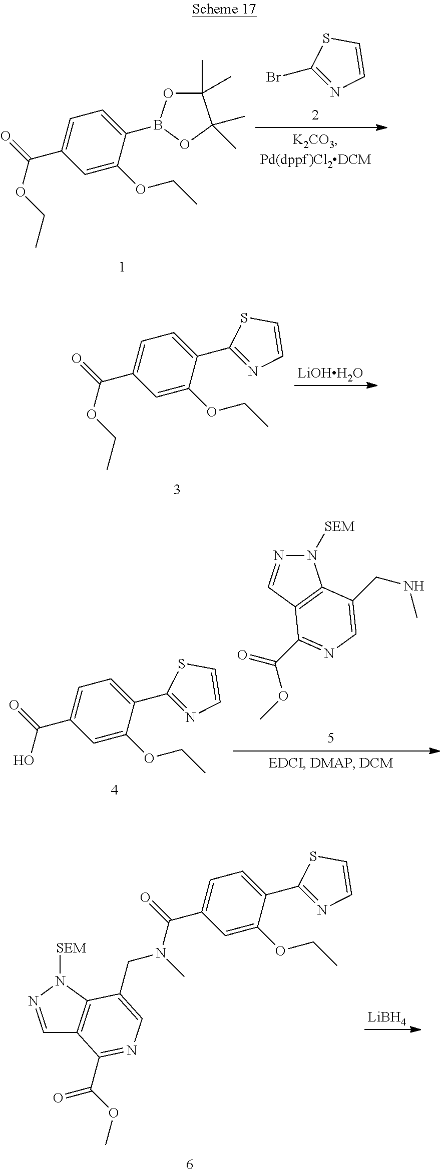

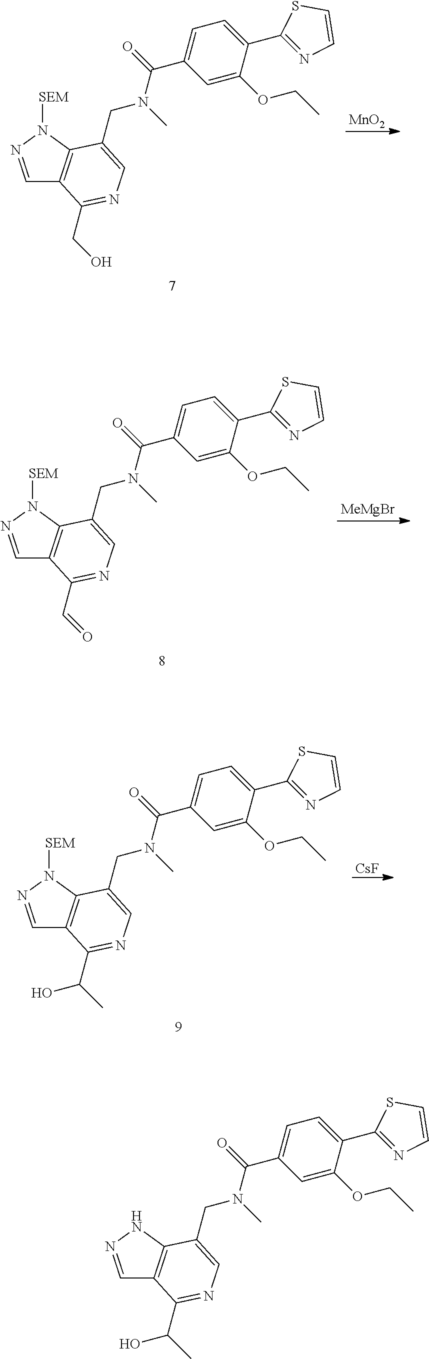

To a solution of Compound 1 (500 mg, 1.56 mmol, 1 eq) and Compound 2 (512.27 mg, 3.12 mmol, 281.47 uL, 2 eq) in dioxane (5 mL) and H₂O (0.5 mL) was added K₂CO₃ (431.64 mg, 3.12 mmol, 2 eq) and Pd(dppf)Cl₂·CH₂Cl₂ (127.52 mg, 156.16 umol, 0.1 eq). The mixture was stirred at 100° C. for 12 hr under N₂ atmosphere. The reaction mixture was poured into water (10 mL) and extracted by EtOAc (10 mL×3), the combined organic phase was dried and concentrated under vacuum to give residue. The residue was purified by column chromatography (SiO₂, PE/EtOAc=1/0 to 10/1), the purified solution was concentrated on vacuum to give Compound 3 (200 mg, 721.14 umol, 46.18% yield) as a white solid. 1H NMR (400 MHz, chloroform-d) δ=8.51 (d, J=8.3 Hz, 1H), 7.98 (d, J=3.3 Hz, 1H), 7.76 (dd, J=1.5, 8.3 Hz, 1H), 7.70 (d, J=1.4 Hz, 1H), 7.49-7.45 (m, 1H), 4.45-4.39 (m, 2H), 4.39-4.34 (m, 2H), 1.65 (t, J=7.0 Hz, 3H), 1.45-1.41 (m, 3H)

To a solution of Compound 3 (200 mg, 721.14 umol, 1 eq) in EtOH (3 mL), H₂O (1 mL) and THF (3 mL) was added LiOH·H₂O (90.79 mg, 2.16 mmol, 3 eq). The mixture was stirred at 20° C. for 12 hr. The solvents was removed by evaporated under reduced pressure, then the mixture was acidified by 1N HCl to pH 4, then the suspension was filtered to give Compound 4 (130 mg, 521.49 umol, 72.31% yield) as a white solid which was used in the next step. LCMS: Rt=0.355 min, m/z=247.9 (M−H⁺)

To a solution of Compound 4 (130 mg, 521.49 umol, 1 eq) and Compound 5 (182.78 mg, 521.49 umol, 1 eq) in DCM (3 mL) was added EDCI (149.96 mg, 782.24 umol, 1.5 eq) and DMAP (6.37 mg, 52.15 umol, 0.1 eq). The mixture was stirred at 20° C. for 1 hr. The reaction mixture was concentrated on vacuum to give a residue. The residue was purified by column chromatography (SiO₂, PE/EtOAc=3/1 to 0/1), the purified solution was concentrated on vacuum to give Compound 6 (180 mg, 309.41 umol, 59.33% yield) as a yellow oil. LCMS: Rt=0.561 min, m/z=582.2 (M+H⁺)

To a solution of Compound 6 (180 mg, 309.41 umol, 1 eq) in MeOH (3 mL) was added LiBH₄ (2 M, 773.52 uL, 5 eq) at 0° C. The mixture was stirred at 30° C. for 12 hr. The reaction mixture was poured into saturated NH₄Cl aqueous (10 mL) and extracted by EtOAc (10 mL×3), the combined organic phase was dried over anhydrous Na₂SO₄, and concentrated to give a residue. The residue was purified by prep-TLC (SiO₂, PE/EtOAc=0:1). The purified solution was concentrated on vacuum to give Compound 7 (95 mg, 171.56 umol, 55.45% yield) as a yellow oil. LCMS: Rt=0.400 min, m/z=554.2 (M+H⁺)

To a solution of Compound 7 (95 mg, 171.56 umol, 1 eq) in DCM (3 mL) was added MnO₂ (298.29 mg, 3.43 mmol, 20 eq). The mixture was stirred at 30° C. for 12 hr. After the reaction was completed, the mixture was filtered and the filtrated was concentrated to give a residue. The residue was purified by prep-TLC (SiO₂, PE/EtOAc=1:1). The purified solution was concentrated on vacuum to give Compound 8 (45 mg, 81.56 umol, 47.54% yield) as a yellow oil.

To a solution of Compound 8 (45 mg, 81.56 umol, 1 eq) in THF (2 mL) in Shrek tube (40 mL) was added MeMgBr (3 M, 81.56 uL, 3 eq) at −70° C. The mixture was stirred at −70° C. for 1 hr. The reaction mixture was poured into saturated NH₄Cl (5 mL). The solvent was extracted by EtOAc (5 mL×3), the combined organic phase was dried and concentrated under vacuum to give (30 mg, 52.84 umol, 64.78% yield) as a yellow solid which was used in the next step. LCMS: Rt=0.401 min, m/z=568.4 (M+H⁺)

To a solution of Compound 9 (30 mg, 52.84 umol, 1 eq) in DMF (1 mL) was added CsF (80.26 mg, 528.38 umol, 19.48 uL, 10 eq). The mixture was stirred at 100° C. for 12 hr.

The reaction mixture was filtered to give a residue. The residue was purified by prep-HPLC (column: Phenomenex C18 150*25 mm*10 um; mobile phase: [water (NH₄HCO₃)-ACN]; B %: 18%-48%, 14 min). The purified solution was lyophilized to give 149 (5 mg, 11.43 umol, 21.63% yield, 100% purity) as a white solid. LCMS: Rt=0.571 min, m/z=438.0 (M+H⁺) 1H NMR (400 MHz, chloroform-d) δ=12.41-12.31 (m, 1H), 8.48 (d, J=8.5 Hz, 1H), 8.33 (s, 1H), 8.26 (s, 1H), 7.95 (d, J=3.1 Hz, 1H), 7.46 (d, J=3.3 Hz, 1H), 7.18-7.13 (m, 2H), 5.39-5.29 (m, 1H), 4.95 (s, 2H), 4.80-4.68 (m, 1H), 4.31 (q, J=6.9 Hz, 2H), 3.08-3.01 (m, 3H), 1.70 (d, J=6.6 Hz, 3H), 1.61 (s, 3H).

Compound 067 was prepared in a similar manner to Scheme 17 and was obtained as a brown solid (32.3 mg, 66.83 umol, 39.62% yield, and 95.7% purity). LCMS: Rt=0.700 min, m/z=463.2 (M+H⁺) ¹H-NMR: (400 MHz, chloroform-d) δ=12.31 (br s, 1H), 8.32 (s, 1H), 8.25 (s, 1H), 7.42-7.35 (m, 2H), 7.34-7.29 (m, 2H), 7.09-7.01 (m, 3H), 5.33 (d, J=6.6 Hz, 1H), 4.99 (s, 2H), 4.07 (q, J=6.9 Hz, 2H), 3.34 (br d, J=7.0 Hz, 2H), 1.70 (d, J=6.6 Hz, 3H), 1.39 (t, J=6.9 Hz, 3H), 1.31 (t, J=7.1 Hz, 3H).

Compound 076 was prepared in a similar manner to Scheme 17 and was obtained as a white solid (12.09 mg, 24.60 umol, 25.49% yield, 100% purity). LCMS: Rt=0.345 min, m/z=492.18 (M+H⁺). ¹H NMR: (400 MHz, chloroform-d) δ=12.16 (br s, 1H), 8.46-8.16 (m, 2H), 7.54 (d, J=1.3 Hz, 1H), 7.45-7.33 (m, 2H), 6.94-6.84 (m, 2H), 5.32 (q, J=6.6 Hz, 1H), 4.94 (s, 2H), 4.07 (q, J=6.9 Hz, 2H), 3.05 (s, 3H), 1.68 (d, J=6.6 Hz, 3H), 1.33 (t, J=7.0 Hz, 3H).

Compound 080 was prepared in a similar manner to Scheme 17 and was obtained as a white solid. LCMS: Rt=0.369 min, m/z=468.1 (M+H+). HNMR: (400 MHz, chloroform-d) δ=12.49-12.28 (m, 1H), 8.37-8.23 (m, 2H), 7.77 (s, 1H), 7.70 (d, J=7.8 Hz, 1H), 7.63 (d, J=7.9 Hz, 1H), 7.52 (d, J=7.8 Hz, 1H), 7.50-7.46 (m, 1H), 7.32 (d, J=7.8 Hz, 1H), 7.16-7.12 (m, 1H), 5.46-5.30 (m, 1H), 4.97 (s, 2H), 3.83-3.74 (m, 1H), 3.09 (s, 3H), 1.71 (br d, J=6.1 Hz, 3H), 0.86-0.81 (m, 2H), 0.77-0.73 (m, 2H).

Compound 081 was prepared in a similar manner to Scheme 17 and was obtained as a white solid (2.6 mg, 5.49 umol, 16.53% yield, 99.7% purity). LCMS: Rt=0.370 min, m/z=473.0 (M+H⁺) 1H NMR (400 MHz, chloroform-d) δ=12.47-12.15 (m, 1H), 8.36 (s, 1H), 8.28 (s, 1H), 7.47-7.43 (m, 1H), 6.88 (s, 2H), 6.82 (s, 1H), 5.42-5.32 (m, 1H), 4.96-4.90 (m, 2H), 4.20 (q, J=7.0 Hz, 2H), 3.05 (s, 3H), 1.71-1.70 (m, 3H), 1.55-1.53 (m, 3H).

Compound 083 was prepared in a similar manner to Scheme 17 and was obtained as a yellow solid (7.1 mg, 15.83 umol, 9.16% yield, and 100% purity). LCMS: Rt=0.380 min, m/z=448.16 (M+H⁺) ¹H NMR: (400 MHz, chloroform-d) δ=12.50-12.18 (m, 1H), 8.29 (s, 1H), 8.24 (s, 1H), 7.68 (d, J=7.9 Hz, 1H), 7.53 (d, J=2.8 Hz, 1H), 7.47 (d, J=1.1 Hz, 1H), 7.37 (d, J=4.5 Hz, 1H), 7.14-7.02 (m, 2H), 5.30 (q, J=6.5 Hz, 1H), 4.94 (s, 2H), 3.95-3.85 (m, 1H), 3.06 (s, 3H), 1.67 (s, 3H), 0.93-0.85 (m, 4H).

Compound 088 was prepared in a similar manner to Scheme 17 and was obtained as a yellow solid (4.6 mg, 10.12 umol, 9.86% yield, 100% purity). LCMS: Rt=0.388 min, m/z=455.2 (M+H⁺) 1H NMR (400 MHz, chloroform-d) δ=8.40 (s, 1H), 8.30 (s, 1H), 7.64 (s, 1H), 7.43-7.40 (m, 1H), 7.38-7.34 (m, 1H), 6.90-6.85 (m, 2H), 5.45-5.35 (m, 1H), 4.95 (s, 2H), 4.09 (q, J=7.0 Hz, 2H), 3.08 (s, 3H), 1.73 (d, J=6.6 Hz, 3H), 1.43-1.40 (m, 3H).

Compound 089 was prepared in a similar manner to Scheme 17 and was obtained as a yellow solid (6.3 mg, 13.26 umol, 19.88% yield, 98.8% purity). LCMS: Rt=0.348 min, m/z=470.3 (M+H⁺) 1H NMR (400 MHz, chloroform-d) δ=8.43 (s, 1H), 8.30 (s, 1H), 7.88-7.83 (m, 1H), 7.79-7.74 (m, 1H), 7.64 (d, J=7.8 Hz, 1H), 7.55-7.50 (m, 1H), 7.36-7.32 (m, 1H), 7.09-7.05 (m, 1H), 7.01 (s, 1H), 5.47-5.41 (m, 1H), 5.00 (d, J=4.5 Hz, 2H), 4.07 (q, J=6.8 Hz, 2H), 3.39-3.28 (m, 2H), 2.28-2.18 (m, 1H), 1.75 (d, J=6.6 Hz, 3H), 1.40-1.35 (m, 3H), 1.34-1.30 (m, 3H).

Compound 090 was prepared in a similar manner to Scheme 17 and was obtained as a yellow solid (2.6 mg, 5.38 umol, 8.12% yield, and 98% purity). LCMS: Rt=0.352 min, m/z=474.2 (M+H⁺). 1H NMR: (chloroform-d): δ 12.07-12.26 (1H, m), 8.23-8.33 (2H, m), 7.61-7.76 (3H, m), 7.51-7.57 (1H, m), 6.84-6.91 (2H, m), 5.31 (1H, q, J=6.5 Hz), 4.94 (2H, s), 4.06 (2H, q, J=7.0 Hz), 3.06 (3H, s), 1.68 (3H, d, J=6.6 Hz), 1.32 (3H, t, J=6.9 Hz).

Compound 091 was prepared in a similar manner to Scheme 17 and was obtained as a yellow solid (9.6 mg, 19.58 umol, 24.11% yield, and 99% purity). LCMS: Rt=0.380 min, m/z=486.3 (M+H⁺). 1H NMR: (chloroform-d): δ 12.27 (1H, s), 8.36 (1H, s), 8.29 (1H, s), 7.63-7.69 (2H, m), 7.57-7.61 (1H, m), 7.50-7.56 (1H, m), 7.25-7.26 (1H, m), 6.88-6.95 (1H, m), 5.37 (1H, q, J=6.5 Hz), 4.96 (2H, s), 3.77 (1H, td, J=2.9 and 5.9 Hz), 3.10 (3H, s), 1.71 (3H, d, J=6.6 Hz), 0.78-0.86 (2H, m), 0.68-0.75 (2H, m).

Compound 101 was prepared in a similar manner to Scheme 17 and was obtained as a white solid (24.6 mg, 50.85 umol, 68.62% yield, 98.594% purity). LCMS: Rt=0.370 min, m/z=477.4 (M+H⁺) 1H NMR: (400 MHz, chloroform-d) δ ppm 8.41 (s, 1H) 8.33 (s, 1H) 7.45 (d, J=1.13 Hz, 2H) 7.30-7.36 (m, 4H) 7.11 (dd, J=7.75, 1.38 Hz, 1H) 5.42 (q, J=6.55 Hz, 1H) 4.98 (d, J=1.25 Hz, 2H) 3.75-3.80 (m, 1H) 3.09 (s, 3H) 2.63 (s, 1H) 1.72 (d, J=6.75 Hz, 3H) 0.79-0.85 (m, 2H) 0.73-0.78 (m, 2H).

Compound 102 was prepared in a similar manner to Scheme 17 and was obtained as a white solid (22 mg, 44.00 umol, 39.30% yield, 99% purity). LCMS: Rt=0.424 min, m/z=459.2 (M+H⁺). ¹H NMR: (chloroform-d): δ ppm 0.75-0.79 (m, 2H) 0.81-0.85 (m, 2H) 1.69 (d, J=6.63 Hz, 3H) 3.07 (s, 3H) 3.78 (tt, J=5.88, 2.94 Hz, 1H) 4.52-4.83 (m, 1H) 4.96 (s, 2H) 5.32 (q, J=6.09 Hz, 1H) 7.05-7.13 (m, 3H) 7.24 (s, 1H) 7.32 (d, J=7.75 Hz, 1H) 7.45 (d, J=1.38 Hz, 1H) 8.26 (s, 1H) 8.31 (s, 1H) 12.30 (br s, 1H).

Compound 115 was prepared in a similar manner to Scheme 17 and was obtained as a white solid (4.6 mg, 9.29 umol, 38.74% yield, 100% purity). LCMS: Rt=0.418 min, m/z=495.4 (M+H⁺) 1H NMR (400 MHz, chloroform-d) δ=12.46-12.00 (m, 1H), 8.33-8.21 (m, 2H), 7.38-7.31 (m, 3H), 7.25-7.20 (m, 2H), 6.91-6.86 (m, 1H), 5.32 (q, J=6.5 Hz, 1H), 4.95 (s, 2H), 3.75 (td, J=3.0, 5.7 Hz, 1H), 3.08 (s, 3H), 1.69 (d, J=6.5 Hz, 3H), 0.83-0.76 (m, 2H), 0.75-0.69 (m, 2H).

Compound 118 was prepared in a similar manner to Scheme 17 and was obtained as a white solid (8.3 mg, 17.74 umol, 35.29% yield, 99.7% purity). LCMS: Rt=0.384 min, m/z=466.9 (M+H⁺) ¹H NMR: (400 MHz, chloroform-d) δ=8.43-8.41 (m, 1H), 8.35-8.31 (m, 1H), 7.43-7.36 (m, 1H), 7.21-7.12 (m, 2H), 7.11-7.05 (m, 1H), 6.90-6.84 (m, 2H), 5.44 (q, J=6.6 Hz, 1H), 4.96 (s, 2H), 4.08-4.01 (m, 2H), 3.09 (s, 3H), 1.76-1.72 (m, 3H), 1.35-1.30 (m, 3H).

Compound 152 was prepared in a similar manner to Scheme 17 and was obtained as a white solid (11.83 mg, 23.81 umol, 49.77% yield, 100% purity). LCMS: Rt=0.379 min, m/z=497.3 (M+H⁺) 1H NMR: (400 MHz, chloroform-d) δ=12.72-12.19 (m, 1H), 8.41-8.39 (m, 1H), 8.34 (s, 1H), 7.39 (d, J=7.9 Hz, 1H), 7.33 (s, 1H), 7.22-7.17 (m, 1H), 7.15-7.10 (m, 2H), 6.62-6.60 (m, 1H), 5.28-5.24 (m, 1H), 5.23 (s, 2H), 4.99-4.95 (m, 4H), 4.74-4.69 (m, 2H), 3.06 (s, 3H).

Compound 156 was prepared in a similar manner to Scheme 17 using TFA instead of CsF and was obtained as a colorless solid (18.7 mg, 38.26 umol, 52.13% yield, 98.8% purity). LCMS: Rt=0.363 min, m/z=483.2 (M+H)⁺ 1H NMR (400 MHz, chloroform-d) δ=8.38 (s, 1H), 8.32 (s, 1H), 7.43-7.36 (m, 1H), 7.15 (d, J=1.3 Hz, 1H), 7.11-7.06 (m, 1H), 7.04 (d, J=7.6 Hz, 1H), 6.99 (td, J=1.8, 9.7 Hz, 1H), 6.94 (d, J=1.1 Hz, 1H), 5.39 (q, J=6.4 Hz, 1H), 4.95 (s, 2H), 3.99 (q, J=7.0 Hz, 2H), 3.08 (s, 3H), 1.70 (d, J=6.6 Hz, 3H), 1.24 (t, J=7.0 Hz, 3H).

Compound 157 was prepared in a similar manner to Scheme 17 and was obtained as a white solid (10.2 mg, 21.29 umol, 25.05% yield, and 95.6% purity). LCMS: Rt=0.356 min, m/z=457.1 (M+H⁺) ¹H NMR: ¹H NMR (400 MHz, chloroform-d) δ=12.36 (d, J=4.1 Hz, 1H), 8.46 (d, J=8.5 Hz, 1H), 8.39-8.20 (m, 2H), 7.19 (s, 1H), 7.16-7.09 (m, 2H), 5.17 (s, 2H), 4.96 (s, 2H), 4.30 (q, J=6.9 Hz, 2H), 3.03 (s, 3H), 1.62 (s, 3H).

Compound 158 was prepared in a similar manner to Scheme 17 and was obtained as a white solid (1.9 mg, 3.73 umol, 4.49% yield, 92.7% purity). LCMS: Rt=0.372 min, m/z=471.11 (M+H⁺) ¹H NMR: (400 MHz, chloroform-d) δ=12.41-12.04 (m, 1H), 8.45 (d, J=8.4 Hz, 1H), 8.29 (s, 1H), 8.24 (s, 1H), 7.19 (s, 1H), 7.16-7.09 (m, 2H), 5.30 (q, J=6.7 Hz, 1H), 4.93 (s, 2H), 4.30 (q, J=7.0 Hz, 2H), 3.02 (s, 3H), 1.67 (d, J=6.6 Hz, 3H), 1.62 (s, 3H).

Compound 163 was prepared in a similar manner to Scheme 17 and was obtained as a white solid (5.3 mg, 11.21 umol, 26.96% yield, 99.6% purity). LCMS: Rt=0.411 min, m/z=471.2 (M+H⁺) 1H NMR (400 MHz, chloroform-d) δ=12.45-12.15 (m, 1H), 8.31-8.20 (m, 2H), 7.64 (d, J=7.9 Hz, 1H), 7.35-7.32 (m, 1H), 7.09-7.04 (m, 2H), 6.94-6.90 (m, 1H), 5.35-5.26 (m, 1H), 4.92 (s, 2H), 4.20 (q, J=6.9 Hz, 2H), 3.07-2.99 (m, 3H), 1.71-1.64 (m, 3H), 1.59-1.54 (m, 3H).

Compound 164 was prepared in a similar manner to Scheme 17 and was obtained as a white solid (3.4 mg, 7.35 umol, 8.63% yield, 98.8% purity). LCMS: Rt=0.409 min, m/z=457.2 (M+H⁺) 1H NMR (400 MHz, chloroform-d) δ=12.36-12.19 (m, 1H), 8.32-8.21 (m, 2H), 7.67-7.61 (m, 1H), 7.34 (d, J=4.0 Hz, 1H), 7.10-7.02 (m, 2H), 6.92 (d, J=4.1 Hz, 1H), 5.14 (br s, 2H), 4.97-4.89 (m, 2H), 4.25-4.15 (m, 2H), 3.03 (s, 3H), 1.56 (t, J=6.9 Hz, 3H).

Compound 166 was prepared in a similar manner to Scheme 17 and was obtained as a white solid (7.8 mg, 15.42 umol, 38.83% yield, 98.7% purity). LCMS: Rt=0.384 min, m/z=499.1 (M+H)⁺ 1H NMR (400 MHz, chloroform-d) δ=12.30-12.00 (m, 1H), 8.24 (s, 1H), 8.18 (br s, 1H), 7.32-7.25 (m, 2H), 7.21-7.20 (m, 1H), 7.11-7.03 (m, 2H), 6.86 (s, 1H), 5.25 (br d, J=5.0 Hz, 1H), 4.86 (br s, 2H), 3.92 (q, J=7.0 Hz, 2H), 2.99 (s, 3H), 1.61 (br d, J=6.5 Hz, 3H), 1.17 (t, J=6.9 Hz, 3H).

Compound 168 was prepared in a similar manner to Scheme 17 and was obtained as a white solid (8.5 mg, 17.44 umol, 35.65% yield, 99.1% purity). LCMS: Rt=0.397 min, m/z=483.2 (M+H)⁺ 1H NMR (400 MHz, chloroform-d) δ ppm 12.14-12.46 (m, 1H) 8.18-8.33 (m, 2H) 7.58-7.64 (m, 1H) 7.42-7.48 (m, 1H) 7.28-7.31 (m, 1H) 7.06-7.12 (m, 1H) 6.88-6.92 (m, 1H) 5.24-5.35 (m, 1H) 4.90-4.99 (m, 2H) 3.84-3.94 (m, 1H) 3.02-3.09 (m, 3H) 1.67 (br d, J=6.38 Hz, 3H) 0.86-0.94 (m, 4H).

Compound 172 was prepared in a similar manner to Scheme 17 and was obtained as a white solid (4.5 mg, 8.48 umol, 21.29% yield, 93.7% purity). LCMS: Rt=0.410 min, m/z=497.1 (M+H⁺) 1H NMR (400 MHz, chloroform-d) δ=8.35 (s, 1H), 8.28-8.25 (m, 1H), 7.42 (s, 1H), 7.37-7.34 (m, 2H), 7.32-7.28 (m, 1H), 6.84-6.80 (m, 1H), 6.78 (s, 1H), 5.39-5.33 (m, 1H), 5.01-4.92 (m, 2H), 4.07-3.99 (m, 2H), 3.37-3.29 (m, 2H), 1.71 (d, J=6.6 Hz, 3H), 1.34-1.29 (m, 6H).

Compound 176 was prepared in a similar manner to Scheme 17 and was obtained as a white solid (6 mg, 12.53 umol, 9.54% yield). H-NMR: (400 MHz, chloroform-d) δ=8.30 (s, 1H), 8.23 (s, 1H), 7.55 (d, J=1.5 Hz, 1H), 7.42 (td, J=1.8, 6.7 Hz, 1H), 7.35-7.32 (m, 3H), 7.04 (dd, J=1.4, 7.8 Hz, 1H), 7.00 (s, 1H), 5.31 (q, J=6.5 Hz, 1H), 4.97 (s, 2H), 4.05 (q, J=6.9 Hz, 2H), 3.31 (q, J=7.1 Hz, 2H), 1.68 (d, J=6.6 Hz, 3H), 1.37 (t, J=7.0 Hz, 3H), 1.29 (t, J=7.1 Hz, 3H).

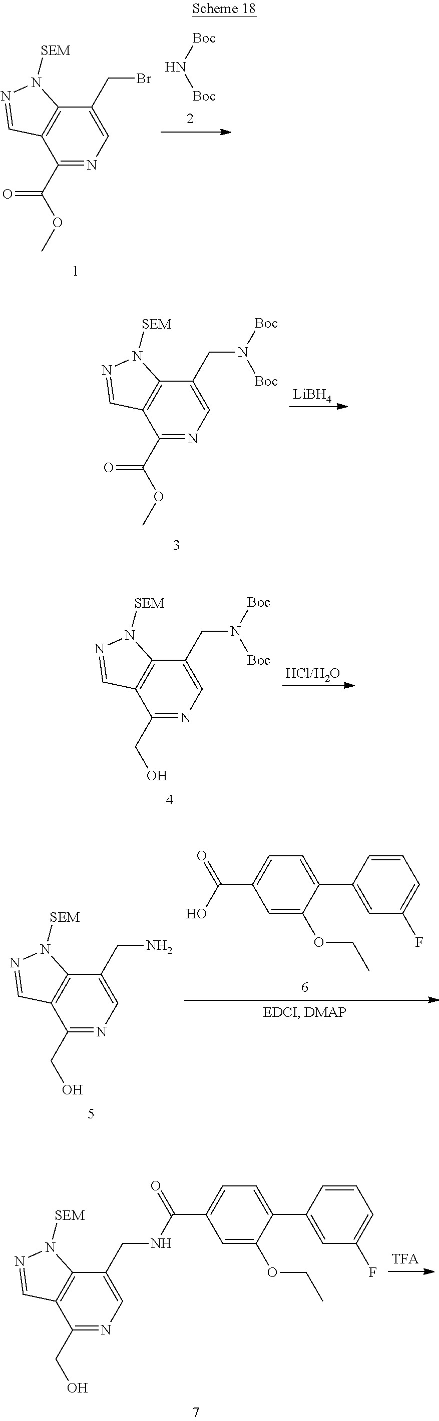

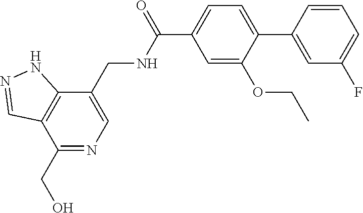 [SMILES: CCOc1cc(C(=O)NCc2cnc(CO)c3cn[nH]c23)ccc1-c1cccc(F)c1]

To a solution of compound 1 (500 mg, 1.25 mmol, 1 eq) in DMF (5 mL) in a 8 mL glass bottle was added compound 2 (325.61 mg, 1.50 mmol, 1.2 eq) and K₂CO₃ (345.22 mg, 2.50 mmol, 2 eq), the mixture was stirred at 20° C. for 12 h. After the reaction was completed, the mixture was poured into water (20 mL), extracted with EtOAc (20 mL×3), the combined organic phase was washed with brine (20 ml×3), dried over anhydrous Na₂SO₄, and concentrated to give a crude product. The crude product was purified by column chromatography (SiO₂, PE:EtOAc=2:1 to 1:1, desired product Rf=0.4). Compound 3 (550 mg, 1.02 mmol, 82.05% yield) was obtained as yellow oil. LCMS: Rt=0.554 min, m/z=537.6 (M+H⁺) ¹H NMR: (400 MHz, chloroform-d) δ=8.67 (s, 1H), 8.43-8.40 (m, 1H), 5.92 (s, 2H), 5.44 (s, 2H), 4.11-4.09 (m, 3H), 3.56-3.49 (m, 2H), 1.46 (s, 18H), 0.93-0.87 (m, 2H), −0.05 (s, 9H).

To a solution of compound 3 (500 mg, 931.63 umol, 1 eq) in MeOH (5 mL) in a 100 mL three-necked flask was added LiBH₄ in THF (2 M, 2.33 mL, 5 eq) at 0° C., the mixture was stirred at 20° C. for 2 h. After the reaction was completed, the reaction mixture was quenched by saturated NH₄Cl aqueous (50 mL) in room temperature. The mixture was EtOAc extracted with (50 mL×3), the combined organic phase was dried over Na₂SO₄, and concentrated on vacuum to give a crude product. The crude product was purified by column chromatography (SiO₂, PE:EtOAc=5:1 to 1:1, desired product Rf=0.4). Compound 4 (200 mg, 393.17 umol, 42.20% yield) was obtained as white solid. LCMS: Rt=0.441 min, m/z=509.5 (M+H⁺)

To a solution of compound 4 (50 mg, 98.29 umol, 1 eq) in THF (1 mL) in an 8 mL glass bottle was added HCl in H₂O (1.02 g, 27.98 mmol, 1 mL, 284.61 eq), the mixture was stirred at 20° C. for 48 h. The reaction mixture was adjusted to pH=7 with Na 2 CO 3 at 20° C. The mixture was extracted with EtOAc (5 mL×3), the combined organic phase was washed with brine (5 mL), dried over anhydrous Na₂SO₄ and concentrated to give a crude product. The compound was used into the next step without further purification. Compound 5 (30 mg, 97.26 umol, 98.95% yield) was obtained as colourless oil. LCMS: Rt=0.235 min, m/z=309.1 (M+Na⁺)

To a solution of compound 5 (30 mg, 97.26 umol, 1 eq) and compound 6 (25.31 mg, 97.26 umol, 1 eq) in in DCM (1 mL) in a 100 mL round-bottom flask was added EDCI (27.97 mg, 145.89 umol, 1.5 eq) and DMAP (1.19 mg, 9.73 umol, 0.1 eq), the mixture was stirred at 20° C. for 12 h. After the reaction was completed, the mixture was concentrated to give a crude product. The crude product was purified by prep-TLC (PE:EtOAc=0:1). Compound 7 (16 mg, 29.05 umol, 29.87% yield) was obtained as colourless oil. LCMS: Rt=0.450 min, m/z=551.1 (M+H⁺)

To a solution of compound 7 (16 mg, 29.05 umol, 1 eq) in DCM (0.5 mL) in a 50 ml round-bottom flask was added TFA (770.00 mg, 6.75 mmol, 0.5 mL, 232.43 eq), the mixture was stirred at 20° C. for 2 h. The mixture was concentrated to give a crude product, the crude product was dissolved in ACN (5 mL), and 1 g Na₂CO₃ was added to the mixture, the mixture was stirred at 20° C. for 0.5 h, then the mixture was filtered and the filtrate was concentrated to give a crude product. The crude product was purified by Prep-HPLC (FA, column: Phenomenex luna C18 150*25 mm*10 um; mobile phase: [water(FA)-ACN]; B %: 15%-45%, 10 min), the purified solution was lyophiilized to give a white solid. Compound 150 (4.9 mg, 11.62 umol, 39.99% yield, 99.7% purity) was obtained as white solid. LCMS: Rt=0.329 min, m/z=421.1 (M+H⁺). ¹H NMR: (400 MHz, chloroform-d) δ=8.24-8.21 (m, 2H), 7.55 (s, 1H), 7.41-7.28 (m, 5H), 7.08-6.92 (m, 2H), 5.10 (s, 2H), 4.93 (br d, J=6.0 Hz, 2H), 4.15 (q, J=6.9 Hz, 2H), 1.40 (br t, J=6.8 Hz, 3H).

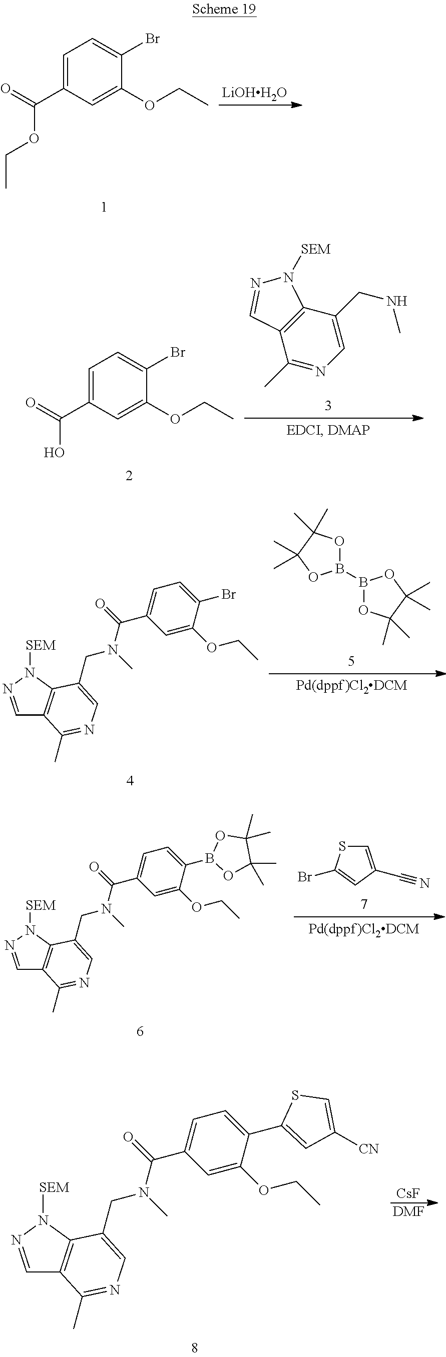

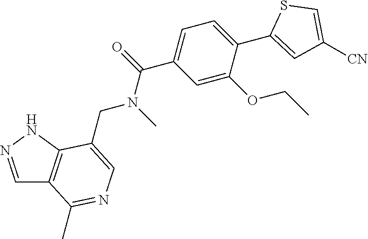 [SMILES: CCOc1cc(C(=O)N(C)Cc2cnc(C)c3cn[nH]c23)ccc1-c1cc(C#N)cs1]

To a solution of compound 1 (2 g, 7.32 mmol, 1 eq) in EtOH (20 mL) and H₂O (20 mL) was added LiOH·H₂O (921.86 mg, 21.97 mmol, 3 eq) in a 250 mL one-necked round bottom flask, the mixture was stirred at 25° C. for 1 h. The solvents was removed by evaporated under reduced pressure, then the mixture was acidified by 1N HCl to pH=4. The mixture was filtered and the filter cake was washed with 20 mL of MeOH, dried and in vacuum to give compound 2 (1.7 g, 6.94 mmol, 94.73% yield) which was obtained as white solid. LCMS: Rt=0.336 min, m/z=244.8 (M−H⁺)

To a solution of compound 3 (430 mg, 1.40 mmol, 1 eq) in DCM (8 mL) was added compound 2 (343.10 mg, 1.40 mmol, 1 eq), EDCI (402.57 mg, 2.10 mmol, 1.5 eq) and DMAP (17.10 mg, 140.00 umol, 0.1 eq) in a 40 mL glass bottle, the mixture was stirred at 25° C. for 12 h. The reaction mixture was filtered and concentrated under reduced pressure to give a residue. The residue was purified by column chromatography (SiO₂, PE/EtOAc=1/0 to 0/1, desired product Rf=0.46) and the purified solution was concentrated under vacuum to give product. Compound 4 (590 mg, 1.11 mmol, 78.99% yield) was obtained as white solid. LCMS: Rt=0.419 min, m/z=535.0 (M+H⁺)

To a solution of compound 4 (450 mg, 843.44 umol, 1 eq) in dioxane (10 mL) was added compound 5 (428.36 mg, 1.69 mmol, 2 eq), AcOK (165.55 mg, 1.69 mmol, 2 eq) and Pd(dppf)Cl₂·CH₂Cl₂ (68.88 mg, 84.34 umol, 0.1 eq) in a 40 mL glass bottle, the mixture was stirred at 25° C. for 12 h. The reaction mixture was quenched by addition H₂O (30 mL) and extracted with dichloromethane (30 mL×3). The combined organic layers were washed with brine (100 mL×1), dried over Na₂SO₄, filtered and concentrated under reduced pressure to give a residue. The residue was purified by column chromatography (SiO₂, PE/EtOAc=1/0 to 0/1, desired product Rf=0.30). Compound 6 (190 mg, 289.99 umol, 62.39% yield) was obtained as yellow oil. LCMS: Rt=0.439 min, m/z=581.2 (M+H⁺)

To a solution of compound 6 (150 mg, 258.35 umol, 1 eq) in dioxane (5 mL) was added compound 7 (48.58 mg, 258.35 umol, 1 eq), K₂CO₃ (71.41 mg, 516.71 umol, 2 eq) and Pd(dppf)Cl₂CH₂Cl₂ (21.10 mg, 25.84 umol, 0.1 eq) in a 40 mL glass bottle. The mixture was stirred at 100° C. for 12 hr under N₂ atmosphere. The reaction mixture was quenched by addition H₂O (30 mL) and extracted with dichloromethane (30 mL×3). The combined organic layers were washed with brine (60 mL×1), dried over Na₂SO₄, filtered and concentrated under reduced pressure to give a residue. Then the residue was purified by prep-TLC (SiO₂, PE:EtOAc=0:1) to give the product. Compound 8 (100 mg, 178.01 umol, 68.90% yield) was obtained as yellow oil. LCMS: Rt=0.448 min, m/z=562.5 (M+H⁺)

To a solution of compound 8 (100 mg, 178.01 umol, 1 eq) in DMF (2 mL) was added CsF (270.40 mg, 1.78 mmol, 65.63 uL, 10 eq) in a 8 mL glass bottle. The mixture was stirred at 100° C. for 12 hr. The reaction mixture was quenched by addition H₂O (20 mL), and extracted with dichloromethane (20 mL×3). The combined organic layers were washed with brine (50 mL×1), dried over Na₂SO₄, filtered and concentrated under reduced pressure to give a residue. The residue was purified by prep-HPLC (FA, column: Phenomenex luna C18 150*25 mm*10 um; mobile phase: [water (FA)-ACN]; B %: 14%-44%, 8 min), the purified product was lyophilized. 153 (20.2 mg, 46.30 umol, 26.01% yield, 98.9% purity) was obtained as yellow solid. LCMS: Rt=0.356 min, m/z=432.3 (M+H⁺) 1H NMR: (400 MHz, chloroform-d) δ=12.15-12.00 (m, 1H), 8.27-8.17 (m, 2H), 7.94 (s, 1H), 7.71-7.64 (m, 2H), 7.12-7.05 (m, 2H), 4.90 (s, 2H), 4.22 (q, J=7.0 Hz, 2H), 3.01 (s, 3H), 2.90-2.84 (m, 3H), 1.57-1.53 (m, 3H).

Compound 099 was prepared in a similar manner to Scheme 19 and was obtained as a white solid (7.2 mg, 16.02 umol, 13.27% yield). ¹H NMR: (400 MHz, chloroform-d) δ=7.99 (s, 1H), 7.37 (dd, J=2.2, 8.2 Hz, 1H), 7.23 (s, 1H), 7.13-7.11 (m, 2H), 7.08-7.03 (m, 3H), 6.97 (d, J=2.1 Hz, 1H), 6.89-6.86 (m, 1H), 4.92 (s, 2H), 3.75-3.72 (m, 1H), 3.05 (s, 3H), 0.80-0.77 (m, 2H), 0.72-0.69 (m, 2H). LCMS: Rt=0.449 min, m/z=449.8 (M+H⁺).

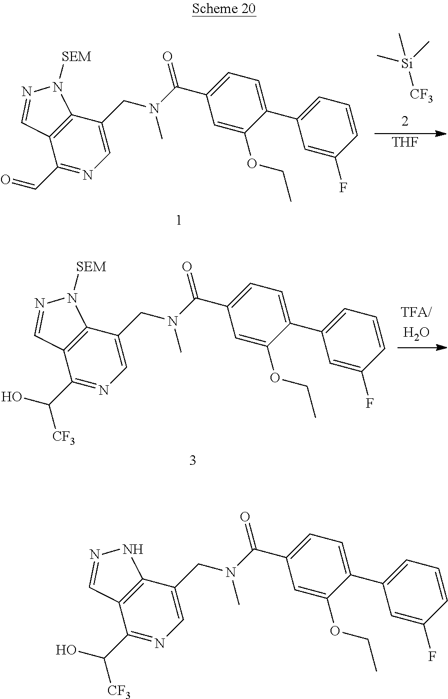

To a solution of Compound 1 in THF (1.5 mL) was added CsF (30.37 mg, 199.93 umol, 7.37 uL, 1.5 eq) and Compound 2 (28.43 mg, 199.93 umol, 1.5 eq) at 0° C. The mixture was stirred at 25° C. for 2 hr. The reaction mixture was poured into H₂O (70 mL), was extracted with DCM (50 mL×3). The combined organics were washed with sat.NaCl.aq (40 mL×3), dried with Na₂SO₄ and concentrated to give a residue. The residue was purified by prep-TLC (SiO₂, PE/EtOAc=1/1). Compound 3 (62 mg, 97.99 umol, 73.52% yield) as a Colorless oil. LCMS: Rt=0.539 min, m/z=633.4 (M+H⁺).

To a 50 mL round-bottomed flask solution of compound 3 in TFA (1 mL) and DCM (1 mL). The mixture was stirred at 25° C. for 1 hr. The reaction mixture was concentrated under vacuum to give residue. The crude product was purified by prep-RP (column: Waters xbridge 150*25 mm 10 um; mobile phase: [water (NH₄HCO₃)-ACN]; B %: 40%-70%, 8 min). After prep-HPLC purification, the eluent was concentrated to remove organic solvents. The residual aqueous solution was lyophilized to give white solid. Compound 171 (22 mg, 43.78 umol, 36.94% yield, 100% purity) as a white solid. LCMS: Rt=0.413 min, m/z=503.1 (M+H⁺). 1H NMR: (400 MHz, chloroform-d) δ ppm 1.38 (t, J=6.94 Hz, 3H) 3.10 (s, 3H) 4.08 (q, J=6.88 Hz, 2H) 4.91-4.98 (m, 1H) 5.02-5.10 (m, 1H) 5.57 (br d, J=6.13 Hz, 1H) 7.02-7.13 (m, 3H) 7.27-7.29 (m, 1H) 7.31 (br d, J=7.50 Hz, 1H) 7.35-7.40 (m, 2H) 8.38 (s, 2H) 12.43-12.74 (m, 1H).

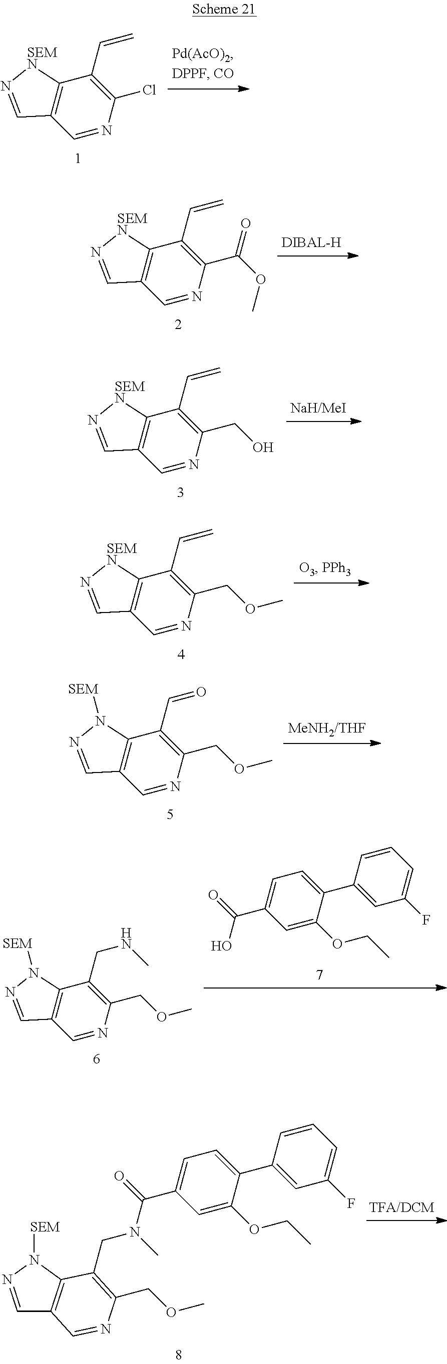

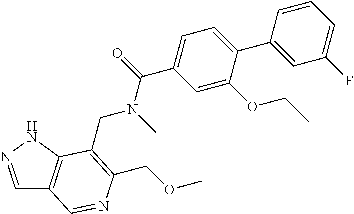 [SMILES: CCOc1cc(C(=O)N(C)Cc2c(COC)ncc3cn[nH]c23)ccc1-c1cccc(F)c1]

To a solution of compound 1 (1.5 g, 4.84 mmol, 1 eq) in MeOH (10 mL) in a 75 mL hydrogenated bottle was added Pd(OAc)₂ (217.36 mg, 968.16 umol, 0.2 eq), DPPF (536.73 mg, 968.16 umol, 0.2 eq) and TEA (1.47 g, 14.52 mmol, 2.02 mL, 3 eq), the mixture was stirred at 80° C. for 48 h under CO (50 psi). Cool to room temperature after the reaction was completed, the mixture was filtered under N₂ and the filtrated was concentrated to give a crude product. The crude product was purified by column chromatography (SiO₂, PE:EtOAc=20:1 to 5:1, desired product Rf=0.5). Compound 2 (1.6 g, 4.80 mmol, 99.12% yield) was obtained as colourless oil. LCMS: Rt=0.463 min, m/z=334.2 (M+H⁺) ¹H NMR: (400 MHz, chloroform-d) δ=9.06-9.03 (m, 1H), 8.20 (s, 1H), 7.42-7.33 (m, 1H), 5.84 (s, 2H), 5.73-5.68 (m, 1H), 5.57-5.49 (m, 1H), 3.99-3.96 (m, 3H), 3.55-3.49 (m, 2H), 1.26 (br t, J=7.1 Hz, 2H), −0.05 (s, 9H)

To a solution of compound 2 (700 mg, 2.10 mmol, 1 eq) in THF (5 mL) in a three-neck round bottle was added DIBAL-H (1 M, 6.30 mL, 3 eq) at −78° C., the mixture was stirred at −78° C. for 1 hr under N₂. The mixture was poured into saturated NH₄Cl (80 mL) solution, and extracted with EtOAc (50 mL×3), the combined organic phase was washed with brine (50 mL), dried over anhydrous Na₂SO₄, and concentrated to give a crude product. The crude product was purified by column chromatography (SiO₂, PE/EtOAc=10/1 to 0/1, desired product Rf=0.6). Compound 3 (63 mg, 206.26 umol, 9.83% yield) was obtained as a yellow oil. LCMS: Rt=0.371 min, m/z=306.0 (M+H⁺)

To a solution of compound 3 (95 mg, 311.02 umol, 1 eq) in THF (5 mL) in a 100 mL three-necked flask was added NaH (18.66 mg, 466.53 umol, 60% purity, 1.5 eq) at 0° C., the mixture was stirred at 0° C. for 1 h. Then was added Mel (88.29 mg, 622.04 umol, 38.72 uL, 2 eq), the mixture was stirred at 20° C. for 1 h. The reaction mixture was poured into saturated NH₄Cl solution (50 mL) at 0° C., and extracted with EtOAc (50 mL×2), the combined organic phase was washed with brine (80 mL), dried over anhydrous Na₂SO₄, and concentrated to give a crude product. The residue was purified by prep-TLC (SiO₂, PE:EtOAc=0:1). Compound 4 (69 mg, 215.98 umol, 69.44% yield, N/A purity) was obtained as yellow oil. LCMS: Rt=0.367 min, m/z=319.9 (M+Na⁺)

To a solution of compound 4 (65 mg, 203.46 umol, 1 eq) in DCM (10 mL) and MeOH (10 mL) in a 100 mL three-necked flask was treated with OZONE (9.77 mg, 203.46 umol, 1 eq) for 30 min (15 psi) at −78° C., then the mixture was purged with nitrogen for 0.5 h, PPh₃ (106.73 mg, 406.92 umol, 2 eq) dissolved in DCM (10 mL) was added dropwise to the mixture. The mixture was warmed to 20° C. and stirred at 20° C. for 1 h. After the reaction was completed, the mixture was concentrated to give a crude product. The residue was purified by prep-TLC (SiO₂, PE:EtOAc=0:1). Compound 5 (49 mg, 152.44 umol, 74.92% yield) was obtained as yellow oil.

To a solution of compound 5 (49 mg, 152.44 umol, 1 eq) in MeOH (4 mL) in a 100 mL round-bottom flask was added MeNH₂ in THF (2 M, 381.09 uL, 5 eq) and AcOH (915.41 ug, 15.24 umol, 8.72e-1 uL, 0.1 eq), the mixture was stirred at 20° C. for 2 h. Then was added NaBH₄ (17.30 mg, 457.31 umol, 3 eq) at 0° C., the mixture was stirred at 20° C. for 1 h. After the reaction was completed, the reaction mixture was poured into NH₄Cl (10 mL) and control the temperature between 1020° C., then the mixture was added Na 2 CO 3 (1 g), the solution was filtered, the filtrate was concentrated under reduced pressure to give a residue. Then the solution was triturated in DCM and MeOH and collected by filtration, the filtrate was concentrated under reduced pressure to give a residue. LCMS: Rt=0.722 min, m/z=377.3 (M+H⁺)

To a solution of compound 6 (30 mg, 89.15 umol, 1 eq) and compound 7 (23.20 mg, 89.15 umol, 1 eq) in DCM (2 mL) in a 50 mL round-bottom flask was added EDCI (25.64 mg, 133.73 umol, 1.5 eq) and DMAP (1.09 mg, 8.92 umol, 0.1 eq), the mixture was stirred at 20° C. for 12 h. Evaporate the solution on a water bath under reduced pressure using a rotary evaporator. The residue was purified by prep-TLC (SiO₂, PE:EtOAc=0:1). Compound 8 (50 mg, 69.11 umol, 77.52% yield, 80% purity) was obtained as colourless oil. LCMS: Rt=0.474 min, m/z=579.3 (M+H⁺)

To a solution of compound 8 (50 mg, 69.11 umol, 80% purity, 1 eq) in DCM (1 mL) in a 100 mL round-bottom flask was added TFA (1.54 g, 13.51 mmol, 1 mL, 195.41 eq), the mixture was stirred at 20° C. for 2 h. The mixture was concentrated to give a crude product, the crude product was dissolved in MeOH (5 mL), and 1 g Na₂CO₃ was added to the mixture, the mixture was stirred at 20° C. for 0.5 h, then the mixture was filtered and the filtrate was concentrated to give a crude product. The crude product was purified by Prep-HPLC (FA, column: Phenomenex luna C18 150*25 mm*10 um; mobile phase: [water (FA)-ACN]; B %: 20%-50%, 10 min), the purified solution was lyophiilized to give a white solid. Compound 077 (16.6 mg, 36.90 umol, 53.39% yield, 99.7% purity) was obtained as white solid. LCMS: Rt=0.384 min, m/z=449.2 (M+H⁺) ¹H NMR: (400 MHz, chloroform-d) δ=12.42-12.26 (m, 1H), 9.10-9.03 (m, 1H), 8.24 (s, 1H), 7.40-7.28 (m, 4H), 7.10-7.01 (m, 3H), 5.22 (s, 2H), 4.91-4.87 (m, 2H), 4.07 (q, J=7.0 Hz, 2H), 3.54-3.47 (m, 3H), 3.05 (s, 3H), 1.37 (t, J=6.9 Hz, 3H).

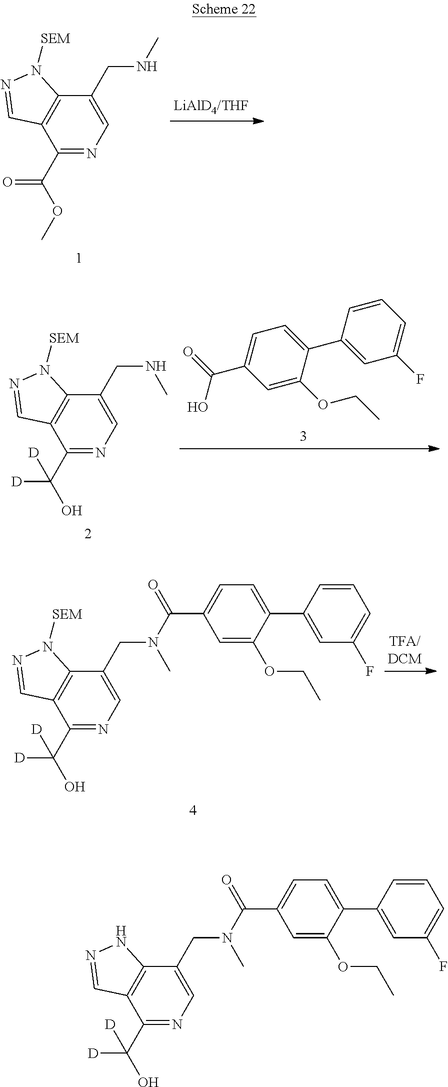

To a solution of Compound 1 (50 mg, 142.66 umol, 1 eq) in THF (1 mL) was added lithium; tetradeuterioalumanuide (19.69 mg, 427.98 umol, 26.75 uL, 3 eq) at 0° C. The mixture was stirred at 0° C. for 1 hr. After the reaction was completed, the resulting mixture was added Na₂SO₄·10H₂O (20 mg). Then the mixture was filtered, and the filtrate was concentrated on vacuum to give a Compound 2 (46 mg, 141.76 umol, 99.37% yield) as a yellow oil which was used in the next step. LCMS: Rt=0.646 min, m/z=325.2 (M+H⁺)

To a solution of Compound 2 (46 mg, 141.76 umol, 1 eq) and Compound 3 (36.89 mg, 141.76 umol, 1 eq) in DCM (3 mL) in round-bottom flask (50 mL) was added EDCI (40.76 mg, 212.64 umol, 1.5 eq) and DMAP (1.73 mg, 14.18 umol, 0.1 eq). The mixture was stirred at 20° C. for 1 hr. The reaction mixture was concentrated on vacuum to give residue. The residue was purified by prep-TLC (SiO₂, DCM:MeOH=10:1). The purified solution was concentrated on vacuum to give Compound 4 (20 mg, 35.29 umol, 24.89% yield) as a colorless oil. LCMS: Rt=0.711 min, m/z=567.2 (M+H⁺) D %=94.6%

To a solution of Compound 4 (20 mg, 35.29 umol, 1 eq) in DCM (0.5 mL) in round-bottom flask (50 mL) was added TFA (770.00 mg, 6.75 mmol, 0.5 mL, 191.36 eq). The mixture was stirred at 20° C. for 1 hr. The reaction mixture was concentrated to give the crude product. The residue was basified with Et₃N to pH about 8. The residue was purified by prep-HPLC (column: Welch Xtimate C18 150*25 mm*5 um; mobile phase: [water (NH₃H₂O)-ACN]; B %: 27%-57%, 8 min). The purified solution was lyophilized to give 086 (2.6 mg, 5.80 umol, 16.44% yield, 97.4% purity) as a white solid. LCMS: Rt=0.651 min, m/z=437.1 (M+H⁺) D %=94.6% 1H NMR (400 MHz, chloroform-d) δ=8.41 (s, 1H), 8.32 (s, 1H), 7.40-7.35 (m, 2H), 7.33-7.29 (m, 2H), 7.11-7.04 (m, 3H), 4.98 (s, 2H), 4.11-4.04 (m, 2H), 3.08 (s, 3H), 1.38 (t, J=7.0 Hz, 3H).

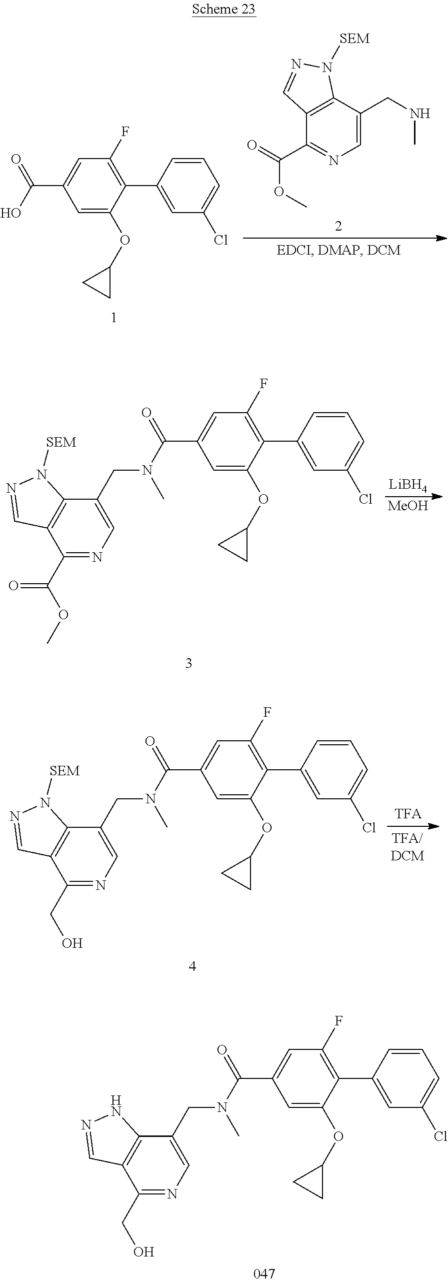

To a solution of Compound 1 (437.56 mg, 1.43 mmol, 1 eq) and Compound 2 (500 mg, 1.43 mmol, 1 eq) in DCM (15 mL) was added EDCI (410.21 mg, 2.14 mmol, 1.5 eq) and DMAP (17.43 mg, 142.66 umol, 0.1 eq). The mixture was stirred at 20° C. for 12 hr. LCMS (5-95AB/0.8 min) showed 68% of desired mass was detected. The reaction mixture was concentrated on vacuum to give residue. The residue was purified by column chromatography (SiO₂, Petroleum ether/Ethyl acetate=5/1 to 2/1), the purified solution was concentrated on vacuum to give Compound 3 (1.1 g, 1.38 mmol, 96.51% yield, 80% purity) as a yellow oil. LCMS: Rt=0.640 min, m/z=639.3 (M+H)⁺

To a solution of Compound 3 (1.1 g, 1.38 mmol, 80% purity, 1 eq) in MeOH (15 mL) in round-bottom flask (100 mL) was added LiBH₄ (149.95 mg, 6.88 mmol, 5 eq) at 0° C. The mixture was stirred at 20° C. for 12 hr. LCMS (5-95AB/0.8 min) showed 50% of desired mass was detected and 33% of Compound 3 was remained. The reaction mixture was poured into saturated NH₄Cl aqueous (30 mL) and extracted by extracted by ethyl acetate (30 mL×3), the combined organic phase was dried and concentrated under vacuum to give a residue. The residue was purified by column chromatography (SiO₂, Petroleum ether/Ethyl acetate=1/1 to Ethyl acetate:Methanol=10/1), the purified solution was concentrated on vacuum to give Compound 4 (330 mg, 539.94 umol, 39.22% yield) as a yellow oil. LCMS: Rt=0.495 min, m/z=611.8 (M+H)⁺

To a solution of Compound 4 (50 mg, 81.81 umol, 1 eq) in DCM (1 mL) in round-bottom flask (50 mL) was added TFA (1.54 g, 13.51 mmol, 1 mL, 165.09 eq). The mixture was stirred at 20° C. for 5 hr. LCMS (5-95AB/0.8 min) showed 86% of desired mass was detected. The reaction mixture was concentrated at 20° C. and under reduced pressure to give a residue. The residue was dissolved with DCM (5 mL) and basified with NaHCO₃ (5 mg) to pH about 7, then filtered, the filtrate was concentrated on vacuum to give a residue. The residue was purified by prep-HPLC (column: Phenomenex luna C18 150*25 mm*10 um; mobile phase: [water (FA)-ACN]; B %: 22%-52%, 9 min). The purified solution was lyophilized to give 047 (12.4 mg, 25.50 umol, 31.17% yield, 98.9% purity) as a white solid. LCMS: Rt=0.400 min, m/z=481.1 (M+H)⁺ 1H NMR (400 MHz, CHLOROFORM-d) δ=8.34 (d, J=14.8 Hz, 2H), 7.38-7.31 (m, 3H), 7.23 (s, 2H), 6.89 (dd, J=1.0, 9.0 Hz, 1H), 5.20 (s, 2H), 4.97 (s, 2H), 3.75 (td, J=3.0, 5.8 Hz, 1H), 3.09 (s, 3H), 0.83-0.77 (m, 2H), 0.76-0.68 (m, 2H).

### Example 2: THP-1 Cell-Based NLRP3 Activation Assay

THP-1 cells are cultured in complete media (CM) until they reach logarithmic growth and achieve a viability >90%. CM is composed of RPMI-1640 (+Glutamax)/10% fetal bovine serum/55 μM β-mercaptoethanol/pen/strep. Cells are spun down and resuspended to 1,000,000 cells/mL in CM containing either 20 nM or 500 nM PMA. 150,000 cells (1504) are then added to each well of a 96-well TC plate and incubated for either 24 hr or 3 hr, respectively, in a standard cell culture incubator (37° C.; 5% CO₂). After this incubation, the plate is tilted and media carefully removed. 200 μL of CM containing 100 ng/mL LPS is then added to the wells and the cells incubated an additional 3 hrs. The media is again removed and replaced with Opti-Mem medium containing pre-determined dilutions of test compounds in replicate wells. After a 30 min pre-incubation with test compound, 10 μM nigericin (final concentration) in Opti-Mem medium with the corresponding concentration of compound is added to the wells for an additional 1 hr. Positive control wells contain 10 μM nigericin in Opti-Mem in the absence of test compound, while negative control wells contain Opti-Mem only. Supernatants are then transferred to a fresh 96-well plate for storage and assayed for IL-1β (human; DuoSet; R&D) and for TNFα (human; DuoSet; R&D) levels and relative pyroptosis using a CytoTox 96 Kit (do not freeze prior to testing; Promega). Once supernatants are removed, the relative viability of adherent cells in the 96-well TC plate are determined using a CellTiter-Glo® luminescent cell viability assay (Promega).

Table 6 below provides IC₅₀ data for the compounds disclosed herein where “+” is indicative of an IC₅₀ value of <50 uM, “++” is indicative of an IC₅₀ value of <10 uM, and “+++” is indicative of an IC₅₀ value of <1 uM.

TABLE 6

| Compound No. | IC₅₀ |
|---|---|
| 001 | +++ |
| 005 | + |
| 006 | +++ |
| 007 | +++ |
| 008 | ++ |
| 009 | +++ |
| 011 | +++ |
| 012 | +++ |
| 013 | +++ |
| 015 | +++ |
| 016 | +++ |
| 017 | +++ |
| 018 | +++ |
| 019 | +++ |
| 020 | +++ |
| 021 | + |
| 023 | +++ |
| 024 | +++ |
| 025 | ++ |
| 026 | +++ |
| 027 | +++ |
| 028 | +++ |
| 029 | +++ |
| 030 | +++ |
| 031 | +++ |
| 032 | +++ |
| 033 | +++ |
| 034 | ++ |
| 035 | +++ |
| 036 | +++ |
| 037 | +++ |
| 038 | +++ |
| 039 | +++ |
| 040 | +++ |
| 041 | +++ |
| 042 | +++ |
| 043 | + |
| 044 | + |
| 045 | ++ |
| 046 | +++ |
| 047 | +++ |
| 048 | +++ |
| 049 | + |
| 050 | +++ |
| 052 | +++ |
| 053 | +++ |
| 054 | +++ |
| 055 | +++ |
| 056 | +++ |
| 057 | + |
| 058 | + |
| 059 | +++ |
| 062 | +++ |
| 064 | +++ |
| 065 | +++ |
| 066 | +++ |
| 067 | +++ |
| 068 | +++ |
| 069 | +++ |
| 070 | +++ |
| 071 | +++ |
| 072 | ++ |
| 073 | +++ |
| 074 | + |
| 075 | + |
| 076 | +++ |
| 077 | +++ |
| 078 | +++ |
| 079 | +++ |
| 080 | + |
| 081 | +++ |
| 082 | + |
| 083 | ++ |
| 084 | +++ |
| 085 | ++ |
| 086 | +++ |
| 087 | +++ |
| 088 | +++ |
| 090 | ++ |
| 091 | +++ |
| 092 | +++ |
| 093 | +++ |
| 094 | +++ |
| 095 | ++ |
| 096 | +++ |
| 097 | +++ |
| 098 | +++ |
| 099 | +++ |
| 100 | +++ |
| 101 | +++ |
| 102 | +++ |
| 103 | ++ |
| 105 | +++ |
| 106 | +++ |
| 107 | +++ |
| 108 | +++ |
| 109 | +++ |
| 111 | +++ |
| 112 | +++ |
| 113 | +++ |
| 114 | +++ |
| 115 | +++ |
| 116 | +++ |
| 117 | +++ |
| 118 | +++ |
| 119 | +++ |
| 120 | +++ |
| 121 | +++ |
| 122 | +++ |
| 123 | +++ |
| 124 | + |
| 125 | ++ |
| 126 | +++ |
| 128 | ++ |
| 129 | ++ |
| 130 | +++ |
| 131 | +++ |
| 132 | +++ |
| 133 | ++ |
| 134 | +++ |
| 135 | +++ |
| 136 | +++ |
| 137 | +++ |
| 138 | +++ |
| 139 | +++ |
| 140 | ++ |
| 141 | +++ |
| 142 | +++ |
| 143 | +++ |
| 144 | +++ |
| 145 | +++ |
| 146 | +++ |
| 147 | +++ |
| 148 | +++ |
| 149 | ++ |
| 150 | +++ |
| 151 | ++ |
| 152 | +++ |
| 153 | ++ |
| 154 | +++ |
| 155 | +++ |
| 156 | + |
| 157 | +++ |
| 158 | +++ |
| 159 | +++ |
| 160 | +++ |
| 161 | +++ |
| 162 | +++ |
| 163 | +++ |
| 164 | +++ |
| 165 | +++ |
| 166 | +++ |
| 167 | +++ |
| 168 | +++ |
| 169 | ++ |
| 170 | +++ |
| 171 | +++ |
| 172 | +++ |
| 173 | +++ |
| 174 | +++ |
| 175 | ++ |
| 176 | +++ |
| 177 | +++ |
| 178 | +++ |
| 179 | +++ |
| 180 | +++ |
| 181 | +++ |
| 182 | +++ |
| 183 | +++ |
| 184 | ++ |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound selected from the group consisting 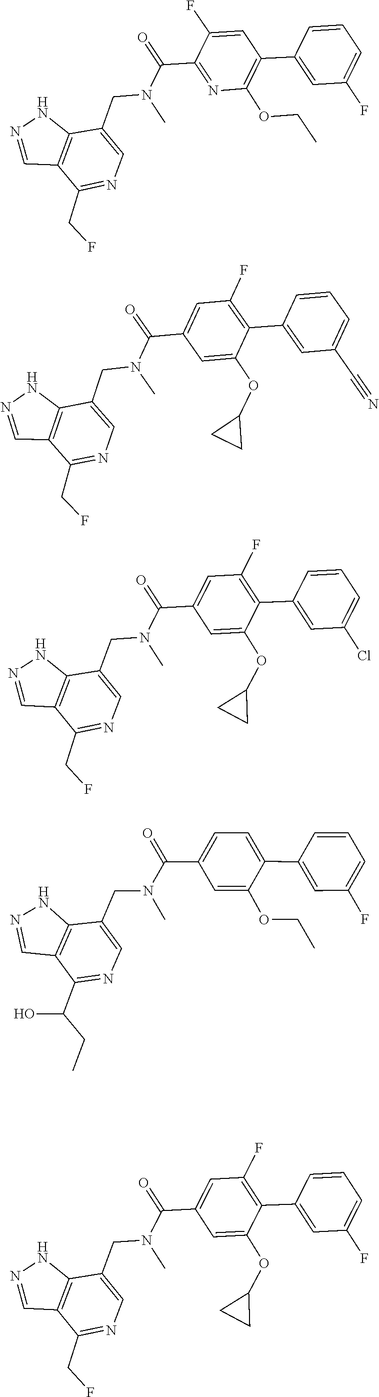 [SMILES: CCOc1nc(C(=O)N(C)Cc2cnc(CF)c3cn[nH]c23)c(F)cc1-c1cccc(F)c1 ; CN(Cc1cnc(CF)c2cn[nH]c12)C(=O)c1cc(F)c(-c2cccc(C#N)c2)c(OC2CC2)c1 ; CN(Cc1cnc(CF)c2cn[nH]c12)C(=O)c1cc(F)c(-c2cccc(Cl)c2)c(OC2CC2)c1 ; CCOc1cc(C(=O)N(C)Cc2cnc(C(O)CC)c3cn[nH]c23)ccc1-c1cccc(F)c1 ; CN(Cc1cnc(CF)c2cn[nH]c12)C(=O)c1cc(F)c(-c2cccc(F)c2)c(OC2CC2)c1] 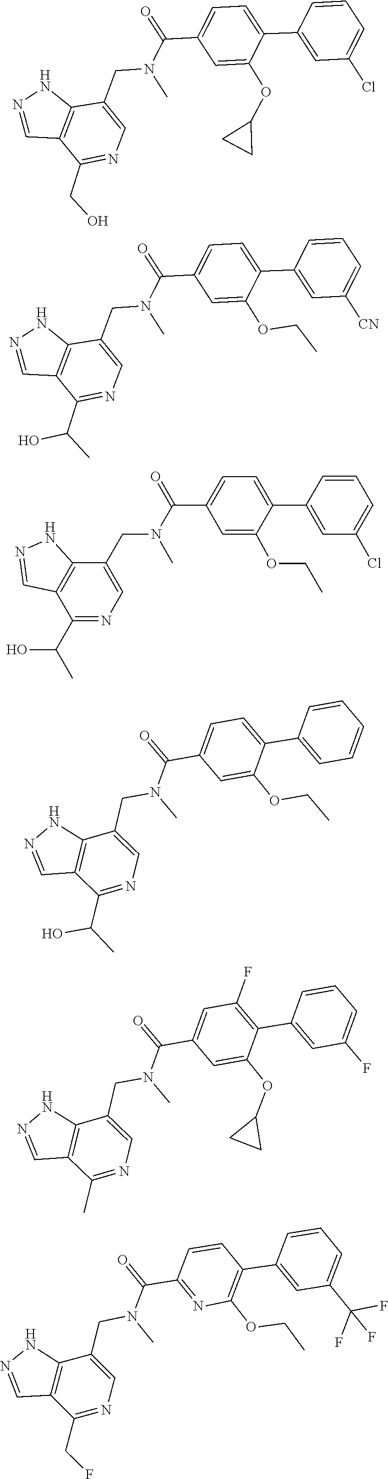 [SMILES: CN(Cc1cnc(CO)c2cn[nH]c12)C(=O)c1ccc(-c2cccc(Cl)c2)c(OC2CC2)c1 ; CCOc1cc(C(=O)N(C)Cc2cnc(C(C)O)c3cn[nH]c23)ccc1-c1cccc(C#N)c1 ; CCOc1cc(C(=O)N(C)Cc2cnc(C(C)O)c3cn[nH]c23)ccc1-c1cccc(Cl)c1 ; CCOc1cc(C(=O)N(C)Cc2cnc(C(C)O)c3cn[nH]c23)ccc1-c1ccccc1 ; Cc1ncc(CN(C)C(=O)c2cc(F)c(-c3cccc(F)c3)c(OC3CC3)c2)c2[nH]ncc12 ; CCOc1nc(C(=O)N(C)Cc2cnc(CF)c3cn[nH]c23)ccc1-c1cccc(C(F)(F)F)c1] 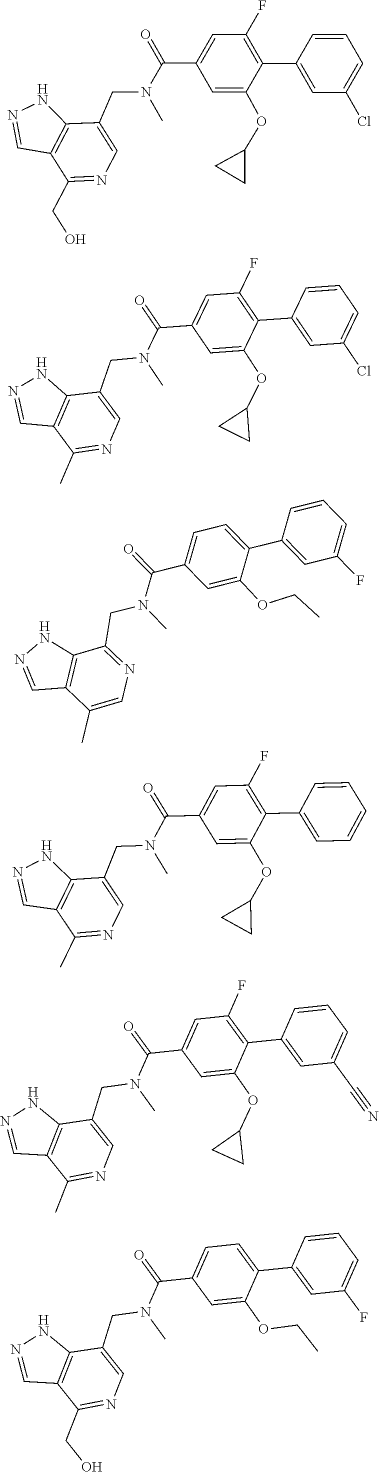 [SMILES: CN(Cc1cnc(CO)c2cn[nH]c12)C(=O)c1cc(F)c(-c2cccc(Cl)c2)c(OC2CC2)c1 ; Cc1ncc(CN(C)C(=O)c2cc(F)c(-c3cccc(Cl)c3)c(OC3CC3)c2)c2[nH]ncc12 ; CCOc1cc(C(=O)N(C)Cc2ncc(C)c3cn[nH]c23)ccc1-c1cccc(F)c1 ; Cc1ncc(CN(C)C(=O)c2cc(F)c(-c3ccccc3)c(OC3CC3)c2)c2[nH]ncc12 ; Cc1ncc(CN(C)C(=O)c2cc(F)c(-c3cccc(C#N)c3)c(OC3CC3)c2)c2[nH]ncc12 ; CCOc1cc(C(=O)N(C)Cc2cnc(CO)c3cn[nH]c23)ccc1-c1cccc(F)c1] 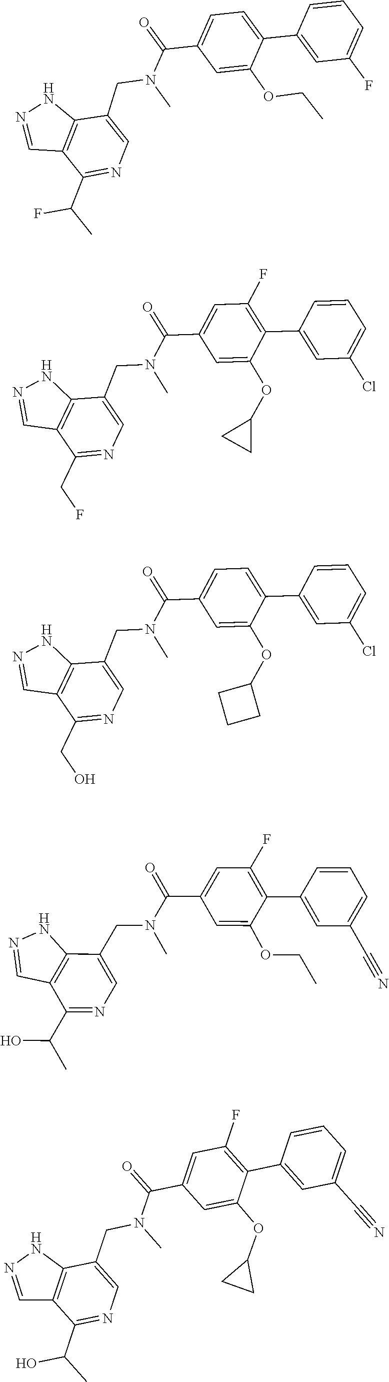 [SMILES: CCOc1cc(C(=O)N(C)Cc2cnc(C(C)F)c3cn[nH]c23)ccc1-c1cccc(F)c1 ; CN(Cc1cnc(CF)c2cn[nH]c12)C(=O)c1cc(F)c(-c2cccc(Cl)c2)c(OC2CC2)c1 ; CN(Cc1cnc(CO)c2cn[nH]c12)C(=O)c1ccc(-c2cccc(Cl)c2)c(OC2CCC2)c1 ; CCOc1cc(C(=O)N(C)Cc2cnc(C(C)O)c3cn[nH]c23)cc(F)c1-c1cccc(C#N)c1 ; CC(O)c1ncc(CN(C)C(=O)c2cc(F)c(-c3cccc(C#N)c3)c(OC3CC3)c2)c2[nH]ncc12] 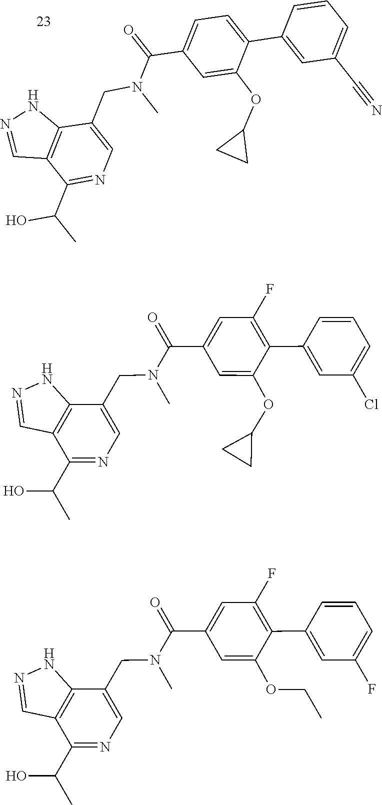 [SMILES: CC(O)c1ncc(CN(C)C(=O)c2ccc(-c3cccc(C#N)c3)c(OC3CC3)c2)c2[nH]ncc12 ; CC(O)c1ncc(CN(C)C(=O)c2cc(F)c(-c3cccc(Cl)c3)c(OC3CC3)c2)c2[nH]ncc12 ; CCOc1cc(C(=O)N(C)Cc2cnc(C(C)O)c3cn[nH]c23)cc(F)c1-c1cccc(F)c1]
or a pharmaceutically acceptable salt thereat.

2. The compound of claim 1, wherein the compound is selected from 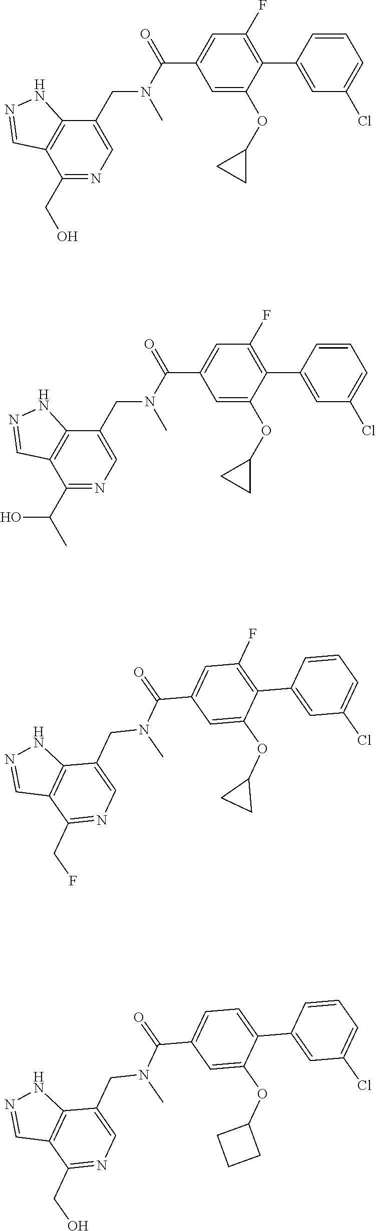 [SMILES: CN(Cc1cnc(CO)c2cn[nH]c12)C(=O)c1cc(F)c(-c2cccc(Cl)c2)c(OC2CC2)c1 ; CC(O)c1ncc(CN(C)C(=O)c2cc(F)c(-c3cccc(Cl)c3)c(OC3CC3)c2)c2[nH]ncc12 ; CN(Cc1cnc(CF)c2cn[nH]c12)C(=O)c1cc(F)c(-c2cccc(Cl)c2)c(OC2CC2)c1 ; CN(Cc1cnc(CO)c2cn[nH]c12)C(=O)c1ccc(-c2cccc(Cl)c2)c(OC2CCC2)c1]
or a pharmaceutically acceptable salt thereof.

3. A pharmaceutical composition comprising a compound of claim 1, or a pharmaceutically acceptable salt thereof, and a pharmaceutically acceptable carrier.

4. A method of inhibiting NLRP3 inflammasome in a subject in need thereof comprising administering to the subject a therapeutically effective amount of a compound of claim 1.

5. A method of treating inflammation in a subject in need thereof comprising administering to the subject a therapeutically effective amount of a compound of claim 1.

6. A method of treating inflammaging in a subject in need thereof comprising administering to the subject a therapeutically effective amount of a compound of claim 1.

7. A method of treating a neurosensory disease in a subject in need thereof comprising administering to the subject a therapeutically effective amount of a compound of claim 1.

8. The method of claim 7, wherein the neurosensory disease is selected from the group consisting of amyotrophic lateral sclerosis (ALS), traumatic brain injury, Parkinson's disease, and Alzheimer's disease.

9. The method of claim 7, wherein the neurosensory disease is ALS.

10. The method of claim 7, wherein the neurosensory disease is traumatic brain injury.

11. The method of claim 7, wherein the neurosensory disease is Parkinson's disease.

12. The method of claim 7, wherein the neurosensory disease is Alzheimer's disease.

13. A method of treating inflammation in a subject in need thereof comprising administering to the subject a therapeutically effective amount of a compound of Formula I: 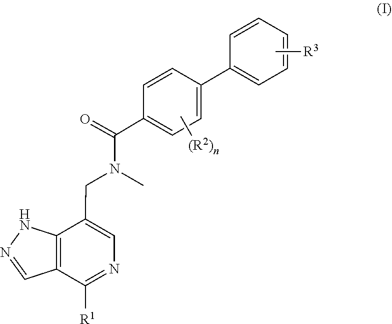
or a pharmaceutically acceptable salt thereof;
wherein
R¹ is C₁₋₆ alkyl optionally substituted with a substituent selected from halo, OH, OC₁₋₃ alkyl, NH₂, N(H)C₁₋₃ alkyl, N(C₁₋₆ alkyl)₂, C(O)C₁₋₃ alkyl, and C₃₋₆ cycloalkyl;
each R² is independently selected from halo, C₁₋₃ haloalkyl, OH, OC₁₋₃ alkyl, OC₃₋₆ cycloalkyl, and C₃₋₆ cycloalkyl;
R³ is selected from H, halo, C₁₋₃ haloalkyl, OH, OC₁₋₃ alkyl, OC₃₋₆ cycloalkyl, CN, and C₃₋₆ cycloalkyl; and
n is 0, 1, or 2.

14. The method of claim 13, wherein
R¹ is C₁₋₆ alkyl optionally substituted with OH;
each R² is independently selected from halo and C₃₋₆ cycloalkyl;
R³ is halo; and
n is 1 or 2.

15. The method of claim 13, wherein the compound of Formula I is selected from 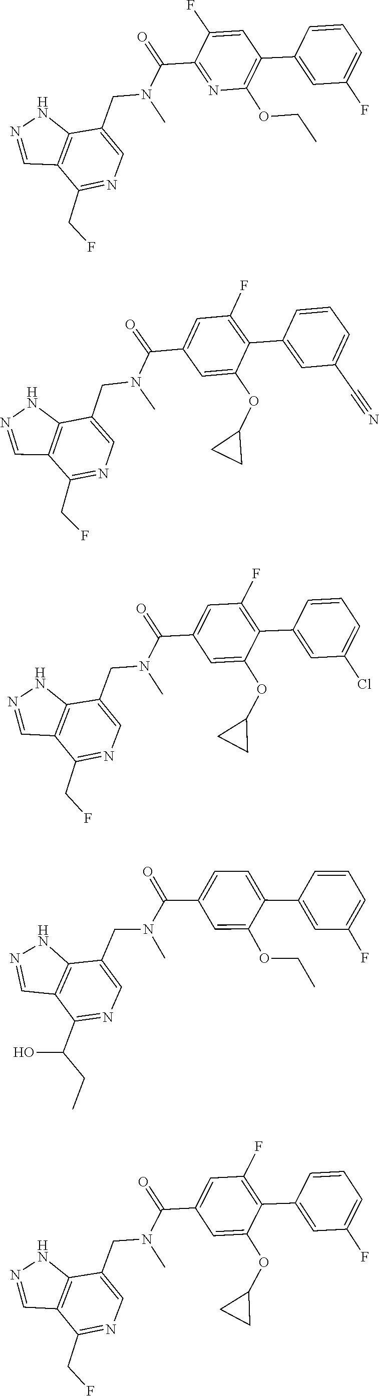 [SMILES: CCOc1nc(C(=O)N(C)Cc2cnc(CF)c3cn[nH]c23)c(F)cc1-c1cccc(F)c1 ; CN(Cc1cnc(CF)c2cn[nH]c12)C(=O)c1cc(F)c(-c2cccc(C#N)c2)c(OC2CC2)c1 ; CN(Cc1cnc(CF)c2cn[nH]c12)C(=O)c1cc(F)c(-c2cccc(Cl)c2)c(OC2CC2)c1 ; CCOc1cc(C(=O)N(C)Cc2cnc(C(O)CC)c3cn[nH]c23)ccc1-c1cccc(F)c1 ; CN(Cc1cnc(CF)c2cn[nH]c12)C(=O)c1cc(F)c(-c2cccc(F)c2)c(OC2CC2)c1] 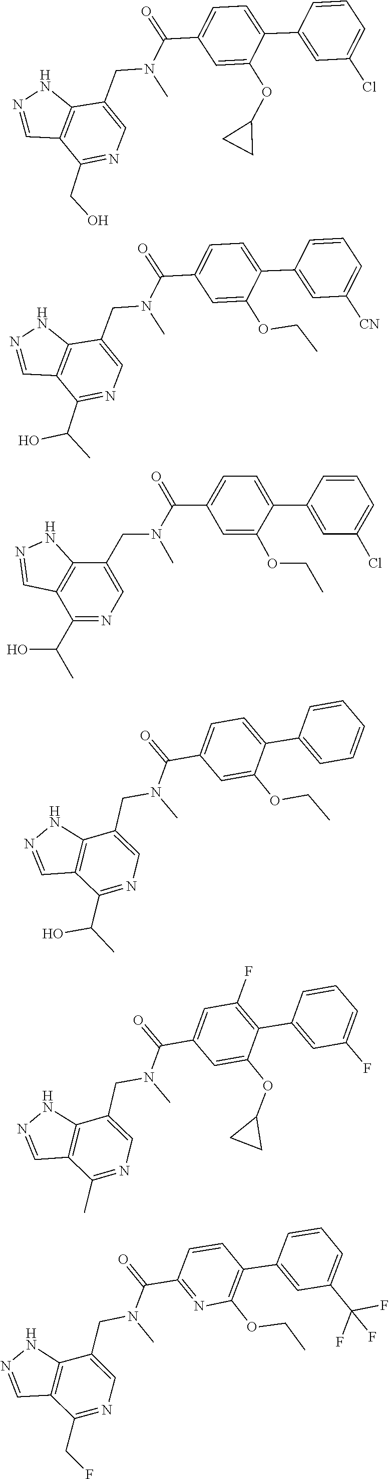 [SMILES: CN(Cc1cnc(CO)c2cn[nH]c12)C(=O)c1ccc(-c2cccc(Cl)c2)c(OC2CC2)c1 ; CCOc1cc(C(=O)N(C)Cc2cnc(C(C)O)c3cn[nH]c23)ccc1-c1cccc(C#N)c1 ; CCOc1cc(C(=O)N(C)Cc2cnc(C(C)O)c3cn[nH]c23)ccc1-c1cccc(Cl)c1 ; CCOc1cc(C(=O)N(C)Cc2cnc(C(C)O)c3cn[nH]c23)ccc1-c1ccccc1 ; Cc1ncc(CN(C)C(=O)c2cc(F)c(-c3cccc(F)c3)c(OC3CC3)c2)c2[nH]ncc12 ; CCOc1nc(C(=O)N(C)Cc2cnc(CF)c3cn[nH]c23)ccc1-c1cccc(C(F)(F)F)c1] 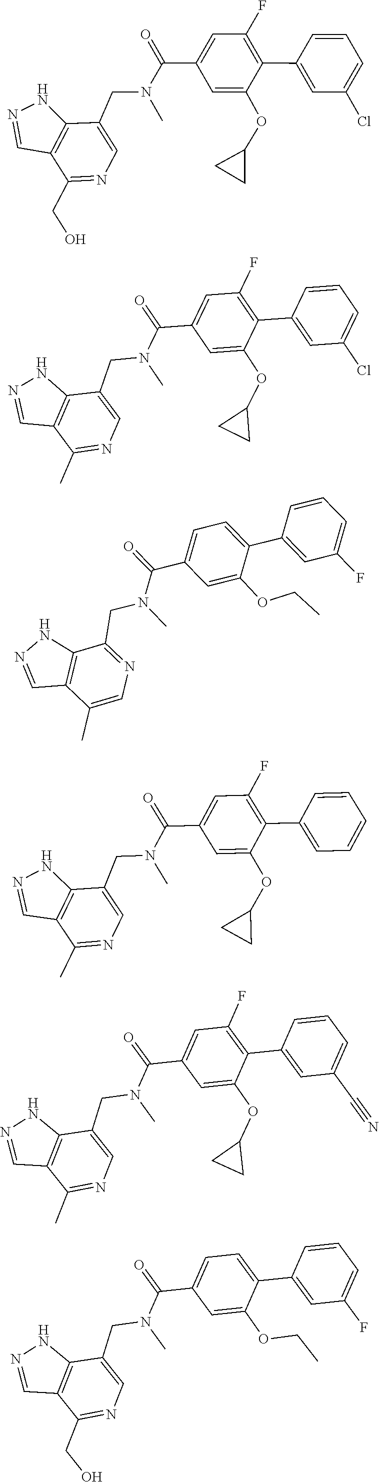 [SMILES: CN(Cc1cnc(CO)c2cn[nH]c12)C(=O)c1cc(F)c(-c2cccc(Cl)c2)c(OC2CC2)c1 ; Cc1ncc(CN(C)C(=O)c2cc(F)c(-c3cccc(Cl)c3)c(OC3CC3)c2)c2[nH]ncc12 ; CCOc1cc(C(=O)N(C)Cc2ncc(C)c3cn[nH]c23)ccc1-c1cccc(F)c1 ; Cc1ncc(CN(C)C(=O)c2cc(F)c(-c3ccccc3)c(OC3CC3)c2)c2[nH]ncc12 ; Cc1ncc(CN(C)C(=O)c2cc(F)c(-c3cccc(C#N)c3)c(OC3CC3)c2)c2[nH]ncc12 ; CCOc1cc(C(=O)N(C)Cc2cnc(CO)c3cn[nH]c23)ccc1-c1cccc(F)c1] 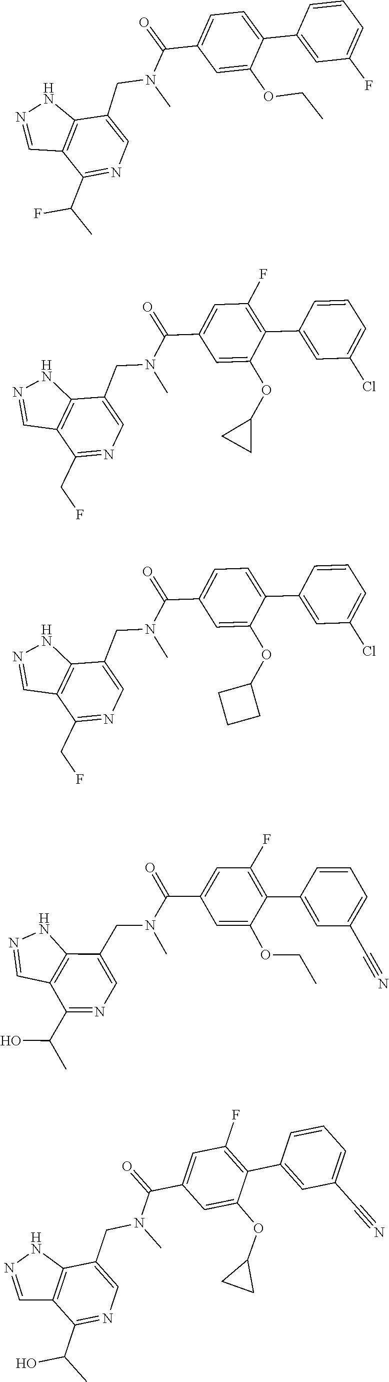 [SMILES: CCOc1cc(C(=O)N(C)Cc2cnc(C(C)F)c3cn[nH]c23)ccc1-c1cccc(F)c1 ; CN(Cc1cnc(CF)c2cn[nH]c12)C(=O)c1cc(F)c(-c2cccc(Cl)c2)c(OC2CC2)c1 ; CN(Cc1cnc(CF)c2cn[nH]c12)C(=O)c1ccc(-c2cccc(Cl)c2)c(OC2CCC2)c1 ; CCOc1cc(C(=O)N(C)Cc2cnc(C(C)O)c3cn[nH]c23)cc(F)c1-c1cccc(C#N)c1 ; CC(O)c1ncc(CN(C)C(=O)c2cc(F)c(-c3cccc(C#N)c3)c(OC3CC3)c2)c2[nH]ncc12] 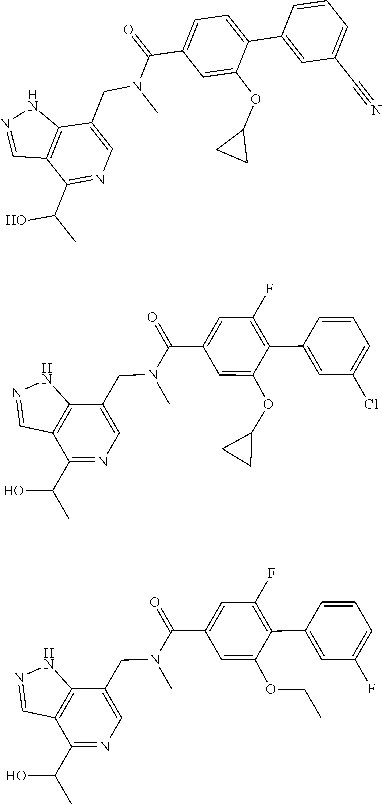 [SMILES: CC(O)c1ncc(CN(C)C(=O)c2ccc(-c3cccc(C#N)c3)c(OC3CC3)c2)c2[nH]ncc12 ; CC(O)c1ncc(CN(C)C(=O)c2cc(F)c(-c3cccc(Cl)c3)c(OC3CC3)c2)c2[nH]ncc12 ; CCOc1cc(C(=O)N(C)Cc2cnc(C(C)O)c3cn[nH]c23)cc(F)c1-c1cccc(F)c1]
or a pharmaceutically acceptable salt thereof.

16. The method of claim 13, wherein the compound of Formula I is selected from 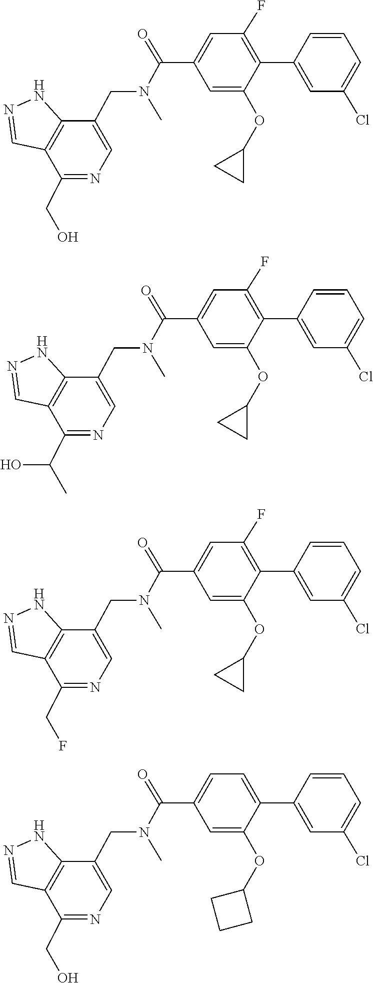 [SMILES: CN(Cc1cnc(CO)c2cn[nH]c12)C(=O)c1cc(F)c(-c2cccc(Cl)c2)c(OC2CC2)c1 ; CC(O)c1ncc(CN(C)C(=O)c2cc(F)c(-c3cccc(Cl)c3)c(OC3CC3)c2)c2[nH]ncc12 ; CN(Cc1cnc(CF)c2cn[nH]c12)C(=O)c1cc(F)c(-c2cccc(Cl)c2)c(OC2CC2)c1 ; CN(Cc1cnc(CO)c2cn[nH]c12)C(=O)c1ccc(-c2cccc(Cl)c2)c(OC2CCC2)c1]
or a pharmaceutically acceptable salt thereof.
